(19) United States
(12) Patent Application Publication
(10) Pub. No.: US 2025/0032542 A1
(43) Pub. Date: Jan. 30, 2025

(54) SYSTEMS AND METHODS FOR TRANS-MODULATION OF IMMUNE CELLS BY GENETIC MANIPULATION OF IMMUNE REGULATORY GENES

(71) Applicant: Targetgene Biotechnologies Ltd., DN Hefer (IL)

(72) Inventors: Dan Michael WEINTHAL, Rehovot (IL); Yoel Moshe SHIBOLETH, DN Hefer (IL); David STEINER, Elkana (IL); Talya KUNIK, Ramat Gan (IL)

(21) Appl. No.: 18/709,233

(22) PCT Filed: Nov. 10, 2022
(86) PCT No.: PCT/IL2022/051204

§ 371 Date: May 10, 2024

Related U.S. Application Data
(60) Provisional application No. 63/278,753, filed on Nov. 12, 2021.

(51) Int. Cl. A61K 35/17; A61K 39/00; A61P 35/00; C12N 5/0783; C12N 9/22; C12N 15/113; C12N 15/90

(52) U.S. Cl. CPC A61K 35/17; A61K 39/4611; A61K 39/4631; A61P 35/00; C12N 5/0638; C12N 5/0646; C12N 9/22; C12N 15/1136; C12N 15/907; C12N 2310/141; C12N 2310/20; C12N 2510/00

(57) ABSTRACT

The present disclosure relates to powerful trans immune-modulatory systems, compositions and methods based on targeted genetic modification of immune-regulatory genes, for modulating immune cells and treating immune-related disorders. The disclosed systems comprise at least one donor nucleic acid molecule that encodes a replacement sequence that specifically integrates in a target sequence within a first immunoregulatory gene. The disclosed system further comprises at least one target recognition element, that targets the donor molecule to the target first immune-modulatory gene. The integration in the first target gene results in the production of at least one immune-modulatory product and/or modulation of the expression and/or activity of the immune-regulatory gene and any product thereof.

### FIELD OF THE INVENTION

The present disclosure relates to immune modulation. More specifically, the present disclosure provides systems and methods for genetic modification of immune-regulatory genes, specifically, checkpoint inhibitors for modulating immune cells and treating immune-related disorders.

### BACKGROUND ART

References considered to be relevant as background to the presently disclosed subject matter are listed below:
[1] Samstein, R. M. et al. Tumor mutational load predicts survival after immunotherapy across multiple cancer types. Nat. Genet. 51, 202-206 (2019).
[2] Topalian, S. L. et al. Safety, activity, and immune correlates of anti-PD-1 antibody in cancer. N. Engl. J. Med. 366, 2443-2454 (2012).
[3] Gao, J. et al. Loss of IFN-gamma pathway genes in tumor cells as a mechanism of resistance to anti-CTLA-4 therapy. Cell 167, 397-404 (2016).
[4] Sade-Feldman, M. et al. Resistance to checkpoint blockade therapy through inactivation of antigen presentation. Nat. Commun. 8, 1136 (2017).
[5] Salvermoser M, et al. Journal for ImmunoTherapy of Cancer (2020).
[6] Luo et al. Clin Cancer Res Oct. 15 2020 (26) (20) 5494-5505.
[7] Fromm et al, 2018, Journal for ImmunoTherapy of Cancer, 6:149.
[8] Kang, L et a. Exp Hematol Oncol 9, 11 (2020)

Acknowledgement of the above references herein is not to be inferred as meaning that these are in any way relevant to the patentability of the presently disclosed subject matter.

### BACKGROUND OF THE INVENTION

The tumor microenvironment (TME) is infiltrated with many types of innate and adaptive immune cells whose immune surveillance functions are often suppressed by multiple mechanisms in a context-dependent manner (Thorsson, V. et al. Immunity 48, 812-830 (2018)). The signaling suppression is reflected by the ways that tumor cells downregulate the activity of stimulatory immunoreceptors while upregulating the activity of inhibitory immunoreceptors. Using T cells as an example for such immune cells, tumor cells can tune down T cell receptor (TCR)-mediated stimulatory signaling by downregulating surface MHC-I level (McGranahan, N. et al. Cell 171, 1259-1271 (2017)). Conversely, tumor cells can tune up PD-1-mediated inhibitory signaling by upregulating surface PD-L1 level (Iwai, Y. et al. Proc. Natl. Acad. Sci. USA 99, 12293-12297 (2002)). The concept that blocking the activation of inhibitory immunoreceptors can reinvigorate antitumor function of immune cells has been demonstrated experimentally and translated to treatment of many types of cancer in the clinic (Ribas, A. & Wolchok, J. D. Science 359, 1350-1355 (2018)).

A number of inhibitory immunoreceptors have been identified and studied in cancer, including but not limited to PD-1, CTLA-4, LAG3, TIM3, TIGIT and BTLA. These are termed “immune checkpoints” referring to proteins that act as gatekeepers of immune responses. These receptors often use mono-tyrosine signaling motifs, such as immunoreceptor tyrosine-based inhibitory motif (ITIM) and immunoreceptor tyrosine-based switch motif (ITSM), to deliver inhibitory signals. As cell-surface proteins, their activity can be inhibited by binding agents such as blocking antibodies that prevent ligand-receptor engagement (He X, Xu C. Immune checkpoint signaling and cancer immunotherapy. Cell Res. 2020; 30(8):660-669. doi:10.1038/s41422-020-0343-4). Immune checkpoint blockade therapy often leads to more durable response than chemotherapy or targeted therapies, perhaps reflecting the memory feature of the immune system. However, as clinical data accumulates worldwide, drawbacks and side effects have begun to emerge. The major bottleneck of immune checkpoint blockade therapy is its low response rate in most cancers, with a range of 10%-30% (Ribas, A. & Wolchok, J. D. Cancer immunotherapy using checkpoint blockade. Science 359, 1350-1355 (2018)). Mechanisms of non-responsiveness have been extensively studied, and many factors have been found to be relevant, such as tumor mutational burden, PD-L1 expression level, IFN signaling and MHC-I loss [1-4]. However, biomarkers that faithfully predict efficacy are still lacking.

It has already become clear that, upon ligand engagement, different checkpoints show distinct signaling mechanisms to suppress antitumor immunity. These checkpoints also differ in regulation of their expression and their cognate ligand. Inhibitory functions of immune checkpoints are tightly regulated by surface expression level, receptor-ligand interactions, and intracellular signal transduction.

There is a need for modulating immune checkpoints in order to convert the TME from suppressing inflammation to an anti-tumor inflammatory environment.

### SUMMARY OF THE INVENTION

In a first aspect, the present disclosure provides an immune trans-regulatory/modulatory system comprising the following components:
(a) at least one promoter-less donor nucleic acid molecule comprising at least one replacement nucleic acid sequence. The replacement nucleic acid sequence and/or the donor nucleic acid molecule comprising the replacement sequence is inserted and/or introduced into at least one target first sequence within a target immune-regulatory gene/s of interest. In some embodiments, the replacement nucleic acid sequence replaces at least part of the endogenous coding region of the at least one target first immune-regulatory gene. In some alternative or additional embodiments, the replacement sequence is introduced into the endogenous non-coding region of the target immune-regulatory gene, such that the target gene is intact. The replacement, and/or introduction of the replacement sequence provided by the donor nucleic acid molecule, results in at least one of: (i) the production of at least one immune-modulatory product, controlled by at least one endogenous control element of the at least one first target immune-regulatory gene; and (ii) inhibition and/or reduction and/or modulation of the expression and/or activity, and/or stability of the at least one target first immune-regulatory gene; and
(b) at least one target recognition element targeted at a target sequence within the at least one first target immune-regulatory gene, or any nucleic acid sequence encoding the target recognition element.

In some embodiments, where nucleic acid sequence/s that encode the target recognition element are used, these sequences may be provided either as part of the same nucleic acid donor molecule, or in at least one separate nucleic acid molecule.

A further aspect of the present disclosure relates to at least one cell comprising and/or modified by at least one immune trans-regulatory/modulatory system or a population of cells comprising the at least one cell. In some embodiments, the system of the disclosed cell comprises the following components: (a) at least one promoter-less donor nucleic acid molecule comprising at least one replacement nucleic acid sequence. The replacement nucleic acid sequence and/or the donor nucleic acid molecule comprising the replacement sequence is inserted and/or introduced into a target sequence within at least one target first immune-regulatory gene/s of interest. In some embodiments, the replacement nucleic acid sequence replaces at least part of the endogenous coding region of the target immune-regulatory gene. In some alternative or additional embodiments, the replacement sequence is introduced into the endogenous non-coding region of the at least one target first immune-regulatory gene, such that the target gene is intact. The replacement, and/or introduction of the replacement sequence provided by the donor nucleic acid molecule, results in at least one of: (i) the production of at least one immune-modulatory product, controlled by at least one endogenous control element of the target immune-regulatory gene; and (ii) inhibition and/or reduction and/or modulation of the expression and/or activity, and/or stability of the target immune-regulatory gene; and (b) at least one target recognition element targeted at a target sequence within the at least one target first immune-regulatory gene, or any nucleic acid sequence encoding the target recognition element.

In some embodiments, where nucleic acid sequences that encode the target recognition element are used, these sequences may be provided either as part of the same nucleic acid donor molecule, or in at least one separate nucleic acid molecule.

A further aspect of the present disclosure relates to a composition comprising at least one of: In one option (I), the disclosed composition may comprise an immune trans-regulatory/modulatory system comprising: (a) at least one promoter-less donor nucleic acid molecule comprising at least one replacement nucleic acid sequence. The replacement nucleic acid sequence and/or the donor nucleic acid molecule comprising the replacement sequence is inserted and/or introduced into a target sequence within at least one target first immune-regulatory gene of interest. In some embodiments, the replacement nucleic acid sequence replaces at least part of the endogenous coding region of the at least one target first immune-regulatory gene. In some alternative or additional embodiments, the replacement sequence is introduced into the endogenous non-coding region of the at least one target first immune-regulatory gene, such that the target gene is intact. The replacement, and/or introduction of the replacement sequence provided by the donor nucleic acid molecule, results in at least one of: (i) the production of at least one immune-modulatory product, controlled by at least one endogenous control element of the target immune-regulatory gene; and (ii) inhibition and/or reduction and/or modulation of the expression and/or activity, and/or stability of the at least one target first immune-regulatory gene; and (b) at least one target recognition element targeted at a target sequence within the at least one target first immune-regulatory gene, or any nucleic acid sequence encoding the target recognition element. In some embodiments, where nucleic acid sequences that encode the target recognition element are used, these sequences may be provided either as part of the same nucleic acid donor molecule, or in at least one separate nucleic acid molecule. In some optional embodiments, the system of the disclosed composition may further comprise (c) at least one nucleic acid guided genome modifier protein, chimeric protein, complex or conjugate, or at least one nucleic acid sequence encoding the guided genome modifier protein. In some embodiments, when the nucleic acid sequence that encodes the nucleic acid guided genome modifier protein is provided in the disclosed system, and when nucleic acid sequences that encode the target recognition element are used, the nucleic acid sequence encoding the at least one nucleic acid guided genome modifier protein may be provided either in a separate nucleic acid molecule, or in the same molecule together with the nucleic acid sequence that encodes the target recognition element, and/or as part of the at least one donor nucleic acid molecule.

In some alternative or additional embodiments, the composition of the present disclosure may comprise (II), at least one nucleic acid cassette or any vector or vehicle comprising at least one of: (i) the nucleic acid sequence of (a); (i) the nucleic acid sequence of (b); (iii) the nucleic acid sequence of (c); and (iv) the nucleic acid sequence of (a) and at least one of (b) and (c). Still further, in some alternative or additional embodiments, the disclosed compositions may comprise (III), at least one cell comprising and/or modified by at least one of: the nucleic acid the at least one system of (I), or the at least one nucleic acid cassette or any vector or vehicle of (II); or any matrix, nano- or micro-particle comprising at least one of (I) and (II), and/or a population of cells comprising at least one of said cell. It should be noted that in some optional embodiments, the composition of the present disclosure may further comprise at least one of pharmaceutically acceptable carrier/s, diluent/s, excipient/s and additive/s.

A further aspect of the present disclosure relates to a method of modulating at least one target cell. The disclosed method comprises the steps of contacting the target cell with at least one of:

In one option (I), the disclosed methods may comprise the step of contacting with the target cell an immune trans-regulatory and/or modulatory system comprising: (a) at least one promoter-less donor nucleic acid molecule comprising at least one replacement nucleic acid sequence. The replacement nucleic acid sequence and/or the donor nucleic acid molecule comprising the replacement sequence is inserted and/or introduced into a target sequence within at least one target first immune-regulatory gene/s of interest. In some embodiments, the replacement nucleic acid sequence replaces at least part of the endogenous coding region of the at least one target first immune-regulatory gene/s. In some alternative or additional embodiments, the replacement sequence is introduced into the endogenous non-coding region of the at least one target first immune-regulatory gene/s, such that the target gene is intact. The replacement, and/or introduction of the replacement sequence provided by the donor nucleic acid molecule, results in at least one of: (i) the production of at least one immune-modulatory product, controlled by at least one endogenous control element of the target immune-regulatory gene; and (ii) inhibition and/or reduction an/or modulation of the expression, and/or activity, and/or the stability of the at least one target first immune-regulatory gene; and (b) at least one target recognition element targeted at a target sequence within the at least one target first immune-regulatory gene, or any nucleic acid sequence encoding the target recognition element. In some embodiments, where nucleic acid sequences that encode the target recognition element are used, these sequences may be provided either as part of the same nucleic acid donor molecule, or in at least one separate nucleic acid molecule. In some optional embodiments, the system that may be used by the disclosed methods may further comprise (c) at least one nucleic acid guided genome modifier protein, chimeric protein, complex or conjugate, or at least one nucleic acid sequence encoding said guided genome modifier protein. In some embodiments, when the nucleic acid sequence encoding the nucleic acid guided genome modifier protein is provided in the disclosed system, and when nucleic acid sequences that encode the target recognition element are used, the nucleic acid sequence encoding the at least one nucleic acid guided genome modifier protein may be provided either in a separate nucleic acid molecule, or in the same molecule together with the nucleic acid sequence that encodes the target recognition element, and/or as part of the at least one donor nucleic acid molecule.

In some alternative or additional embodiments, the disclosed methods may comprise the step of contacting with the target cell of the present disclosure with (II), at least one nucleic acid cassette or any vector or vehicle comprising at least one of: (i) the nucleic acid sequence of (a); (i) the nucleic acid sequence of (b); (iii) the nucleic acid sequence of (c); and (iv) the nucleic acid sequence of (a) and at least one of (b) and (c). Still further, in some alternative or additional embodiments, disclosed methods may comprise the step of contacting with the target cell (III), at least one cell comprising and/or modified by at least one of: the nucleic acid the at least one system of (I), or the at least one nucleic acid cassette or any vector or vehicle of (II); or any matrix, nano- or micro-particle comprising at least one of (I) and (II), and/or a population of cells comprising at least one of the cell/s. In yet some further alternative or additional embodiments, the disclosed methods may comprise the step of contacting with the target cell (IV), at least one composition comprising at least one of (I), (II), (III) or any combinations thereof.

A further aspect of the present disclosure relates to a method of curing or treating, preventing, inhibiting, reducing, eliminating, protecting or delaying the onset of a pathologic disorder in a mammalian subject. More specifically, the method of the invention comprises the step of administering to the subject an effective amount of at least one of: In one option (I), the disclosed therapeutic methods may comprise the step of administering to the subject an effective amount of an immune trans-regulatory/modulatory system comprising: (a) at least one promoter-less donor nucleic acid molecule comprising at least one replacement nucleic acid sequence. The replacement nucleic acid sequence and/or the donor nucleic acid molecule comprising the replacement sequence is inserted and/or introduced into a target sequence within at least one target first immune-regulatory gene of interest. In some embodiments, the replacement nucleic acid sequence replaces at least part of the endogenous coding region of the target immune-regulatory gene. In some alternative or additional embodiments, the replacement sequence is introduced into the endogenous non-coding region of the at least one target first immune-regulatory gene, such that the target gene is intact. The replacement, and/or introduction of the replacement sequence provided by the donor nucleic acid molecule, results in at least one of: (i) the production of at least one immune-modulatory product, controlled by at least one endogenous control element of the target immune-regulatory gene; and (ii) inhibition and/or reduction and/or modulation of the expression and/or activity, and/or the stability of the immune-regulatory gene; and (b) at least one target recognition element targeted at a target sequence within the at least one target first immune-regulatory gene, or any nucleic acid sequence encoding the target recognition element. In some embodiments, where nucleic acid sequences that encode the target recognition element are used, these sequences may be provided either as part of the same nucleic acid donor molecule, or in at least one separate nucleic acid molecule. In some optional embodiments, the system that may be used by the disclosed methods may further comprise (c) at least one nucleic acid guided genome modifier protein, chimeric protein, complex or conjugate, or at least one nucleic acid sequence encoding the guided genome modifier protein. In some embodiments, when the nucleic acid sequence encoding the nucleic acid guided genome modifier protein is provided in the disclosed system, and when nucleic acid sequences that encode the target recognition element are used, the nucleic acid sequence encoding the at least one nucleic acid guided genome modifier protein may be provided either in a separate nucleic acid molecule, or in the same molecule together with the nucleic acid sequence that encodes the target recognition element, and/or as part of the at least one donor nucleic acid molecule.

In some alternative or additional embodiments, the disclosed therapeutic methods may comprise the step of administering to the subject an effective amount of (II), at least one nucleic acid cassette or any vector or vehicle comprising at least one of: (i) the nucleic acid sequence of (a); (i) the nucleic acid sequence of (b); (iii) the nucleic acid sequence of (c); and (iv) the nucleic acid sequence of (a) and at least one of (b) and (c). Still further, in some alternative or additional embodiments, the disclosed therapeutic methods may comprise the step of administering to the subject an effective amount of (III), at least one cell comprising and/or modified by at least one of: the nucleic acid the at least one system of (I), or the at least one nucleic acid cassette or any vector or vehicle of (II); or any matrix, nano- or micro-particle comprising at least one of (I) and (II), and/or a population of cells comprising at least one of the cell/s. In yet some further alternative or additional embodiments, the disclosed therapeutic methods may comprise the step of administering to the subject an effective amount of (IV), at least one composition comprising at least one of (I), (II), (III) or any combinations thereof.

A further aspect of the present disclosure relates to an effective amount of at least one of: (I), an effective amount of an immune trans-regulatory and/or modulatory system comprising: (a) at least one promoter-less donor nucleic acid molecule comprising at least one replacement nucleic acid sequence. The replacement nucleic acid sequence and/or the donor nucleic acid molecule comprising the replacement sequence is inserted and/or introduced into a target sequence within at least one target first immune-regulatory gene of interest. In some embodiments, the replacement nucleic acid sequence replaces at least part of the endogenous coding region of the target immune-regulatory gene. In some alternative or additional embodiments, the replacement sequence is introduced into the endogenous non-coding region of the at least one target first immune-regulatory gene, such that the target gene is intact. The replacement, and/or introduction of the replacement sequence provided by the donor nucleic acid molecule, results in at least one of: (i) the production of at least one immune-modulatory product, controlled by at least one endogenous control element of the at least one target first immune-regulatory gene; and (ii) inhibition and/or reduction and/or modulation of the expression and/or activity, and/or the stability of the at least one target first immune-regulatory gene, The systems used herein further comprise (b), at least one target recognition element targeted at a target sequence within the at least one target first immune-regulatory gene/s, or any nucleic acid sequence encoding the target recognition element. In some embodiments, where nucleic acid sequences that encode the target recognition element are used, these sequences may be provided either as part of the same nucleic acid donor molecule, or in at least one separate nucleic acid molecule. In some optional embodiments, the system that may be used by the disclosed methods may further comprise (c) at least one nucleic acid guided genome modifier protein, chimeric protein, complex or conjugate, or at least one nucleic acid sequence encoding said guided genome modifier protein. In some embodiments, when the nucleic acid sequence encoding the nucleic acid guided genome modifier protein is provided in the disclosed system, and when nucleic acid sequences that encode the target recognition element are used, the nucleic acid sequence encoding the at least one nucleic acid guided genome modifier protein may be provided either in a separate nucleic acid molecule, or in the same molecule together with the nucleic acid sequence that encodes the target recognition element, and/or as part of the at least one donor nucleic acid molecule.

In some alternative or additional embodiments, the disclosure provides an effective amount of (II), at least one nucleic acid cassette or any vector or vehicle comprising at least one of: (i) the nucleic acid sequence of (a); (i) the nucleic acid sequence of (b); (iii) the nucleic acid sequence of (c); and (iv) the nucleic acid sequence of (a) and at least one of (b) and (c). Still further, in some alternative or additional embodiments, the disclosure provides an effective amount of (III), at least one cell comprising and/or modified by at least one of: the nucleic acid the at least one system of (I), or the at least one nucleic acid cassette or any vector or vehicle of (II); or any matrix, nano- or micro-particle comprising at least one of (I) and (II), and/or a population of cells comprising at least one of the cell/s.

In yet some further alternative or additional embodiments, the disclosure provides an effective amount of (IV), at least one composition comprising at least one of (I), (II), (III) or any combinations thereof, or any combinations thereof; for use in a method of curing or treating, preventing, inhibiting, reducing, eliminating, protecting or delaying the onset of a pathologic disorder in a mammalian subject.

These and other aspects of the invention will become apparent by the hand of the following figures.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1: Schematic representation of an HDR integration cassette

An HDR integration cassette that contains an in-frame 41BB transmembrane domain and intracellular domain (ICD) replaces the corresponding region from PD-1. IL7 (SEQ ID: 1) and CCL21 (SEQ ID:2) are also expressed, separated by 2A self-cleaving peptides (T2A, SED ID:3; P2A, SEQ ID:4), with transcription terminated by BGHT (SEQ ID:5).

FIG. 2. Schematic representation of an NHEJ integration cassette

An NHEJ integration cassette containing an IRES (internal ribosome entry site) allowing translation from the same mRNA as the disrupted PD1 gene. The integration cassette contains extracellular PD-1 fused to a 41BB transmembrane domain and intracellular domain (TCD). CCL21 (SEQ ID:2) is also expressed, separated by 2A self-cleaving peptides (T2A, SED ID NO:3; P2A, SEQ ID NO:4), with transcription terminated by BGHT (SEQ ID:5). An additional terminator (SV40 terminator, SEQ ID NO:101) was added in the reverse direction to prevent transcription of anti-sense RNA if the construct is inserted in the reverse direction. The cassette is flanked on either side by 6-frame stop codons. The DNA sequence for the full construct (integration cassette) is as denoted by SEQ ID NO:6.

FIG. 3. PD1 exon 3 NHEJ editing

The figure shows ddPCR analysis of gDNA extracted from Hek293 cells 72 Hrs after transfection with a plasmid encoding the nuclease (TG-14663 or spCas9) and a second plasmid encoding the RNA guide or guide pairs.

FIG. 4A-4C: HDR-enhanced gene replacement comprising donor attachment domain (DAD) and repair factor recruitment domain (RFRD)

FIG. 4A-I-4A-II. The figure shows schematic presentation of the PD1 gene, the cut site and five dsDNA donor cassettes comprising an insert of 1025 bp encoding GFP in frame with the PD1 start codon. FIG. 4A-I shows HDR dsDNA donor cassettes, and FIG. 4A-II shows NHEJ dsDNA donor cassettes.

FIG. 4B. illustrates the PCR results comparing NHEJ vs HDR insertion in a human cell line.

FIG. 4C. illustrates the PCR results comparing HDR with nucleases comprising DAD and RFRD domains in primary human T-cells.

FIG. 5. Autoregulated CAR T genetic manipulation scheme

“Driver” gene (hatched) permanently replaced with miRNA precursor under the Driver promoter. The miRNA precursor is processed to artificial miRNAs (amiRNAs) conditionally knocking down mRNA levels of exhaustion causing “Target” genes (light and dark dotted bars).

### DETAILED DESCRIPTION OF THE INVENTION

The present disclosure provides immune-trans-regulatory systems, compositions, as well as methods and uses thereof, in immune-modulation of target immune-cells. More specifically, the immune-trans-regulatory systems as disclosed by the present disclosure are based on knocking out and/or modulating a target “first” immuno-regulatory gene, while using the endogenous regulatory elements of this at least one target first immuno-regulatory gene, as a “driver” to control transcription of any knocked-in at least one modulatory sequence/s that act either as at least one second immuno regulatory gene/s, and/or as modulator/s that trans-modulate the expression and/or activity and/or stability of at least one other second immuno-regulatory gene/s. This trans regulatory system allows a specific, temporally and spatially controlled expression and/or activity, and/or repression of at least one immune-regulatory gene (also referred to herein as a “second” immune-regulatory gene”), at the specific stage, cell, tissue, or organ, and for the appropriate time frame. Thus, genetically edited target first immuno modulatory gene enables the regulation of itself (e.g., KO, truncation, creation of chimeras etc.), and further provides in some embodiments, the modulation of other genes or a second allele of the same gene (e.g., by knock-down [KD]) “in trans”. Still further, in one configuration of the present disclosure, immuno modulatory targets, for example, immune checkpoints are modulated by using genome editing tools to make the cells express truncated Immune Checkpoint proteins. The goal is to express the genes without the transmembrane and/or the native intracellular signaling domains. The expression of the proteins remains regulated by the native promoters and cellular signaling of the target first immuno regulatory gene, that is therefore referred to herein as a “Driver”. The resulting expression is secreted proteins that are not attached to the cell membrane and can still attach to their cognate suppressor ligand. As a result, like anti Immune Checkpoints antibodies that block the receptor-ligand engagement, the secreted extracellular domains of the modulated Immune Checkpoints would mask and/or compete with their ligands and thus protect from the inhibitory signals of other unmanipulated immune cell migrating to the same sites. In many cases, increased expression of Immune Checkpoint receptors is a result of prolonged extensive stimulation. Therefore, systemic secretion will be minimized, and expression is expected to be spatially and temporally controlled, at the desired sites and at the right time. Moreover, the penetration of the modified Immune Checkpoint inhibitor would be even more effective compared to equivalent antibodies, since the latter, unlike the NK and T-cells have poor penetration capabilities into solid tissues and tumors. On the other hand, immune cells have proven capabilities to infiltrate into the relevant sites, for example, tumor derived Tumor Infiltrating Lymphocytes (TILs), which are the patient's own immune cells, are an established anti-tumor therapy, in a more regulated, safe, and effective manner.

Therefore, these secreted proteins would be more effective in protecting other immune cells from inhibition signaling. It may also give wider protection in comparison to a single anti-Immune Checkpoint Antibody. For example, PD1 can engage with both PD-L1 and PD-L2. On the other hand, PD-L1 can interact in order to transmit inhibition signals with both PD1 and B7.1. Therefore, providing anti PD1 antibody would not block the PD-L1/B7.1 interaction, and conversely providing an anti-PD-L1 antibody will not block the PDL2/PD1 interaction. Thus, secretion of a soluble, PD1 intracellular domain, may have the advantage of blocking all these interactions.

As a further enhanced option, in addition to truncating immune checkpoints upstream to their transmembrane domain, specific integration of donor DNA that comprises the at least one “replacement nucleic acid sequence” that may be in some embodiments also at least one “additional sequence”, by homologous recombination (HDR) into the target site within the at least one target first immune-regulatory gene, may be added to this manipulation. The donor DNA comprising the at least one replacement and/or additional sequence, is fused in-frame to the secreted fragment. The final result is a chimeric gene that has modified and/or added characteristics to the secreted fragment. Additional separate genes may also be included in the same donor DNA cassette. The goal of the checkpoint-fusion DNA is to modify the truncated protein or to modify the cells in order to create a more effective anti-tumor agent, as well as to alter the TME to become a more inflammatory environment that can support wider anti-tumor effects. These modifications can be, for example, in form of a secreted soluble agent such as PD1-FC, which both blocks suppressor ligands via the PD1 domain, and additionally activates immune cells such as NK, via the FC tail. Alternatively, it can be in a form of a membrane anchored receptor that switches the inhibitory signal of PD1 into a stimulatory signal, by fusing transmembrane and intracellular domains from stimulatory receptors such as 41BB or CD28. In addition, the HDR cassette may contain combinations of cytokines that recruit and support immune cells. Non-limiting embodiments for such cytokines that act as second immune-regulatory gene/s and encoded protein/s include IL1, IL7, IL15, CC119, and CC121. Those cytokines would be regulated to be expressed and secreted, by the native promoter of the immune checkpoint genes (acting in some embodiments as the target first immune-regulatory genes, and also referred to herein as “driver”), i.e., within the TME upon activation.

In a further embodiment of the present disclosure, the inventors modulate immune cells by using genome editing tools to express non-coding RNAs such as silencing RNAs directed at genes such as immune checkpoint genes, or any other immune-regulatory gene/s (also referred to herein as “second” immune-regulatory gene/s). These silencing RNAs may be processed from double strand RNA to create classical shRNA or from miRNA-like precursors to create miRNA-like short RNAs. These constructs may be integrated into the genome to be transcribed from endogenous promoters (e.g., the endogenous promoters of PD-1, LIF, or TIGIT), or exogenous polII or polIII promoters.

The advantage of use of endogenous promoters for expression of foreign DNA, including short RNA cassettes, are numerous: a) control of expression, whether induction or suppression, at relevant, natural, spatial and temporal instances within the cell or tissue or tumor microenvironment; b) delivery of promoterless DNA donor constructs adds an important safety measure in case of unintended mis-insertion. Exogenously delivered promoters may unintentionally turn on genes in which they inadvertently land or mistakenly silence them by transcription of an antisense RNA; c) allows knockout of the gene (e.g., the at least one target first immune-regulatory gene) that the construct is set to insert into (or replace). This also simplifies selection of properly edited cells; d) promoterless constructs are shorter and thus are expected to be easier to produce, transfect and are expected to have higher insertion efficiencies.

As indicated above, the replacement sequence may be in some embodiments a sequence encoding regulatory elements (e.g., inhibitory or modulatory RNA molecules). Use of shRNA or miRNA-like short RNAs can be selected according to desired insertion strategy: shRNAs created from perfectly complementary RNA strands can be produced, for example, by positioning two PolIII promoters such as U6 and H1 in opposing directions and flanking a short DNA sequence i.e., of 18 or 19 bases or longer. Multiple shRNAs can be expressed from concatenated dual-promotor cassettes (Deng F, Chen X, Liao Z, Yan Z, Wang Z, Deng Y, et al. (2014) A Simplified and Versatile System for the Simultaneous Expression of Multiple siRNAs in Mammalian Cells Using Gibson DNA Assembly. PLoS ONE 9(11): e113064. https://doi.org/10.1371/journal.pone.0113064). The advantages are a) that expression levels of polIII promoters result in 15-30 fold higher expression than the strong CMV polII promoter (Liu et al NAR 36:2811-2824 2008); b) the construct is not directional and thus can be inserted in either direction into a NHEJ mediated cut site. This results in shorter Donor DNA without the homology arms necessary for HDR. The disadvantages however are lack of spatial or temporal control of transcription due to the exogenous PolIII promoters and that shRNA are ˜12 times less active than miRNA-like short RNAs (Liu et al NAR 36:2811-2824 2008). miRNA-like short RNAs have several advantages a) they can be expressed under polII promoters which as stated above can be controlled by endogenous or exogenous cues; b) multiple miRNA-like precursors can be placed in an artificial polycistron on a single polII transcribed RNA allowing multiple miRNA-like shRNAs to be processed from the same transcript in equimolar proportions (Liu et al NAR 36:2811-2824 2008); c) While it is probably necessary to insert such a precursor directionally (i.e via HDR) there is no coding frame requirement allowing greater choice in insertion site location.

The advantages of a combined insertion and silencing approach, as opposed to silencing only or permanent gene knockout include the (a) ability to regulate when a gene is turned on or off following biologically and clinically relevant cellular cues; (b) an enduring approach as opposed to transient silencing (c) multiple gene-knockdown with a single dsDNA genomic cleavage as opposed to multiple dsDNA breaks in multiple gene-knockout; d) RNA-silencing, as opposed to single allele knockout, silences both alleles.

Thus, in some embodiments, a replacement nucleic acid sequence inserted and/or integrated into the target sequence within the at least one target first immunoregulatory gene (e.g., a “driver” such as PD1, LIF, TIGIT), may be any sequence encoding at least one inhibitory or modulatory nucleic acids, e.g., miRNAs, for example, any of those disclosed by Example 10 and Table 3. The target for such inhibitory nucleic acid molecules may be any gene involved either directly or indirectly in an immuno modulatory process that should be either inhibited or enhanced. List of more genes for which amiRs and/or siRNA constructs may be designed to modulate the human immune system include according to some embodiments: SIGLEC7; VISTA (V-domain immunoglobulin suppressor of T cell activation) (V-set immunoregulatory receptor (VSIR)); PVR/CD155; CD3 epsilon; CD3 zeta; CD160; CD244; A2AR Adenosine A2A receptor; TCR-beta constant region; Leukemia inhibitory factor (LIF); glycogen synthase kinase (GSK)-3/p (GSK3); Vaccinia related kinase 2 (VRK2); BARKI; HIPK2; CDK7; RPS6 KB; CK2A1; CDK3; Src homology region 2 domain-containing phosphatase-1 (SHP-1) (PTPN6); PTPN11 (SH2 domain-containing protein tyrosine phosphatase-2 (SHP-2)); Hematopoietic progenitor kinase 1 (HPK1 or MAP4K1); Monoamine oxidase A (MAO-A); Diacylglycerol kinase a (DGK-Alpha); CD73; Natural killer group protein 2A (NKG2A); Poliovirus receptor-related immunoglobulin domain containing (PVRIG/CD112R); C—C motif chemokine receptor 2 (CCR2);

In some embodiments, knockdown of the following target genes is useful for creation of allogeneic CAR-T: TCR-alpha constant region; TCR-beta constant region; B2M; PVR/CD155; CD3 Gamma; CD3 Delta; CD3 Epsilon and CD3 Zeta.

In some embodiments, knockdown of IL-6 as a target, is useful for alleviation of cytokine release syndrome.

In some embodiments, knockdown of the following targets is useful for alleviation of immune cell exhaustion specifically, CD160; CD244; A2AR Adenosine A2A receptor; VISTA (V-domain immunoglobulin suppressor of T cell activation) (V-set immunoregulatory receptor (VSIR)); SIGLEC9 (Sialic acid-binding immunoglobulin-type lectin 9, also designated as CD329); SIGLEC7; Vaccinia related kinase 2 (VRK2); Src homology region 2 domain-containing phosphatase-1 (SHP-1) (PTPN6); PTPN11 (SH2 domain-containing protein tyrosine phosphatase-2 (SHP-2)); Hematopoietic progenitor kinase 1 (HPK1 or MAP4K1); Monoamine oxidase A (MAO-A); Diacylglycerol kinase a (DGK-Alpha); CD73; Natural killer group protein 2A (NKG2A); Leukemia inhibitory factor (LIF); C—C motif chemokine receptor 2 (CCR2); Poliovirus receptor-related immunoglobulin domain containing (PVRIG/CD112R); glycogen synthase kinase (GSK)-3a/P (GSK3); PD1; TIGIT; BTLA; CTLA4; Lymphocyte activation gene 3 (LAG-3) (CD223); T cell immunoglobulin and mucin domain 3 (TIM-3)(HAVCR2) and TGF-beta receptor. Still further, in some embodiments, T cells that are trans-regulated using the systems of the present disclosure may be either genetically edited cells (e.g., CAR-T cells) or non-edited cells. In case of CAR-T cells, the present disclosure provides the use of the disclosed systems for the production of genetically rewired CAR-T cells resistant to exhaustion in the TEM.

In yet some further embodiments, knockdown of CD3 Delta, CD3 Gamma and/or CD38; is useful to alleviate fratricide in CAR-T.

Thus, in a first aspect, the present disclosure provides an immune trans-regulatory/modulatory system comprising the following components:

One component (a), of the disclosed systems comprises at least one promoter-less donor nucleic acid molecule comprising at least one replacement nucleic acid sequence. The replacement nucleic acid sequence and/or the donor nucleic acid molecule comprising the replacement sequence is inserted and/or introduced into a target sequence within a target immune-regulatory gene of interest. In some embodiments, when introduced into the target site, the replacement nucleic acid sequence replaces at least part of the endogenous coding region of the target immune-regulatory gene. In some alternative or additional embodiments, the replacement sequence is introduced into the endogenous non-coding region of the target immune-regulatory gene, such that the target gene is intact. In yet some further embodiments, the replacement sequence is introduced into the target site within the coding or non-coding sequences of the at least one target first immune-regulatory gene, without replacing any endogenous sequences in the coding and/or non-coding sequences of the at least one target first immune-regulatory gene. The replacement, and/or introduction of the replacement sequence provided by the donor nucleic acid molecule/s, results in at least one of: (i) the production of at least one immune-modulatory product. In some embodiments, the production of the product encoded by the replacement sequence is controlled by at least one endogenous control element of the target first immune-regulatory gene/s; and (ii) inhibition and/or reduction and/or modulation of the expression and/or activity, and/or stability of the target first immune-regulatory gene (also referred to herein as “driver”).

Another component of the disclosed system (b), comprise at least one target recognition element targeted at the at least one target sequence within the at least one target first immune-regulatory gene, or any nucleic acid sequence encoding the target recognition element.

In some embodiments, where nucleic acid sequences that encode the target recognition element are used, these sequences may be provided either as part of the same nucleic acid donor molecule, or in at least one separate nucleic acid molecule.

As disclosed herein, the immune trans-regulatory systems of the present disclosure target sequences of immune-regulatory genes, that may be according to some embodiments either the first immune-regulatory genes that are also referred to herein as “driver”, or alternatively, the second immune-regulatory genes, that are either expressed under the regulatory elements (e.g., promoter, upon insertion thereof into the target site within the first immune-regulatory gene), or manipulated and/or inhibited by inhibitory and/or modulatory elements inserted into a target site within the first immune-regulatory gene.

Both, the first and second targets are in some embodiments also referred to herein as a target gene of interest. The terms “target nucleic acid sequence of interest”, “gene of interest”, “a target gene of interest”, “a target gene”, are used interchangeably, and refer in some embodiments to a nucleic acid sequence that may comprise or comprised within a gene or any fragment or derivative thereof that is comprised by the target cell (or host cell) of the invention and is intended to be replaced, repressed, enhanced or modified.

The target nucleic acid sequence or gene of interest may comprise coding or non-coding DNA regions, or any combination thereof.

In some embodiments, the gene of interest may comprise coding sequences, for example, exons or fragments thereof that encode any product, for example, a protein or an enzyme (or fragments thereof). In other embodiments, the target nucleic acid sequence of interest may comprise non-coding sequences, such as for example start codons, 5′ un-translated regions (5′ UTR), 3′ un-translated regions (3′ UTR), or other regulatory sequences. In particular, regulatory sequences that are capable of increasing or decreasing the expression of specific genes within an organism. By way of example, regulatory sequences may be selected from, but are not limited to, transcription factors, activators, repressors and promoters. In further embodiments, the target nucleic acid sequence or gene of interest may comprise a combination of coding and non-coding regions.

Still further, the term “target gene of interest” or “target nucleic acid sequence of interest” as used herein refers to a gene in a eukaryotic cell or any fragment thereof to be replaced or modified by the replacement sequence according to the invention.

The system of the present disclosure comprises at least one nucleic acid donor molecule that in some embodiments, targets the target gene of interest, and comprise at least one replacement nucleic acid sequence. “Donor nucleic acid” or “donor nucleic acid molecule” is defined herein as any nucleic acid supplied to an organism or receptacle to be inserted, incorporated or recombined wholly or partially into the target sequence either by DNA repair mechanisms, homologous recombination (HR), or by non-homologous end-joining (NHEJ). A Donor nucleic acid molecule, may be a nucleic acid sequence (either RNA or DNA or a modified nucleic acid or a combination thereof). Donor nucleic acid consisting of DNA or modified DNA may also be referred to as “donor DNA”.

It should be appreciated that in some embodiments, the donor nucleic acid molecules of the systems of the present disclosure may be provided in one or more nucleic acid cassette.

In some embodiments, specifically when the donor nucleic acid molecule is incorporated into the target nucleic acid sequence via homologous recombination (HR), the donor nucleic acid sequence may also comprise, or specifically flanked by at least one homology arm, that displays complementarity to a nucleic acid sequence flanking the target site for incorporation.

In some further embodiments, the at least one nucleic acid sequence for incorporation of the donor nucleic acid molecule of the system of the invention may be a replacement sequence for the target immune-regulatory gene.

In some embodiments, such “replacement sequence” may comprise at least one nucleic acid sequence encoding a product (e.g., protein and/or RNA) that modulates directly or indirectly the immune response of a subject or immune-mediating signaling of at least one immune-cell, thereby affecting other immune and/or non-immune cell/s. In some embodiments, such replacement sequence may comprise the native, non-mutated, or alternatively, modified version of at least one product, any fragments thereof. In some embodiments, the replacement sequence may be any nucleic acid sequence that should replace the target immune-regulatory gene or parts thereof (e.g., thereby creating a fusion thereof), or alternatively, be expressed together with the intact target immune-regulatory gene, in the target cell. In some embodiments, the systems of the invention further provide a tool that enables manipulation of genes or gene fragments that do not necessarily comprise any mutation. The replacement gene may be in some embodiment, a gene or fragment thereof that may comprise mutation or any manipulation that may improve and/or change the native nucleic acid sequence within the target cell, or even modulate the expression of a target nucleic acid sequence, e.g., at least one gene or any fragments thereof. In some embodiments, the length of such donor nucleic acid molecule, or specifically, replacement nucleic acid sequence may range between about 100,000 nucleotides or more, to about 10 nucleotides or less. More specifically, the length of the nucleic acid sequence of interest may be about 100,000 nucleotides in length, or less than 75,000 nucleotides in length or less than 50,000 nucleotides in length, or less than 40,000 nucleotides in length, or less than 30,000 nucleotides in length, or less than 20,000 nucleotides in length, or less than 15,000 nucleotides in length, or less than 10,000 nucleotides in length, or less than 5000 nucleotides in length, or less than 1000 nucleotides in length, or less than 900 nucleotides in length, or less than 800 nucleotides in length, or less than 700 nucleotides in length, or less than 600 nucleotides in length, or less than 500 nucleotides in length, or less than 450 nucleotides in length, or less than 400 nucleotides in length, or less than 300 nucleotides in length, or less than 200 nucleotides in length, or less than 100 nucleotides in length, or less than 50 nucleotides in length, or less than 40 nucleotides in length, or less than 30 nucleotides in length, or less than 20 nucleotides in length, or less than 10 nucleotides in length. In some embodiments, the replacement nucleic acid sequence may be in the length of 20,000 (20 Kb) nucleotides or more.

Still further, it should be understood that the present disclosure also encompasses replacement nucleic acid sequence that comprise sequences encoding or forming more than one product, for example, the polycistronic cassette disclosed by Example 11. Thus, in some embodiments, the replacement nucleic acid sequence of the disclosed systems may comprise or encode 1 to 1000 products, specifically, 1, 2, 3, 4, 5, 6, 7, 8, 9, 10, 15, 20, 25, 30, 35, 40, 45, 50, 55, 60, 65, 70, 75, 80, 85, 90, 95, 100, 20, 300, 400, 500, 600, 700, 800, 900, 1000, or more products.

In some embodiments, the system disclosed herein may further comprise (c), at least one nucleic acid guided genome modifier protein, chimeric protein, complex or conjugate, or at least one nucleic acid sequence encoding the guided genome modifier protein.

In some embodiments, in case where the nucleic acid sequence encoding the nucleic acid guided genome modifier protein is provided by the disclosed system, and further when nucleic acid sequences encoding the target recognition element are used, the nucleic acid sequence encoding the at least one nucleic acid guided genome modifier protein may be provided either in a separate nucleic acid molecule, or in the same molecule together with the nucleic acid sequence encoding the target recognition element, and/or as part of the at least one donor nucleic acid molecule of the disclosed system/s.

Thus, the disclosed systems may comprise in addition to the at least one donor molecule, at least one nucleic acid sequence encoding the target recognition element, and/or at least one nucleic acid sequence encoding the at least one modifier, and any combinations thereof, that can be provided in one, two or more separated nucleic acid molecules.

Designing and preparing synthetic target recognition element/s is relatively simple, rapid and relatively inexpensive. It is also possible, in some embodiments of this invention, to produce target recognition elements in-vivo, circumventing the necessity to deliver chemically synthesized target recognition elements to a cell. Furthermore, these elements can be designed to base pair to almost any desired target sequence, and thus, can direct the molecular complex to almost any target sequence. Moreover, several sequences may be targeted in the same cell concomitantly. For example, in editing functions which require more than one cleavage site, such as deletion or replacement of specific stretches of nucleic acid, by simply providing different target recognition elements and one effector moiety, (e.g., and Cas protein, Cas-FokI chimera, or any improved variants, e.g., Cas protein having abolished or reduced PAM-restriction, and/or Cas protein having enhanced HDR or any fusion protein thereof, in accordance with the invention). The systems of the invention may comprise the nucleic acid guided effector/modifier and the at least one target recognition element, either in a mature form, for example, as a protein moiety and as a target recognition element (e.g., gRNA, split-gRNA), or as a Ribonucleoprotein (RNP). However, the invention further encompasses the option of a system comprising nucleic acid sequences encoding each of these components, specifically, nucleic acid sequence encoding the nucleic acid guided effector/modifier of the invention, and nucleic acid sequence encoding the at least one target recognition element. Thus, in some embodiments, the systems of the invention may comprise at least one nucleic acid sequence encoding the modifier protein, for example, Cas protein or any variant, mutant, fusion/chimeric protein thereof (e.g., Cas9-FokI) and at least one nucleic acid sequence encoding at least one target recognition element, specifically, at least one gRNA. In some embodiments, the nucleic acid sequence encoding the Cas protein of the invention or any chimeric or fusion protein thereof, and the nucleic acid sequence encoding the target recognition element may be comprised in one or more nucleic acid cassette or any vector or vehicle.

It should be understood that in some embodiments, for modifying the target nucleic acid sequence within the target first immune-regulatory gene/s, by the nucleic acid guided genome modifier chimeric/fusion protein complex or conjugate of the systems of the invention, at least two target recognition elements may be used to guide the at least two nucleic acid guided genome modifier protein, chimeric protein, complex or conjugate thereof to a single target site within the target nucleic acid sequence. According to such embodiments, each of the respective target recognition elements (e.g., SCNAs or gRNAs discussed herein after), may be directed to a target sequence that is located at a distance of between about 5 to 50 nucleotides from the target sequence/s recognized by at least one other targeting recognition element/s. In some embodiments, the distance may be any one of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more. In some embodiments, the target recognition elements used in the systems of the invention may be designed for targeting target sequences that are located at a distance of about 10 to 30 nucleotides from each other, specifically, 10, 11, 12, 13, 14, 15, 16, 17, 18, 19, 20, 21, 22, 23, 24, 25, 26, 27, 28, 29, 30, nucleotides, more specifically, 15 nucleotides or more, or 27 nucleotides or more. In some embodiments, the target recognition elements used in the systems of the invention may be designed for targeting target sequences that are located at a distance of about 15 nucleotides.

In some embodiments, the target recognition element/s, disclosed herein, that are also referred to herein as the programming oligonucleotides or SCNAs, have an infinite repertoire of sequences, thus conceivably achieving extreme sequence specificity in high complexity genomes. Moreover, as many programming oligonucleotides used in the present systems, can be supplied concomitantly with a single protein effector moiety, e.g., the nucleic acid guided genome modifier protein (disclosed herein after) of the systems of the invention or any effector chimeric protein, complex or conjugate thereof, it is possible to modify more than one target at the same time, providing additional advantages over methods known in the art. This can be useful, for example, for rapidly knocking out a multiplicity of genes, or for inserting several different traits in different locations, or for tagging several different locations with one donor nucleotide tag.

In yet some further embodiments, the system is further characterized by that the insertion of the at least one replacement nucleic acid sequence into said target sequence is mediated by at least one of homology-directed repair (HDR) recombination and Non-Homologous End Joining (NHEJ).

Still further, in certain embodiments, the insertion of the at least one replacement nucleic acid sequence into the target sequence is mediated by HDR. In such case, the donor nucleic acid molecule is flanked by at least one homology arm. In some embodiments, the at least one homology arm displays complementarity to sequences that flank the target site within the target nucleic acid sequence of the target immune-regulatory gene of interest.

In some embodiments, the at least one replacement nucleic acid sequence of the donor nucleic acid molecule comprised within the disclosed system, encodes at least one of: at least one molecule participating in at least one immune-related signal transducing pathway, or any parts or fragments thereof, at least one inhibitory and/or modulatory non-coding nucleic acid molecule, at least one modulator of hematopoietic cell proliferation, recruitment and/or survival (specifically, cytokines, chemokine and the like), at least one detectable moiety, or any combinations thereof. In some embodiments, the at least one molecule participating in at least one immune-related signal transducing pathway, and/or the at least one modulator of hematopoietic cell proliferation, recruitment and/or survival, and/or the target for the at least one inhibitory and/or modulatory non-coding nucleic acid molecule, that are encoded by the replacement nuclei acid sequence of the donor molecule of the disclosed system, are also referred to herein as the “second immune-regulatory” gene/s.

In more specific embodiments, the at least one replacement nucleic acid sequence of the donor nucleic acid molecule comprised within the disclosed system comprises at least one inhibitory and/or modulatory non-coding nucleic acid molecule. In some embodiments, such inhibitory and/or modulatory non-coding nucleic acid molecule is a ribonucleic acid (RNA) molecule. In yet some further specific embodiments, the RNA molecule may be at least one of a double-stranded RNA (dsRNA), an antisense RNA, a single-stranded RNA (ssRNA), and a Ribozyme.

In some embodiments, the oligonucleotide aptamers and ODNs may be also applicable.

In more specific embodiments, the at least one inhibitory and/or modulatory non-coding nucleic acid molecule of the replacement sequence of the donor nucleic acid molecule disclosed by the system of the invention, may be at least one of a microRNA (miRNA), MicroRNA-like RNAs (milRNA), artificial miRNAs (amiRNA), small interfering RNA (siRNA) and short hairpin RNA (shRNA).

As indicated above and exemplified by Examples 9-11, in some embodiments, the donor molecules of the systems of the present disclosure may comprise as a replacement nucleic acid sequence, inhibitory and/or modulatory non-coding nucleic acid molecule. Thus, in some embodiments, the at least one inhibitory and/or modulatory non-coding nucleic acid molecule comprised in the donor nucleic acid molecules of the systems of the present disclosure, may be dsRNA molecules participating in RNA interference. More specifically, the dsRNA encompassed by the invention may be selected from the group consisting of small interfering RNA (siRNA), MicroRNA (miRNA), artificial miRNA (amiRNA), miRNA-like RNAs (mlRNA), short hairpin RNA (shRNA), PIWI interacting RNAs (piRNAs). RNA interference (RNAi) is a general conserved eukaryotic pathway which down regulates gene expression in a sequence specific manner.

It is the process of sequence-specific, post-transcriptional gene silencing in animals and plants, initiated by siRNA that is homologous in its duplex region to the sequence of the silenced gene. Gene silencing is induced and maintained by the formation of partly or perfectly double-stranded RNA (dsRNA) between the target RNA and the siRNA/shRNA derived ‘guide” RNA strand. The expression of the gene is either completely or partially inhibited. As known in the art RNAi is a multistep process. In a first step, there is cleavage of large dsRNAs into 21-23 ribonucleotides-long double-stranded effector molecules called “small interfering RNAs” or “short interfering RNAs” (siRNAs). These siRNAs duplexes then associate with an endonuclease-containing complex, known as RNA-induced silencing complex (RISC). The RISC specifically recognizes and cleaves the endogenous mRNAs/RNAs containing a sequence complementary to one of the siRNA strands. One of the strands of the double-stranded siRNA molecule (the “guide” strand) comprises a nucleotide sequence that is complementary to a nucleotide sequence of the target gene, or a portion thereof, and the second strand of the double-stranded siRNA molecule (the passenger” strand) comprises a nucleotide sequence substantially similar to the nucleotide sequence of the target gene, or a portion thereof. After binding to RISC, the guide strand is directed to the target mRNA cleaved between bases 10 and 11 relative to the 5′ end of the siRNA guide strand by the cleavage enzyme Argonaute-2 (AGO2).

Thus, the process of mRNA translation can be interrupted by siRNA.

In more particular embodiments, siRNAs comprise a duplex, or double-stranded region, of about 5-50 or more, 10-50 or more, 15-50 or more, 5-45, 10-45, 15-45, 5-40, 10-40, 15-40, 5-35, 10-35, 15-35, 5-30, 10-30 and 15-30 or more nucleotides long. In yet some more particular embodiments, the siRNAs of the invention comprise a nucleic acid sequence comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides. Often, siRNAs contain from about two to four unpaired nucleotides at the 3′ end of each strand. At least a portion of one strand of the duplex or double-stranded region of a siRNA is substantially homologous to or substantially complementary to a target sequence within the gene product (i.e., RNA) molecule as herein defined. The strand complementary to a target RNA molecule is the “antisense guide strand”, the strand homologous to the target RNA molecule is the “sense passenger strand” (which is also complementary to the siRNA antisense guide strand). siRNAs may also be contained within structured such as miRNA and shRNA which has additional sequences such as loops, linking sequences as well as stems and other folded structures.

More specifically, the strands of a double-stranded interfering RNA (e.g., siRNA) may be connected to form a hairpin or stem-loop structure (e.g., shRNA). Thus, as mentioned above the at least one inhibitory and/or modulatory non-coding nucleic acid molecule comprised in the donor nucleic acid molecules of the systems of the present invention may also be short hairpin RNA (shRNA).

According to other embodiments the at least one inhibitory and/or modulatory non-coding nucleic acid molecule comprised in the donor nucleic acid molecules of the systems according to the present disclosure may be a micro-RNA (miRNA). miRNAs are small RNAs made from genes encoding primary transcripts of various sizes. They have been identified in both animals and plants. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA. The pre-miRNA is a substrate for a form of dicer that removes the miRNA duplex from the precursor, after which, similarly to siRNAs, the duplex can be taken into the RISC complex. Unlike, siRNAs, miRNAs bind to transcript sequences with only partial complementarity and usually repress translation without affecting steady-state RNA levels. Both miRNAs and siRNAs are processed by Dicer and associate with components of the RNA-induced silencing complex (RISC). More specifically, microRNAs (miRNAs) form a class of endogenous, 20-22 nt long regulatory RNA molecules. They exert their function of post-transcriptional gene regulation through mRNA cleavage, RNA degradation, and translation inhibition. Most canonical miRNAs are transcribed by RNA polymerase II (Pol II) to produce pri-miRNA transcripts, which are then cleaved by RNase III-type enzymes called Dicer-like proteins into stem-loop structured precursors in the nucleus.

Stem-loop pre-miRNAs are subsequently cleaved into miRNA/miRNA* duplexes by Dicer or Dicer-like enzymes in the cytoplasm. The mature miRNAs are then incorporated into ARGONAUTE (AGO)-containing RNA-induced silencing complexes (RISC) in the cytoplasm to exert their regulatory effects by guiding the RISC to target transcripts through perfect or partially complementary base pairing. Non-canonical microRNAs have been discovered in various organisms. Non-canonical miRNAs have structural and function similar with canonical miRNAs, but they can skip one or more steps of classic miRNA biogenesis pathway. Small nucleolar RNA-derived miRNAs, endogenous short hairpin RNAs derived miRNAs and tRNA-derived miRNA are three Dicer-dependent, Dgcr8-Independent miRNAs. Multiple distinct miRNA-like RNAs can arise from a single miRNA precursor and they have been reported in plant species and mammals. The replacement nucleic acid sequence of the disclosed system may encode miRNA-like RNAs. Still further, in some embodiments, the replacement sequence may encode artificial miRNA (amiRNA). amiRNAs have been explored as alternative RNAi-triggering molecules and are designed to mimic primary miRNA stem-loops. The mature miRNA duplex in the central stem is replaced by sequences specifically designed for a specific target transcript, but the native flanking recognition sequences for cleavage by Drosha and Dicer are preserved. The artificial miRNAs are transcribed in larger transcripts and can be linked to RNA polymerase II-based expression systems.

More specific embodiments relate to the at least one inhibitory and/or modulatory non-coding nucleic acid molecule comprised in the donor nucleic acid molecules of the systems of the invention that may be at least one shRNA molecule. The term “shRNA”, as used herein, refers to an RNA agent having a stem-loop structure, comprising a first and second region of complementary sequence. The degree of complementarity and orientation of the regions being sufficient such that base pairing occurs between the regions. The first and second regions being joined by a loop region, the loop resulting from a lack of base pairing between nucleotides (or nucleotide analogs) within the loop region. Some of the nucleotides in the loop can be involved in base-pair interactions with other nucleotides in the loop.

An “antisense RNA” is a single strand RNA (ssRNA) molecule that is complementary to an mRNA strand of a specific target gene product. Antisense RNA may inhibit the translation of a complementary mRNA by base-pairing to it and physically obstructing the translation machinery. By “complementary” it is meant the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. Still further, in some embodiments, at least one inhibitory and/or modulatory non-coding nucleic acid molecule comprised in the donor nucleic acid molecules of the systems of the invention may comprise an antisense oligonucleotide, or any derivatives thereof. In more specific embodiments such oligonucleotide is an antisense oligonucleotide (ASO). As used herein, “oligonucleotide” means a compound comprising a plurality of linked nucleosides. In certain embodiments, an oligonucleotide comprises one or more unmodified ribonucleosides (RNA) and/or unmodified deoxyribonucleosides (DNA) and/or one or more modified nucleosides. As used herein, “modified oligonucleotide” means an oligonucleotide comprising at least one modified nucleoside and/or at least one modified internucleoside linkage.

In some particular and non-limiting embodiments, the at least one inhibitory and/or modulatory nucleic acid molecule is specific for at least one nucleic acid sequence encoding and/or controlling the expression of at least one second immune-regulatory gene (also referred to herein as the knockdown (KD) targets). More specifically, in some embodiments, the inhibitory and/or modulatory nucleic acid molecule/s act as at least one of: (i) an allogeneic T cell safety component; (ii) Fratricide reducing/preventing component; (iii) cytokine release syndrome (CRS) preventing component; (iv) activating component for nuclear factor kappa-light-chain-enhancer of activated B cells (NF-κB) signaling pathway; and (v) T cell Exhaustion tolerating component.

Thus, in certain specific embodiments, the at least one inhibitory and/or modulatory non-coding nucleic acid molecule of the replacement sequence of the donor nucleic acid molecule disclosed by the system of the invention may be, may act as, may lead to and/or may comprise at least one of the following options. In some embodiments (i), the at least one inhibitory and/or modulatory nucleic acid molecule acts as a T cell Exhaustion tolerating, inhibiting and/or reducing element. T cell Exhaustion as used herein refers to a state of T cell dysfunction that arises during many chronic infections and cancer. It is defined by poor effector function, sustained expression of inhibitory receptors and a transcriptional state distinct from that of functional effector or memory T cells. By “dysfunction” here it is understood that some T cells, after activation and proliferation, do not fulfill the functions they are expected to perform as effector T cells-typically, they fail to eliminate cancerous or infected cells and control the tumor or the virus respectfully. As originally described, antigen-specific T cells become “dysfunctional” during the chronic phase of high viral load infections, with progressive loss of interleukin (IL)-2, then tumor necrosis factor alpha (TNFα), and, finally, interferon gamma (IFNγ).

Accordingly, in some embodiments, such inhibitory and/or modulatory non-coding nucleic acid molecule encoded by the replacement sequence (e.g., miRNA encoded by the replacement sequence that is incorporated into at least one target site within the target first immuno-regulatory gene) is specifically directed against, and thus, may target any nucleic acid sequence encoding or acting as a component that participates either directly or indirectly in T cell exhaustion. In some embodiments, the inhibitory and/or modulatory non-coding nucleic acid molecule is specifically targeted against at least one inhibitory receptor, at least one phosphatase (e.g., SHP1, SHP2), at least one methylase (e.g., DNMT3A). In some optional embodiments, the inhibitory receptor targeted by the at least one inhibitory and/or modulatory non-coding nucleic acid molecule encoded by the replacement sequence, is at least one of Programmed Death-1 (PD-1, CD279), tumor necrosis factor receptor (TNFR)-associated factor 4 (TRAF4), cytotoxtic T lymphocyte antigen-4 (CTLA-4, CD152), Lymphocyte Activating 3 (Lag-3), T-cell immunoglobulin and mucin domain-3 (TIM-3), Cluster of Differentiation 244 (CD244/2B4), Cluster of Differentiation 160 (CD160) and T cell immunoreceptor with Ig and ITIM domains (TIGIT). In some embodiments, the DNMT3A may be used as a second regulatory protein. In yet some further embodiments, the DNMT3A as used herein refers to the human DNMT3A as denoted by NC_000002.12, that in some embodiments encodes the cDNA as denoted by SEQ ID NO: 176. In yet some further embodiments, the human DNMT3A protein is denoted by NP_001307821.1, as also denoted by SEQ ID NO: 177.

The primary goal of the present disclosure is to improve efficacy, safety, scalability and cost of solid tumor cell-therapies.

As indicated above and disclosed by Examples 10-12, the present disclosure provides a novel concept of building auto-regulated genetic circuits into immune-cells, for example, non-manipulated or alternatively, genetically manipulated T cells, e.g., CAR T cells, for trans modulating the immune-system. In some embodiments, the inhibitory and/or modulatory non-coding nucleic acid molecule encoded by the replacement sequence of the donor of the disclosed system targets modified T cells to selectively circumvent their exhaustion upon activation in the TME. Genetic rewiring is achieved by precisely inserting artificial miRNAs under endogenous exhaustion-related “Driver” promoters to downregulate “Target” genes that cause exhaustion. In some embodiments, use of a pair of two target recognition elements, as also disclosed previously by the present inventors (WO 2013/088446), enables specific replacement of the “Driver” gene limiting the risk of off-target mutations. Further advantages of combined insertion and silencing are (i) the ability to regulate when a gene is turned on/off by biologically and clinically relevant cellular cues, and (ii) multiple gene-knockdown with a single dsDNA cleavage and RNA-silencing of both alleles.

Proof of concept of the rewired miRNA auto-regulatory method, that is also exemplified by Examples 10-12, allows the development, of a wide range of cancer and non-cancer cell-therapies in both autologous and allogeneic settings.

The present disclosure enables the design of gene modified CAR T cells resistant to exhaustion and/or showing enhanced activation and expansion kinetics.

The current state-of-the-art is the permanent modification of CAR T cells to knock out negative regulators (e.g., PD1, DNMT3A) or to drive expression of molecules promoting efficacy upon CAR engagement. However, these strategies provide limited or no adaptation to the location of T cells, and, most importantly, do not limit the activation to the TME where exhaustion should be countered.

The main concept of this unique approach utilizes endogenous genomic promoters that are selectively turned on in the activated CAR T cells within the TME to physiologically drive genes related to immune suppression or exhaustion. This gene/s (“Driver/s”, also referred to herein as the “first” immunoregulatory genes) are permanently replaced (knock-out [KO]) in the genome by a control element such as an artificial microRNA (amiRNA) or siRNA which in turn knocks-down (KD) other immunosuppressive genes, genes that contribute to exhaustion or genes that normally downregulate inflammation (“Target”). The foundation of this approach is that the Driver's promoter is turned on in the TME, at the correct time and place. When that promoter drives the expression of the amiRNAs, the progression of exhaustion process is prevented and, therefore, the cell is prevented from entering the terminal exhaustion states. The strength of this approach is that the stronger the inhibition pressure, the higher the expression of anti-inhibitory trans-modulation elements and vice-versa, when the tumor shrinks and pressure is relieved, expression is suspended. This auto-modulated approach is expected to be significantly safer than permanent KO of target(s) that is more likely to be associated with persistent resistance to repression and more harmful in cases of on-target off-tumor immune effector function.

In some additional or alternative embodiments (ii), the at least one inhibitory and/or modulatory nucleic acid molecule acts as an allogeneic T cell safety element. More specifically, the inhibitory and/or modulatory nucleic acid molecule encoded by the replacement sequence of the donor of the disclosed system, may target any nucleic acid sequence encoding or acting as a component that participates either directly or indirectly, in allogeneic T cell safety. More specifically, such safety component may be according to some embodiments specifically directed against at least one of β2 microglobulin (B2M), T-cell receptor alpha (TCRα) and/or beta (TCRβ). In yet some additional or alternative embodiments (iii), the at least one inhibitory and/or modulatory nucleic acid molecule acts as Fratricide reducing and/or preventing element. The term Fratricide, as used herein, refers to a process where CAR T cells engineered to recognize T-cell antigens will inevitably turn on themselves, resulting in CAR T-cell lysis, termed “fratricide”.

More specifically, the at least one inhibitory and/or modulatory nucleic acid molecule encoded by the replacement sequence of the donor of the disclosed system may target any nucleic acid sequence encoding or acting as a component that participates either directly or indirectly in Fratricide. In some embodiments, such component is specifically directed against at least one of cluster of differentiation 38 (CD38), T-cell receptor alpha (TCRα) and/or beta (TCRO). Still further, in some alternative or additional embodiments (iv), the at least one inhibitory and/or modulatory nucleic acid molecule acts as a CRS preventing component. Cytokine release syndrome (CRS) as used herein, refers to an acute systemic inflammatory syndrome characterized by fever and multiple organ dysfunction that is associated with chimeric antigen receptor (CAR)-T cell therapy, therapeutic antibodies, and haploidentical allogeneic transplantation. More specifically, the at least one inhibitory and/or modulatory nucleic acid molecule encoded by the replacement sequence of the donor of the disclosed system may target any nucleic acid sequence encoding or acting as a component that participates either directly or indirectly in CRS. Accordingly, in some embodiments, such component is specifically directed against Interleukin 6 (IL-6). In some alternative or additional embodiments (v), the at least one inhibitory and/or modulatory nucleic acid molecule acts as an activating element for NF-κB signaling pathway. In some embodiments, the at least one inhibitory and/or modulatory nucleic acid molecule encoded by the replacement sequence of the donor of the disclosed system may target any nucleic acid sequence encoding or acting as a component that participates either directly or indirectly in activating the NF-κB signaling pathway. In some embodiments, such component is specifically directed against tumor necrosis factor receptor (TNFR)-associated factor 4 (TRAF4). In some optional and non-limiting embodiments, the inhibitory and/or modulatory nucleic acid molecule is miR-4443.

In some embodiments, the TRAF4 as used herein refers to the human TRAF4, that comprises the nucleic acid sequence as denoted by SEQ ID NO: 192. Still further, in some embodiments, the human TRAF4 protein comprises the amino acid sequence as denoted by SEQ ID NO: 193.

The advantages of a combined insertion and silencing approach, as opposed to silencing only by permanent gene knockout include the a) ability to regulate when a gene is turned on or off following biologically and clinically relevant cellular cues; b) multiple gene-knockdown with a single dsDNA genomic cleavage as opposed to multiple dsDNA breaks in multiple gene-knockout; c) RNA-silencing, as opposed to single allele knockout, silences both alleles.

Still further, in some embodiments, the at least one target first immune-regulatory gene, also referred to herein as “driver” gene, is at least one gene encoding an undesired product that is targeted for knocking out, and/or modulation of the expression and/or activity. Still further, naturally, such first immune-regulatory gene is upregulated during an immune-related process that is targeted and modulated by the disclosed systems. Therefore, in some embodiments, the replacement sequence provided by the donor molecule of the disclosed system is inserted into the coding or non-coding sequences of the target first regulatory gene. This insertion results in inhibition, knockout, and/or modulation of the expression and/or activity of the target first regulatory gene. Still further, the replacement sequence is inserted and expressed under at least one regulatory element of such target first regulatory gene, e.g., under the promoter of the target first immunoregulatory gene, that acts as a “driver”. Thus, since the target first regulatory gene is upregulated in an immunological process, e.g., exhaustion of T cells (or any of the immunological processes targeted and modulated by the disclosed methods), and in some embodiments, is involved in such process, insertion of the replacement sequence into coding and/or non-coding sequences of such target gene results in inhibition or modulation of the expression of such target first immune-modulator. Alternatively, or additionally, such insertion may further result in expression of the replacement sequence that may be either a second immune-regulatory gene, and/or an inhibitory or regulatory nucleic acid sequence, that is expressed under the endogenous promoter of the target “driver” during the immunological process (e.g., T cell exhaustion), thereby modulating and/or inhibiting the process. In some embodiments, a target gene that can act as a “driver” (first immune-regulatory gene) is one of: at least one gene encoding at least one immune-checkpoint protein, at least one gene encoding an immune-modulator of hematopoietic cell proliferation, recruitment and/or survival (for example, at least one cytokine and/or chemokine), at least one hypoxia inducible gene, at least one gene encoding at least one signal transduction molecule, at least one gene encoding at least one immuno-modulatory receptor.

The disclosed systems, compositions, cells and methods provide a powerful therapeutic tool for modulating immune response by modulating immune-regulatory genes, for example, at least one immune checkpoint proteins and/or cytokines and chemokines.

More specifically, T cell activation is tightly regulated by co-stimulators or co-inhibitors known as immune checkpoints. Accordingly, when referring to “immune checkpoints”, it should be understood to relate to any molecule which act as gatekeepers of immune responses. In the past decades, a number of immune checkpoint proteins have been identified and studied in cancer, including but not limited to programmed cell death protein 1 (PD-1), cytotoxic T lymphocyte-associated antigen-4 (CTLA-4), lymphocyte activation gene-3 (LAG3), T cell immunoglobulin and mucin-domain containing-3 (TIM3), T cell immunoglobulin and ITIM domain (TIGIT), V-domain Ig suppressor of T cell activation (VISTA), B7 homolog 3 protein (B7-H3) and B and T cell lymphocyte attenuator (BTLA).

In more specific and non-limiting embodiments, the target first immune-regulatory gene (“driver”) targeted by the disclosed system is at least one gene encoding at least one immune-checkpoint protein. In some optional embodiments, the immune-checkpoint protein may be at least one of Programmed cell death protein 1 (PDCD1/PD1); T Cell Immunoreceptor With Ig And ITIM Domains (TIGIT); B And T Lymphocyte Associated (BTLA); Cytotoxic T-Lymphocyte-Associated protein 4 (CD152/CTLA4); Lymphocyte activation gene 3 (LAG-3) (CD223); and T cell immunoglobulin and mucin domain 3 (TIM-3)(HAVCR2).

In some embodiments, the at least one immune-checkpoint protein encoding gene that are targeted by the system disclosed herein, and acting as a “driver”, may be at least one of the Programmed cell death protein 1 (PDCD1/PD1); T Cell Immunoreceptor With Ig And ITIM Domains (TIGIT); B And T Lymphocyte Associated (BTLA); Cytotoxic T-Lymphocyte-Associated protein 4 (CD152/CTLA4); Lymphocyte activation gene 3 (LAG-3) (CD223); T cell immunoglobulin and mucin domain 3 (TIM-3)(HAVCR2); TGF-beta receptor; hsa-miRNA-4443 (miR-4443); Interleukin-6 (IL-6); CD38; TCR-alpha constant region; TCR-beta constant region; Beta-2 Microglobulin (B2M); CD3 epsilon; CD3 zeta; CD160; CD244; Adenosine A2A receptor (A2AR); B7-H3 (CD276); B7-H4/V-Set Domain Containing T Cell Activation Inhibitor 1 (VTCN1); Indoleamine 2,3-dioxygenase (IDO); Killer cell Immunoglobulin-like Receptor (KIR); V-domain immunoglobulin suppressor of T cell activation/V-set immunoregulatory receptor (VSIR/VISTA); Sialic acid-binding ImmunoGlobulin-type LEctin 9/CD329 (SIGLEC9); CD3 Gamma; CD3 Delta; Glycogen Synthase Kinase (GSK)-3a/D (GSK3); Vaccinia Related Kinase 2 (VRK2); Src Homology region 2 domain-containing Phosphatase-1 (SHP-1/PTPN6); SH2 domain-containing protein tyrosine phosphatase-2 (SHP-2); Hematopoietic Progenitor Kinase 1 (HPK1 or MAP4K1); Monoamine Oxidase A (MAO-A); p38 kinase/Crebl/Klf4 axis in tumor-associated macrophages; DiacylGlycerol Kinase a (DGK-Alpha); CD73; Natural Killer Group protein 2A (NKG2A); Poliovirus receptor-related immunoglobulin domain containing (PVRIG/CD112R); CEACAMI; CEACAM 5; CEACAM6 (CD66c); FAK; C—C motif chemokine ligand 2 (CCL2); C—C motif chemokine receptor 2 (CCR2); Leukemia inhibitory factor (LIF); CD47/SIRPa; Colony-Stimulating Factor-1 (CSF-1)(M-CSF); Interleukin-1R3 (IL-1R3) (IL-1R accessory chain (IL-1RAP)); Interleukin-8 (IL-8/CXCL8); SEMA4D; Ang-2; Common lymphatic endothelial and vascular endothelial receptor-1 (CLEVER-1); AXL receptor tyrosine kinase (Ax1/UFO/ARK/Tyro7/JTK11); Phosphatidylserine.

In some embodiments, the target first immuno-regulatory gene is the gene encoding PD-1. More specifically, PD-1 (programmed cell death protein 1 precursor), belongs to CD28 superfamily and is an immune-inhibitory receptor expressed in activated T cells and is involved in the regulation of T-cell functions, including those of effector CD8+ T cells.

The binding of PD-1 to its ligands PD-L1 and PD-L2 suppress the activation and function of T cells to downregulate the immune response (Qin et al, Molecular Cancer 2019. 18:155). In some specific embodiments, the PD-1 gene as disclosed herein refers to the human PD-1 gene and gene product. Still further, in some specific embodiments, the PD-1 gene as referred to in the present disclosure is denoted by NC_000002.1 and is located at c241858894-241849884 of the Homo sapiens chromosome 2. Still further, in some specific embodiments, the PD-1 encoding gene comprises the nucleic acid sequence as denoted by SEQ ID NO: 183. In yet some further embodiments, the cDNA encoded by the human PD-1 gene may comprise the nucleic acid sequence as denoted by SEQ ID NO: 199. Still further, in some embodiments, the PD-1 protein as referred to herein is the human protein as denoted by NP_005009.2. In more specific embodiments, the PD-1 protein comprises the amino acid sequence as denoted by SEQ ID NO: 200.

Still further, the invention further provides modulating other immune-checkpoint proteins.

In some embodiments, the target first immuno-regulatory gene is the gene encoding LAG3. More specifically, LAG3 (lymphocyte activation gene 3) is an inhibitory receptor on antigen activated T-cells. It delivers inhibitory signals upon binding to ligands, such as FGL1. Other known ligands for LAG 3 are MHC-II, galectin-3, LSECtin, and a-synuclein. LAG-3 signaling play a negative regulatory role in T helper 1 (Th1) cell activation, proliferation and cytokine secretion. During tumorigenesis and cancer progression, tumor cells exploit this pathway to escape from immune surveillance. In some specific embodiments, the LAG3gene as disclosed herein refers to the human LAG3 gene and gene product. Still further, in some specific embodiments, the LAG3 gene as referred to in the present disclosure is denoted by NC_000012.12 and is located at 6772520-6778455 of the Homo sapiens chromosome 12. In yet some further embodiments, the cDNA encoded by the human LAG3 gene may comprise the nucleic acid sequence as denoted by SEQ ID NO: 190. Still further, in some embodiments, the LAG3 protein as referred to herein is the human protein as denoted by NP_002277.4. In more specific embodiments, the LAG3 protein comprises the amino acid sequence as denoted by SEQ ID NO: 191.

In some embodiments, the target first immuno-regulatory gene is the gene encoding TIM-3. More specifically, TIM-3 (HAVCR2, hepatitis A virus cellular receptor 2 precursor), is classified as immune checkpoint molecule similar to CTLA-4 and PD-1. TIM-3 is a cell surface receptor implicated in modulating innate and adaptive immune responses.

Four distinct ligands have been reported to TIM-3, including galectin-9, high-mobility group protein B1 (HMGB1), carcinoembryonic antigen cell adhesion molecule 1 (Ceacam-1), and phosphatidyl serine (PtdSer), and the function of ligand-receptor interaction is coinhibitory. In some specific embodiments, the TIM-3 gene as disclosed herein refers to the human TIM-3 gene and gene product. Still further, in some specific embodiments, the TIM-3 gene as referred to in the present disclosure is denoted by NC_000005.10. In yet some further embodiments, the cDNA encoded by the human TIM-3 gene may comprise the nucleic acid sequence as denoted by SEQ IS NO: 188. Still further, in some embodiments, the TIM-3 protein as referred to herein is the human protein as denoted by NP_116171.3. In more specific embodiments, the TIM-3 protein comprises the amino acid sequence as denoted by SEQ ID NO: 189.

In some embodiments, the target first immuno-regulatory gene is the gene encoding TIGIT. More specifically, TIGIT (T-cell immunoreceptor with Ig and ITIM domains) is another coinhibitory immune checkpoint receptor that suppress the activation of T cells. TIGIT binds two ligands, CD155 and CD112. It exerts its immunosuppressive effects by competing with other counterparts, CD266 (DNAM-1) or CD96; specifically, CD226 delivered a positive co-stimulatory signal, while TIGIT delivered inhibitory signals. In some specific embodiments, the TIGIT gene as disclosed herein refers to the human TIGIT gene and gene product. Still further, in some specific embodiments, the TIGIT gene as referred to in the present disclosure is denoted by NC_000003.12 and is located at 114294028-114310288 of the Homo sapiens chromosome 3. In yet some further embodiments, the cDNA encoded by the human TIGIT gene may comprise the nucleic acid sequence as denoted by SEQ IS NO: TIGIT. Still further, in some embodiments, the TIGIT protein as referred to herein is the human protein as denoted by NP_776160.2. In more specific embodiments, the TIGIT protein comprises the amino acid sequence as denoted by SEQ ID NO: 198.

In some embodiments, the target first immuno-regulatory gene is the gene encoding CTLA-4. More specifically, CTLA-4 is a cell-surface receptor related to CD28, which binds to the ligands CD80 (B7-1) and CD86 (B7-2). The binding of CTLA-4 to CD80/CD86 delivers a negative signal to T cells activation (Qin et al, Molecular Cancer 2019. 18:155).

In some embodiments, the target first immuno-regulatory gene is the gene encoding VISTA. More specifically, VISTA is a type I transmembrane protein that harbors some identity with PD-1, its ligand VSIG-3 was recently reported. VISTA was shown to negatively regulate T cell responses in mouse. VISTA blockade, in preclinical in mouse, showed improved infiltration, proliferation, and effector function of tumor-infiltrating T cells within the tumor microenvironment.

In some embodiments, the target first immuno-regulatory gene is the gene encoding B7-H3. More specifically, B7-H3, is yet another type I transmembrane glycoprotein, which was reported as positive co-stimulator but also shown to be involved in the inhibition of T cells. Indeed, its binding partner is still unknown. Nevertheless, the expression of B7-H3 protein can be detected on activated immune cells such as T cells, NK cells, and APCs and more importantly, it was overexpressed on a wide spectrum of tumor tissue and linked to disease states and prognosis.

In some embodiments, the target first immuno-regulatory gene is the gene encoding BTLA. More specifically, BTLA is identified as another inhibitory receptor that belongs to CD28 superfamily and its expression was detected on mature lymphocytes (such as B cells, T cells, and Tregs), macrophages, and mature bone marrow-derived DCs. Its ligand is the Herpesvirus entry mediator (HVEM) and was reported to negatively regulate antigen receptor signaling via PTPN6/SHP-1 and PTPN11/SHP-2.

In some specific embodiments, the target immune-regulatory gene of interest is Programmed cell death protein 1 (PDCD1/PD-1). In yet some more specific embodiments, the target sequence within the target immune-regulatory gene is a target sequence within exon 3 of the PD-1 gene. In some further specific embodiments, the target sequence within the target immune-regulatory gene is a target sequence within exon 1 of the PD-1 gene. In some embodiment, the insertion is mediated by HDR or NHEJ.

In yet some alternative and/or additional embodiments, the target sequence within the target immune-regulatory gene targeted by the donor nucleic acid molecule of the disclosed system is within exon 3 of the PD-1 gene. In yet some further embodiments, the insertion is mediated by NHEJ. In such case, the insertion results in production of a soluble PD-1 truncated product.

In some particular and non-limiting embodiments, the target sequence within the at least one target first immune-regulatory gene targeted by the donor nucleic acid molecule of the disclosed system, is within exon 3 of the PD-1 gene. In some embodiments, the insertion of the replacement sequence is mediated by NHEJ, and the insertion results in knockout of the endogenous PD-1 gene. As indicated in Example 1, Since the premature stop codon in the −1 frame shift product occurs in exon 3, it is predicted not to express due to Nonsense Mediated Decay (NMD), thereby resulting in knockout of the endogenous PD-1 gene.

In yet some further alternative or additional embodiments, the target sequence within the target immune-regulatory gene targeted by the donor nucleic acid molecule of the disclosed system is within exon 3 of the PD-1 gene, and the insertion is mediated by HDR.

According to these embodiments, the donor nucleic acid molecule further comprises at least one homology arm that flank the replacement sequence.

According to some particular and non-limiting embodiments, the replacement sequence comprises an in-frame nucleic acid sequence encoding a hematopoietic cell signaling domain. Accordingly, the insertion of such replacement sequence into the target sequence in exon 3 of PD-1 results in formation of at least one soluble or non-soluble PD-1 chimeric protein. In some embodiments, such chimeric protein comprises PD-1 extracellular domain and an exogenous hematopoietic cell signaling domain encoded by the replacement sequence.

In some embodiments, the hematopoietic cell signaling domain encoded by the replacement sequence of the donor nucleic acid molecule of the disclosed system may comprise at least one transmembrane and/or intracellular domain of at least one co-stimulatory receptor. In such embodiments, the insertion of such replacement sequence into the target sequence within exon 3, results in the formation of at least one membrane-anchored chimera that transduces stimulatory signals. In some optional embodiments, the co-stimulatory receptor is at least one of 4-1BB (CD137; TNFRS9), Cluster of Differentiation 28 (CD28) and Tumor necrosis factor receptor superfamily, member 4 (TNFRSF4, OX40).

In yet some alternative embodiments, the hematopoietic cell signaling domain encoded by the replacement sequence of the donor nucleic acid molecule of the disclosed system is at least one immunoglobulin Fragment crystallizable region (Fc region). In such case, the insertion of such replacement sequence into the target sequence within exon 3, results in the formation of a soluble chimera that binds PD-1 ligand, blocks inhibitory signals and/or activates immune cells.

As shown in the following Examples, several integration cassettes were designed.

It should be understood that the present disclosure encompasses any systems comprising donor molecules that comprise any of the constructs disclosed by the present disclosure, for example each one of SEQ ID NO: 22 to 26, or any of the constructs indicated herein below. Still further, the invention further encompasses any cell, composition and methods and uses of any of the disclosed systems. Thus, in some embodiments an integration cassette that is also referred to herein as a donor nucleic acid molecule, may comprise the cytoplasmic domain of 41BBI, that will replace the sequence encoding the intracellular domain of the target PD-1, to produce a chimeric produce. This cassette may further comprise additional immune-modulators, specifically, cytokines such as IL7 and CCL21.

More specifically, in some embodiments, the 41BBICD-T2A-CCL21-P2A-TL7 PD1 integration cassette, that in some particular and non-limiting embodiments comprise the sequence as denoted by SEQ ID NO:26, contains all elements described above and encodes the protein PD1Nterm-41BBICD-T2A-CCL21-P2A-IL7. In some embodiments, such chimera comprises the amino acid sequence as denoted by SEQ ID NO: 27.

Still further, in some embodiments, the cassettes of the invention may comprise at least one detectable moiety. For example, fluorescent proteins (e.g., GFP, luciferase) that can be detected with flow cytometry. Thus, in some embodiments, the cassettes of the invention may comprise:

41BBtransmembrane-EGFP-T2A-EBFP-P2A-luciferase PD1 integration cassette. In yet some further embodiments the cassette comprises the sequence of SEQ ID NO:22. replaces the 41BB ICD with EGFP (as denoted by SEQ ID NO:28), CCL21 with EBFP (as denoted by SEQ ID NO:29), and IL7 with luciferase (as denoted by SEQ ID NO:30).

The complete protein product of the construct is as denoted by in SEQ ID NO:31.

Still further in some embodiments, the disclosed cassette may comprise 41BBtransmembrane-EGFP-T2A-IL7-P2A-luciferase PD1 integration cassette (as denoted by SEQ ID NO:23) replaces the 41BB ICD with EGFP, and the IL7 with luciferase. The complete protein product of the construct is as denoted by SEQ ID NO:32.

Still further in some embodiments, the disclosed cassette may comprise 41BBICD-T2A-EBFP-P2A-luciferase PD1 integration cassette (as denoted by SEQ ID NO:24) replaces CCL21 with EBFP and IL7 with luciferase. The complete protein product of the construct is as denoted by SEQ ID NO:33.

In some embodiments, the disclosed cassette may comprise 41BBtransmembrane-EGFP-T2A-mCherry-P2A-luciferase PD1 integration cassette (as denoted by SEQ ID NO:25) replaces 41BB ICD with EGFP, CCL21 with mCherry (as denoted by SEQ ID NO:34) and IL7 with luciferase. The complete protein product of the construct is as denoted by SEQ ID NO:35.

Still further, in some embodiments, the target sequence within the PD-1 that is used herein as the target first immune-regulatory gene, is within exon 1. In some embodiments, the insertion of the replacing nucleic acid sequence is mediated by HDR. Accordingly, such insertion results in at least one of: (i) the production of at least one immune-modulatory product, controlled by at least one endogenous control element of said PD-1 gene; and (ii) inhibition and/or reduction and/or modulation of the expression and/or activity and/or stability of said PD-1 gene.

Still further, in some embodiments, the target sequence within the TIGIT gene (GeneBank accession number NC_000003.12) that is used herein as the target first immune-regulatory gene, is within exon 2 which encodes the start of the protein, between guides sgRNA1: cctgctgctcccagttgacc (also denoted by SEQ ID NO: 201) and sgRNA2: tttgtaatgctgacttgggg (also denoted by SEQ ID NO: 201), that are targeted at a target sequence around nucleotide 194-195 of the TIGIT nucleic acid sequence, as denoted by SEQ ID NO: 197).

In some embodiments, the insertion of the replacing nucleic acid sequence is mediated by HDR. Accordingly, such insertion results in at least one of: (i) the production of at least one immune-modulatory product, controlled by at least one endogenous control element of the TIGIT gene; and (ii) inhibition and/or reduction and/or modulation of the expression and/or activity and/or stability of the TIGIT gene.

In some further embodiments, the target immune-regulatory gene targeted by the system of the invention is at least one gene encoding an immune-modulator of hematopoietic cell proliferation, recruitment and/or survival. In more specific embodiments, such at least one target first immune-regulatory gene may be at least one cytokine and/or chemokine.

In yet some further specific and non-limiting embodiments, the target gene is Leukemia inhibitory factor (LIF-1).

In some embodiments, the target sequence within the at least one target first immune-regulatory gene targeted by the disclosed system is a target sequence within at least one of exon 2 and exon 3 of the LIF-1 gene. More specifically, the insertion of the replacement sequence is mediated by HDR or NHEJ. In some specific embodiments, the LIF-1 gene as disclosed herein refers to the human LIF-1 gene and gene product. Still further, in some specific embodiments, the LIF-1 gene as referred to in the present disclosure is denoted by NC_000022.11 and is located at c30246759-30240453 of the Homo sapiens chromosome 22. Still further, in some specific embodiments, the LIF-1 encoding gene comprises the nucleic acid sequence as denoted by SEQ ID NO: 194. In yet some further embodiments, the cDNA encoded by the human LIF-1 gene may comprise the nucleic acid sequence as denoted by SEQ ID NO: 195. Still further, in some embodiments, the LIF-1 protein as referred to herein is the human protein as denoted by NP_001244064.1.

In more specific embodiments, the LIF-1 protein comprises the amino acid sequence as denoted by SEQ ID NO: 196.

Still further, in some embodiments, the target sequence within the at least one target first immune-regulatory gene targeted by the disclosed system is a target sequence within at least one target sequence in the coding or non-coding regions of the C—X—C Motif Chemokine Receptor 6 (CXCR6) gene. More specifically, the insertion of the replacement sequence is mediated by HDR or NHEJ.

In some specific embodiments, the CXCR6 gene as disclosed herein refers to the human CXCR6 gene and gene product. Still further, in some specific embodiments, the CXCR6 encoding gene comprises the nucleic acid sequence as denoted by AF007545. In yet some further embodiments, the nucleic acid sequence of the cDNA encoded by the human CXCR6 gene are denoted by SEQ ID NO: 186. Still further, in some embodiments, the CXCR6 protein as referred to herein is the human protein that comprises the amino acid sequence as denoted by SEQ ID NO: 187.

In yet some further embodiments, the replacement nucleic acid sequence (also referred to herein as knocked-in (KI) sequence), further comprises at least one nucleic acid sequence encoding at least one modulator of hematopoietic cell proliferation, recruitment and/or survival. Specifically, in some embodiments, such modulator may be at least one cytokines, chemokine. In some specific and optional embodiments, such modulator may comprise at least one of Interleukin 1 (IL-1), Interleukin 7 (IL-7), Interleukin 15 (IL-15), Chemokine ligand 19 (CCL19) and Chemokine ligand 21 (CCL21).

In some embodiments, the at least one target recognition element of the disclosed system may be at least one of a single strand ribonucleic acid (RNA) molecule, a double strand RNA molecule, a single-strand DNA molecule (ssDNA), a double strand DNA (dsDNA), a modified deoxy ribonucleotide (DNA) molecule, a modified RNA molecule, a locked-nucleic acid molecule (LNA), a peptide-nucleic acid molecule (PNA) and any hybrids or combinations thereof.

Still further, as discussed above, in some embodiments, the systems of the present disclosure may comprise at least one nucleic acid guided genome modifier protein, chimeric protein, complex or conjugate. In some embodiments, such nucleic acid guided genome modifier protein comprises at least one nucleic acid modifier component and at least one component capable of binding the at least one target recognition element.

In some embodiments, the at least one nucleic acid modifier component is a protein-based modifier, a nucleic acid-based modifier or any combinations thereof, and wherein said protein-based modifier is at least one of a nuclease, a methyltransferase, a methylated DNA binding factor, a transcription factor, a transcription repressor, a chromatin remodeling factor, a polymerase, a demethylase, an acetylase, a deacetylase, a kinase, a phosphatase, an integrase, a recombinase, a ligase, a topoisomerase, a girase, a helicase, and any combinations thereof.

It should be appreciated that the at least one nucleic acid guided genome modifier protein of the systems of the present disclosure or any chimeric or fusion protein thereof, must comprise at least one effector or modifier component, or act as a effector or modifier component. In some embodiments, such effector or modifier component may be a protein-based modifier, a nucleic acid-based modifier or any combinations thereof. In some embodiments, “the nucleic acid modifier or effector” component may be any component, element or specifically protein, polypeptide or nucleic acid sequence or oligonucleotide that upon direct or indirect interaction with a target nucleic acid sequence (e.g., the first immune-regulatory gene), modify or modulate the structure, function (e.g., expression), or stability thereof. Such modification may include the modification of at least one functional group, addition or deletion of at least one chemical group by modifying an existing functional group or introducing a new one such as methyl group.

The modifications may include cleavage, methylation, demethylation, deamination and the like. Specific modifier component applicable in the present invention may include but are not limited to a protein-based modifier, for example, a nuclease, a methyltransferase, a methylated DNA binding factor, a transcription factor, transcription repressor, a chromatin remodeling factor, a polymerase, a demethylase, an acetylase, a deacetylase, a kinase, a phosphatase, an integrase, a recombinase, a ligase, a topoisomerase, a gyrase, a helicase, any combinations thereof or any fusion proteins comprising at least one of the modifier proteins disclosed by the invention.

As will be elaborated herein below, “activity” of the nucleic acid modifier or effector component in the nucleic acid guided genome modifier/effector chimeric protein, complex or conjugate of the invention, referred to herein may relate in some embodiments to any modification performed in any nucleic acid molecule or sequence, for example, any sequence encoding a product, e.g., the target immune-regulatory gene, or alternatively any non-coding sequences. Such modification in some embodiments may result (specifically in case performed on a coding sequence, or alternatively in a regulatory non-coding sequence), in modulation of the expression, stability or activity of the encoded product. Non-limiting examples for such modification may be nucleolytic distraction, methylation, demethylation, acetylation and the like. In some specific embodiments, such nucleic acid modifier protein may be a nuclease, and the activity referred to herein may be the nucleolytic activity of the nuclease. However, in some alternative embodiments, the invention further encompasses other activities that do not relate to nucleolytic activity.

Still further, in case of a nucleic acid-modifier that is a protein such as a nuclease, the target recognition element may be a nucleic acid guide that targets the nuclease to a specific target position within a target nucleic acid sequence (e.g., SCNA, gRNA). The recognition of the target by the target recognition element is facilitated in some embodiments by base-pairing interactions. These target recognition elements are specifically relevant in case of guided nucleases. In some embodiments, for nucleases displaying a nucleolytic activity, directing the nuclease to a specific predetermined target site in the target nucleic acid may result in cleaving the phosphodiester bonds between monomers of nucleic acids (e.g., DNA and/or RNA) that may lead in some embodiments to specific modifications thereof, such as mutations, deletions, frame-shifts, insertion of a Donor nucleic acid, or replacement of the target or a portion thereof with an alternative Donor nucleic acid.

In more specific embodiments, the nucleic acid modifier component of the disclosed system is at least one nuclease. In some optional embodiments, such nuclease is a Type IIS restriction endonuclease or any fragment, variant, mutant, fusion protein or conjugate thereof.

As discussed herein, the systems of the disclosure may further comprise the at least one nucleic acid modifier component. In some embodiments, the at least one nucleic acid modifier component suitable for the nucleic acid guided genome modifier chimeric protein of the invention may be any nucleic acid guided modifier, for example, protein-based modifier, a nucleic acid-based modifier or any combinations thereof.

As indicated above, in some specific embodiments, the nucleic acid modifier component may be at least one nuclease. More specifically, as used herein, the term “nuclease” refers to an enzyme that in some embodiments display a nucleolytic activity, specifically, capable of cleaving the phosphodiester bonds between monomers of nucleic acids (e.g., DNA and/or RNA). Nucleases variously effect single and double stranded breaks in their target molecules. There are two primary classifications based on the locus of activity. Exonucleases digest nucleic acids from the ends. Endonucleases act on regions in the middle of target molecules. They are further subcategorized as deoxyribonucleases and ribonucleases. The former acts on DNA, the latter on RNA. The nucleases belong just like phosphodiesterase, lipase and phosphatase to the esterases, a subgroup of the hydrolases. This subgroup includes the Exonucleases which are enzymes that work by cleaving nucleotides one at a time from the end (exo) of a polynucleotide chain. A hydrolyzing reaction that breaks phosphodiester bonds at either the 3′ or the 5′ end occurs.

Eukaryotes and prokaryotes have three types of exonucleases involved in the normal turnover of mRNA: 5′ to 3′ exonuclease (Xrn1), which is a dependent decapping protein; 3′ to 5′ exonuclease, an independent protein; and poly (A)-specific 3′ to 5′ exonuclease.

Members of this family include Exodeoxyribonucleases producing 5′-phosphomonoesters, Exoribonucleases producing 5′-phosphomonoesters, Exoribonucleases producing 3′-phosphomonoesters and Exonucleases active with either ribo- or deoxy-. Members of this family include exonuclease, II, III, IV, V, VI, VII, and VIII. As noted above, Endonucleases are enzymes that cleave the phosphodiester bond within a polynucleotide chain. Some endonucleases, such as deoxyribonuclease I, cut DNA relatively nonspecifically (without regard to sequence), while many, typically called restriction endonucleases or restriction enzymes, cleave only at very specific nucleotide sequences.

In some embodiment, the nuclease may be an active enzyme having a nucleolytic activity as specified above.

A restriction enzyme is an embodiment for endonuclease that cleaves DNA into fragments at or near its specific recognition sites within the molecule. To cut DNA, most restriction enzymes make two incisions, through each sugar-phosphate backbone (i.e. each strand) of the DNA double helix.

In some embodiments, Type IIS restriction enzymes recognize asymmetric DNA sequences and cleave outside of their recognition sequence, which can be removed, and can thus be used. Non-limiting examples of such restriction enzymes may include, but are not limited to FokI, AcuI, AlwI, BaeI, BbsI, BbvI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, HgaI, HphI, HpyAV, MboII, MlyI, MmeI, MnlI, NmeAIII, PleI, SapI, SfaNI, and T-TEVI.

In some specific embodiments, the nuclease used as the effector/modifier component in the modifier provided by the systems of the present disclosure may be at least one typeIIS nuclease or any cleavage domains thereof. These may include cleavage domains from Type IIS restriction endonucleases: AarI, Acc36I, AceIII, AclWI, AcuI, Ajul, AloI, AlwI, Alw26I, AmaCSI, ApyPI, AquII, AquIII, AquIV, ArsI, AsuHPI, BaeI, BarI, Bbr7I, BbsI, BbvI, BbvII, Bbv16II, BccI, BccI, Bce83I, BceAI, BceSIII, BceSIV, Beefl, BcgI, BciVI, Bco5I, Bcol161, BcoDI, BcoKI, BfiI, BfuI, BfuAI, BinI, Bli736I, Bme585I, BmrI, BmsI, BmuI, BpiI, BpmI, BpuAI, BpuEI, BpuSI, BsaI, BsaXI, BsbI, Bsc91I, BscAI, BseKI, BseMI, BseMII, BseRI, BseXI, BseZI, BsgI, BslFI, BsmAI, BsmBI, BsmFI, Bso31I, BsoMAI, Bsp4231, BspCNI, BspD6I, BspIS4I, BspKT5I, BspLU11III, BspQI, BspST5I, BspTNI, BspTS514I, Bst6I, Bst12I, Bst19I, Bst71I, BstBS32I, BstFZ438I, BstGZ53I, BstH9I, BstMAI, BstOZ6161, Bst31TI, BstTS5I, BstV1I, BstV2I, BsuI, Bsu6I, Bsu5371, BtgZI, BtsI, BtsIMutI, BtsCI, BveI, BvelB23I, CatHI, CchII, CchIII, Cco14983111, CdpI, CjeI, CjeF38011111, CjeIAIII, CjeNII, CjeNIII, CjePI, CjeP659IV, CjeYH002IV, CjuII, CseI, CspCI, CstMI, DraRI, DrdIV, EacI, EamI104I, Earl, EciI, Eco311, Eco57I, FaqI, FauI, Fph2801I, GeoICI, GsuI, HgaI, Hin4I, Hin4II, HphI, Hpy99XXII, HpyAV, HpyC1I, Ksp6321, LguI, Lsp11091, LweI, MaqI, MboII, Mcr10I, MlyI, MmeI, MnlI, NcuI, NgoAVII, NgoAVIII, NlaCI, NmeAIII, NmeA6CIII, PciSI, PcoI, PhaI, PlaDI, PleI, PpiI, PpsI, PspOMII, PspPRI, PsrI, RceI, RdeGBII, RlaII, RleAI, RpaI, RpaBI, RpaB5I, Rtr19531, SapI, SchI, SdeAI, SdeOSI, SfaNI, SmuI, SspD5I, SstE37I, Sth1321, StsI, TaqII, TaqIII, TsoI, TspDTI, TspGWI, TstI, Tth111II, TthHB27I, UbaF9I, UbaF11I, UbaF12I, UbaF13I, UbaF14I, Vga4394211, VpaK32I, WviI.

In yet some further embodiments, the Type IIS restriction endonuclease is FokI or any fragment, variant, mutant, fusion protein or conjugate thereof.

In some alternative embodiments, the Type IIS restriction endonuclease applicable for the systems disclosed herein may be at least one of MmeI, MnlI, BfiI or any fragment, variant, mutant, fusion protein or conjugate thereof.

The enzyme FokI (Fok-1), naturally found in Flavobacterium okeanokoites, is a bacterial type IIS restriction endonuclease consisting of an N-terminal DNA-binding domain and a non-specific DNA cleavage domain at the C-terminal. Once the protein is bound to duplex DNA via its DNA-binding domain at the 5′-GGATG-3′ recognition site, the DNA cleavage domain is activated through dimerization and cleaves, without further sequence specificity, the first strand 9 nucleotides downstream and the second strand 13 nucleotides upstream of the nearest nucleotide of the recognition site leaving a typical 4 base overhang. DNA cleavage is mediated through the non-specific cleavage domain which also includes the dimerization surface. The dimer interface is formed by the parallel helices α4 and α5 and two loops P1 and P2 of the cleavage domain. The FokI cleavage domain's molecular mass is 21.8 kDa, being composed of 194 amino acids. In some embodiments, FokI may comprise the amino acid sequence as denoted by SEQ ID NO: 102, or any fragments, derivatives and variants thereof. In yet some further embodiments, a FokI variant useful in the present invention may comprise ancestral mutations. In some specific embodiments such FokI variant may comprise the amino acid sequence as denoted by SEQ ID NO. 103 (also referred to herein as “ancestral FokI”, or as “consensus FokI”). In yet some further embodiments, a FokI variant may comprise the amino acid sequence as denoted by SEQ ID NO. 104 (also referred to herein as “enhanced FokI”). It should be appreciated that the present disclosure further encompasses any variations of the specified FokI variants.

As described herein after, the nucleic acid modifier protein used in the disclosed systems may be based on FokI. In some embodiments, such modifier may be a fusion protein or chimera that may further comprise various additional components. Thus, in some further embodiments, the nucleic acid guided genome modifier chimeric protein of the systems of the present disclosure may further comprise additional elements, for example, at least one cellular localization domain such as Nuclear localization signal (NLS), at least one Mitochondrial leader sequence (MLS), for example, at least one Chloroplast leader sequence; and/or any sequences designed to transport or lead or localize a protein to a nucleic acid containing organelle, a cellular compartment or any subdivision of a cell.

According to some embodiments, a “cellular localization domain” which can localize the nucleic acid guided genome modifier chimeric protein of the invention or a system comprising the modifier/effector chimeric protein and at least one target recognition element, or any complex thereof, to a specific cellular or sub cellular localization in a living cell, may optionally be part of the modifier/effector component of the nucleic acid guided genome modifier chimeric protein of the invention. The cellular localization domain may be constructed by fusing the amino-acid sequence of one of these components to amino-acids incorporating a domain comprising a Nuclear localization signal (NLS); a Mitochondrial leader sequence (MLS); a Chloroplast leader sequence; and/or any sequences designed to transport or lead or localize a protein to a nucleic acid containing organelle, a cellular compartment or any subdivision of a cell. In some exemplary embodiments, the organism is a eukaryotic organism and the cellular localization domain comprises a nuclear localization domain (NLS) which allows the protein access to the nucleus and the genomic DNA within.

In yet some further embodiments, at least one of the nucleic acid modifier component, and at least one component capable of binding the at least one target recognition element of said at least one nucleic acid guided genome modifier protein, chimeric protein, complex or conjugate, comprises at least one clustered regularly interspaced short palindromic repeats (CRISPR)-Cas protein, cas protein derived domain and/or any variant and mutant thereof.

In some embodiments, the at least one nucleic acid guided genome modifier protein used for the systems disclosed herein may comprise at least one component of the CRISPR-Cas system. The Clustered Regularly Interspaced Short Palindromic Repeats (CRISPR) system is a bacterial immune system that has been modified for genome engineering. CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI.

It should be understood that the invention contemplates the use of any of the known CRISPR systems, particularly and of the CRISPR systems disclosed herein. The CRISPR-Cas system has evolved in prokaryotes to protect against phage attack and undesired plasmid replication by targeting foreign DNA or RNA. In bacterial immunity, the CRISPR-Cas system, targets DNA molecules based on short homologous DNA sequences, called spacers that have previously been extracted by the bacterium from the foreign pathogen sequence and inserted between repeats as a memory system. These spacers are transcribed and processed and this RNA, named crRNA or guide-RNA (gRNA), guides CRISPR-associated (Cas) proteins to matching (and/or complementary) sequences within the foreign DNA, called proto-spacers, which are subsequently cleaved.

The spacers, or other suitable constructs or RNAs can be rationally designed and produced to target any DNA sequence. Moreover, this recognition element may be designed separately to recognize and target any desired target including outside of a bacterium.

In some specific embodiment, the CRISPR-Cas proteins used as the at least one nucleic acid guided genome modifier protein in the systems of the present disclosure may be of a CRISPR Class 2 system. In yet some further particular embodiments, such class 2 system may be any one of CRISPR type II, and type V systems. In certain embodiments, the Cas applicable in the present invention may be any Cas protein of the CRISPR type II system. In more specific embodiments, the nucleic acid guided DNA binding protein nuclease may be CRISPR-associated endonuclease 9 (Cas9) system. The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas1 and Cas2. Cas9 contains at least two nuclease domains, a RuvC-like nuclease domain near the amino terminus and the HNH (or McrA-like) nuclease domain in the middle of the protein. It should be appreciated that any type II CRISPR-Cas systems may be applicable in the present invention, specifically, any one of type II-A or B. Thus, in yet some further and alternative embodiments, at least one cas gene used in the methods and systems of the invention may be at least one cas gene of type II CRISPR system (either typeII-A or typeII-B). In more particular embodiments, at least one cas gene of type II CRISPR system used by the methods and systems of the invention may be the cas9 gene.

According to such embodiments, the CRISPR-Cas proteins used in the systems of the invention is a CRISPR-associated endonuclease 9 (Cas9). Double-stranded DNA (dsDNA) cleavage by Cas9 is a hallmark of “type II CRISPR-Cas” immune systems. The CRISPR-associated protein Cas9 is an RNA-guided DNA endonuclease that uses RNA:DNA complementarity to a target site (proto-spacer). After recognition between Cas9 and the target sequence double stranded DNA (dsDNA) cleavage occur, creating the double strand breaks (DSBs).

CRISPR type II system as used herein requires the inclusion of two essential components: a “guide” RNA (gRNA) and a CRISPR-associated endonuclease (Cas9). The gRNA is an RNA molecule composed of a “scaffold” sequence necessary for Cas9-binding (also named tracrRNA) and about 20 nucleotide long “spacer” or “targeting” sequence, which defines the genomic target to be modified. Guide RNA (gRNA), as used herein refers to a synthetic fusion or alternatively, annealing of the endogenous tracrRNA with a targeting sequence (also named crRNA), providing both scaffolding/binding ability for Cas9 nuclease and targeting specificity. Also referred to as “single guide RNA” or “sgRNA” or as a specificity conferring nucleic acid (SCNA).

In yet some further particular embodiments, the class 2 system in accordance with the invention, may be a CRISPR type V system. In a more specific embodiment, the RNA guided DNA binding protein nuclease may be CRISPR-associated endonuclease X (CasX) system or CRISPR-associated endonuclease 14 (Cas14) system or CRISPR-associated endonuclease F (CasF, also known as Cas12j) system. The type V CRISPR-Cas systems are distinguished by a single RNA-guided RuvC domain-containing nuclease. As with type II CRISPR-Cas systems, CRISPR type V system as used herein requires the inclusion of two essential components: a gRNA and a CRISPR-associated endonuclease (CasX/Cas14/CasF). The gRNA is a short synthetic RNA composed of a “scaffold” sequence necessary for CasX/Cas14/CasF-binding and about 20 nucleotide long “spacer” or “targeting” sequence, which defines the genomic target to be modified.

In yet some alternative embodiments, where the modifier used performs a modulation other than nucleolytic activity, directing the modifier to the target site may result in targeted modulation (e.g., activation or repression, methylation or demethylation and the like) of the target nucleic acid sequence targeted by the target recognition element. It should be noted that a target recognition element may comprise between about 3 nucleotides to about 100 nucleotides, specifically, 3, 4, 5, 6, 7, 8, 9, 10, 15, 20, 25, 30, 35, 40, 45, 50, 55, 60, 65, 70, 75, 80, 85, 90, 95 100 or more. More specifically between about 10 nucleotides to 70 nucleotides or more.

It should be noted that any CRISPR/Cas proteins may be used by the invention, in some embodiments of the present disclosure, the endonuclease may be a Cas9, CasX, Cas12, Cas13, Cas14, Cas6, Cpf1, CMS1 protein, or any variant thereof that is derived or expressed from Methanococcus maripaludis C7, Corynebacterium diphtheria, Corynebacterium efficiens YS-314, Corynebacterium glutamicum (ATCC 13032), Corynebacterium glutamicum (ATCC 13032), Corynebacterium glutamicum R, Corynebacterium kroppenstedtii (DSM 44385), Mycobacterium abscessus (ATCC 19977), Nocardia farcinica IFM10152, Rhodococcus erythropolis PR4, Rhodococcus jostii RFIA1, Rhodococcus opacus B4 (uid36573), Acidothermus cellulolyticus 11 B, Arthrobacter chlorophenolicus A6, Kribbella flavida (DSM 17836), Thermomonospora curvata (DSM43183), Bifidobacterium dentium Bd1, Bifidobacterium longum DJO10A, Slackia heliotrinireducens (DSM 20476), Persephonella marina EX H 1, Bacteroides fragilis NCTC 9434, Capnocytophaga ochracea (DSM 7271), Flavobacterium psychrophilum JIP02 86, Akkermansia muciniphila (ATCC BAA 835), Roseiflexus castenholzii (DSM 13941), Roseiflexus RS1, Synechocystis PCC6803, Elusimicrobium minutum Pei191, uncultured Termite group 1 bacterium phylotype Rs D17, Fibrobacter succinogenes S85, Bacillus cereus (ATCC 10987), Listeria innocua, Lactobacillus casei, Lactobacillus rhamnosus GG, Lactobacillus salivarius UCC118, Streptococcus agalactiae-5-A909, Streptococcus agalactiae NEM316, Streptococcus agalactiae 2603, Streptococcus dysgalactiae equisimilis GGS 124, Streptococcus equi zooepidemicus MGCS10565, Streptococcus gallolyticus UCN34 (uid46061), Streptococcus gordonii Challis subst CH1, Streptococcus mutans NN2025 (uid46353), Streptococcus mutans, Streptococcus pyogenes M1 GAS, Streptococcus pyogenes MGAS5005, Streptococcus pyogenes MGAS2096, Streptococcus pyogenes MGAS9429, Streptococcus pyogenes MGAS 10270, Streptococcus pyogenes MGAS6180, Streptococcus pyogenes MGAS315, Streptococcus pyogenes SSI-1, Streptococcus pyogenes MGAS10750, Streptococcus pyogenes NZ131, Streptococcus thermophiles CNRZ1066, Streptococcus thermophiles LMD-9, Streptococcus thermophiles LMG 18311, Clostridium botulinum A3 Loch Maree, Clostridium botulinum B Eklund 17B, Clostridium botulinum Ba4 657, Clostridium botulinum F Langeland, Clostridium cellulolyticum H10, Finegoldia magna (ATCC 29328), Eubacterium rectale (ATCC 33656), Mycoplasma gallisepticum, Mycoplasma mobile 163K, Mycoplasma penetrans, Mycoplasma synoviae 53, Streptobacillus, moniliformis (DSM 12112), Bradyrhizobium BTAi1, Nitrobacter hamburgensis X14, Rhodopseudomonas palustris BisB18, Rhodopseudomonas palustris BisB5, Parvibaculum lavamentivorans DS-1, Dinoroseobacter shibae. DFL 12, Gluconacetobacter diazotrophicus Pal 5 FAPERJ, Gluconacetobacter diazotrophicus Pal 5 JGI, Azospirillum B510 (uid46085), Rhodospirillum rubrum (ATCC 11170), Diaphorobacter TPSY (uid29975), Verminephrobacter eiseniae EFO1-2, Neisseria meningitides 053442, Neisseria meningitides alpha14, Neisseria meningitides Z2491, Desulfovibrio salexigens DSM 2638, Campylobacter jejuni doylei 269 97, Campylobacter jejuni 81116, Campylobacter jejuni, Campylobacter lari RM2100, Helicobacter hepaticus, Wolinella succinogenes, Tolumonas auensis DSM 9187, Pseudoalteromonas atlantica T6c, Shewanella pealeana (ATCC 700345), Legionella pneumophila Paris, Actinobacillus succinogenes 130Z, Pasteurella multocida, Francisella tularensis novicida U 112, Francisella tularensis holarctica, Francisella tularensis FSC 198, Francisella tularensis, Francisella tularensis WY96-3418, or Treponema denticola (ATCC 35405).

In some embodiments, the at least one nucleic acid guided genome modifier protein of the systems of the present disclosure, and specifically chimeras thereof, may comprise at least one defective enzyme. A defective enzyme (e.g., a defective mutant, variant or fragment) may relate to an enzyme that displays an activity reduced in about 1%, 2%, 3%, 4%, 5% to about 100%, specifically, about 5% to about 10%, about 10% to about 15%, about 15% to about 20%, about 20% to about 25%, about 25% to about 30%, about 35% to about 40%, about 40% to about 45%, about 45% to about 50%, about 50% to about 55%, about 55% to about 60%, about 65% to about 70%, about 75% to about 80%, about 80% to about 85%, about 85% to about 90%, about 90% to about 95%, about 95% to about 99.9%, more specifically, reduced activity of about 98% to about 100%, as compared to the wild type active nuclease. More specifically, an enzyme that displays an activity reduced in about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, 99.9999% or about 100%, as compared to the wild type active nuclease.

In some embodiments, the at least one nucleic acid guided genome modifier protein used in the systems of the present disclosure may be or may comprise a CRISPR-Cas protein or cas protein derived domain having reduced or abolished Protospacer Adjacent Motif (PAM) constraint or any fragment, variant, mutant, fusion/chimeric protein, complex or conjugate thereof. In more specific embodiments, at least one of the PAM binding domain (PBD) and/or the PAM recognition motif, and/or the HNH-nuclease domain of the Cas protein, any fragment of the PBD, and/or of the PAM recognition motif, and/or of the HNH-nuclease domain, and at least one amino acid residue adjacent to the PBD, and/or to the PAM recognition motif, and/or to the HNH-nuclease domain, is deleted, substituted, mutated or replaced.

The protospacer adjacent motif (PAM), as used herein, is a short DNA sequence (usually 2-6 base pairs in length) that follows the DNA region targeted for cleavage by the CRISPR system, serving as a binding signal. The PAM is required for a Cas nuclease to cut and is generally found 3-4 nucleotides downstream from the cut site. The canonical PAM (of SpCas9) is the sequence 5′-NGG-3′ where “N” is any nucleobase followed by two guanine (“G”) nucleobases. This short DNA sequence, the PAM, is frequently used to mark proper target sites and discriminate between ‘self’ and ‘non-self’ potential target sequences.

The Cas proteins used by the disclosed systems display reduced or abolished PAM restriction, constraint, requirement or limitation. More specifically, a Cas protein displaying a “PAM-reduced” or “PAM abolished” requirement, restriction, constraint, requirement or limitation as used herein, is considered as a Cas protein having either (a) a less stringent PAM requirement than that of the wild-type PAM requirement of the corresponding wild-type Cas protein; or (b) substitution of a PAM-requiring Cas protein by a less stringent Cas protein or portion thereof. It should be noted that the fewer nucleotides that are required for recognition, the less stringent the PAM is. In some embodiments, PAM-reduced or abolished Cas protein may require three or less, two or less or one or less nucleotides in a PAM sequence adjacent to the target site. In certain embodiments, PAM-reduced or abolished Cas protein binds the target site recognized by the targeting elements, with no further requirements of specific nucleotides in sequences adjacent to the target site. In some embodiments a PAM-reduced Cas protein, may be a protein that display reduced restriction, constraint, requirement or limitation in about 1%, 2%, 3%, 4%, 5% to about 100%, specifically, about 5% to about 10%, about 10% to about 15%, about 15% to about 20%, about 20% to about 25%, about 25% to about 30%, about 35% to about 40%, about 40% to about 45%, about 45% to about 50%, about 50% to about 55%, about 55% to about 60%, about 65% to about 70%, about 75% to about 80%, about 80% to about 85%, about 85% to about 90%, about 90% to about 95%, about 95% to about 99.9%, more specifically, reduced, inhibited, decreased, eliminated, restriction, constraint, requirement or limitation of about 98% to about 100% as compared to that of the wild-type PAM requirement of the corresponding wild-type Cas protein. More specifically, a protein that display reduced restriction, constraint, requirement or limitation in about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, 99.9999% or about 100%, as compared to that of the wild-type PAM requirement of the corresponding wild-type Cas protein. It should be further noted that a “PAM-free” system would have no PAM requirement. Thus, in some embodiments, the invention further provides systems comprising Cas protein variants that display no PAM requirement, referred to herein as PAM-abolished or PAM-free Cas proteins.

As indicated above, the systems of the invention use and comprise Cas protein-derived domains that display PAM-reduced or abolished PAM restriction, constraint, requirement or limitation. In some embodiments such domain may comprise any domain of the Cas protein that maintain the ability of such Cas protein to bind at least one target recognition element (e.g., gRNA), that guide and direct the Cas protein to a predetermined target nucleic acid sequence. In some embodiments, a cas-protein-derived domain applicable in the present invention may be any protein fragment of Cas comprising between about 50 to 500 amino acid residues or more, that display at least 50-100% homology or identity to at least 50 consecutive amino acid residues of the entire Cas-protein.

In some alternative embodiments, the nucleic acid modifier protein that can be used in the disclosed systems, may be a nucleic acid guided genome modifier chimeric or fusion protein, complex or conjugate having enhanced homology-directed repair. More specifically, the nucleic acid guided genome modifier protein of the invention may comprise: (a) at least one defective CRISPR-Cas (CRISPR-dCas) protein devoid of a nucleolytic activity or any variant or mutant thereof; and (b) at least one nucleic acid modifier component. The nucleic acid guided genome modifier chimeric or fusion protein of the systems of the present disclosure further comprises at least one of: (c) at least one donor attachment domain (DAD) for binding at least one Donor nucleic acid molecule; and/or (d) at least one repair factor recruitment domain (RFRD).

In some embodiments, the DAD of such nucleic acid modifier protein used in the disclosed systems, may be at least one of a sequence specific donor attachment domain, a non-sequence specific donor attachment domain and a covalent interaction domain.

In some further embodiments, the DAD may be a sequence specific DAD comprising at least one of a zinc finger DNA binding domain, a lambda repressor DNA binding domain, a Gal4 DNA binding domain and a protection of telomeres 1 protein (Pot1) ssDNA binding domain. In some embodiments, Cys₂His₂ zinc fingers, may be used in this regard.

Specifically, any one of the Zif-QQR and Zif-QNK.

In some further embodiments, the DAD may be a covalent interaction domain comprising a virD2 domain.

In some further embodiments, the DAD may be a non-sequence specific donor attachment domain, for example, at least one domain of an affinity pair. In some embodiments, the affinity pair is avidin-biotin (e.g., streptavidin domain for biotinylated donor molecules).

In yet some further embodiments, any tag-anti-tag pair (including antigen-antibody, or ligand-receptor may be used). In some particular embodiments, non-sequence specific donor attachment domain may comprise a streptavidin domain. In yet some further embodiments, binding-pair may further include Agrobacterium VirD2-VirD2 binding protein; antibody-antigen; single chain antibody-antigen interaction; anti-Fluorescein single-chain variable fragment antibody (anti-FAM ScFV), Fluorescein; anti-DIG single-chain variable fragment (scFv) immunoglobin (DIG-ScFv), Digoxigenin (DIG) and IgG-protein A.

In some embodiments, the Cas protein is at least one of Cas9, CasX, Cas14a1, Cas14b5, CasF, an ancestral Cas9, and Cas12a.

In yet some further embodiments, the at least one nucleic acid guided genome modifier protein, chimeric protein, complex or conjugate, comprised within the system of the present disclosure may comprise at least one CRISPR-Cas protein or cas protein derived domain having reduced or abolished Protospacer Adjacent Motif (PAM) constraint or any variant, mutant, fusion/chimeric protein, complex or conjugate thereof, wherein at least one of: the PAM binding domain (PBD) and/or the PAM recognition motif, and/or the HNH-nuclease domain of the Cas protein, any fragment of the PBD, and/or of said PAM recognition motif, and/or of the HNH-nuclease domain, and at least one amino acid residue adjacent to the PBD, and/or to the PAM recognition motif, and/or to the HNH-nuclease domain of the Cas protein is deleted, replaced or substituted.

Still further, in some embodiments, the Cas protein is at least one of Streptococcus canis Cas9 (ScCas9), Streptococcus pyogenes Cas9 (SpCas9), an ancestral Cas9, deltaproteobacteria CasX, Cas12a, CasF-1, CasF-2, CasF-3, Cas14a1, or Cas14b5, and wherein at least one PAM-interacting Arginine and/or Lysine residue of the PBD of said Cas protein is deleted, substituted or replaced.

In some particular embodiments, the nucleic acid guided genome modifier is a chimeric protein. In some embodiments, such chimeric protein useful in the present disclosure may comprise at least one Cas protein, specifically, a defective or deficient Cas nuclease (dCas), and FokI. More specifically, endonuclease deficient Cas9, is a mutant form of Cas9, whose endonuclease activity is removed by mutating the endonuclease domains.

The dCas9 can still bind to its guide RNA (target recognition nucleic acid molecule), and the DNA strand that is being targeted. Still further, in some specific and non-limiting embodiments, the chimeric protein useful as the modifier protein of the disclose systems is any one of the following chimeras: dScCasFok, SV40 NLS; dScCasFok, SV40+SV40 bipartite NLS; dCasFok, FokI consensus, SV40+bipartiteSV40; dCasFok, FokI consensus, SV40+bipartiteSV40, ancestral mutations in RuvC+REC domain, HNH deletion; dScCasFok, SV40+nucleoplasmin, ancestral mutations in RuvC+REC domain, Scloop QQmutation HNH deletion, whole PAMBD replaced with LacI DNA binding domain; dScCasFok, SV40+nucleoplasmin, ancestral mutations in RuvC+REC domain, Scloop SpReplacement, HNH deletion, PAMBD loop replaced with Zinc finger; dScCasFok, SV40+nucleoplasmin, ancestral mutations in RuvC+REC domain, Scloop SpReplacement HNH deletion, whole PAMBD replaced with SSO7D; dScCasFok, SV40+nucleoplasmin, ancestral mutations in RuvC+REC domain, Scloop SpReplacement HNH deletion, PAMBD loop replaced with HMGN; and dScCasFok, SV40+nucleoplasmin, ancestral mutations in RuvC+REC domain, Scloop SpReplacement HNH deletion, whole PAMBD replaced with STO7.

In yet some further embodiments, the nucleic acid guided genome modifier chimeric or fusion protein, complex or conjugate used in the system of the present disclosure may display enhanced homology-directed repair (HDR). In some specific embodiments, such nucleic acid guided genome modifier protein may comprise: (a) at least one defective CRISPR-Cas protein (CRISPR-dCas) devoid of a nucleolytic activity or any variant or mutant thereof; and (b) at least one nucleic acid modifier component; and at least one of: (c) at least one donor attachment domain (DAD) for binding at least one Donor nucleic acid molecule; and (d) at least one repair factor recruitment domain (RFRD).

In some specific embodiments, nucleic acid guided genome modifier chimeric or fusion protein used by the systems of the present disclosure is a chimeric protein, that is HDR enhanced. It should be appreciated that the HDR enhanced nucleic acid guided genome modifier chimeric or fusion protein of the present disclosure may be also referred to as the “chimeric protein of the present disclosure”, and also as the “machine”, as “HDR enhanced nucleic acid guided genome modifier”, and the like.

In some specific and non-limiting embodiments, the nucleic acid guided genome modifier used in the disclosed systems, compositions, cells and methods is a chimeric protein, said chimeric protein is any one of: dScCas9-FokI-ZFQ variant; dScCas9-FokI-Lam variant; dScCas9-FokI-Strep variant; dScCas9-FokI-Vir variant; dScCas9-FokI-BRCA2 variant; dScCas9-FokI-DSS1 variant; dScCas9-FokI-BRCA2-Strep variant; dScCas9-FokI-DSS1-Strep variant; dScCas9-FokI-BRCA2-virD2 variant; dScCas9-FokI-DSS1-virD2 variant; dCas9-BfiI variant, dCas9-MnlI variant; dCas9-MmeI variant; dCas9-FokI-RAD54ntd variant; dCasFok-BRCA2 3NLS variant; dCasFok-DSS1 3NLS variant; dCasFok-ZFQ 1NLS variant; dCasFok-Strep 1NLS variant; dCasFok, 2NLS, N-terminal BRCA2 variant; dCasFok, 2NLS, N-terminal BRCA2, 6His variant; dCasFok, 2NLS, N-terminal Streptavidin variant; dCasFok, 2NLS, N-terminal Streptavidin, 6His variant; dCasFok, 2NLS, N-terminal Pot variant; dCasFok, 2NLS, N-terminal Pot, 6His variant; dCasFok, 1NLS, N-terminal Streptavidin, C-terminal BRCA2 variant; dCasFok, 1NLS, N-terminal Pot, C-terminal BRCA2 variant; dCasFok, 1NLS, ancestral RuvC+REC1/2, N- and C-terminal BRCA2 variant; dCasFok, 2NLS variant, ancestral RuvC+REC1/2, N-terminal BRCA2 variant; dCasFok, 2NLS, ancestral RuvC+REC1/2, N-terminal BRCA2, 6His variant; ancestral dCas9-FokI-RAD52id variant; DSS1 peptide (n-term), ancestral dCasFok variant; BRCA2 peptide 2(N-term), ancestral dCasFok variant; RAD52 peptide (n-term), ancestral dCasFok variant; Streptavidin (n-term), ancestral dCasFok variant; BRCA2 peptide (c-term), ancestral dCasFok variant; DSS1 peptide (c-term), ancestral dCasFok variant; BRCA2 peptide 2 (c-term), ancestral dCasFok variant; RAD54 peptide (c-term), ancestral dCasFok; RAD52 peptide (c-term), ancestral dCasFok variant; Mdm2 peptide (c-term), ancestral dCasFok variant; Streptavidin (c-term), ancestral dCasFok variant; BRCA2 peptide (n-term), ancestral dCasFok, his-tagged variant; DSS1 peptide (n-term), ancestral dCasFok, his-tagged variant; BRCA2 peptide 2(n-term), ancestral dCasFok, his-tagged variant; RAD54 peptide (n-term), ancestral dCasFok, his-tagged variant; RAD52 peptide (n-term), ancestral dCasFok, his-tagged variant; Streptavidin (n-term), ancestral dCasFok, his-tagged variant; BRCA2 peptide (n and c-term), ancestral dCasFok variant; BRCA2 peptide (n-term), DSS1 peptide (c-term), ancestral dCasFok variant; BRCA2 peptide (n-term), BRCA2 peptide 2 (c-term), ancestral dCasFok variant; BRCA2 peptide (n-term), RAD52 peptide (c-term), ancestral dCasFok variant; BRCA2 peptide (n-term), Mdm2 peptide (c-term), ancestral dCasFok variants; BRCA2 peptide (n-term), Streptavidin (c-term), ancestral dCasFok variant; DSS1 peptide (n-term), BRCA2 peptide (c-term), ancestral dCasFok variant; DSS1 peptide (n-term), BRCA2 peptide 2 (c-term), ancestral dCasFok variant; DSS1 peptide (n-term), RAD54 peptide (c-term), ancestral dCasFok variant; DSS1 peptide (n-term), Streptavidin (c-term), ancestral dCasFok variant; Streptavidin (n-term), BRCA2 peptide (c-term), ancestral dCasFok variant; Streptavidin (n-term), DSS1 peptide (c-term), ancestral dCasFok variant; Streptavidin (n-term), BRCA2 peptide 2 (c-term), ancestral dCasFok variant; Streptavidin (n-term), RAD52 peptide (c-term), ancestral dCasFok variant; and ancestral dCasFok, his-tagged variant. In more specific and non-limiting embodiments, the nucleic acid guided genome modifier used in the disclosed systems, compositions, cells and methods may be any of the modifiers and machines disclosed herein, having the amino acid sequences as denoted by any one of SEQ ID NO: 111 TO 174. In some embodiments, dScCas9-FokI-ZFQ variant may comprise an amino acid sequence as denoted by SEQ ID NO. 111. In some embodiments, dScCas9-FokI-Lam variant (dScCas9-FokI fused to Lambda repressor DNA binding domain) may comprise an amino acid sequence as denoted by SEQ ID NO. 112. In some embodiments, dScCas9-FokI-Strep variant (dScCas9-FokI fused to monomeric streptavidin) may comprise an amino acid sequence as denoted by SEQ ID NO. 113. In some embodiments, dScCas9-FokI-Vir variant (dScCas9-FokI fused to monomeric virD2) may comprise an amino acid sequence as denoted by SEQ ID NO. 114. In some embodiments, dScCas9-FokI-BRCA2 variant may comprise an amino acid sequence as denoted by SEQ ID NO. 115. In some embodiments, dScCas9-FokI-DSS1 variant may comprise an amino acid sequence as denoted by SEQ ID NO. 116. In some embodiments, dScCas9-FokI-BRCA2-Strep variant (dScCas9-FokI fused to a BRCA2 peptide and to a monomeric streptavidin) may comprise an amino acid sequence as denoted by SEQ ID NO. 117. In some embodiments, dScCas9-FokI-DSS1-Strep variant (dScCas9-FokI fused to a DSS1 peptide and to a monomeric streptavidin), as denoted by SEQ ID NO. 118. In some embodiments, dScCas9-FokI-BRCA2-virD2 variant (dScCas9-FokI fused to a BRCA2 peptide and to a monomeric virD2) may comprise an amino acid sequence as denoted by SEQ ID NO. 119. In some embodiments, dScCas9-FokI-DSS1-virD2 variant (dScCas9-FokI fused to a DSS1 peptide and to a monomeric virD2) may comprise an amino acid sequence as denoted by SEQ ID NO. 120. In some embodiments, dCas9-BfiI variant may comprise an amino acid sequence as denoted by SEQ ID NO: 121. In some embodiments, dCas9-MnlI variant may comprise an amino acid sequence as denoted by SEQ ID NO:122. In some embodiments, dCas9-MmeI variant may comprise an amino acid sequence as denoted by SEQ ID NO: 123. In some embodiments, dCas9-FokI-RAD54ntd variant may comprise an amino acid sequence as denoted by SEQ ID NO: 124. In some embodiments, ancestral dCas9-FokI-RAD54ntd variant (ancestral dCas9-FokI fused to a RAD54 N-terminal domain peptide) may comprise an amino acid sequence as denoted by SEQ ID NO: 125. In some embodiments, dCasFok-BRCA2, 3NLS variant may comprise an amino acid sequence as denoted by SEQ ID NO: 126. In some embodiments, dCasFok-DSS1, 3NLS variant may comprise an amino acid sequence as denoted by SEQ ID NO: 127. In some embodiments, dCasFok-ZFQ, 1NLS variant may comprise an amino acid sequence as denoted by SEQ ID NO: 128. In some embodiments, dCasFok-Strep, 1NLS variant may comprise an amino acid sequence as denoted by SEQ ID NO: 129. In some embodiments, dCasFok, 2NLS, N-terminal BRCA2 variant may comprise an amino acid sequence as denoted by SEQ ID NO:130. In some embodiments, dCasFok, 2NLS, N-terminal BRCA2, 6His variant may comprise an amino acid sequence as denoted by SEQ ID NO:131. In some embodiments, dCasFok, 2NLS, N-terminal Streptavidin variant may comprise an amino acid sequence as denoted by SEQ ID NO:132. In some embodiments, dCasFok, 2NLS, N-terminal Streptavidin, 6His variant may comprise an amino acid sequence as denoted by SEQ ID NO: 133. In some embodiments, dCasFok, 2NLS, N-terminal Pot variant may comprise an amino acid sequence as denoted by SEQ ID NO:134. In some embodiments, dCasFok, 2NLS, N-terminal Pot, 6His variant may comprise an amino acid sequence as denoted by SEQ ID NO:135. In some embodiments, dCasFok, 1NLS, N-terminal Streptavidin, C-terminal BRCA2 variant may comprise an amino acid sequence as denoted by SEQ ID NO: 136. In some embodiments, dCasFok, 1NLS, N-terminal Pot, C-terminal BRCA2 variant may comprise an amino acid sequence as denoted by SEQ ID NO:137. In some embodiments, dCasFok, 1NLS, ancestral RuvC+REC1/2, N- and C-terminal BRCA2 variant may comprise an amino acid sequence as denoted by SEQ ID NO:136. In some embodiments, dCasFok, 2NLS, ancestral RuvC+REC1/2, N-terminal BRCA2 variant may comprise an amino acid sequence as denoted by SEQ ID NO: 139.

In some embodiments, dCasFok, 2NLS, ancestral RuvC+REC1/2, N-terminal BRCA2, 6His variant may comprise an amino acid sequence as denoted by SEQ ID NO:140. In some embodiments, ancestral dCas9-FokI-RAD52id variant may comprise an amino acid sequence as denoted by SEQ ID NO: 141; In some embodiments, DSS1 peptide (n-term), ancestral dCasFok variant may comprise an amino acid sequence as denoted by SEQ ID NO: 142; BRCA2 peptide 2(N-term), ancestral dCasFok variant may comprise an amino acid sequence as denoted by SEQ ID NO: 143; In some embodiments, RAD52 peptide (n-term), ancestral dCasFok variant may comprise an amino acid sequence as denoted by SEQ ID NO: 144; In some embodiments, Streptavidin (n-term), ancestral dCasFok variant may comprise an amino acid sequence as denoted by SEQ ID NO: 145; In some embodiments, BRCA2 peptide (c-term), ancestral dCasFok variant may comprise an amino acid sequence as denoted by SEQ ID NO: 146; In some embodiments, DSS1 peptide (c-term), ancestral dCasFok variant may comprise an amino acid sequence as denoted by SEQ ID NO: 147; In some embodiments, BRCA2 peptide 2 (c-term), ancestral dCasFok variant may comprise an amino acid sequence as denoted by SEQ ID NO: 148; In some embodiments, RAD54 peptide (c-term), ancestral dCasFok variant may comprise an amino acid sequence as denoted by SEQ ID NO: 149; RAD52 peptide (c-term), ancestral dCasFok variant may comprise an amino acid sequence as denoted by SEQ ID NO: 150; In some embodiments, Mdm2 peptide (c-term), ancestral dCasFok variant may comprise an amino acid sequence as denoted by SEQ ID NO: 151; In some embodiments, Streptavidin (c-term), ancestral dCasFok variant may comprise an amino acid sequence as denoted by SEQ ID NO: 152; In some embodiments, BRCA2 peptide (n-term), ancestral dCasFok, his-tagged variant may comprise an amino acid sequence as denoted by SEQ ID NO: 153; In some embodiments, DSS1 peptide (n-term), ancestral dCasFok, his-tagged variant may comprise an amino acid sequence as denoted by SEQ ID NO: 154; In some embodiments, BRCA2 peptide 2(n-term), ancestral dCasFok, his-tagged variant may comprise an amino acid sequence as denoted by SEQ ID NO: 155; In some embodiments, RAD54 peptide (n-term), ancestral dCasFok, his-tagged variant may comprise an amino acid sequence as denoted by SEQ ID NO: 156; In some embodiments, RAD52 peptide (n-term), ancestral dCasFok, his-tagged variant may comprise an amino acid sequence as denoted by SEQ ID NO: 157; In some embodiments, Streptavidin (n-term), ancestral dCasFok, his-tagged variant may comprise an amino acid sequence as denoted by SEQ ID NO: 158; In some embodiments, BRCA2 peptide (n and c-term), ancestral dCasFok variant may comprise an amino acid sequence as denoted by SEQ ID NO: 159; BRCA2 peptide (n-term), DSS1 peptide (c-term), ancestral dCasFok variant may comprise an amino acid sequence as denoted by SEQ ID NO: 160; In some embodiments, BRCA2 peptide (n-term), BRCA2 peptide 2 (c-term), ancestral dCasFok variant may comprise an amino acid sequence as denoted by SEQ ID NO: 161; In some embodiments, BRCA2 peptide (n-term), RAD52 peptide (c-term), ancestral dCasFok variant may comprise an amino acid sequence as denoted by SEQ ID NO: 162; In some embodiments, BRCA2 peptide (n-term), Mdm2 peptide (c-term), ancestral dCasFok variants may comprise an amino acid sequence as denoted by SEQ ID NO: 163; In some embodiments, BRCA2 peptide (n-term), Streptavidin (c-term), ancestral dCasFok variant may comprise an amino acid sequence as denoted by SEQ ID NO: 164; In some embodiments, DSS1 peptide (n-term), BRCA2 peptide (c-term), ancestral dCasFok variant may comprise an amino acid sequence as denoted by SEQ ID NO: 165; In some embodiments, DSS1 peptide (n-term), BRCA2 peptide 2 (c-term), ancestral dCasFok variant may comprise an amino acid sequence as denoted by SEQ ID NO: 166; In some embodiments, DSS1 peptide (n-term), RAD54 peptide (c-term), ancestral dCasFok variant may comprise an amino acid sequence as denoted by SEQ ID NO: 167; DSS1 peptide (n-term), Streptavidin (c-term), ancestral dCasFok variant may comprise an amino acid sequence as denoted by SEQ ID NO: 168; In some embodiments, Streptavidin (n-term), BRCA2 peptide (c-term), ancestral dCasFok variant may comprise an amino acid sequence as denoted by SEQ ID NO: 169; In some embodiments, Streptavidin (n-term), DSS1 peptide (c-term), ancestral dCasFok variant may comprise an amino acid sequence as denoted by SEQ ID NO: 170; Streptavidin (n-term), BRCA2 peptide 2 (c-term), ancestral dCasFok variant may comprise an amino acid sequence as denoted by SEQ ID NO: 171; In some embodiments, Streptavidin (n-term), RAD52 peptide (c-term), ancestral dCasFok variant may comprise an amino acid sequence as denoted by SEQ ID NO: 172.

In some embodiments, ancestral dCasFok, his-tagged variant may comprise an amino acid sequence as denoted by SEQ ID NO: 173. In yet some further embodiments, the effector may be the Cas9, as denoted for example by the amino acid sequence as denoted by SEQ ID NO: 174.

As indicated above, the systems of the present disclosure comprise at least one donor nucleic acid molecule. The term “nucleic acid”, “nucleic acid sequence”, or “polynucleotide” and “nucleic acid molecule” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides, wherein the attachment of various entities or moieties to the nucleotide units at any position are included. The terms should also be understood to include, as equivalents, analogs of either RNA or DNA made from nucleotide analogs, and, as applicable to the embodiment being described, single-stranded (such as sense or antisense) and double-stranded polynucleotides. Preparation of nucleic acids is well known in the art.

It should be noted that the nucleic acid molecules (or polynucleotides) according to the invention can be produced synthetically, or by recombinant DNA technology. Methods for producing nucleic acid molecules are well known in the art.

The nucleic acid molecule according to the invention may be of a variable nucleotide length. For example, in some embodiments, the nucleic acid molecule according to the invention comprises 1-100 nucleotides, e.g., about 10, 20, 30, 40, 50, 60, 70, 80, 90 or 100 nucleotides. In other embodiments the nucleic acid molecule according to the invention comprises 100-1,000 nucleotides, e.g., about 100, 200, 300, 400, 500, 600, 700, 800, 900 or 1000 nucleotides. In further embodiments the nucleic acid molecule according to the invention comprises 1,000-10,000 nucleotides, e.g., about 1,000, 2,000, 3,000, 4,000, 5,000, 6,000, 7,000, 8,000, 9,000 or 10,000 nucleotides. In yet further embodiments the nucleic acid molecule according to the invention comprises more than 10,000 nucleotides, for example, 20,000, 30,000, 40,000, 50,000, 60,000, 70,000, 80,000, 90,000 or 100,000 nucleotides.

Still further, the donor nucleic acid molecules of the systems of the invention or any cassettes thereof, or any of the disclosed systems, may be comprised within vector/s. It should be noted that the methods for immune modulation and treatment disclosed herein after are based on contacting the cells, or administering to the treated subject, the disclosed systems or any cassettes, constructs or vectors comprising the system or any donor nucleic acid molecules thereof. Vector/s, as used herein, are nucleic acid molecules of particular sequence that can be introduced into a host cell, thereby producing a transformed host cell or be transiently expressed in the cell. A vector may include nucleic acid sequences that permit it to replicate in a host cell, such as an origin of replication. A vector may also include one or more selectable marker genes and other genetic elements known in the art, including promoter elements that direct nucleic acid expression. Many vectors, e.g. plasmids, cosmids, minicircles, phage, viruses, (as detailed below) useful for transferring nucleic acids into target cells may be applicable in the present invention. The vectors comprising the nucleic acid(s) may be maintained episomally, e.g. as plasmids, minicircle DNAs, viruses such cytomegalovirus, adenovirus, or they may be integrated into the target cell genome, through homologous recombination or random integration, e.g. retrovirus-derived vectors such as AAV, MMLV, HIV-1, ALV, etc.

As indicated above, in some embodiments, viral vectors may be applicable in the present invention. The term “viral vector” refers to a replication competent or replication-deficient viral particle which are capable of transferring nucleic acid molecules into a host.

In some embodiments such viral vectors may be used for transient expression of the components of the invention in the cell and may or may not be present in the cells ultimately delivered to the patient.

The term “virus” refers to any of the obligate intracellular parasites having no protein-synthesizing or energy-generating mechanism. The viral genome may be RNA or DNA contained with a coated structure of protein of a lipid membrane. Examples of viruses useful in the practice of the present invention include baculoviridiae, parvoviridiae, picornoviridiae, herepesviridiae, poxviridiae, adenoviridiae, picotmaviridiae. The term recombinant virus includes chimeric (or even multimeric) viruses, i.e., vectors constructed using complementary coding sequences from more than one viral subtype. In yet some particular embodiments, such viral vector may be any one of recombinant adeno associated vectors (rAAV), single stranded AAV (ssAAV), self-complementary rAAV (scAAV), Simian vacuolating virus 40 (SV40) vector, Adenovirus vector, helper-dependent Adenoviral vector, retroviral vector and lentiviral vector.

More specifically, in some embodiments, the donor nucleic acid molecules suitable to systems, compositions, cells and methods of the invention may be comprised within an Adeno-associated virus (AAV). The term “adenovirus” is synonymous with the term “adenoviral vector”. AAV is a single-stranded DNA virus with a small (˜20 nm) protein capsule that belongs to the family of parvoviridae, and specifically refers to viruses of the genus adenoviridiae. The term adenoviridiae refers collectively to animal adenoviruses of the genus mastadenovirus including but not limited to human, bovine, ovine, equine, canine, porcine, murine and simian adenovirus subgenera. In particular, human adenoviruses includes the A-F subgenera as well as the individual serotypes thereof the individual serotypes and A-F subgenera including but not limited to human adenovirus types 1, 2, 3, 4, 4a, 5, 6, 7, 8, 9, 10, 11 (AdllA and Ad IIP), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91.

Due to its inability to replicate in the absence of helpervirus coinfections (typically Adenovirus or Herpesvirus infections) AAV is often referred to as dependovirus. AAV infections produce only mild immune responses and are considered to be nonpathogenic, a fact that is also reflected by lowered biosafety level requirements for the work with recombinant AAVs (rAAV) compared to other popular viral vector systems. Due to its low immunogenicity and the absence of cytotoxic responses AAV-based expression systems offer the possibility to express genes of interest for months in quiescent cells.

Production systems for rAAV vectors typically consist of a DNA-based vector containing a transgene expression cassette, which is flanked by inverted terminal repeats (payload). Construct sizes are limited to approximately 4.7-5.0 kb, which corresponds to the length of the wild-type AAV genome. In some embodiments it would thus be advantageous to have a payload smaller than this upper limit. rAAVs are produced in cell lines. The expression vector is co-transfected with a helper plasmid that mediates expression of the AAV rep genes which are important for virus replication and cap genes that encode the proteins forming the capsid. Recombinant adeno-associated viral vectors can transduce dividing and non-dividing cells, and different rAAV serotypes may transduce diverse cell types. These single-stranded DNA viral vectors have high transduction rates and have a unique property of stimulating endogenous Homologous Recombination without causing double strand DNA breaks in the host genome.

It should be appreciated that many intermediate steps of the wild-type infection cycle of AAV depend on specific interactions of the capsid proteins with the infected cell. These interactions are crucial determinants of efficient transduction and expression of genes of interest when rAAV is used as gene delivery tool. Indeed, significant differences in transduction efficacy of various serotypes for particular tissues and cell types have been described. Thus, in some embodiments AAV serotype 6 may be suitable for the systems, compositions, cells and methods of the invention. In yet some further embodiments, AAV serotype 8 may be suitable for the methods, systems, and the nucleic acid guided genome modifier chimeric protein of the invention.

It is believed that a rate-limiting step for the AAV-mediated expression of transgenes is the formation of double-stranded DNA. Recent reports demonstrated the usage of rAAV constructs with a self-complementing structure (scAAV) in which the two halves of the single-stranded AAV genome can form an intra-molecular double-strand. This approach reduces the effective genome size usable for gene delivery to about 2.3 kB but leads to significantly shortened onsets of expression in comparison with conventional single-stranded AAV expression constructs (ssAAV). Thus, in some embodiments, ssAAV may be applicable as a viral vector by the methods of the invention.

In yet some further embodiments, HDAd vectors may be suitable for the systems, compositions, cells and methods of the invention. The Helper-Dependent Adenoviral (HDAd) vectors HDAds have innovative features including the complete absence of viral coding sequences and the ability to mediate high level transgene expression with negligible chronic toxicity. HDAds are constructed by removing all viral sequences from the adenoviral vector genome except the packaging sequence and inverted terminal repeats, thereby eliminating the issue of residual viral gene expression associated with early generation adenoviral vectors. HDAds can mediate high efficiency transduction, do not integrate in the host genome, and have a large cloning capacity of up to 37 kb, which allows for the delivery of multiple transgenes or entire genomic loci, or large cis-acting elements to enhance or regulate tissue-specific transgene expression. One of the most attractive features of HDAd vectors is the long-term expression of the transgene.

Still further, in some embodiments, SV40 may be used as a suitable vector by the systems, compositions, cells and methods of the invention. SV40 vectors (SV40) are vectors originating from modifications brought to Simian virus-40 an icosahedral papovavirus.

Recombinant SV40 vectors are good candidates for gene transfer, as they display some unique features: SV40 is a well-known virus, non-replicative vectors are easy-to-make, and can be produced in titers of 10(12) IU/ml. They also efficiently transduce both resting and dividing cells, deliver persistent transgene expression to a wide range of cell types, and are non-immunogenic. Present disadvantages of rSV40 vectors for gene therapy are a small cloning capacity and the possible risks related to random integration of the viral genome into the host genome.

In certain embodiments, an appropriate vector that may be used by the invention may be a retroviral vector. A retroviral vector consists of proviral sequences that can accommodate the gene of interest, to allow incorporation of both into the target cells. The vector may also contain viral and cellular gene promoters, to enhance expression of the gene of interest in the target cells. Retroviral vectors stably integrate into the dividing target cell genome so that the introduced gene is passed on and expressed in all daughter cells. They contain a reverse transcriptase that allows integration into the host genome.

In yet some alternative embodiments, lentiviral vectors may be used in the present invention. Lentiviral vectors are derived from lentiviruses which are a subclass of Retroviruses. Commonly used retroviral vectors are “defective”, i.e. unable to produce viral proteins required for productive infection. Rather, replication of the vector requires growth in a packaging cell line. To generate viral particles comprising the nucleic acids sequence of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing the retroviral vectors comprising the nucleic acid molecules of the invention that contains the nucleic acids sequence of interest into packaging cell lines and of collecting the viral particles that are generated by the packaging lines are well known in the art.

In some alternative embodiments, the vector may be a non-viral vector. More specifically, such vector may be in some embodiments any one of plasmid, minicircle and linear DNA, ssDNA (that are especially useful for donor integration at cleavage site) or RNA (useful to avoid long term expression and or integration) or a modified polynucleotide (mainly chemically protective modifications to protect RNA or DNA-RNA chimeras to enhance specificity and or stability).

Nonviral vectors, in accordance with the invention, refer to all the physical and chemical systems except viral systems and generally include either chemical methods, such as cationic liposomes and polymers, or physical methods, such as gene gun, electroporation, particle bombardment, ultrasound utilization, and magnetofection. Efficiency of this system is sometimes less than viral systems in gene transduction, but their cost-effectiveness, availability, and more importantly reduced induction of immune system and no limitation in size of transgenic DNA compared with viral system have made them attractive also for gene delivery.

For example, physical methods applied for in vitro and in vivo gene delivery are based on making transient penetration in cell membrane by mechanical, electrical, ultrasonic, hydrodynamic, or laser-based energy so that DNA, RNA or RNP entrance into the targeted cells is facilitated.

In more specific embodiments, the vector may be a naked DNA vector. More specifically, such vector may be for example, a plasmid, minicircle or linear DNA.

Naked DNA alone may facilitate transfer of a nucleic acid sequence (2-200 Kb or more) into skin, thymus, cardiac muscle, and especially skeletal muscle and liver cells when directly injected. It enables also long-term expression. Although naked DNA injection is a safe and simple method, its efficiency for gene delivery is quite low.

Minicircles are modified plasmid in which a bacterial origin of replication (ori) was removed, and therefore they cannot replicate in bacteria.

Linear DNA or Doggybone™ are double-stranded, linear DNA construct that solely encodes an payload expression cassette, comprising antigen, promoter, polyA tail and telomeric ends.

It should be appreciated that all DNA vectors disclosed herein, may be also applicable for the methods, systems and compositions of the present disclosure.

Still further, it must be appreciated that the invention further provides any vectors or vehicles that comprise any of the donor nucleic acid molecules disclosed by the invention, as well as any host cell expressing the replacement and/or additional nucleic acid molecules disclosed by the invention.

It should be understood that any of the viral vectors disclosed herein may be relevant to any of the nucleic acid molecules discussed in other aspects of the invention, specifically to nucleic acid molecules encoding the SCNA (gRNA), the Donor or the protein components as described by the invention.

As indicated above, vectors may be provided directly to the subject cells thereby being contacted with the cell/s. In other words, the cells are contacted with vectors comprising the nucleic acid molecules of the invention that comprise the nucleic acid sequence of interest such that the vectors are taken up by the cells. Methods for contacting cells with nucleic acid vectors that are plasmids, such as electroporation, calcium chloride transfection, and lipofection (e.g. using Lipofectamin), are well known in the art. DNA can be introduced as naked nucleic acid, as nucleic acid complexed with an agent such as a liposome, nanoparticles or poloxamer, or can be delivered by viruses (e.g., adenovirus, AAV).

As indicated above, in some embodiments, the systems of the present disclosure further comprise at least one modifier protein or polypeptide. Still further, in some embodiments any of the modifier proteins disclosed by the present disclosure may be applicable in the present disclosure, or any variants, derivatives or fragments thereof. It should be noted that “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide. Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that in some embodiments may undergo post-translational modifications, such as phosphorylation, acetylation, glycosylation, manosylation, amidation, carboxylation, sulfhydryl bond formation, cleavage and the like.

By “fragments or peptides” it is meant a fraction of the protein of the invention. A “fragment” of a molecule, such as any of the amino acid sequences of the present invention, is meant to refer to any amino acid subset. This may also include “variants” or “derivatives” thereof. A “peptide” is meant to refer to a particular amino acid subset having a functional, structural activity or function displayed by the protein disclosed by the invention.

It should be appreciated that the invention encompasses any variant or derivative of the chimeric protein of the invention and any polypeptides that are substantially identical or homologue. The term “derivative” is used to define amino acid sequences (polypeptide), with any insertions, deletions, substitutions and modifications to the amino acid sequences (polypeptide) that either do not alter the activity of the original polypeptides or alter it purposefully.

By the term “derivative” it is also referred to homologues, variants and analogues thereof.

Proteins orthologs or homologues having a sequence homology or identity to the proteins of interest in accordance with the invention, specifically that may share at least 50%, at least 60% and specifically 65%, 70%, 75%, 80%, 85%, 90%, 91%, 92%, 93%, 94%, 95%, 96%, 97%, 98%, 99%, 100%, specifically as compared to the entire sequence of the proteins of interest in accordance with the invention. Specifically, homologs that comprise or consists of an amino acid sequence that is identical in at least 50%, at least 60% and specifically 65%, 70%, 75%, 80%, 85%, 90%, 91%, 92%, 93%, 94%, 95%, 96%, 97%, 98%, 99%, or higher to SEQ ID NO: 9111-174.

In some embodiments, derivatives refer to polypeptides, which differ from the polypeptides specifically defined in the present invention by insertions, deletions or substitutions of amino acid residues. It should be appreciated that by the terms “insertion/s”, “deletion/s” or “substitution/s”, as well as “substituted, “deleted”, “inserted”, as used herein it is meant any addition, deletion or replacement, respectively, of amino acid residues to the polypeptides disclosed by the invention, of between 1 to 50 amino acid residues, between 20 to 1 amino acid residues, and specifically, between 1 to 10 amino acid residues. More particularly, insertion/s, deletion/s or substitution/s may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N′ or C′ termini thereof.

With respect to amino acid sequences, one of skill will recognize that individual substitutions, deletions or additions to a nucleic acid, peptide, polypeptide, or protein sequence which alters, add or deletes a single amino acid or a small percentage of amino acids in the encoded sequence is a “conservatively modified variant” where the alteration results in the substitution of an amino acid with a chemically similar amino acid.

Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q).

Variants of the polypeptides of the invention may have at least 80% sequence similarity or identity, often at least 85% sequence similarity or identity, 90% sequence similarity or identity, or at least 95%, 96%, 97%, 98%, or 99% sequence similarity or identity at the amino acid level, with the protein of interest, such as the various polypeptides of the invention.

A further aspect of the present disclosure relates to at least one cell comprising and/or modified by at least one immune trans-regulatory/modulatory system or a population of cells comprising the at least one cell. In some embodiments, the system of the disclosed cell comprises the following components: one component (a), comprises at least one promoter-less donor nucleic acid molecule comprising at least one replacement nucleic acid sequence. The replacement nucleic acid sequence and/or the donor nucleic acid molecule comprising the replacement sequence is inserted and/or introduced into a target sequence within at least one target first immune-regulatory gene of interest. In some embodiments, the replacement nucleic acid sequence replaces at least part of the endogenous coding region of the target first immune-regulatory gene. In some alternative or additional embodiments, the replacement sequence is introduced into the endogenous non-coding region of the target first immune-regulatory gene, such that the target gene is intact. The replacement, and/or introduction of the replacement sequence provided by the donor nucleic acid molecule results in at least one of: (i) the production of at least one immune-modulatory product, controlled by at least one endogenous control element of said target immune-regulatory gene; and (ii) inhibition and/or reduction and/or modulation of the expression and/or activity and/or stability of the target first immune-regulatory gene. Another component (b), comprises at least one target recognition element targeted at a target sequence within the at least one target first immune-regulatory gene, or any nucleic acid sequence encoding the target recognition element. In some embodiments, where nucleic acid sequences that encode the target recognition element are used, these sequences may be provided either as part of the same nucleic acid donor molecule, or in at least one separate nucleic acid molecule.

In yet some further embodiments, the system comprised within or editing the disclosed cell, further comprising (c), at least one nucleic acid guided genome modifier protein, chimeric protein, complex or conjugate, or at least one nucleic acid sequence encoding said guided genome modifier protein.

In yet some further embodiments, the system used for the cell of the present disclosure is any one of the systems disclosed by the present disclosure.

In some embodiments, the disclosed cell is at least one hematopoietic cell and/or at least one stem cell.

In yet some further specific embodiments, the hematopoietic cell is at least one lymphocyte. More specifically, the lymphocyte may be at least one genetically modified or unmodified cell of the T-cell-lineage.

Still further, in certain embodiment, the T cell is at least one of regulatory T cell (Tregs), tumor-infiltrating lymphocyte (TIL) cell, cytotoxic T lymphocyte (CTL), and Natural Killer (NK) cells.

The present disclosure provides at least one cell or any populations comprising said cell, that comprises or is modified by the systems of the present invention. Such cell may be also referred to herein as a host cell. The term “host cell” includes a cell into which a heterologous (e.g., exogenous) nucleic acid and/or protein (e.g., the donor nucleic acid molecule, the target recognition element and the nucleic acid guided genome modifier/effector chimeric protein, complex or conjugate of the invention) or Ribonucleoprotein (RNP) thereof (specifically, the system of the invention), has been introduced. Persons of skill upon reading this disclosure will understand that such terms refer not only to the particular subject cell, but also is used to refer to the progeny of such a cell. Because certain modifications may occur in succeeding generations due to either mutation or environmental influences, developmental maturation, or due to the intended action of the invention, such progeny may not, in fact, be identical to the parent cell, but are still included within the scope of the term “host cell”. In some embodiments, the host cells provided by the invention are transduced or transfected by the nucleic acid sequences provided by the invention that encode the PAM abolished or reduced CRISPR-Cas proteins of the invention, any chimeric proteins thereof, and systems. This may refer in some embodiments, to cells that underwent a transfection procedure, meaning the introduction of a donor nucleic acid, e.g., an expression vector, or a replicating vector, into recipient cells by nucleic acid-mediated gene transfer. Alternatively, or in combination with nucleic acids encoding components of the invention, all or part of the components may be delivered as an RNA, as a protein, or as a preassembled RNP.

Transfection of eukaryotic cells may be either transient or stable, and is accomplished by various ways known in the art.

For example, transfection of eukaryotic cells may be chemical, e.g., via a cationic polymer (such as DEAE-dextran, polyethyleneimine, dendrimer, polybrene, calcium), calcium phosphate (e.g., phosphate, lipofectin, DOTAP, lipofectamine, CTAB/DOPE, DOTMA) or via a cationic lipid. Transfection of eukaryotic cells may also be physical, e.g. via a direct injection (for example, by Micro-needle, AFM tip, Gene Gun,), via biolistic particle delivery (for example, phototransfection, Magnetofection), or via electroporation (i.e., Lonza Nucleofector), laser-irradiation, sonoporation or a magnetic nanoparticle. Transfection of eukaryotic cells may also be biological (i.e., use of Agrobacterium in plants).

The term “host cells” as used herein refers to any cell known to a skilled person wherein the functional fragments or peptides thereof or any nucleic acid molecule or combination thereof according to the invention may be introduced. For example, a host cell may be any eukaryotic cell of a multi-cellular organism having an immune system that is to be modified by the disclosed systems and methods. More specifically, eukaryotic host cell/s in accordance with the invention may include, but is not limited to a plant, an insect cell, an invertebrate cell, vertebrate cell, mammalian cell and the like. It is understood that such terms refer not only to the particular subject cells but to the progeny or potential progeny of such a cell. Because certain modification may occur in succeeding generation due to either mutation or environmental influences, such progeny may not, in fact, be identical to the parent cell, but are still included within the scope of the term as used herein.

The “host cell” as used herein refers also to cells which can be transformed or transfected with naked DNA, any plasmid or expression vectors constructed using recombinant DNA techniques. A drug resistance or other selectable marker carried on the transforming or transfecting plasmid is intended in part to facilitate the selection of the transformants.

Additionally, the presence of a selectable marker, such as drug resistance marker may be of use in keeping contaminating microorganisms from multiplying in the culture medium.

Such a pure culture of the transformed host cell would be obtained by culturing the cells under conditions which require the phenotype for survival.

Eukaryotic cells may be mammalian cells, plant cells, or cells of any organism having an immune system that can be modulated by the disclosed systems and methods. As used herein, the term “eukaryotic cell” refers to any cell type known to a person skilled in the art which is suitable for genetic manipulation. It should be noted that the term “eukaryotic cells” as used herein, further encompasses the autologous cells or allogeneic cells used by the methods of the invention via adoptive transfer, as discussed herein after in connection with other aspects of the invention. Thus, eukaryote cells as herein defined may be derived from animals, any plants, for example, but not limited to, insect cells, or mammalian cells, specifically, human cells.

It should be further understood that the term “Cell”, is defined here as to comprise any type of cell, r a eukaryotic cell, isolated or not, cultured or not, differentiated or not, and comprising also higher levels organizations of cells such as tissues, organs, calli, organisms or parts thereof. Exemplary cells include, but are not limited to vertebrate cells, mammalian cells, human cells, plant cells, animal cells, invertebrate cells, nematodal cells, insect cells, stem cells, and the like.

In some embodiments, there are several types of target cells, specifically, eukaryotic cells, that may be used by the compositions and methods of the invention. By way of example, eukaryotic cells may be, but are not limited to, stem cells, e.g. hematopoietic stem cells (HSCs), embryonic stem cells, totipotent stem cells, pluripotent stem cells or induced pluripotent stem cells and multipotent progenitor cells.

Stem cells are generally known for their three unique characteristics: (i) they have the unique ability to renew themselves continuously; (ii) they have the ability to differentiate into somatic cell types; and (iii) they have the ability to limit their own population into a small number. In mammals, there are two broad types of stem cells, namely embryonic stem cells (ESCs), and adult stem cells. Stem cells may be autologous or heterologous to the subject. In order to avoid rejection of the cells by the subject's immune system, autologous stem cells are usually preferred.

Thus, in some embodiments, the target cells according to the invention may be embryonic stem cells, or human embryonic stem cells (hESCs), that were obtained from self-umbilical cord blood just after birth. Embryonic stem cells are pluripotent stem cells derived from the early embryo that are characterized by the ability to proliferate over prolonged periods of culture while remaining undifferentiated and maintaining a stable karyotype, with the potential to differentiate into derivatives of all three germ layers. hESCs may be also derived from the inner cell mass (ICM) of the blastocyst stage (100-200 cells) of embryos generated by in vitro fertilization. However, methods have been developed to derive hESCs from the late morula stage (30-40 cells) and, recently, from arrested embryos (16-24 cells incapable of further development) and single blastomeres isolated from 8-cell embryos.

In further embodiments, the target cells according to the invention are totipotent stem cells. Totipotent stem cells are versatile stem cells, and have the potential to give rise to any and all human cells, such as brain, liver, blood or heart cells or to an entire functional organism (e.g. the cell resulting from a fertilized egg). The first few cell divisions in embryonic development produce more totipotent cells. After four days of embryonic cell division, the cells begin to specialize into pluripotent stem cells. Embryonic stem cells may also be referred to as totipotent stem cells.

In further embodiments, the target cells according to the invention are pluripotent stem cells. Similar to totipotent stem cells, a pluripotent stem cell refers to a stem cell that has the potential to differentiate into any of the three germ layers: endoderm (interior stomach lining, gastrointestinal tract, the lungs), mesoderm (muscle, bone, blood, urogenital), or ectoderm (epidermal tissues and nervous system). Pluripotent stem cells can give rise to any fetal or adult cell type. However, unlike totipotent stem cells, they cannot give rise to an entire organism. On the fourth day of development, the embryo forms into two layers, an outer layer which will become the placenta, and an inner mass which will form the tissues of the developing human body. These inner cells are referred to as pluripotent cells. In still further embodiments, the target cells that may be applicable for the compositions and methods according to the present disclosure, are multipotent progenitor cells. Multipotent progenitor cells have the potential to give rise to a limited number of lineages. As a non-limiting example, a multipotent progenitor stem cell may be a hematopoietic cell, which is a blood stem cell that can develop into several types of blood cells but cannot into other types of cells. Another example is the mesenchymal stem cell, which can differentiate into osteoblasts, chondrocytes, and adipocytes. Multipotent progenitor cells may be obtained by any method known to a person skilled in the art.

In yet further embodiments, the target cells according to the invention are induced pluripotent stem cells. Induced pluripotent stem cells, commonly abbreviated as iPS cells are a type of pluripotent stem cell artificially derived from a non-pluripotent cell, typically an adult somatic cell, even a patient's own. Such cells can be induced to become pluripotent stem cells with apparently all the properties of hESCs. Induction requires only the delivery of four transcription factors found in embryos to reverse years of life as an adult cell back to an embryo-like cell. For example, iPS cells could be used for autologous transplantation in a patient. The manipulation of the cells may be performed in some embodiments, ex vivo in the iPS cells obtained from the patient as performed by the methods of the invention and the cells may be then implanted back into the patient (i.e. autologous transplantation). It should be understood that any of the cells disclosed herein may be used by the methods of the invention for ex vivo therapy as disclosed herein after.

In some embodiments the cell/s disclosed by the present disclosure may be any hematopoietic cell/s. “Hematopoietic cells” are cellular blood components all derived from hematopoietic stem cells in the bone marrow. It should be appreciated that in certain embodiments, hematopoietic cells as used herein include cells of the myeloid and the lymphoid lineages of blood cells. More specifically, myeloid cells include monocytes, (macrophages and dendritic cells (DCs)), granulocytes (neutrophils), basophils, eosinophils, erythrocytes, and megakaryocytes or platelets. The Lymphoid cells include T cells, B cells, and natural killer (NK) cells. Thus, in certain embodiments, the cells modified by the systems of the invention may be any hematopoietic cell described herein.

Generally, blood cells are divided into three lineages: red blood cells (erythroid cells) which are the oxygen carrying, white blood cells (leukocytes that are further subdivided into granulocytes, monocytes and lymphocytes) and platelets (thrombocytes).

In more specific embodiments, the target cells may be lymphocytes. “Lymphocytes” as used herein, are mononuclear nonphagocytic leukocytes found in the blood, lymph, and lymphoid tissues. They comprise the body's immunologically competent cells and their precursors. They are divided on the basis of ontogeny and function into two classes, B and T lymphocytes, responsible for humoral and cellular immunity, respectively. Most are small lymphocytes 7-10 m in diameter with a round or slightly indented heterochromatic nucleus that almost fills the entire cell and a thin rim of basophilic cytoplasm that contains few granules.

When “activated” by contact with antigen, small lymphocytes begin macromolecular synthesis, the cytoplasm enlarges until the cells are 10-30 m in diameter, and the nucleus becomes less completely heterochromatic; they are then referred to as large lymphocytes or lymphoblasts. These cells then proliferate and differentiate into B and T memory cells and into the various effector cell types: B cells into plasma cells and T cells into helper, cytotoxic, and suppressor cells.

In yet some further embodiments, the target hematopoietic cell modified by the systems, compositions and methods of the invention may be at least one T cell. More specifically, a “T cell” or “T lymphocyte” as used herein is characterized by the presence of a T-cell receptor (TCR) on the cell surface. It should be noted that T-cells include helper T cells (“effector T cells” or “Th cells”), cytotoxic T cells (“Tc,” “CTL” or “killer T cell”), memory T cells, and regulatory T cells as well as Natural killer T cells, Mucosal associated invariants and Gamma delta T cells. More specifically, thymocytes are hematopoietic progenitor cells present in the thymus. Thymopoiesis is the process in the thymus by which thymocytes differentiate into mature T lymphocytes. The thymus provides an inductive environment, which allows for the development and selection of physiologically useful T cells. The processes of beta-selection, positive selection, and negative selection shape the population of thymocytes into a peripheral pool of T cells that are able to respond to foreign pathogens and are immunologically tolerant towards self-antigens.

Thymocytes are classified into a number of distinct maturational stages based on the expression of cell surface markers. The earliest thymocyte stage is the double negative (DN) stage (negative for both CD4 and CD8), which more recently has been better described as Lineage-negative, and which can be divided into four sub-stages. The next major stage is the double positive (DP) stage (positive for both CD4 and CD8). The final stage in maturation is the single positive (SP) stage (positive for either CD4 or CD8).

Still further, in some embodiments, the target cells may be NK cells. Naturalkiller cells or NK cells are a type of cytotoxic lymphocyte critical to the innate immune system. NK cells (belonging to the group of innate lymphoid cells) are defined as large granular lymphocytes (LGL). The role NK cells play is analogous to that of cytotoxic T cells in the vertebrate adaptive immune response. NK cells provide rapid responses to viral-infected cells, acting at around three days after infection, and respond to tumor formation.

Typically, immune cells detect major histocompatibility complex (MHC) presented on infected cell surfaces, triggering cytokine release, causing lysis or apoptosis. NK cells are unique, however, as they have the ability to recognize stressed cells in the absence of antibodies and MHC, allowing for a much faster immune reaction. They were named “natural killers” because of the initial notion that they do not require activation to kill cells that are missing “self” markers of MHC class 1.

As indicated above, NK cells are critical to the innate immune system in providing rapid responses to viral-infected cells and tumor formation. In contrast to CTLs, NK cells do not express T cell antigen receptors (TCR) or pan T marker CD3 or surface immunoglobulins (Ig) B cell receptors, instead they express the surface markers CD16 (FcγRIII) and CD56 in humans (NK1.1 or NK1.2 in mice), up to 80% of human NK cells also express CD8. Further, NK cells are effectors of innate immunity in expressing activating and inhibitory NK receptors, which play an important function in self-tolerance and in sustaining NK activity.

In yet some further embodiments, the genetically modified cell/s express at least one Chimeric Antigen Receptors (CAR) molecule.

In some embodiments, the cell of the present disclosure may be said cell is of an autologous or an allogeneic source.

In some embodiments, the “host cells” provided herein, specifically, the cells transduced or transfected with or comprising the systems provided by the invention, may be cells of an autologous source. The term “autologous” when relating to the source of cells, refers to cells derived or transferred from the same subject that is to be treated by the in vivo or ex vivo the method of the invention.

The term “allogeneic” when relating to the source of cells, refers to cells derived or transferred from a different subject, referred to herein as a donor, of the same species.

The term “syngeneic” when relating to the source of cells, refers to cells derived or transferred from a genetically identical, or sufficiently identical and immunologically compatible subject (e.g., an identical twin).

In certain embodiments, specifically when the cell is of an autologous source, the cell may be of a subject suffering from at least one of an immune-related disorder or condition, and a proliferative disorder.

A further aspect of the present disclosure relates to a composition comprising at least one of: In one option (I), the disclosed composition may comprise an immune trans-regulatory and/or modulatory system comprising: as one component (a), at least one promoter-less donor nucleic acid molecule comprising at least one replacement nucleic acid sequence.

The replacement nucleic acid sequence and/or the donor nucleic acid molecule comprising the replacement sequence is inserted and/or introduced into a target sequence within at least one target first immune-regulatory gene/s of interest. In some embodiments, the replacement nucleic acid sequence replaces at least part of the endogenous coding region of the target first immune-regulatory gene. In some alternative or additional embodiments, the replacement sequence is introduced into the endogenous non-coding region of the target first immune-regulatory gene, such that the target gene is intact. The replacement, and/or introduction of the replacement sequence provided by the donor nucleic acid molecule, results in at least one of: (i) the production of at least one immune-modulatory product, controlled by at least one endogenous control element of said target first immune-regulatory gene; and (ii) inhibition and/or reduction and/or modulation of the expression and/or activity, and/or stability of the at least one target first immune-regulatory gene; and in another component (b), at least one target recognition element targeted at a target sequence within the target first immune-regulatory gene, or any nucleic acid sequence encoding the target recognition element. In some embodiments, where nucleic acid sequences that encode the target recognition element are used, these sequences may be provided either as part of the same nucleic acid donor molecule, or in at least one separate nucleic acid molecule. In some optional embodiments, the system of the disclosed composition may further comprise as another component (c), at least one nucleic acid guided genome modifier protein, chimeric protein, complex or conjugate, or at least one nucleic acid sequence encoding said guided genome modifier protein. In some embodiments, when the nucleic acid sequence that encoding the nucleic acid guided genome modifier protein is provided in the disclosed system, and when nucleic acid sequences that encode the target recognition element are used, the nucleic acid sequence encoding the at least one nucleic acid guided genome modifier protein may be provided either in a separate nucleic acid molecule, or in the same molecule together with the nucleic acid sequence that encodes the target recognition element, and/or as part of the at least one donor nucleic acid molecule.

In some alternative or additional embodiments, the composition of the present disclosure may comprise (II), at least one nucleic acid cassette or any vector or vehicle comprising at least one of: (i) the nucleic acid sequence of (a); (i) the nucleic acid sequence of (b); (iii) the nucleic acid sequence of (c); and (iv) the nucleic acid sequence of (a) and at least one of (b) and (c). Still further, in some alternative or additional embodiments, the disclosed compositions may comprise (III), at least one cell comprising and/or modified by at least one of: the nucleic acid the at least one system of (I), or the at least one nucleic acid cassette or any vector or vehicle of (II); or any matrix, nano- or micro-particle comprising at least one of (I) and (II), and/or a population of cells comprising at least one of said cell. It should be noted that in some optional embodiments, the composition of the present disclosure may further comprise at least one of pharmaceutically acceptable carrier/s, diluent/s, excipient/s and additive/s.

In some embodiments, the disclose compositions may comprise at least one of: any of the systems disclosed by the present discloser, and/or any of the cells disclosed by the present disclosure.

In some aspects thereof, the present disclosure further provides compositions that comprise any of the systems disclosed herein and any of the modified cells of the invention. In yet some further embodiments, the composition of the invention may optionally further comprise at least one of pharmaceutically acceptable carrier/s, excipient/s, additive/s diluent/s and adjuvant/s.

The pharmaceutical compositions of the invention can be administered and dosed by the methods of the invention, in accordance with good medical practice, systemically, for example by parenteral intravenous. It should be noted however that the invention may further encompass additional administration modes. In other examples, the pharmaceutical composition can be introduced to a site by any suitable route including intraperitoneal, subcutaneous, transcutaneous, topical, intramuscular, intraarticular, subconjunctival, or mucosal, e.g. oral, intranasal, or intraocular administration.

Local administration to the area in need of treatment may be achieved by, for example, by local infusion during surgery, topical application, direct injection into the specific organ. More specifically, the compositions used in any of the methods of the invention, described herein before, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s).

In yet some further embodiments, the composition of the invention may optionally further comprise at least one of pharmaceutically acceptable carrier/s, excipient/s, additive/s diluent/s and adjuvant/s.

More specifically, pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients, specifically the systems, protein, nucleic acid, host cell of the invention with liquid carriers or finely divided solid carriers or both, and then, if necessary, shaping the product.

The compositions may be formulated into any of many possible dosage forms such as, but not limited to, tablets, capsules, liquid syrups, soft gels, suppositories, and enemas.

The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

It should be understood that in addition to the ingredients particularly mentioned above, the formulations may also include other agents conventional in the art having regard to the type of formulation in question.

Still further, pharmaceutical preparations are compositions that include the protein, nucleic acid, host cell of the invention present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid or liquid such as tablets, capsules, powders, granules, ointments, solutions, suppositories, injections, inhalants, gels, microspheres, and aerosols. As such, administration of the protein, nucleic acid, host cell of the invention can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

Still further, the composition/s of the invention and any components thereof may be applied as a single one-time dose, as a single daily dose or multiple daily doses, preferably, every 1 to 7 days. It is specifically contemplated that such application may be carried out once or several times in the lifetime of a patient, once, twice, thrice, four times, five times or six times daily, or may be performed once daily, once every 2 days, once every 3 days, once every 4 days, once every 5 days, once every 6 days, once every week, two weeks, three weeks, four weeks or even more than a month. The application of the chimeric proteins of the invention, systems thereof, nucleic acid sequences or any vectors thereof, host cell/s transformed or transfected by said nucleic acid sequence, in accordance with the invention or of any component thereof, or the effects thereof, may last up to the lifetime of the patient, a day, two days, three days, four days, five days, six days, a week, two weeks, three weeks, four weeks, a month, two months three months or even more. More specifically, for one, two, three, four, five, six, seven, eight, nine, ten, eleven, twelve months of more or for several years.

A further aspect of the present disclosure relates to a method of modulating at least one target cell. The disclosed method comprises the steps of contacting the target cell with at least one of:

In one option (I), the disclosed methods may comprise the step of contacting with the target cell an immune trans-regulatory/modulatory system comprising: in one component (a), at least one promoter-less donor nucleic acid molecule comprising at least one replacement nucleic acid sequence. The replacement nucleic acid sequence and/or the donor nucleic acid molecule comprising the replacement sequence is inserted and/or introduced into a target sequence within at least one target first immune-regulatory gene of interest. In some embodiments, the replacement nucleic acid sequence replaces at least part of the endogenous coding region of the target first immune-regulatory gene. In some alternative or additional embodiments, the replacement sequence is introduced into the endogenous non-coding region of the target first immune-regulatory gene, such that the target gene is intact. The replacement, and/or introduction of the replacement sequence provided by the donor nucleic acid molecule, results in at least one of: (i) the production of at least one immune-modulatory product, controlled by at least one endogenous control element of the target first immune-regulatory gene; and (ii) inhibition and/or reduction and/or modulation of the expression and/or activity and/or stability of the target first immune-regulatory gene. Another component (b), is at least one target recognition element targeted at a target sequence within the target first immune-regulatory gene, or any nucleic acid sequence encoding the target recognition element. In some embodiments, where nucleic acid sequences that encode the target recognition element are used, these sequences may be provided either as part of the same nucleic acid donor molecule, or in at least one separate nucleic acid molecule. In some optional embodiments, the system that may be used by the disclosed methods may further comprise as another component (c), at least one nucleic acid guided genome modifier protein, chimeric protein, complex or conjugate, or at least one nucleic acid sequence encoding said guided genome modifier protein. In some embodiments, when the nucleic acid sequence encoding the nucleic acid guided genome modifier protein is provided in the disclosed system, and when nucleic acid sequences that encode the target recognition element are used, the nucleic acid sequence encoding the at least one nucleic acid guided genome modifier protein may be provided either in a separate nucleic acid molecule, or in the same molecule together with the nucleic acid sequence that encodes the target recognition element, and/or as part of the at least one donor nucleic acid molecule.

In some alternative or additional embodiments, the disclosed methods may comprise the step of contacting with the target cell of the present disclosure with (II), at least one nucleic acid cassette or any vector or vehicle comprising at least one of: (i) the nucleic acid sequence of (a); (i) the nucleic acid sequence of (b); (iii) the nucleic acid sequence of (c); and (iv) the nucleic acid sequence of (a) and at least one of (b) and (c). Still further, in some alternative or additional embodiments, disclosed methods may comprise the step of contacting with the target cell (III), at least one cell comprising and/or modified by at least one of: the nucleic acid the at least one system of (I), or the at least one nucleic acid cassette or any vector or vehicle of (II); or any matrix, nano- or micro-particle comprising at least one of (I) and (II), and/or a population of cells comprising at least one of the cell/s.

In yet some further alternative or additional embodiments, the disclosed methods may comprise the step of contacting with the target cell (IV), at least one composition comprising at least one of (I), (II), (III) or any combinations thereof.

In some embodiments, the disclosed methods may comprise the step of contacting with the target cell any of the systems disclosed by the present disclosure. In yet some further embodiments, the disclosed methods may comprise the step of contacting with the target cell any of the cell/s as define by the present disclosure. Still further, the disclosed methods may comprise the step of contacting with the target cell any of the compositions defined by the present disclosure.

In certain embodiments, the target cell modulated by the disclosed methods, and/or the genetically modified cell used by the disclosed methods for contacting with the target cell, may be at least one hematopoietic cell and/or at least one stem cell.

In more specific embodiments, the hematopoietic cell may be at least one lymphocyte. In more specific embodiments, the lymphocyte may be at least one genetically modified or unmodified cell of the T-cell-lineage.

Still further, in some embodiments, such T cell may be at least one of Tregs, TIL cell, CTL, and NK cells.

In some embodiments, the genetically modified cells used by the disclosed methods express at least one CAR molecule.

In some embodiments, the target cell for modulation by the disclosed method may be any cell of a subject suffering from at least one immune-related disorder and/or at least one proliferative disorder. Accordingly, in some embodiments, the contacting step of the disclosed method is performed by administering to the subject an effective amount of at least one of the systems of (I), at least one nucleic acid cassette or any vector or vehicle of (II), at least one genetically modified cell of (III), at least one composition of (IV), or any combinations thereof.

In some embodiments of the disclosed methods, the proliferative disorder is at least one neoplastic disorder, specifically, cancer. In yet some further embodiments, the immune-related disorder is at least one of an infectious disease, a graft versus host disease, an inflammatory disorder, an immune-cell mediated disorder and an autoimmune disorder.

According to certain embodiments, the target cell modulated by the disclosed methods may be an immune cell or alternatively, a non-immune cell. In more specific embodiments, the target cell for the disclosed modulatory methods may be a cell that resides within at least one tissue of the subject affected by the immune-related disorder, and/or proliferative disorder.

In some embodiments, the target cell may be any cancerous cell of a cancer tissue of the subject, or any immune-cell of the subject.

In some embodiments, the target first immune-regulatory gene targeted by the systems used by the disclosed methods is at least one gene encoding at least one immune-checkpoint protein, optionally the immune-checkpoint protein is at least one of PDCD1/PD1; TIGIT; BTLA; CD152/CTLA4; LAG-3 (CD223); and TIM-3 (HAVCR2).

In yet some further embodiments, the target immune-regulatory gene of interest is PDCD1/PD-1. In more specific embodiments, the target sequence within the target immune-regulatory gene is a target sequence within exon 3 of said PD-1 gene. In some other alternative or additional specific embodiments, the target sequence within the target immune-regulatory gene is a target sequence within exon 1 of said PD-1 gene. In yet some further embodiments, the insertion is mediated by HDR or NHEJ.

In some embodiments, the target first immune-regulatory gene targeted by the systems used by the disclosed methods is at least one gene encoding an immune-modulator of hematopoietic cell proliferation, recruitment and/or survival, optionally the target gene is Leukemia inhibitory factor (LIF-1).

In some embodiments, the target first immune-regulatory gene targeted by the systems used by the disclosed methods is at least one gene encoding a cytokine and/or chemokine, optionally the target gene is CXCR6.

A further aspect of the present disclosure relates to a method of curing or treating, preventing, inhibiting, reducing, eliminating, protecting or delaying the onset of a pathologic disorder in a mammalian subject. More specifically, the method of the invention comprises the step of administering to the subject an effective amount of at least one of: In one option (I), the disclosed therapeutic methods may comprise the step of administering to the subject an effective amount of an immune trans-regulatory/modulatory system comprising: (a) at least one promoter-less donor nucleic acid molecule comprising at least one replacement nucleic acid sequence. The replacement nucleic acid sequence and/or the donor nucleic acid molecule comprising the replacement sequence is inserted and/or introduced into a target sequence within at least one target first immune-regulatory gene of interest. In some embodiments, the replacement nucleic acid sequence replaces at least part of the endogenous coding region of the target first immune-regulatory gene. In some alternative or additional embodiments, the replacement sequence is introduced into the endogenous non-coding region of the target first immune-regulatory gene, such that the target gene is intact. The replacement, and/or introduction of the replacement sequence provided by the donor nucleic acid molecule, results in at least one of: (i) the production of at least one immune-modulatory product, controlled by at least one endogenous control element of said target immune-regulatory gene; and (ii) inhibition and/or reduction and/or modulation of the expression and/or activity and/or stability of the target immune-regulatory gene; and (b) at least one target recognition element targeted at a target sequence within the target immune-regulatory gene, or any nucleic acid sequence encoding the target recognition element. In some embodiments, where nucleic acid sequences that encode the target recognition element are used, these sequences may be provided either as part of the same nucleic acid donor molecule, or in at least one separate nucleic acid molecule. In some optional embodiments, the system that may be used by the disclosed methods may further comprise (c) at least one nucleic acid guided genome modifier protein, chimeric protein, complex or conjugate, or at least one nucleic acid sequence encoding the guided genome modifier protein. In some embodiments, when the nucleic acid sequence encoding the nucleic acid guided genome modifier protein is provided in the disclosed system, and when nucleic acid sequences that encode the target recognition element are used, the nucleic acid sequence encoding the at least one nucleic acid guided genome modifier protein may be provided either in a separate nucleic acid molecule, or in the same molecule together with the nucleic acid sequence that encodes the target recognition element, and/or as part of the at least one donor nucleic acid molecule.

In some alternative or additional embodiments, the disclosed therapeutic methods may comprise the step of administering to the subject an effective amount of (II), at least one nucleic acid cassette or any vector or vehicle comprising at least one of: (i) the nucleic acid sequence of (a); (i) the nucleic acid sequence of (b); (iii) the nucleic acid sequence of (c); and (iv) the nucleic acid sequence of (a) and at least one of (b) and (c). Still further, in some alternative or additional embodiments, disclosed therapeutic methods may comprise the step of administering to the subject an effective amount of (III), at least one cell comprising and/or modified by at least one of: the nucleic acid the at least one system of (I), or the at least one nucleic acid cassette or any vector or vehicle of (II); or any matrix, nano- or micro-particle comprising at least one of (I) and (II), and/or a population of cells comprising at least one of the cell/s. In yet some further alternative or additional embodiments, the disclosed therapeutic methods may comprise the step of administering to the subject an effective amount of (IV), at least one composition comprising at least one of (I), (II), (III) or any combinations thereof.

In some embodiments, the disclosed therapeutic methods may comprise the step of administering to the subject an effective amount of any of the systems disclosed by the present disclosure. In yet some further embodiments, the disclosed methods may comprise the step of administering to the subject an effective amount of any of the cell/s as define by the present disclosure. Still further, the disclosed methods may comprise the step of administering to the subject an effective amount of any of the compositions defined by the present disclosure.

In some embodiments, the therapeutic methods disclosed herein may be applicable for any pathologic disorder. In yet some further embodiments, such pathologic disorder is at least one immune-related disorder and/or at least one proliferative disorder. In more specific embodiments, a proliferative disorder is at least one neoplastic disorder, specifically, cancer. Still further, in some embodiments, the immune-related disorder is at least one of an infectious disease, a graft versus host disease, an inflammatory disorder, an immune-cell mediated disorder and an autoimmune disorder.

Thus, the immune-trans-regulatory systems disclosed by the present disclosure, as well as the methods disclose herein may be applicable for treating and/or for modulating various disorders or conditions associated or affected directly or indirectly by the immune system. In some embodiments, the present disclosure is applicable for treating and modulating proliferative disorders.

Proliferative disorders, may include in some embodiments neoplastic disorders, but also encompasses other disorders involving cell proliferation. More specifically, neoplastic disorders as used herein encompass malignant or benign hyperplasia. In some embodiments the proliferative disorders as used herein refer to cancer.

More specifically, as used herein to describe the present invention, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the systems, compositions, cells and methods of the present invention may be applicable for treatment of a patient suffering from any one of non-solid and solid tumors.

Malignancy, as contemplated in the present invention may be any one of carcinomas, melanomas, lymphomas, leukemias, myeloma and sarcomas.

Carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Melanoma as used herein, is a malignant tumor of melanocytes. Melanocytes are cells that produce the dark pigment, melanin, which is responsible for the color of skin. They predominantly occur in skin, but are also found in other parts of the body, including the bowel and the eye. Melanoma can occur in any part of the body that contains melanocytes.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency.

Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Further malignancies that may find utility in the present invention can comprise but are not limited to hematological malignancies (including lymphoma, leukemia and myeloproliferative disorders, as described above), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The invention may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma. Still further, the present disclosure (systems, compositions, cells and methods), may be applicable in some embodiments to cancers that include, but are not limited to, Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia and Wilms tumor (kidney cancer).

In yet some further embodiments, the systems, compositions, cells and methods of the invention may be applicable for any of the proliferative disorders discussed herein.

In some embodiments, the methods of the invention may be used to treat a proliferative disorder, cancer, tumor and malignancy by activating/enhancing antitumor immunity. The term “antitumor immunity” refers to innate and adaptive immune responses which may lead to tumor control.

The immune system can be activated by the disclosed systems and methods and, once primed, can elicit an antitumor response. Genetically modified target cells, such as T regulatory cells, or NK cells are a front-line defense against tumors and can provide tumoricidal activity to enhance tumor immune surveillance. Cytokines like IFN-γ or TNF play a crucial role in creating an immunogenic microenvironment and therefore are key players in the fight against metastatic cancer.

As noted above, the methods of the invention may be relevant for treating any immune-related disorder, for example, an infectious disease, specifically, a viral infection, cancer or any other proliferative disorder, a graft versus host disease, an inflammatory disorder, an immune-cell mediated disorder and an autoimmune disorder. An “Immune-related disorder” or “Immune-mediated disorder”, as used herein encompasses any condition that is associated with the immune system of a subject, more specifically through inhibition or the activation of the immune system, or that can be treated, prevented or ameliorated by reducing degradation of a certain component of the immune response in a subject, such as the adaptive or innate immune response. More specifically, an ‘immune-related disorder’, as meant herein, encompasses a range of dysfunctions of the innate and adaptive immune systems. In more specific terms, immune-related disorder can be characterized, for example, (1) by the component(s) of the immune system; (2) by whether the immune system is overactive or underactive; (3) by whether the condition is congenital or acquired, as will be specified herein after.

An immune-related disorder may include infectious condition (e.g., viral infections), metabolic disorders, auto-immune disorders, vasculitis, inflammation and proliferative disorders, specifically, cancer. In some embodiments, the immune-related disorder may be an autoimmune disease. In accordance with some embodiments, the methods of the invention are applicable in treating autoimmune disorders. An autoimmune disease is a condition arising from an abnormal immune response to a normal body part.

Still further, of particular relevance for the methods of the invention are patients' populations diagnosed with one of autoimmune disorders, also referred to as disorders of immune tolerance, when the immune system fails to properly distinguish between self and non-self-antigens. It has been well established that T cells lymphocytes, and the NK cells in particular, play a pivotal role in the control of immune tolerance under normal conditions, and in T- and B-cell mediated human autoimmune disorders.

Thus, according to some embodiments, the methods of the invention, as well as any systems, cells and compositions of the invention may be used for the treatment of a patient suffering from any autoimmune disorder. In some specific embodiments, the methods as well as any systems, cells, cell populations and compositions of the invention may be used for treating an autoimmune disease such as for example, but not limited to, inflammatory bowel disease (IBD), ulcerative colitis, Crohn's disease, fatty liver disease, Lymphocytic colitis, Ischaemic colitis, Diversion colitis, Behget's syndrome, Indeterminate colitis, rheumatoid arthritis, systemic lupus erythematosus (SLE), Eaton-Lambert syndrome, Goodpasture's syndrome, Greave's disease, Guillain-Barr syndrome, autoimmune hemolytic anemia (AIHA), Idiopathic thrombocytopenic purpura (ITP), hepatitis, insulin-dependent diabetes mellitus (IDDM) and NIDDM, multiple sclerosis (MS), myasthenia gravis, plexus disorders e.g. acute brachial neuritis, polyglandular deficiency syndrome, primary biliary cirrhosis, scleroderma, thrombocytopenia, thyroiditis e.g. Hashimoto's disease, Sjogren's syndrome, allergic purpura, psoriasis, mixed connective tissue disease, polymyositis, dermatomyositis, vasculitis, polyarteritis nodosa, arthritis, alopecia areata, polymyalgia rheumatica, Wegener's granulomatosis, Reiter's syndrome, ankylosing spondylitis, pemphigus, bullous pemphigoid, dermatitis herpetiformis, psoriatic arthritis, reactive arthritis, and ankylosing spondylitis, inflammatory arthritis, including juvenile idiopathic arthritis, gout and pseudo gout, as well as arthritis associated with colitis or psoriasis, Pernicious anemia, some types of myopathy and Lyme disease (Late).

Particular examples of an autoimmune disorder include Rheumatoid arthritis (RA), Multiple sclerosis (MS), Systemic lupus erythematosus (lupus), Type 1 diabetes, Psoriasis/psoriatic arthritis, Inflammatory bowel disease including Crohn's disease and Ulcerative colitis, and Vasculitis.

In some specific embodiments, the methods of the invention may be particularly applicable for autoimmune disorder such as multiple sclerosis (MS), Anti-neutrophil cytoplasmic antibodies (ANCAs)-related disorder, and systemic lupus erythematosus (SLE).

In some further embodiments, the methods of the invention may be applicable for treating immune-related disorder such as an inflammatory disorder. In accordance with some embodiments, the methods of the invention are applicable in treating an inflammatory disorder. The terms “inflammatory disease” or “inflammatory-associated condition” refers to any disease or pathologically condition which can benefit from the reduction of at least one inflammatory parameter, for example, induction of an inflammatory cytokine such as IFN-gamma and IL-2 and reduction in IL-6 levels. The condition may be caused (primarily) from inflammation, or inflammation may be one of the manifestations of the diseases caused by another physiological cause. In some embodiments, an inflammatory disease that may be applicable for the methods of the invention may be any one of atherosclerosis, Rheumatoid arthritis (RA) and inflammatory bowel disease (IBD).

Of particular interest to the present context is a condition denoted Graft versus Host Disease (GvHD) that may occur after an allogeneic transplant, wherein the donated transplant cells view the recipient's body as foreign. GvHD is a possible complication of high dose cancer treatment. It also happens after an allogeneic bone marrow or stem cell transplant that use very high doses of chemotherapy, sometimes with radiotherapy. The term ‘GvHD’ as meant herein encompasses all known form of GvHD, namely the acute GvHD (aGvHD), the chronic GvHD (cGvHD), and the late acute GVHD and overlap syndrome (with features of both aGvHD and cGvHD).

More specifically, the pathophysiology of aGvHD has been tightly linked to the activity and maturation of the donor T cells and NK cells that are transferred along with the marrow graft, i.e. cells that are directly responsible for recognition of antigenic differences on antigen-presenting cells of the host. Once activated, donor anti-host-specific T cells can mediate tissue destruction. GvHD continues to be a major life-threatening complication after allogeneic bone marrow transplantation.

In some embodiments, the disclosed systems, cells compositions and methods may be applicable for the treatments of pulmonary arterial hypertension (PAH), Pulmonary arterial hypertension (PAH), as used herein, is a progressive disease without a cure, characterized by remodeling and narrowing of the pulmonary arteries, which lead to elevation of right ventricular pressure, heart failure, and death.

In yet some further embodiments, the disclosed methods may be applicable for any infectious diseases. Thus, in some embodiments, the therapeutic methods of the invention may be applicable for any condition caused by at least one pathogen. More specifically, any immune-related disorder or condition that may be a pathologic condition caused by any of the pathogens disclosed by the invention, for example, an infectious disease caused by a pathogenic agent, specifically, a viral, bacterial, fungal, parasitic pathogen and the like. Pathogenic agents include prokaryotic microorganisms, lower eukaryotic microorganisms, complex eukaryotic organisms, viruses, fungi, prions, parasites, yeasts, toxins and venoms. Still further, in some embodiments, the methods of the invention may be applicable for disorders caused by a viral pathogen. A viral pathogen, as used herein, may be in some embodiments, of any of the following orders, specifically, Herpesvirales (large eukaryotic dsDNA viruses), Ligamenvirales (linear, dsDNA (group I) archaean viruses), Mononegavirales (include nonsegmented (−) strand ssRNA (Group V) plant and animal viruses), Nidovirales (composed of (+) strand ssRNA (Group IV) viruses), Ortervirales (single-stranded RNA and DNA viruses that replicate through a DNA intermediate (Groups VI and VII)), Picornavirales (small (+) strand ssRNA viruses that infect a variety of plant, insect and animal hosts), Tymovirales (monopartite (+) ssRNA viruses), Bunyavirales contain tripartite (−) ssRNA viruses (Group V) and Caudovirales (tailed dsDNA (group I) bacteriophages). In yet some more specific embodiments, the methods of the invention may be applicable for a viral disorder such as a Foot and Mouth Disease.

In yet some other specific embodiments, the systems, methods and composition of the invention may be applicable for treating an infectious disease caused by bacterial pathogens. More specifically, a prokaryotic microorganism includes bacteria such as Gram positive, Gram negative and Gram variable bacteria and intracellular bacteria.

Examples of bacteria contemplated herein include the species of the genera Treponema sp., Borrelia sp., Neisseria sp., Legionella sp., Bordetella sp., Escherichia sp., Salmonella sp., Shigella sp., Klebsiella sp., Yersinia sp., Vibrio sp., Hemophilus sp., Rickettsia sp., Chlamydia sp., Mycoplasma sp., Staphylococcus sp., Streptococcus sp., Bacillus sp., Clostridium sp., Corynebacterium sp., Proprionibacterium sp., Mycobacterium sp., Ureaplasma sp. and Listeria sp.

Particular species include Treponema pallidum, Borrelia burgdorferi, Neisseria gonorrhea, Neisseria meningitidis, Legionella pneumophila, Bordetella pertussis, Escherichia coli, Salmonella typhi, Salmonella typhimurium, Shigella dysenteriae, Klebsiella pneumoniae, Yersinia pestis, Vibrio cholerae, Hemophilus influenzae, Rickettsia rickettsii, Chlamydia trachomatis, Mycoplasma pneumoniae, Staphylococcus aureus, Streptococcus pneumoniae, Streptococcus pyogenes, Bacillus anthracis, Clostridium botulinum, Clostridium tetani, Clostridium perfringens, Corynebacterium diphtheriae, Proprionibacterium acnes, Mycobacterium tuberculosis, Mycobacterium leprae and Listeria monocytogenes.

A lower eukaryotic organism includes a yeast or fungus such as but not limited to Pneumocystis carinii, Candida albicans, Aspergillus, Histoplasma capsulatum, Blastomyces dermatitidis, Cryptococcus neoformans, Trichophyton and Microsporum, are also encompassed by the invention.

A complex eukaryotic organism includes worms, insects, arachnids, nematodes, aemobe, Entamoeba histolytica, Giardia lamblia, Trichomonas vaginalis, Trypanosoma brucei gambiense, Trypanosoma cruzi, Balantidium coli, Toxoplasma gondii, Cryptosporidium or Leishmania.

More specifically, in certain embodiments the methods and compositions of the invention may be suitable for treating disorders caused by fungal pathogens. The term “fungi” (or a “fungus”), as used herein, refers to a division of eukaryotic organisms that grow in irregular masses, without roots, stems, or leaves, and are devoid of chlorophyll or other pigments capable of photosynthesis. Each organism (thallus) is unicellular to filamentous and possess branched somatic structures (hyphae) surrounded by cell walls containing glucan or chitin or both, and containing true nuclei. It should be noted that “fungi” includes for example, fungi that cause diseases such as ringworm, histoplasmosis, blastomycosis, aspergillosis, cryptococcosis, sporotrichosis, coccidioidomycosis, paracoccidio-idoinycosis, and candidiasis.

As noted above, the present invention also provides for the methods and compositions for the treatment of a pathological disorder caused by “parasitic protozoan”, which refers to organisms formerly classified in the Kingdom “protozoa”. They include organisms classified in Amoebozoa, Excavata and Chromalveolata. Examples include Entamoeba histolytica, Plasmodium (some of which cause malaria), and Giardia lamblia. The term parasite includes, but not limited to, infections caused by somatic tapeworms, blood flukes, tissue roundworms, ameba, and Plasmodium, Trypanosoma, Leishmania, and Toxoplasma species.

As used herein, the term “nematode” refers to roundworms. Roundworms have tubular digestive systems with openings at both ends. Some examples of nematodes include, but are not limited to, basal order Monhysterida, the classes Dorylaimea, Enoplea and Secernentea and the “Chromadorea” assemblage.

Still further, in certain embodiments, the systems, compositions, cells and methods of the invention may be applicable for treating disorders associated with immunodeficiency.

In some specific embodiments wherein the immune-related disorder or condition may be a primary or a secondary immunodeficiency. ‘Immunodeficiency’, primary or secondary, meaning inherited or acquired, respectively. The term ‘immunodeficiency’ is intended to convey a state of an organism, wherein the immune system's ability for immuno-surveillance of infectious disease or cancer is compromised or entirely absent.

More than 150 primary immunodeficiency diseases (PIDs) have been characterized, and the number of acquired (or secondary) immuno-deficiencies exceeds the number of PIDs.

PIDs are those caused by inherited genetic mutations. Secondary immuno-deficiencies are caused by various conditions, aging or agents such as viruses or immune suppressing drugs. A number of notable examples of PIDs include Severe combined immunodeficiency (SCID), DiGeorge syndrome, Hyperimmunoglobulin E syndrome (also known as Job's Syndrome), Common variable immunodeficiency (CVID): B-cell levels are normal in circulation but with decreased production of IgG throughout the years, so it is the only primary immune disorder that presents onset in the late teens.

Chronic granulomatous disease (CGD): a deficiency in NADPH oxidase enzyme, which causes failure to generate oxygen radicals. Classical recurrent infection from catalase positive bacteria and fungi. Wiskott-Aldrich syndrome (WAS); autoimmune lymphoproliferative syndrome (ALPS); Hyper IgM syndrome: X-linked disorder that causes a deficiency in the production of CD40 ligand on activated T-cells. This increases the production and release of IgM into circulation. The B-cell and T-cell numbers are within normal limits. Increased susceptibility to extracellular bacteria and opportunistic infections. Leukocyte adhesion deficiency (LAD); NF-κB Essential Modifier (NEMO) Mutations; Selective immunoglobulin A deficiency: the most common defect of the humoral immunity, characterized by a deficiency of IgA. Produces repeating sino-pulmonary and gastrointestinal infections. X-linked agammaglobulinemia (XLA; also known as Bruton type agammaglobulinemia): characterized by a deficiency in tyrosine kinase enzyme that blocks B-cell maturation in the bone marrow. No B-cells are produced to circulation and thus, there are no immunoglobulin classes, although there tends to be a normal cell-mediated immunity. X-linked lymphoproliferative disease (XLP); and Ataxia-telangiectasia.

Thus, patients' populations diagnosed with one of PIDs can particularly benefit from the systems, compositions, cells, and methods according to the present invention.

With respect to secondary immunodeficiencies, those can be manifested in both the young and the elderly. Under normal conditions immune responses are beginning to decline at around 50 years of age, what is called immunosenescence. The term ‘immunosenescence’ refers to the gradual deterioration of the immune system brought on by natural age advancement. It involves both the host's capacity to respond to infections and the development of long-term immune memory. Additional common causes of secondary immunodeficiency include severe burns, malnutrition, certain types of cancer, and chemotherapy in cancer patients.

More specifically, in developed countries, obesity, alcoholism, and drug use are common causes of poor immune function. However, malnutrition is the most common cause of immunodeficiency in developing countries. Diets lacking sufficient protein are associated with impaired cell-mediated immunity, complement activity, phagocyte function, IgA antibody concentrations, and cytokine production. Additionally, the loss of the thymus at an early age through surgical removal, for example, results in severe immunodeficiency and high susceptibility to infections.

Of particular relevance to the present context are cellular immunodeficiencies associated with cancer and certain viral pathogens. A cellular immunodeficiency refers to a deficiency the count or function of T lymphocytes, which are the main type of cells responsible for the cellular adaptive immune response in attacking viruses, cancer cells and other parasites. Extensive research has reasonably well established the role of immunodeficiency in cancers of the head and neck, lung, esophagus and breast. Among virally induced immunodeficiencies, the most notable example is AIDS (Acquired Immunodeficiency Syndrome) cause by HIV infection. The role of HIV as a direct cause of cellular immunodeficiency, particularly the deficiency of the CD4+ T helper lymphocyte population, has been well established. Additional examples of viral- or pathogen-induced immunodeficiencies include, although not limited to chickenpox, cytomegalovirus, German measles, measles, tuberculosis, infectious mononucleosis (Epstein-Barr virus), chronic hepatitis, lupus, and bacterial and fungal infections. One of the most recent examples is virus-induced Severe Acute Respiratory Syndrome (SARS).

These and additional examples of disorders related to cellular immunodeficiency may include Aplastic anemia, Leukemia, Multiple myeloma, Sickle cell disease, chromosomal disorders such as Down syndrome, infectious diseases caused by pathogens such as Cytomegalovirus, Epstein-Barr virus, Human immunodeficiency virus (HIV), Measles and certain bacterial infections. Chronic kidney disease, Nephrotic syndrome, Hepatitis, Liver failure and other conditions caused by Malnutrition, alcoholism and burns.

Thus patients' populations diagnosed with one of the secondary immunodeficiencies, and particularly one of the cellular immunodeficiencies as above, can particularly benefit from the systems, compositions, cells and methods of the present invention. Differential diagnosis of such immunodeficient patients is routinely performed in various clinical settings.

Additional secondary immunodeficiencies may result following bone marrow (BM) transplantation, gene therapy or adaptive cell transfer.

Hematopoietic stem cell transplantation (HSCT) is the transplantation of multipotent hematopoietic stem cells, usually derived from bone marrow, peripheral blood, or umbilical cord blood. It may be autologous (the patient's own stem cells are used), allogeneic (the stem cells come from a donor) or syngeneic (from an identical twin).

Performance of this medical procedure usually requires the destruction of the recipient's immune system using radiation or chemotherapy before the transplantation. To limit the risks of transplanted stem cell rejection or of severe graft-versus-host disease in allogeneic HSCT, the donor should preferably have the same human leukocyte antigens (HLA) as the recipient. In the case of a bone marrow transplant, the HSC are removed from a large bone of the donor, typically the pelvis, through a large needle that reaches the center of the bone. Peripheral blood stem cells are now the most common source of stem cells for HSCT. They are collected from the blood through a process known as apheresis. The donor's blood is withdrawn through a sterile needle in one arm and passed through a machine that removes white blood cells. The red blood cells are returned to the donor. The peripheral stem cell yield is boosted with daily subcutaneous injections of Granulocyte-colony stimulating factor (G-CSF), serving to mobilize stem cells from the donor's bone marrow into the peripheral circulation. It should be noted that amniotic fluid as well as umbilical cord blood may be also used as a source of stem cells for HSCT.

It should be appreciated that the methods of the invention enable in vivo editing of a target nucleic acid sequence of interest in cells of the treated subjects, by administering to the treated subject the systems, compositions and cells disclosed herein, preferably, using he disclosed methods.

In some embodiments, the cell or population of cells comprising the indicated cell used by the disclosed therapeutic methods may be at least one hematopoietic cell and/or at least one stem cell.

In some embodiments, the hematopoietic cell used by the therapeutic methods disclosed herein may be at least one lymphocyte. In more specific embodiments, the lymphocyte may be at least one genetically modified or unmodified cell of the T-cell-lineage.

In yet some further embodiments, the T cell used by the therapeutic methods at least one of Tregs, TIL cell, CTL, and NK cells.

In some embodiments, the method comprises the step of administering to the subject a therapeutically effective amount of at least one of any of the genetically modified cells as defined by the present disclosure, any population of cells or of any composition comprising the at least one cell or any population thereof. In some embodiments, the at least one cell is of an autologous or allogeneic source.

In some embodiments, the disclosed therapeutic methods comprise the step of administering to the subject a therapeutically effective amount of the immune trans-regulatory/modulatory system, or any vehicle or vector comprising the system or any composition thereof.

A further aspect of the present disclosure relates to an effective amount of at least one of: (I), an effective amount of an immune trans-regulatory/modulatory system comprising: in one component (a), at least one promoter-less donor nucleic acid molecule comprising at least one replacement nucleic acid sequence. The replacement nucleic acid sequence and/or the donor nucleic acid molecule comprising the replacement sequence is inserted and/or introduced into a target sequence within at least one target first immune-regulatory gene of interest. In some embodiments, the replacement nucleic acid sequence replaces at least part of the endogenous coding region of the target first immune-regulatory gene.

In some alternative or additional embodiments, the replacement sequence is introduced into the endogenous non-coding region of the target first immune-regulatory gene, such that the target gene is intact. The replacement, and/or introduction of the replacement sequence provided by the donor nucleic acid molecule, results in at least one of: (i) the production of at least one immune-modulatory product, controlled by at least one endogenous control element of the target first immune-regulatory gene; and (ii) inhibition and/or reduction and/or modulation of the expression of the target first immune-regulatory gene; and in another component (b), at least one target recognition element targeted at a target sequence within the target first immune-regulatory gene, or any nucleic acid sequence encoding the target recognition element. In some embodiments, where nucleic acid sequences that encode the target recognition element are used, these sequences may be provided either as part of the same nucleic acid donor molecule, or in at least one separate nucleic acid molecule. In some optional embodiments, the system that may be used by the disclosed methods may further comprise in another component (c), at least one nucleic acid guided genome modifier protein, chimeric protein, complex or conjugate, or at least one nucleic acid sequence encoding said guided genome modifier protein. In some embodiments, when the nucleic acid sequence encoding the nucleic acid guided genome modifier protein is provided in the disclosed system, and when nucleic acid sequences that encode the target recognition element are used, the nucleic acid sequence encoding the at least one nucleic acid guided genome modifier protein may be provided either in a separate nucleic acid molecule, or in the same molecule together with the nucleic acid sequence that encodes the target recognition element, and/or as part of the at least one donor nucleic acid molecule.

In some alternative or additional embodiments, the disclosure provides an effective amount of (II), at least one nucleic acid cassette or any vector or vehicle comprising at least one of: (i) the nucleic acid sequence of (a); (i) the nucleic acid sequence of (b); (iii) the nucleic acid sequence of (c); and (iv) the nucleic acid sequence of (a) and at least one of (b) and (c). Still further, in some alternative or additional embodiments, the disclosure provides an effective amount of (III), at least one cell comprising and/or modified by at least one of: the nucleic acid the at least one system of (I), or the at least one nucleic acid cassette or any vector or vehicle of (II); or any matrix, nano- or micro-particle comprising at least one of (I) and (II), and/or a population of cells comprising at least one of the cell/s.

In yet some further alternative or additional embodiments, the disclosure provides an effective amount of (IV), at least one composition comprising at least one of (I), (II), (III) or any combinations thereof, or any combinations thereof; for use in a method of curing or treating, preventing, inhibiting, reducing, eliminating, protecting or delaying the onset of a pathologic disorder in a mammalian subject.

However, in some alternative embodiments, the desired editing of the target nucleic acid sequence, may be performed ex vivo. In such option, the editing, or genetic manipulation of the nucleic acid sequence of interest is performed in cells of an autologous or allogeneic source, that are then administered to the subject.

In some embodiments, such cell has been ex vivo modified using the systems of the invention. Thus, in some embodiments thereof, the methods of the invention may comprise the step of administering to the treated subject a therapeutically effective amount of at least one cell as defined by the invention or of any composition comprising any of the cells disclosed by the invention.

As discussed herein, the present disclosure provides modulatory and therapeutic methods that can be performed either in vivo, by providing the disclosed systems, specifically, by contacting with cells, and/or administration of the system to the treated subject, or ex vivo, by manipulating and editing the target cells (from autologous or alternatively allogeneic source) out of the donor's body, and returning and/or transplanting the edited cells, to the subject. In some embodiments, such administration of the edited cells is indicted herein as ACT. Adoptive cell transfer (ACT) is the transfer of cells into a patient. The cells may have originated from the patient or from another individual. The cells are most commonly derived from the immune system, with the goal of improving immune functionality and characteristics. In cancer immunotherapy, T cells are extracted from the patient, genetically modified and cultured in vitro and returned to the same patient.

An “effective amount” of the systems, nucleic acids, cell/s (and/or populations of cells that comprise at least one of the cells disclosed by the present disclosure) and compositions of the invention, is in some embodiments, any amount effective for modulating a target cell [e.g., to secrete immuno-modulatory components, to display an immuno-modulated action (e.g., activation or inhibition)], and/or for modulating an immune response in a treated subject (a therapeutically effective amount). The effective amount, that in some embodiments may be a therapeutically effective amount, can be administered in one administration, or through multiple administrations of an amount that total an effective amount, preferably within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

It is to be understood that the terms “treat”, “treating”, “treatment” or forms thereof, as used herein, mean curing, preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease.

The term “treatment or prevention” as used herein, refers to the complete range of therapeutically positive effects of administrating to a subject including inhibition, reduction of, alleviation of, and relief from, an immune-related condition and illness, immune-related symptoms or undesired side effects or immune-related disorders. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation”, “prevention”, “suppression”, “repression”, “elimination” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the immune-related disorders and/or proliferative disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described herein.

The terms “delay”, “delaying the onset”, “retard” and all variations thereof are intended to encompass the slowing of the progress and/or exacerbation of a disorder associated with the immune-related disorders and their symptoms slowing their progress, further exacerbation or development, so as to appear later than in the absence of the treatment according to the invention.

As indicated above, the methods and compositions provided by the present invention may be used for the treatment of a “pathological disorder”, specifically, immune-related disorders and/or proliferative disorders as specified by the invention, which refers to a condition, in which there is a disturbance of normal functioning, any abnormal condition of the body or mind that causes discomfort, dysfunction, or distress to the person affected or those in contact with that person. It should be noted that the terms “disease”, “disorder”, “condition” and “illness”, are equally used herein.

It should be appreciated that any of the methods and compositions described by the invention may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

The present invention relates to the treatment of subjects or patients, in need thereof. By “patient” or “subject in need” it is meant any organism who may be affected by the above-mentioned conditions, and to whom the therapeutic and prophylactic methods herein described are desired, including humans, domestic and non-domestic mammals such as canine and feline subjects, bovine, simian, equine and rodents, specifically, murine subjects. More specifically, the methods of the invention are intended for mammals. By “mammalian subject” is meant any mammal for which the proposed therapy is desired, including human, livestock, equine, canine, and feline subjects, most specifically humans.

It should be further appreciated that the present disclosure further encompasses any nucleic acid cassette, construct, vehicle or vector that comprise at least one of the donor molecules of the disclosed systems.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

### Experimental Procedures

### Plasmids and Donor Construction

Synthetically produced by Thermo scientific

Nuclease mRNA Production

In Vitro transcription: Capped polyA mRNA is prepared using T7 mScript™ Standard mRNA Production System (CellScript C-MSC100625), according to the manufacturer's instructions.

### Cells Protocols

Fresh PBMCs from by-products of blood donations approved by the Helsinki Committee of the medical institution were used as starting material for T-cell selection using RosetteSep protocol of STEMCELL Technologies Inc.

### Improved Editing in Primary T Cells—Electroporation Protocol

1 Activation:
a. Dilute cells to 1 million/ml in X-VIVO 20 media.
b. Add CD3/CD28—25 μl/ml
c. and 300 IU/ml IL2 (3 μl/ml)
2 Electroporation 72h after Activation (Lonza Nucleofector 4D)
a. RNA preparation:
Mix RNA in separate tubes per treatment:
   Nuclease encoding mRNA 3 μg
   Synthetic Guide 7 μg
   Synthetic Guide 7 μg
   Donor Variable 2.5-4 μg
Total volume—up to 6 ul
b. In 12 well plates:
i. 3 ul IL2/ml X-VIVO 20 media (final conc. 300 IU/ml)
ii. Add 900 ul medium/well
iii. Incubate to 37° C.
c. Incubate at 37 C large amount of X-vivo 20+IL2 medium
d. Start 4D-Nucleofector™ System
e. Count and pellet cells (Spin—100/200 g 10 min)
i. Wash cells with PBS first time
ii. pellet (Spin—100/200 g 10 min)
iii. Wash cells with PBS second time
iv. pellet (Spin—100/200 g 10 min)
v. Resuspend in nucleofector solution—1 million cells/20 μl/sample
f. Add 20 μl cells to RNA already prepared in Eppendorf tube and transfer to cuvettes.
g. Nucleofect cells (program EO-115)
h. Add 80 μl warm X-VIVO 20+IL2 recovery medium.
i. Transfer cells from each cuvette to an Eppendorf tube (Using rubber pipette).
Total well volume is 1 ml media with 1 million cells
Grow cells for 96 h at 37° C. before harvest. Fresh medium with IL2 is added every other day.

### Reverse Transfection

Reverse transfection to HEK-293 cells was done using TransIT® LT1 transfection reagent (Mirus Bio) diluted in serum-free medium Opti-MEM® I (Gibco). Transfection reagent:DNA ratio of 5 μl per 1 g DNA was used. For reverse transfection in a well in 96 wells plate: 100 ng plasmid DNA was used in 3 ul total volume (33.3 ng/ul). Transfection reagent was first diluted (1:19) in Opti-MEM® I. Then, 3 ul DNA (33.3 ng/ul) was added to the diluted transfect reagent, mixed gently and incubated 15-30 min in room temperature. Then, HEK-293 cells (3.6×10⁴) were gently added on the top of the TransIT®-DNA complexes and mixed as is customary. Cells were incubated 72h in 37° C. in a CO2 incubator (HERACELL 150i, Thermo Scientific). If co-transfection was done, plasmids were equally mixed in advance to final DNA concentration of 100 ng/3 ul. (for example: if two plasmids were used, 50 ng from each plasmid was mixed in 3 ul final volume 33.3 ng/ul DNA, if three plasmids were used, 33.3 ng from each was mixed in 3 ul final volume 33.3 ng/ul DNA).

### Genomic DNA Preparation

Genomic DNA (gDNA) from HEK-293/t-cells cells was extracted 72 hours post transfection, using the Quick-DNA™ 96 Kit (Zymo Research). Medium was removed using vacuum and glass Pasteur pipette. Then, 200 μl Genomic Lysis Buffer were added and using mechanic pipetation up and down, cells were directly lysed in the 96 plate well. The concentration of gDNA was determined using NanoDrop 2000 (Thermo Scientific).

### Droplet Digital PCR Design and Execution.

QX200™ Droplet Digital™ PCR system (BIORAD) was used for the mutations analysis in the target site on the gDNA.

Assay designed, TGEE50-52

Detailed reaction particulars and primers can be found in Table 2. As TGEE3 and TGEE4 are assays to analyze mutation in adjacent sites, same primers were used for the amplification of the target site were in both assays. In addition, the same reference probe was used for both assays. The drop-off probes were different and specific for each site.

TABLE 1
guides and primers

| Seq | | | | | | | | |
|---|---|---|---|---|---|---|---|---|
| ID | | | | | | | reference | drop off |
| guides | Guide name | gene | exon | assay | primerF | primerR | probe | probe |
| 7 | TGEEpd1ex3.1 | PD1 | EX3 | TGEE52 | 3733 | 3718 | 3762 | 3737 |
| 9 | TGEEpd1ex3.3 | PD1 | EX3 | TGEE51 | 3733 | 3718 | 3762 | 3736 |
| 10 | TGEEpd1ex3.4 | PD1 | EX3 | TGEE51 | 3733 | 3718 | 3762 | 3736 |
| 12 | TGEEpd1ex3.6 | PD1 | EX3 | TGEE50 | 3733 | 3718 | 3762 | 3732 |
| 13 | TGEEepd1x3.7 | PD1 | EX3 | TGEE50 | 3733 | 3718 | 3762 | 3732 |
| 14 | TGEEpd1ex3.8 | PD1 | EX3 | TGEE50 | 3733 | 3718 | 3762 | 3732 |
| 11 | TGEEpd1ex3.16 | PD1 | EX3 | TGEE51 | 3733 | 3718 | 3762 | 3736 |

TABLE 2
Primers and probes sequences

| SEQ ID NO. | PRIMER | SEQUENCE |
|---|---|---|
| SEQ ID NO: | 3718 | tgggatgacgttacctcgt |
| 105 | | |
| SEQ ID NO: | 3732 | /5HEX/CC AGT TCC +A+AA CCC TGG |
| 106 | | T/3IABKFQ/ |
| SEQ ID NO: | 3733 | cctctagctctggaagcac |
| 107 | | |
| SEQ ID NO: | 3736 | /5HEX/AG CCG GCC A+G+T TCC AAA |
| 108 | | C/3IABKFQ/ |
| SEQ ID NO: | 3737 | /5HEX/TC ACC CAG +G+CC AGC CG/3IABKFQ/ |
| 109 | | |
| SEQ ID NO: | 3762 | /56-FAM/CC TGA CCT T/ZEN/T GTG CCC |
| 110 | | TT/3IABKFQ/ |

Then, the reference probe was ordered from IDT with FAM™ modification in the 5′ end and with Iowa Black® Quencher in the 3′ end. The drop off probe was ordered from IDT with HEX™ modification in the 5′ end and with Iowa Black® Quencher in the 3′ end with 2 locked nucleic acid (LNA) bases inside the target site. Tm of reference and drop off probes were designed to be higher in 3-10° C. than 55° C.

### Data Analysis

QX200™ Droplet Reader and QuantaSoft™ software (BIORAD) were used for Data analysis.

For each assay and each experiment, a threshold was determined in relation to all experiment treatments, controls and no DNA control sample.

Thresholds were analyzed separately for each experiment and for each assay.

Poisson correction was done according to manufacturer's instructions (Droplet Digital PCR Applications Guide, BioRad, p7-8). Briefly, a Poisson correction factor is inferred by modeling a Poisson distribution from the fraction of empty cells. Explicitly, the Poisson correction factor is the infinite sum of the probability of a cell containing 1 DNA molecule only, plus two times the probability of two DNA molecules, plus three times the probability of three DNA molecules, and so on. This correction factor is multiplied with the observed number of hits to find the true number of DNA molecules.

### Methods for Off-Target Detection Generated by the Nucleases of the Invention

Off-target mutations may cause genomic instability and disrupt the functionality of otherwise normal genes. Therefore, it is important to be able to detect the presence of off-target cleavage. Methods for off-target detection fall into two categories, biased and unbiased. Biased methods designed to detect mutations at predicted potential off-target sites whereas unbiased methods will ideally locate this kind of mutations anywhere in the genome.

All biased methods are PCR based assays and share the same initial steps. First, prediction of off-target sites will be made using different In Silico CRISPR\Cas9 design tools or other relevant bioinformatics methods. Next, primers are designed for those specific sites and amplified by PCR in order to validate the cleavage. From this step forward, other than the obvious sequencing of the PCR products, there are several other methods to detect those anticipate off-target mutations, such as High-resolution melting analysis (HRMA), Mismatch cleavage with T7 or Surveyor endonuclease and mobility assay by PAGE

All biased methods are cheap, simple and fast but suffer from an inability to detect off-target mutations that occur at frequencies <1%, and suffer from the fact that there is a good portion of the off-target sites that cannot be predicted by the design tools available today. Furthermore these methods are not practical for large scale screening. However, when a specific oncogene or important regulatory element has been even weakly predicted to be under risk, such targeted PCR based assays should be employed.

Among the many unbiased methods existing, listed here are few of the more efficient and more relevant to the examples of the invention:

### 1. LAM-HTGTS—Linear Amplification-Mediated High-Throughput Genome-Wide Translocation Sequencing

LAM PCR can detect unknown DNA sequence that is in proximity to a known one. LAM-HTGTS is based on the translocation between known DNA added to the cells (refer to as ‘bait’) and the unknown fragments of DNA where the off-target DSB have occur (refer to as ‘prey’). LAM-PCR oriented toward the ‘bait’ known DNA will also amplify the ‘prey’ sequence thus allowing us to locate and identify off target cleavage in the genome.

### 2. BLESS—Direct In Situ Breaks Labeling, Enrichment on Streptavidin, and Next-Generation Sequencing

The DSB are directly labeled using Biotinylated pinheads' linkers, enriches them on streptavidin and eventually analyzes the fragments using NGS and PCR with linker-specific primers.

### 3. GUIDE-Seq—Genome-Wide Unbiased Identification of DSBs Enabled by Sequencing

In this method the identification of the DSBs occurs via blunt double-stranded oligodeoxynucleotide (dsODN) that integrate with blunt DSBs in the genome (caused by RNA guided nuclease) through end joining processes such as NHEJ. The dsODN integration sites then amplified using NGS in order to locate the off-target DSBs.

### 4. Digenome-Seq—In Vitro Nuclease-Digested Genomes (Digenomes).

The genome is digested in vitro, using the guided nuclease of the invention, into smaller fragments with identical 5′ ends (sequence reads). After applying whole genome sequencing (WGS) on those fragments they will vertically align at cleavage sites, while uncut sequences will be aligned in a staggered pattern. Hence, off-target DSBs can be identified.

### 5. Whole Genome Sequencing

High throughput sequencing of the entire genome and comparison to a reference sequence.

The entire genome is screened for off-target mutations therefore it is very accurate but also very expensive. This method has serious drawbacks in discerning SNPs and sequencing errors from bona fide off-target mutations and in the limited number of genomes that can be sequenced.

### Example 1

### Direct Editing of the PD1 Locus

Programmed cell death protein 1 (PDCD1 or PD-1) is a protein expressed on the surface of immune cells that allows downregulation of the immune system. The PD1 receptor on lymphocytes has two ligands: PDL1 and PDL2. PDL1 is typically expressed on immune cells so that they don't destroy each other in inflammation and is also expressed in tissue (and tumors) during inflammation. The PD1-PDL1 axis is the most important “break” (tolerance) at peripheral sites of inflammation. PDL1 expression allows tumors to hide from the immune system. During inflammation, interferon gamma will upregulate PDL1 expression. Anti-PD1 mAbs have been developed that suppress the PD1-PDL1 axis thus potentiating the immune response to cancer, the first being Nivolumab and Pembrolizumab (Topalian et al, 2012, NEJM, 366:2443-2454).

Ex-vivo gene knock-out of PD-1 may allow sustained suppression of the PD1-PDL1 axis thus increasing cancer immunotherapy efficacy, has been shown for Cas9-based knock-out of PD-1 (Lu et al, 2020, Nat Med, 26:732-40).

Rewiring the PD1-PDL1 axis may yield further improved outcomes. For example, combining PD1 extracellular domain with 41BB intracellular domain has been proposed to enhance T-cell targeting due to the stimulatory effect of 41BB [5]. In this case, PD1-41BB fusion protein was delivered via retroviral transduction. Viral delivery has the disadvantage that it is not the genomic DNA fusion with the endogenous PD1 gene of the current invention and thus not under temporal and localized control of the relevant promoter.

Direct editing of the PD1 locus to simultaneously remove the natural intracellular PD1 domain (which would have repressed the lymphocyte) and replace it with the intracellular domain of 41BB (which now activates the lymphocyte) is effective with efficient HDR-based gene editing. Moreover, additional stimulatory genes may be delivered in a large construct for optimized activity. Here Homology Directed DNA Repair (HDR) editing of PD-1 is proposed to create a PD1-41BB fusion in an operon with CCL21 and IL7 for enhanced cancer immunotherapy. Co-expression of IL7 and CCL21 has been shown to increase activity of CAR-T Cells in solid tumors [6].

PD-1 exon 3 is targeted via guides for dCasFok (SEQ ID NO:7-15) and for Cas9 (SEQ ID NO:16-18). Gene editing efficiency was tested by measuring NHEJ erroneous repair by ddPCR, as shown by FIG. 3, and TIDE ((Tracking of Indels by DEcomposition) Brinkman et al, 2014, NAR, e168).

NHEJ may result in base pair insertion/deletions (indels) depending on the particular guide and editing system (dCasFok or Cas9). An indel with a size of a multiple of three will not change the reading frame of the resulting PD-1 protein, but instead cause amino acid deletions or insertions. An indel with a size that is not a multiple of three will change the reading frame, resulting in an altered amino-acid sequence of the protein and/or premature stop codons.

In PD-1, reframing at exon 3 (SEQ ID NO: 19) results in two products: a −1 (or +2) frame shift results in a −183 amino acid truncated protein (example product shown in SEQ ID NO: 20), and a −2 (or +1) frame shift results in a ˜242 amino acid truncated protein (example product shown in SEQ ID NO:21). For comparison, native PD-1 is 288 amino acids. Since the premature stop codon in the −1 frame shift product occurs in exon 3, it is predicted not to express due to Nonsense Mediated Decay (Baker and Parker, 2004, Curr Opin in Cell Biol, 16:293-99). The premature stop codon in the −2 frame shift occurs in exon 5 so it is expected to express, generating a soluble protein since the PD-1 transmembrane domain is removed.

An integration cassette that contains an in-frame 41BB transmembrane domain and intracellular domain (ICD) replaces the corresponding region from PD-1. IL7 (SEQ ID NO: 1) and CCL21 (SEQ ID NO:2) are also expressed, separated by 2A self-cleaving peptides (T2A, SED ID NO:3; P2A, SEQ ID NO:4), with transcription terminated by BGHT (SEQ ID NO:5), as illustrated in FIG. 1.

Five integration cassettes were designed (SEQ ID NO:22-26). 41BBICD-T2A-CCL21-P2A-IL7 PD1 integration cassette (SEQ ID NO:26) contains all elements described above and encodes the protein PD1Nterm-41BBICD-T2A-CCL21-P2A-IL7 (SEQ ID NO:27).

Four additional constructs test functionality of the individual components, replacing them with fluorescent proteins that can be detected with flow cytometry. 41BBtransmembrane-EGFP-T2A-EBFP-P2A-luciferase PD1 integration cassette (SEQ ID NO:22) replaces the 41BB ICD with EGFP (SEQ ID NO:28), CCL21 with EBFP (SEQ ID NO:29), and IL7 with luciferase (SEQ ID NO:30). The complete protein

construct is shown in SEQ ID:31. 41BBtransmembrane-EGFP-T2A-IL7-P2A-luciferase PD1 integration cassette (SEQ ID NO:23) replaces the 41BB ICD with EGFP, and the IL7 with luciferase. The complete protein product of the construct is shown in SEQ ID NO:32.

41BBICD-T2A-EBFP-P2A-luciferase PD1 integration cassette (SEQ ID NO:24) replaces CCL21 with EBFP and IL7 with luciferase. The complete protein construct is shown in SEQ ID NO:33.

41BBtransmembrane-EGFP-T2A-mCherry-P2A-luciferase PD1 integration cassette (SEQ ID NO:25) replaces 41BB TCD with EGFP, CCL21 with mCherry (SEQ ID NO:34) and IL7 with luciferase. The complete protein product of the construct is shown in SEQ ID NO:35.

Constructs were assembled by gene synthesis.

In an alternative formulation, a soluble fusion protein of human PD1 and Fc (SEQ ID NO: 36) is created from HDR at the PD1 locus. A fusion between PD1, Fc, and OX40L may also be created (SEQ ID NO:37) [7]. This is in order to switch the PDL1/2 suppressory signaling to an activating signal for immune cell encountering this ligand.

Thus, upon activation of the modified cells, the gene is expressed and the fusion protein is secreted. The trans-activated target cells for such secreted fusion proteins can include NK or macrophage cells (PD1-FC) via their activating FC-receptors or T-cells (PD1-FC-OX40L), via their relevant receptors.

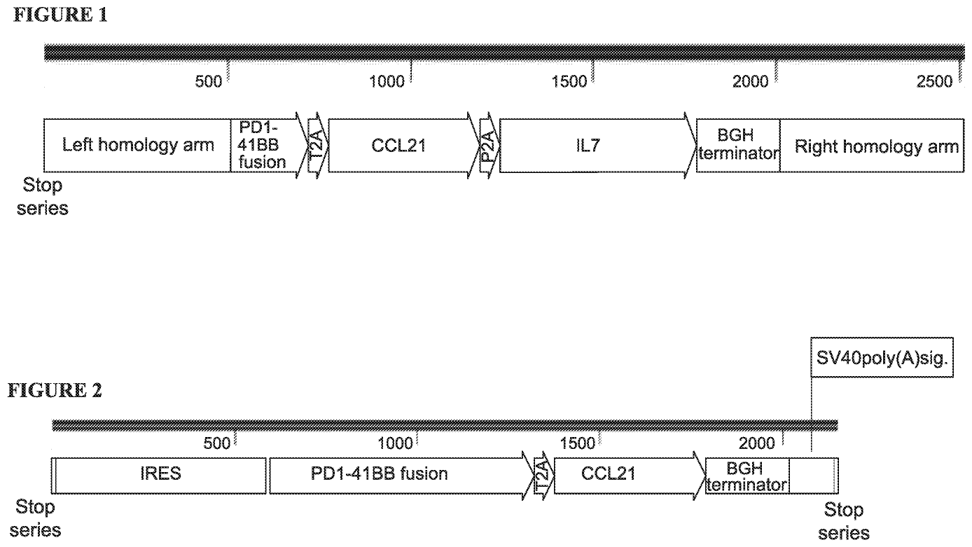

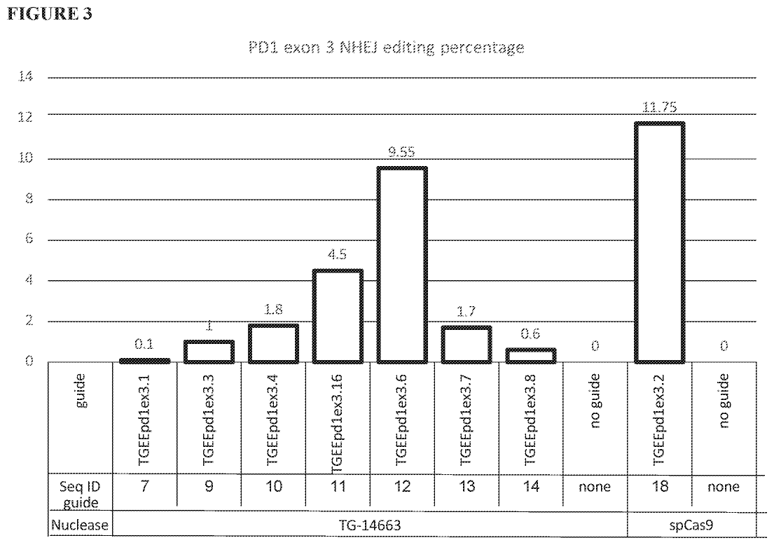

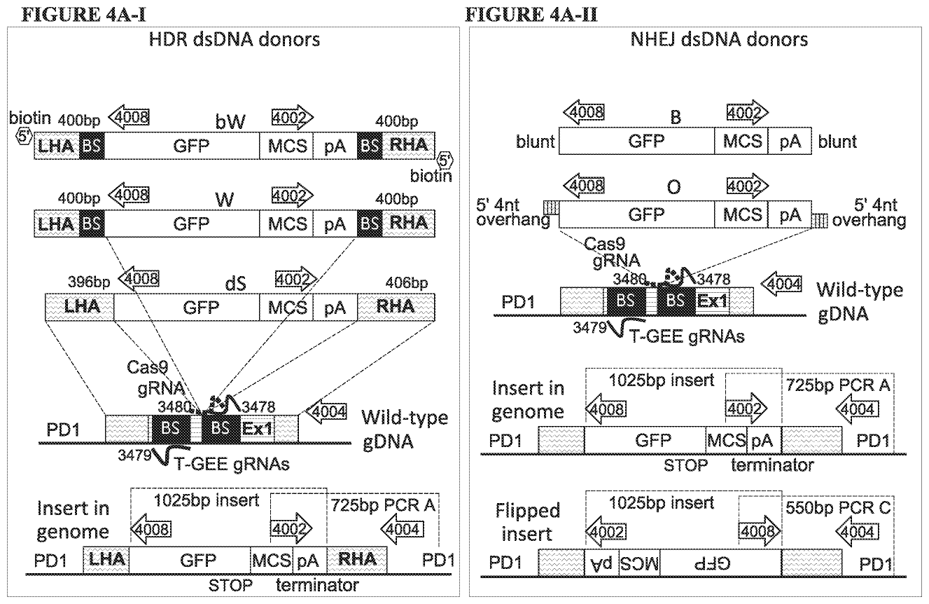

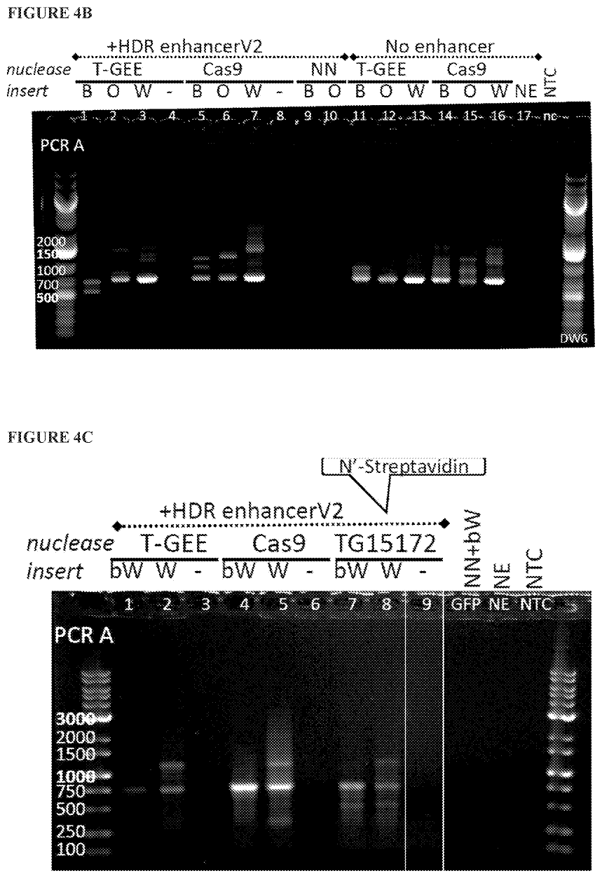

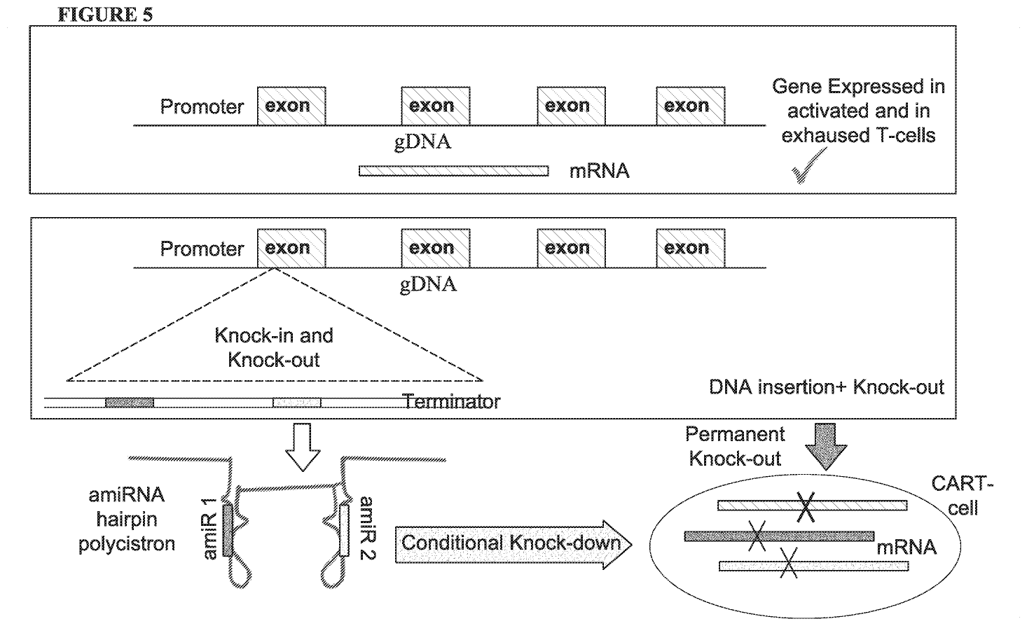

In a further alternative formulation, integration of the donor cassette is carried out with Non Homologous End Joining (NHEJ) instead of HDR. A cassette containing stop codon series followed by an IRES (internal ribosome entry site) allowing translation from the same mRNA as the disrupted PD1 gene. The integration cassette contains extracellular PD-1 fused to a 41BB transmembrane domain and intracellular domain (ICD). IL7 (SEQ ID NO: 1) and CCL21 (SEQ ID NO:2) are also expressed, separated by 2A self-cleaving peptides (T2A, SED ID NO:3; P2A, SEQ ID NO:4), with transcription terminated by BGHT (SEQ ID NO:5) as illustrated in FIG. 2. The DNA sequence for the full construct is shown in SEQ ID NO:6. A control construct expressing GFP is shown in SEQ ID NO:38.

Testing is carried out by flow cytometry to assess expression of fluorescent proteins upon stimulation both in inhibited as well as in exhausted cells. Similarly, the cells are tracked in animal models by imaging the luciferase gene. Thus, the trafficking, persistence, expansion, level of expression at relevant site and condition, and the fate of the cells would be revealed. In addition, the activity of the modified cells would be analyzed upon repeated stimulation without the presence PDL1/2 using ELISA tests to follow the activity states of the cells as well as the secretion pattern of the cytokines at the integration cassettes. Killing assays would be performed as well. The effect on other cells, in terms of recruiting and supporting, would be evaluated using transwell migration assays. Using in vivo animal models will reveal the level of recruitment and migration of the different cell to the tumors. In addition, the effect on tumor survival and control is evaluated by its size, survival and by tumor biopsy analysis of the pattern of angiogenesis as well the morphological structure and immune-cell penetration and activation.

Targeting of the PD1 Exon 1 in HEK293 and Activated T Cells Results in Successful Insertion that Allow Expression Under the Endogenous PD-1 Promoter

The inventors next evaluated the option of targeted insertion of a replacement sequence (GFP-donor constructs) into a target sequence within the PD-1 gene, using various machines disclosed by the present disclosure. More specifically, as gene editing tools the following machines were used: Cas9 protein, Cas9 protein-FokI chimera, and Cas9 protein comprising a streptavidin domain that can bind to biotinylated donor DNA (SEQ ID NO: 175) (Shiboleth and Weinthal, 2015, Compositions and Methods for Modifying a Predetermined Target Nucleic Acid Sequence), that acts as an HDR enhanced machine.

To test HDR vs NHEJ mediated insertion in HEK293 and T-cells the inventors constructed a set of donors (FIG. 4A-I, 4A-II, for HDR and NHEJ, respectively). This set of donors when correctly inserted into the human PDCD1 (PD1) exon 1 locus, allow expression of GFP from the endogenous PD1 promoter and abolish PD1 protein, together, a functional gene replacement. An exception is the Blunt (B) NHEJ donor denoted by SEQ ID NO:181 (1053 bp) which is inserted out-of-frame. Overhang (O) NHEJ donor has an identical slightly shorter sequence (1029 bp) derived from donor B after digestion by BsaI from both ends. This results in four single strand nucleotides at the 5′ of each side, corresponding to the expected sticky overhang in PD1 exon1 at the nuclease cleavage site of the HDR enhanced nucleic acid guided genome modifier chimeric or fusion protein used by the systems of the present disclosure, creating a seamless in-frame fusion.

Cleavage of this gene with spCas9 does not create a compatible sticky site.

To first compare between blunt (B), overhang (O) and an HDR donor (W) (SEQ ID NO: 182) (FIG. 4B), an experiment was conducted in Hek293 cells. W denotes the wSCNA donor—with full SCNA (guide RNA) 3478 (SEQ ID NO: 178) and 3479 (SEQ ID NO: 179) binding sites (BS). In W the 3480 (SEQ ID NO: 180) Cas9 guide has only a 9 bp overlap+PAM within the Right Homology Arm (RHA) designed so it cannot cut the donor. Results showed that in all treatments HDR resulted in properly recombined GFP in the PD1 locus, with a dominant product at the correct size as compared to the NHEJ donors B and O which had a much more “messy” outcome with probable concatamers and mis-inserted products (FIGS. 4B-4C). Analysis of flipped inserts using primers 4004 and 4008 (SEQ ID NOs: 184, 185, respectively) also confirmed that HDR donors resulted in much less mis-inserted donor than the NHEJ donors. Only minor differences for the HDR donor were observed between monomeric spCAS9 (Cas9) and dimeric T-GEE (the HDR enhanced nucleic acid guided genome modifier chimeric or fusion protein used by the systems of the present disclosure). More specifically, T-GEE used in this example, denotes mRNA transcribed from TG14664 (SEQ ID NO: 173) which is an ancestral dCasFok, his-tagged, Cas9 denotes mRNA transcribed from TG7665-Cas9 (SEQ ID NO: 174) which comprises spCas9. HDR enhancer (Alt-R HDR Enhancer V2, IDT) had no significant effect in this cell line in this experiment (FIG. 4B).

Next, the inventors examined the targeted insertion of the replacement sequence into exon 1 of the PD-1 gene in T cells. FIG. 4C shows successful HDR directed replacement of PD1 by GFP in activated human T-cells, using a non-biotinylated donor (W), Or biotinylated dsDNA donor DNA (bW, the HDR improved construct TG15172, the T-GEE construct, as well as Cas9. As shown by the figure, the use of construct TG15172 (Streptavidin (n-term), ancestral dCasFok, his-tagged, SEQ ID NO: 158) harboring the DAD N′-streptavidin domain, improved the gene editing, as the biotinylated donor bW (lane 7) display higher HDR efficiency than the same donor with the non-streptavidin fused nuclease T-GEE (lane 1) ostensibly due to the possibility that the streptavidin binds the biotin on the donor dsDNA bringing the donor in proximity to the dsDNA break site.

Strengthening this hypothesis is the observation that the non-biotinylated donor W does not exhibit this difference (compare lanes 2 and 8).

Taken together, these results establish the feasibility of using various genome modifiers, (e.g., HDR enhanced nucleic acid guided genome modifier chimeric or fusion proteins that contain DAD, T-GEE, as well as Cas9), for targeted replacement of PD-1 with a replacement sequence, by targeting exon 1.

### Example 2

### Direct Editing of the LIF Locus

An additional example for an immune-regulatory target gene of interest, is Leukemia inhibitory factor (LIF) and the promoter region of this gene. LIF is a cytokine which has autocrine and paracrine functions, and thus, may be one of the major driving factors for the recruitment of immunosuppressive subpopulations of lymphocytes into the TME [Wrona, E. et al. (2021) ‘Leukemia Inhibitory Factor: A Potential Biomarker and Therapeutic Target in Pancreatic Cancer’, Archivum Immunologiae et Therapiae Experimentalis, 69(1). doi: 10.1007/s00005-021-00605-w]. LIF can create an immunosuppressive microenvironment. LIF stimulates FoxP3 which upregulates the development and proliferation of Tregs. LIF is a strong chemoattractant for immunosuppressive cell recruitment into the TME.

LIF inhibition using humanized anti-LIF antibodies has been shown to have positive effect in orthotopic mouse cancer models including Pancreatic Cancer, Glioblastoma, NSCLC, Ovarian cancer and Colorectal cancer. Thus, reduction of LIF expression in the TME is expected to have a positive effect on treatment of solid tumors. While LIF is expressed by the tumor cells themselves it is differentially expressed by T-cells (Expression Atlas—E-MTAB-6865), whereby T cells sorted from human PBMCs had extremely low (1.44) average expression levels, T cells stimulated with CD3/CD28 beads for 4 days had a high (979.25) average expression levels, a 681-fold increase. Thus, the promoter driving LIF may be used to drive transcription of inserted genetic payload upon T-cell stimulation but not in naïve cells.

To knockout LIF or utilize the LIF promoter to drive expression of a genetic payload in T-cells, two alternative exons were targeted, exon 2 and exon 3.

Exon 2 of the LIF gene was targeted using one of the gRNA pairs (#6 and #5) of SEQ ID NO: 41 and 42 (LIF-ex2-6L and LIF-ex2-6R, respectively) or of SEQ ID NO: 43 and 44 (LIF-ex2-5L and LIF-ex2-5R, respectively). The gRNA pairs are used with the nucleoproteins of the invention.

One of these guide pairs, or other guide pairs that target a target sequence in exon 2, can be used for example to knockout the gene via NHEJ-ER or insert a genetic payload via NHEJ or via HDR. NHEJ has less predictable editing sequence results and may or may not conserve the coding frame, and thus, is highly suitable for non-coding RNA payloads such as those encoding siRNAs or artificial miRNA hairpins.

An example for a non-coding payload to be inserted via NHEJ into the LIF gene is “siPolycistron PD1/TIGIT/MIR4443/BTLA/LAG3” (SEQ ID NO: 100).

In order to insert via HDR into exon 2 of LIF, while removing most of the LIF protein and retaining the LIF coding frame, a construct carrying a payload is inserted between the exon 2 left and right homology arms. In this non-limiting example, the payload comprises P2A protease cleavage signal; CCL21; P2A; IL7 followed by a terminator of transcription. The HDR Donor Construct LIF exon 2, as used herein, is denoted by SEQ ID NO: 45. The LIF Left homology arm exon 2 is denoted by SEQ ID NO: 46, followed by the P2A sequence as denoted by SEQ ID NO: 47, the CCL21 encoding sequence as denoted by SEQ ID NO: 48, followed by a second P2A sequences, as denoted by SEQ ID NO: 49, and the IL7 encoding sequence as denoted by SEQ ID NO: 50, and terminated by the BGH-T terminator of SEQ ID NO: 5. The LIF-Right homology arm exon 2 is denoted by SEQ ID NO: 51.

In another example, the LIF gene exon 3 is next targeted for payload expression without knockout of the LIF gene using one of the following guide pairs (#5 and #1) as denoted by SEQ ID NOs: 52 and 53 (LIF-ex3-5L, LIF-ex3-5R, respectively), or by SEQ ID NOs: 54 and 55 (LIF-ex3-1L, LIF-ex3-1R, respectively), or using any other guide pairs targeting exon 3:

In order to insert via HDR into exon 3 of LIF, retaining both the intact LIF protein and the LIF coding frame, a construct carrying a payload is inserted between the exon 3 left and right homology arms. In this non-limiting example the payload comprises P2A protease cleavage signal; CCL21; P2A; IL7 followed by a terminator of transcription, as denoted by SEQ ID NO: 56. The LIF Left homology arm exon 3 is denoted by SEQ ID NO: 57, followed by the P2A sequence as denoted by SEQ ID NO: 47, the CCL21 encoding sequence as denoted by SEQ ID NO: 48, followed by a second P2A sequences, as dented by SEQ ID NO: 49, and the IL7 encoding sequence as denoted by SEQ ID NO: 50, and terminated by the BGH-T terminator of SEQ ID NO: 5. The LIF-Right homology arm exon 2 is denoted by SEQ ID NO: 58.

### Example 3

### Adoptive Cell Therapy (ACT)—Tumor-Infiltrating Lymphocyte (TIL) Therapy

Adoptive Cell Therapy (ACT) is a type of cellular immunotherapy that uses the cells of the immune system to treat diseases such as cancer, immune-related diseases and heritable deficiencies such as sickle cell disease. In Cancer, for example, T cells are taken from the patient's own blood or tumor tissue, grown in large numbers in laboratory, and then transplanted back to the patient to help the immune system fight the cancer.

Sometimes, the T cells are modified in the laboratory to make them better able to target the patient's cancer cells and kill them. Types of adoptive cell therapy include chimeric antigen receptor T-cell (CAR T-cell) therapy, tumor-infiltrating lymphocyte (TIL) therapy, cytotoxic T lymphocytes (CTL) therapy as well as Natural Killer (NK) cells therapy or even combinations (such as CAR-NK or CAR-CTLs). Allogeneic ACT that uses T cells or NK cells from a donor is being studied in the treatment of some types of cancer and some infections.

TIL populations are generated from single cell suspensions or tumor fragments from freshly resected tumor material of melanoma patients, through an initial expansion phase in the presence of interleukin-2 (IL-2). Subsequently, the cultured TIL are expanded to very large numbers (approximately 1×10¹⁰ to 1×10¹¹ cells) in a 14 day rapid expansion protocol (REP), thereby yielding the cell product that is used for infusion. No enrichment for tumor reactivity is included in this so-called ‘young’ TIL protocol (Itzhaki O, et al. J Immunother 2011; 34:212-20). Prior to infusion of the resulting TIL products, patients are treated with lymphodepleting, but non-myeloablative, chemotherapy and following intravenous administration of the cell product, high-dose (HD) bolus IL-2 infusions are given to support the growth and survival of the infused T cells. On average, TIL therapy has shown clinical responses in approximately 50% of treated individuals, mostly in anti-PD-1 treatment naïve patients, with durable complete remissions (CR) in 10%-15% of patients with treatment-refractory metastatic melanoma (Rosenberg S A, et al. Clin Cancer Res 2011; 17:4550-7).

In addition to the described above, the genetic modulation of the current invention is expected to have several advantages. First, it is expected to spare the need, or at least reduce the severity of the non-myeloablative lymphodepletion chemotherapy administration. This reduction would ease this treatment for patients and will avoid some of the adverse events related to this treatment. Along with that, due to the ability of the modified cells to change the tumor microenvironment to support inflammation and to recruit APCs, the efficacy of the treatment is expected to increase. In addition, due to the increased efficacy, the infused cell number required is expected to be reduced. Therefore, the culturing time also can be shortened allowing prompter treatment.

### Example 4

### Adoptive Cell Therapy (ACT)—NK and CAR-NK

Using an array of germline-encoded surface receptors, NK cells are able to recognize and rapidly act against malignant cells without prior sensitization. Upon activation, NK cells release cytotoxic granules containing perforin and granzymes to directly lyse tumor cells, in a similar fashion to activated cytotoxic T cells. NK cells are also potent producers of chemokines and cytokines such as interferon gamma (IFN-γ) and tumor necrosis factor alpha (TNF-α) and thereby are essential in modulating adaptive immune responses. Clinical trials have demonstrated the overall safety of NK cell infusion, even in the allogeneic setting (Sakamoto N, et al. J Transl Med. 2015; 13:277; Iliopoulou E G, et al. Cancer Immunol Immunother. 2010; 59(12):1781-9), in hematologic and solid tumors. The feasibility of utilizing allogeneic NK cells, as well as their established safety profiles, and their fast-acting nature have led to the development of “off-the-shelf” NK cell-based cancer immunotherapy. However, there are many challenges to overcome, such as difficulty achieving sufficient ex vivo expansion, limited in vivo persistence, limited infiltration to solid tumors, and tumor editing to evade NK cell activity.

With the remarkable success of CAR T-cells for treating hematological malignancies, a rapid growing interest in developing CAR-NK cells for cancer therapy emerged.

Compared to CAR-T cells, CAR-NK cells could offer some significant advantages, including: (1) better safety, such as a lack or minimal cytokine release syndrome and neurotoxicity in autologous setting and graft-versus-host disease in allogenic setting, (2) multiple mechanisms for activating cytotoxic activity, and (3) high feasibility for ‘off-the-shelf’ manufacturing. Besides target diverse antigens, CAR-NK cells could be a solution to the challenges mentioned above, such as enhance proliferation and persistence in vivo, increase infiltration into solid tumors, overcome resistant tumor microenvironment, and ultimately achieve an effective anti-tumor response.

The described genetic immune checkpoint modulations can be performed in NK cells. A generally induced immune checkpoint such as PD1 can be used, via it's promoter, to drive regulation or trans-regulation of an immune response. Likewise, NK-specific immune checkpoints such as NKG2A or to any of the KIRs receptors (that normally are stably expressed, and not as a sequel of signaling of activation) may be used to achieve the same.

This is expected to spare the need, or at least reduce the severity, of non-myeloablative lymphodepletion chemotherapy administration that would ease this treatment for patients and will avoid some of the adverse events related to this procedure. Along with that, due to the ability of the modified cells to change the tumor microenvironment to support inflammation and to recruit APCs, the efficacy of the treatment is expected to increase.

In addition, due to the increased efficacy, the infused cell number required is expected to be reduced. Therefore, the culturing time also can be shortened.

### Example 5

### Adoptive Cell Therapy (ACT)—Tregs and CAR-Tregs Therapy

Cell therapy with polyclonal regulatory T cells (Tregs) has been translated into the clinic for patients suffering from autoimmune diseases, for avoiding rejection in solid organ transplantation, or for avoiding GVH in patients transplanted with hematopoietic stem cells (HSCs). However, antigen-specific Tregs are functionally superior to polyclonal Tregs. For that end, CAR-Tregs, have been successfully demonstrated to be robust in preclinical studies across various animal disease models. With the increasing of the specificity CAR-Tregs could boost their therapeutic efficacy, assisted by activation and co-stimulatory signals generated by the engineered CAR. However, different co-stimulatory molecules provide different functions. Thus, in each therapeutic setting different networks of signals may be required. Therefore, it is likely that for optimal function and persistence of therapeutic CAR-Tregs, a combination of co-stimulation signals are required, and possibly at different time points to achieve a robust or efficient CAR-Treg therapy for patients. That goal can be achieved using the described modulation. Fusing the necessary co-stimulatory endo-domains to the different immune-checkpoint protein while relying on their native promoter's regulation, would enable support of the desired signaling network in the proper time and place. In addition, augmented co-secretion of relevant agents from the same genetic module could maximize the efficacy and relevant genetic regulation of this therapeutic approach.

### Example 6

### Small RNA Regulation Via Endogenously Regulated Short RNAs for the Creation of Immune Modulated Allogeneic CAR-T Cells

Chimeric antigen receptors (CAR) can be used to direct a T-cell to a predetermined antigen, for example, on a tumor. CARs can be expressed in said T-cells via viruses such as lentiviral vectors or other methods. CARs can replace the endogenous TCR to retarget and activate a T-cell.

T-cell receptor proteins alpha and beta (TCRαβ or TCR) are proteins that together are necessary to specifically recognize antigens in the context of MHC. Foreign T-cells introduced from an allogeneic donor will thus recognize host cells as non-self, eliciting graft versus host disease (GVHD). Thus, is advantageous to remove the native TCR from donated T-cells. An anti-TCR alpha or TCR beta siRNA or artificial miRNA encoding construct is here expressed by insertion of this construct under an endogenous checkpoint protein promoter that is conditionally transcribed, for example PD1 which is upregulated following T-cell activation. This is inserted to knockout the native PD1 gene creating a novel combination where the inhibiting checkpoint protein is replaced with a RNA silencing other genes.

B2M is a protein essential for assembly of the MHC class I (MHC-I) complex. Thus knockout or knockdown of the gene encoding this protein will render cells invisible, and thus tolerant to host T-cells and are thus useful in allogeneic transplantation. An anti-B2M siRNA or artificial miRNA encoding construct is here expressed by insertion of this construct under an endogenous checkpoint protein promoter, for example PD1.

Thus, to achieve a safe and effective allogeneic CAR-T, concomitant expression of a CAR, checkpoint knockout (i.e. PD1) and knock-down of B2M and/or TCR, together or separately, is performed.

Without being bound to a specific mechanism or method, T-cells derived from a healthy donor are first activated using anti-CD3 and CD28 antibodies. Activating these cells causes their proliferation and activation of genes including PD1. Following activation, these cells are electroporated or otherwise transfected with a gene-editing nuclease of the invention or nucleic acid encoding said nuclease and guide RNAs targeting the checkpoint gene (i.e. PD1 exon1), together with a donor DNA encoding the siRNA/miRNA construct. The CAR expressing virus or construct may be previously, concomitantly, or after this step be introduced to these cells. Subsequently these T-cells will be further cultured and readied for testing and transfusion to allogeneic patients.

### Example 7

### Fratricide in CAR-T

CD8+ CTL mediate the destruction of cells displaying foreign peptides in association with class I MHC molecules. Since CD8+ CTL themselves express class I MHC molecules, a phenomenon known as “fratricide” can be elicited by T cells presenting antigens to other CTL. In certain circumstances, the target antigen of a CAR may be constitutively or transiently expressed on a T cell (or CAR-T cell), meaning that the CAR T cell may undergo fratricide. Thus, designing CARs remains a challenge because many targetable antigens are shared by T cells and tumor cells. This shared expression of antigens can cause CAR T cell fratricide. Thus it is advantageous to create CAR-T cells devoid of fratricide. Here removal of the self-antigen recognized by the CAR for example CD3 or CD38 (Gao et al., Journal of Genetics and Genomics 2019 46:3367-377) is employed. To remove presentation of CD3 complex on the cell membrane, a siRNA/miRNA is used to knock down TCR alpha or TCR beta. For example, in T-cell malignancies constitutive knockdown of TCR is expected to prevent CAR-T fratricide.

To remove presentation of CD38, a siRNA/miRNA is used to directly knock down CD38.

CD38 silencing construct can be inserted after the promoter of a checkpoint protein, knocking out the expression of that checkpoint protein (i.e., PD-1). One advantage is that an insertion in a single allele of PD-1 can knock-down the transcripts from both CD38 alleles.

### Example 8

### Avoidance of Cytokine Release Syndrome by Conditional Knockdown of IL6

Cytokine release syndrome (CRS) is an adverse effect of over-activation of the immune response which may be lethal. CAR T cells, activated by CAR mediated signals, will proliferate and release a variety of inflammatory factors to trigger a systemic inflammatory response. Interleukin 6 (IL-6) is an interleukin that acts as a pro-inflammatory cytokine. IL-6 triggers more IL-6 release by monocytes [8]. This study showed that expression of shRNA targeting IL6 on the viral-delivered CAR construct improves in vivo safety and preserves antitumor efficacy in a mouse xenograft model. In the current invention, the anti-IL6 siRNA/miRNA constructs are expressed under an endogenous promoter and not under the U6 promoter used in the construct carried by the CAR viral vector. Advantages of such an approach are activation only upon stimulation of the CAR-T cells allowing promoter free directed insertion of this construct.

### Example 9

Creation of Immune Modulated CD4+ by Downregulation of TRAF4 Using Controlled miR-4443 Overexpression

MicroRNAs are important regulators of T cell activation, proliferation and the cytokine production. For example, miR-125a, miR-21, miR-31, miR-23b, and miR-142-3p/5p can alter the expression of proinflammatory and anti-inflammatory cytokines via immune pathways (Qi Y, et al 0.2017 Front. Immunol. 8:1440).

miR-4443 binds TRAF4 to activate the NF-κB signal pathway. Here a miR-4443 precursor RNA as denoted by SEQ ID NO: 39 is expressed under the PD1 promoter.

Upon activation of the promoter the precursor is transcribed and processed to produce miR-4443 as denoted by SEQ ID NO: 40. In the cell, this microRNA is loaded onto RISC to downregulate mRNA levels of its target gene TRAF4. As TRAF4 downregulates the NF-κB signal pathway this pathway will now be induced. NF-κB is a major transcription factor that regulates genes responsible for both the innate and adaptive immune response.

Once induced NF-κB is able to enter the nucleus to upregulate genes involved in T-cell development, maturation, and proliferation. Genes upregulated in CD4+ cells following miR-4443 overexpression include CCL21, IL-1, IL-6, IL-17, and IFN-γ. For example CCL21 (in combination with IL7) has been shown to increase activity of CAR-T Cells in solid tumors [6].

### Example 10

### Exhaustion Tolerant TIL or CAR-T

T cell exhaustion is a broad term describing a state of T cell dysfunction resulting from chronic stimulation. Exhaustion is defined by poor effector function, sustained expression of inhibitory receptors and a transcriptional state distinct from that of functional effector or memory T cells. Exhausted T cells overexpress inhibitory receptors such as Programmed Death-1 (PD-1, CD279), cytotoxic T lymphocyte antigen-4 (CTLA-4, CD152), Lag-3, Tim-3, CD244/2B4, CD160, TIGIT, and others and are impaired in their ability to release pro-inflammatory cytokines such as IFNγ and TNFα. Exhaustion commonly occurs in the tumor microenvironment where T cells suffer a loss of their cytotoxic function and become ineffective in their ability to kill cancerous cells. Thus it is advantageous to prevent T-cells from becoming exhausted and advantageous to reinvigorate exhausted T-cells.

The present disclosure thus provides a novel concept of building auto-regulated genetic circuits into CAR T cells to selectively circumvent their exhaustion upon activation in the TME. In some embodiments, genetic rewiring is achieved by precisely inserting modulatory sequences as the replacement sequences of the disclosed donor of the system). Non-limiting embodiments for such modulatory sequences may be inhibitory elements, e.g., artificial miRNAs under endogenous “Driver” promoters to downregulate “Target” genes that cause exhaustion. In some embodiments, such promoters are of genes involved either directly or indirectly in exhaustion. Optionally, use of a pair of two target recognition elements (e.g., as previously disclosed by the present inventors WO 2013/088446) enables specific replacement of the “Driver” gene minimizing the risk of off-target mutations. Further advantages of combined insertion and silencing approach for the preparation of the exhaustion tolerant T cells are (i) the ability to regulate when a gene is turned on/off by biologically and clinically relevant cellular cues, and (ii) multiple gene-knockdown with a single dsDNA cleavage as opposed to multiple DNA breaks in multiple gene-KO and (iii) RNA-silencing, as opposed to single allele knockout, silences both alleles.

The present disclosure thus enables the design of gene modified TILs or CAR T cells resistant to exhaustion and/or showing enhanced activation and expansion kinetics.

As illustrated by FIG. 5, the inventors picked endogenous genomic promoters that are selectively turned on in the activated CAR T cells within the TME physiologically, and drive genes related to immune suppression or exhaustion. This gene/s (“Driver/s”, also referred to herein as the “first” immunoregulatory genes) are permanently replaced (knock-out [KO]) in the genome by a modulatory element such as an artificial microRNA (amiRNA) or siRNA, which in turn knocks-down (KD) it's second allele and/or other immunosuppressive genes, genes that contribute to exhaustion, or genes that normally downregulate inflammation (“Target”). The foundation of this approach is that the Driver's promoter is turned on in the TME, at the correct time and place. When that promoter drives the expression of the amiRNAs, the progression of exhaustion process is prevented and, therefore, the cell is prevented from entering the terminal exhaustion states. The strength of this approach is that the stronger the inhibition pressure, the higher the expression of anti-inhibitory trans-modulation elements and vice-versa, when the tumor shrinks and pressure is relieved, expression is suspended. This auto-modulated approach is expected to be significantly safer than permanent KO of target(s) that is more likely to be associated with persistent resistance to repression and more harmful in cases of on-target off-tumor immune effector function. As an example for an optional target, DNA Methyltransferase 3 Alpha (DNMT3A) is next used by the inventors. DNMT3A KO using CRISPR-Cas9 has been shown to prevent exhaustion and enhance antitumor activity, enhancing CAR T-cell survival during prolonged antigen exposure [B. Prinzing et al., 10.1126/scitranslmed.abh0272]. However, this KO prior art approach resulted in high level off-target editing events.

As indicated above, the advantages of a combined insertion and silencing approach of the present disclosure, as opposed to silencing only by permanent gene knockout include the (a) ability to regulate when a gene is turned on or off following biologically and clinically relevant cellular cues; (b) multiple gene-knockdown with a single dsDNA genomic cleavage as opposed to multiple dsDNA breaks in multiple gene-knockout; and (c) RNA-silencing, as opposed to single allele knockout, silences both alleles.

Thus, in some embodiments, DNMT3A is used as a target (also referred to herein as a second regulatory gene) modulated by the replacement sequence inserted into the first regulatory target (Drivers).

Potential Drivers (also denoted herein as first immune-regulatory genes) useful in the present disclosure include T Cell Immunoreceptor with Ig And ITIM Domains (TIGIT), which promotes CD8+ T cell exhaustion and predicts poor prognosis of colorectal cancer.

Another applicable Driver is Leukaemia-inhibitory Factor (LIF), that is also described by Example 2, a cytokine with autocrine and paracrine functions, that may be one of the major driving factors for the recruitment of immunosuppressive subpopulations of lymphocytes into the TME. Thus, reduction of LIF expression in the TME is expected to have a positive effect on treatment of solid tumors. LIF is highly (681x) increased in T-cells after CD3/CD28 beads stimulation. LIF's promoter may be thus used to selectively drive transcription of inserted genetic payload in stimulated T-cells, as it has some background expression but increases mightily upon activation.

Still further, since the checkpoint protein PD1, and specifically, the PD-1:PD-L1 pathway is a central regulator of T cell exhaustion, PD1 is also used in the present disclosure as a driver (a first immune-regulatory gene). Still further, in human melanoma and ovarian cancer, PD-1+ Tim-3+ and PD-1+ Lag-3+ tumor specific CD8+ T cells showed more severe signs of dysfunction in terms of effector cytokine production than cells expressing only PD-1 or neither receptor. Thus, any one of PD-1, combined knockout and/or knockdown of PD1 and Tim-3 and/or Lag-3 may be beneficial to reverse or prevent exhaustion of TILs or CAR-T cells. Likewise, also B and T lymphocyte attenuator (BTLA), a co-inhibitory receptor structurally related to PD-1, may contribute to the resistance to PD-1 targeted therapy and co-blockade of BTLA can enhance the efficacy of anti-PD-1 immunotherapy. Thus, combined knockout and/or knockdown of PD1 and TIGIT and/or BTLA may also be beneficial to reverse or prevent exhaustion of TILs or CAR-T cells.

Non-limiting embodiments for Guide pairs useful in for knocking out these genes in their coding sequence, targets exon 2 of LAG3, using the LAG3ex2p1_L and LAG3ex2p1_R guide pair as denoted by SEQ ID NO: 59 and 60, respectively, and for exon 2 in the HAVCR2 gene, HAVCR2ex2p1_L and HAVCR2ex2p1_R, as denoted by SEQ ID NO: 61 and 62, respectively.

Alternatively, guide pairs directed at the 5′-UTR of these genes may be used for utilizing their promoters before the coding sequence in the 5′-UTR, specifically, for LAG3, the LAG3-5UTR1_L and LAG3-5UTR1_R pair of SEQ ID NO: 63 and 64, and for the HAVCR2 gene, the HAVCR2-5UTR1_L and HAVCR2-5UTR1_R as denoted by SEQ ID NO: 65 AND 66, respectively. A terminator in the 3′ of the insert will ensure knockout of the gene into which it is inserted.

Methods for testing different combinations of checkpoint protein knockout/knockdown are performed for testing their efficacy and safety in TIL or autologous CAR-T or allogeneic CAR-T for treatment of different cancer indications. It is conceivable that different combinations will have different effects in different cancers.

Artificial miRNA/siRNA for immune modulation Table 3 (except for those referenced, the sequence thereof which may also be inserted into a scaffold or under endogenous promoters) and artificial hairpin constructs for their expression. The target genes for which amiRs/siRNA can be designed according to the instructions in example 6 to modulate immune response or function are disclosed by the following table.

TABLE 3
amiRs/siRNA for immuno-modulatory targets

| | silencing | | | | | | |
|---|---|---|---|---|---|---|---|
| targeted | artificial | | amiR/si | pre/pri | | | Pre/Pri- |
| gene, | mi/siRNA | amiR/siRNA | RNA | miRNA | pri-amiR | pre-amiRNA | miR |
| exon | name | sequence | SEQID | name | sequence | sequence | SEQID |
| PD1, | amiR- | AAGCUGC | 67 | Pre- | | GTAGCACAAGCTGCA | 68 |
| exon2 | PD1ex2 | AGGUGAA | | amiR- | | GGTGAAGGTGGCGTT | |
| | | GGUGGCG | | PD1ex2 | | TGTTTAGTTATAACCC | |
| | | UU | | | | ACCTTCACCTGCAGC | |
| | | | | | | AATGTACTGC | |
| PD1, | amiR- | UUCUAAG | 69 | Pri- | CTCGAGAAG | | 70 |
| exon1 | PD1ex1 | AACCAUC | | amiR- | AAGGTATAT | | |
| | | CUGGC | | PD1ex1 | TGCTGTTGAC | | |
| | | | | | AGTGAGCGA | | |
| | | | | | CGCCAGGAT | | |
| | | | | | GGTTCTTAG | | |
| | | | | | AATAGTGAA | | |
| | | | | | GCTTCAGAT | | |
| | | | | | GTATTCTAA | | |
| | | | | | GAACCATCC | | |
| | | | | | TGGCGCTGC | | |
| | | | | | CTACTGCCTC | | |
| | | | | | GGACTTCAA | | |
| | | | | | GGGGTCAGT | | |
| | | | | | CAGAATTC | | |
| TIGIT, | ami-R | UUUCUAU | 71 | Pre- | | TGTTCTTTCTATTGTG | 72 |
| exon 2 | TIGITex2 | UGUGCCU | | amiR- | | CCTGTCATCATGTGA | |
| | | GUCAUCA | | TIGITex2 | | AGTAGATTAGCATCA | |
| | | U | | | | TGATGTCAGGGTCAA | |
| | | | | | | TAGAATGGCAT | |
| BTLA, | amiR- | UAUAUAA | 73 | Pri- | CTCGAGAAG | | 74 |
| exon 1 | BTLAex1 | AGCUGUA | | amiR- | AAGGTATAT | | |
| | | CAUC | | BTLAex1 | TGCTGTTGAC | | |
| | | | | | AGTGAGCGA | | |
| | | | | | CGGATGTAC | | |
| | | | | | AGCTTTATAT | | |
| | | | | | ATAGTGAAG | | |
| | | | | | CTTCAGATGT | | |
| | | | | | ATATATAAA | | |
| | | | | | GCTGTACAT | | |
| | | | | | CCGCTGCCT | | |
| | | | | | ACTGCCTCG | | |
| | | | | | GACTTCAAG | | |
| | | | | | GGGTCAGTC | | |
| | | | | | AGAATTC | | |
| CTLA4, | amiR- | UAUAGUG | 7 | Pri- | CTCGAGAAG | | 76 |
| exon2 | CTLA4ex2 | AGGUUCA | | amiRCT | AAGGTATAT | | |
| | | CUUGC | | LA4ex2 | TGCTGTTGAC | | |
| | | | | | AGTGAGCGA | | |
| | | | | | CGCAAGTGA | | |
| | | | | | ACCTCACTAT | | |
| | | | | | ATAGTGAAG | | |
| | | | | | CTTCAGATGT | | |
| | | | | | ATATAGTGA | | |
| | | | | | GGTTCACTTG | | |
| | | | | | CGCTGCCTA | | |
| | | | | | CTGCCTCGG | | |
| | | | | | ACTTCAAGG | | |
| | | | | | GGTCAGTCA | | |
| | | | | | GAATTC | | |
| Lympho- | amiR- | UUAUACA | 77 | Pre- | | GCAGTCCTCTGCAAC | 78 |
| cyte | LAG3ex1 | UGAUGGA | | amiR- | | GTCTCCACATGTATA | |
| activation | | GACGUUG | | LAG3ex1 | | ACGAAGAATGTAGTT | |
| gene 3 | | | | | | TATACATGATGGAGA | |
| (LAG-3) | | | | | | CGTTGTGGTGGCCTG | |
| (CD223), | | | | | | C | |
| exon1 | | | | | | | |
| T cell | amiR- | UAAUAAU | 79 | Pri- | CTCGAGAAG | | 80 |
| immuno- | TIM3ex2 | UCACAUC | | amiR- | AAGGTATAT | | |
| globulin | | CCUUC | | TIM3ex2 | TGCTGTTGAC | | |
| and | | | | | AGTGAGCGA | | |
| mucin | | | | | CGAAGGGAT | | |
| domain 3 | | | | | GTGAATTATT | | |
| (TIM3) | | | | | ATAGTGAAG | | |
| (HAVCR2), | | | | | CTTCAGATGT | | |
| exon2 | | | | | ATAATAATT | | |
| | | | | | CACATCCCTT | | |
| | | | | | CGCTGCCTA | | |
| | | | | | CTGCCTCGG | | |
| | | | | | ACTTCAAGG | | |
| | | | | | GGTCAGTCA | | |
| | | | | | GAATTC | | |
| TGF-beta | si-TGFBR | S; 5′- | 81 | | | | |
| receptor | duplex | CCAUCGA | | | | | |
| | | GUGCCAA | | | | | |
| | | AUGAA | | | | | |
| | | [dT][dT]-3′; | | | | | |
| | | AS, 5′- | | | | | |
| | | UUCAUUU | | | | | |
| | | GGCA | | | | | |
| | | UUCGAUG | | | | | |
| | | G[dT][dT]- | | | | | |
| | | 3′ (Int Urol | | | | | |
| | | Nephrol | | | | | |
| | | (2010) | | | | | |
| | | 42:315-323) | | | | | |
| miR-4443 | miR-4443 | UUGGAGG | 82 | pre- | | GGTGGGGGTTGGAGG | 83 |
| | | CGUGGGU | | miR4443 | | CGTGGGTTTTAGAAC | |
| | | UUU | | | | CTATCCCTTTCTAGCC | |
| | | | | | | CTGAGCA | |
| IL-6 | si-IL6 | seq ID 1, | 84 (#1) | | | | |
| CD38 | duplex#1; | 2 from | and | | | | |
| | si-IL6 | WO200612 | 85 (#2) | | | | |
| | duplex#2 | 3867A1 | | | | | |
| | | #1 sense | | | | | |
| | | sequence of | | | | | |
| | | human IL-6 | | | | | |
| | | siRNA | | | | | |
| | | agacauguaa | | | | | |
| | | caagaguaa | | | | | |
| | | #2 sense | | | | | |
| | | sequence of | | | | | |
| | | human IL-6 | | | | | |
| | | siRNA | | | | | |
| | | ggagacuugc | | | | | |
| | | cuggugaaa | | | | | |
| CD38 | si-CD38 | UUUGGCA | 86 | | | | |
| ex1/2 | | GUCUACA | | | | | |
| | | UGUCUCA | | | | | |
| | | UCUC | | | | | |
| | | (Sieck GC, | | | | | |
| | | et.al. | | | | | |
| | | Regulation | | | | | |
| | | of store- | | | | | |
| | | operated | | | | | |
| | | Ca2+ entry | | | | | |
| | | by CD38 in | | | | | |
| | | human | | | | | |
| | | airway | | | | | |
| | | smooth | | | | | |
| | | muscle. Am | | | | | |
| | | J Physiol | | | | | |
| | | Lung Cell | | | | | |
| | | Mol | | | | | |
| | | Physiol. | | | | | |
| | | 2008 | | | | | |
| | | Feb;294(2): | | | | | |
| | | L378-85. | | | | | |
| | | doi: | | | | | |
| | | 10.1152/ | | | | | |
| | | ajplung. | | | | | |
| | | 00394.2007. | | | | | |
| | | Epub | | | | | |
| | | 2008 Jan 4. | | | | | |
| | | PMID: | | | | | |
| | | 18178673.) | | | | | |
| TCR- | amiR- | UAUAUAC | 87 | pri- | CTCGAGAAG | | 88 |
| alpha | TCRa- | ACAUCAG | | amiR- | AAGGTATAT | | |
| constant | const | AAUCC | | TCRa- | TGCTGTTGAC | | |
| region | | | | const | AGTGAGCGA | | |
| | | | | | CGGATTCTG | | |
| | | | | | ATGTGTATAT | | |
| | | | | | ATAAGTGAA | | |
| | | | | | GCTTCAGAT | | |
| | | | | | GTTATATATA | | |
| | | | | | CACATCAGA | | |
| | | | | | ATCCGCTGC | | |
| | | | | | CTACTGCCTC | | |
| | | | | | GGACTTCAA | | |
| | | | | | GGGGTCAGT | | |
| | | | | | CAGAATTC | | |
| B2M | amiR- | UAGAAAG | 89 | pri-miR- | CTCGAGAAG | | 90 |
| | B2Mex1 | AGAGAGU | | B2Mex 1 | AAGGTATAT | | |
| | | AGCGC | | | TGCTGTTGAC | | |
| | | | | | AGTGAGCGA | | |
| | | | | | CGCGCTACT | | |
| | | | | | CTCTCTTTCT | | |
| | | | | | ATAGTGAAG | | |
| | | | | | CTTCAGATGT | | |
| | | | | | ATAGAAAGA | | |
| | | | | | GAGAGTAGC | | |
| | | | | | GCGCTGCCT | | |
| | | | | | ACTGCCTCG | | |
| | | | | | GACTTCAAG | | |
| | | | | | GGGTCAGTC | | |
| | | | | | AGAATTC | | |
| KIR | | UAUAUUU | 91 | | CTCGAGAAG | | 92 |
| (killer | | GCAGGAG | | | AAGGTATAT | | |
| cell | | AUGCC | | | TGCTGTTGAC | | |
| Immunog | | | | | AGTGAGCGA | | |
| lobulin- | | | | | CGGCATCTC | | |
| like | | | | | CTGCAAATA | | |
| receptor) | | | | | TATAGTGAA | | |
| | | | | | GCTTCAGAT | | |
| | | | | | GTATATATTT | | |
| | | | | | GCAGGAGAT | | |
| | | | | | GCCGCTGCC | | |
| | | | | | TACTGCCTCG | | |
| | | | | | GACTTCAAG | | |
| | | | | | GGGTCAGTC | | |
| | | | | | AGAATTC | | |
| SIGLEC9 | | TTTATAC | 93 | | | AGATCTGGGAGAAAG | 94 |
| (Sialic | | TTCCTTTC | | | | GAAGTATAAATACCT | |
| acid- | | TCCC | | | | GACCCATATTTATAC | |
| binding | | | | | | TTCCTTTCTCCCTTTT | |
| immunog | | | | | | TGGTACC | |
| lobulin- | | | | | | | |
| type | | | | | | | |
| lectin 9, | | | | | | | |
| also | | | | | | | |
| des- | | | | | | | |
| ignated | | | | | | | |
| as | | | | | | | |
| CD329) | | | | | | | |
| CD3 | | UUAUCUU | 95 | | | GTAGCACTTATCTTC | 96 |
| Gamma | | CAGUUAG | | | | AGTTAGGAAGCCGTG | |
| | | GAAGCCG | | | | TTTAGTTATCGGTTCC | |
| | | | | | | TAACTGAAGATTTAG | |
| | | | | | | TACTGC | |
| CD3 | | AUUCACA | 97 | | | GTAGCACATTCACAA | 98 |
| Delta | | AACACUC | | | | ACACTCTGTCCTCTGT | |
| | | UGUCCUC | | | | TTAGTTATGAGACAG | |
| | | | | | | AGTGTTTGTGATTTGT | |
| | | | | | | ACTGC | |

The disclosed approach prepares the ground for specific and regulated genome editing (KO with no off-target mutations, KI at optimal genomic regions, adaptive regulation of cellular activity) making the design of optimally enhanced CAR T cells possible and radically improving safety by expressing genes under endogenous genetic control.

### Example 11

### Multi-Gene Knockdown Cassette

Multiple genes or multiple targets within a gene or combinations thereof simultaneously knocked down under a common regulatory element such as a checkpoint gene promoter.

Such artificial polycistron cassettes are created within a scaffold, for example, by replacing one or several or all the miRNA and miRNA* sequences in the Homo sapiens mir-17-92 microRNA cluster (genbank NG_032702.1) and according to the number of artificial miRNAs desired shortening the unnecessary sequences. In the example below 5 artificial miRNAs are co-transcribed and processed from hairpins in a living cell from a single endogenous promoter. A PolII promoter and Terminator are positioned up- and down-stream respectively. Alternatively exogenous promoters may also be used. A reverse-complement sequence inverted terminator (such as SV40 poly-A signal terminator) may be placed after the last hairpin to prevent transcription of reverse-complement flipped inserts and subsequent erroneous processing of the antisense artificial miRNAs.

In bold and underline are the miR and miR* insertion sites that should be replaced by the desired artificial miRNA and it's semi-complementary artificial miR*. Number in square parenthesis following the N denotes recommended number of nucleotides to insert. The miR* sequence should be modified to conserve the original (respective native miR hairpin) secondary structure and thermodynamic properties to promote preferential RISC loading of the miR and not the miR* strand.

The best miR design locations in this polycistron are the 2^{nd} and 3^{rd} hairpins, thus to express less than 5 miRNAs one may delete prior or later sequences. The sequence between the deleted miR precursor and the desired miR structure should be maintained for good expression.

Sequence “siPolycistron scaffold” (SEQ ID NO: 99):
CTGTAAAGAATTCTTAAGGCATAAATACGTGTCTAAATGGACCTCATATCTTTGAGATAATT
AAACTAATTTTTTCTTCCCCATTAGGGATTATGCTGAATTTGTATGGTTTATAGTTGTTAGAG
TTTGAGGTGTTAATTCTAATTATCTATTTCAAATTTAGCAGGAAAAAAGAGAACATCACCTT
GTAAAACTGAAGATTGTGACCAGTCAGAATAATGT(N[23])TGATATGTGCATCT(N[22])CATT
ATGGTGACAGCTGCCTCGGGAAGCCAAGTTGGGCTTTAAAGTGCAGGGCCTGCTGATGTTGA
GTGCTTTTTGTTC(N[23])TGAAGTAGATTAGCATCT(N[23])CATAAGAAGTTATGTATTCATCC
AATAATTCAAGCCAAGCAAGTATATAGGTGTTTTAATAGTTTTTGTTTGCAGTCCTCTGTT(N[22])
AGAAGAATGTAGT(N[23])TGGTGGCCTGCTATTTCCTTCAAATGAATGATTTTTACTAATT
TTGTGTACTTTTATTGTGTCGATGTAGAATCTGCCTGGTCTATCTGATGTGACAGCTTCTGTA
GCAC(N[23])TGTTTAGTTATCT(N[22])TACTGCTAGCTGTAGAACTCCAGCTTCGGCCTGTCGC
CCAATCAAACTGTCCTGTTACTGAACACTGTTCTATGGTT(N[23])TGTGTGATATTCTGC(N[23])
CTGTGGTAGTGAAAAGTCTGTAGAAAAGTAAGGGAAACTCAAACCCCTTTCTACACAGGTT
GGGATCGGTTGCAATGCTGTGTTTCTGTATGGTATTGCACTTGTCCCGGCCTGTTGAGTTTGG
TGGGGATTGTGACCAGAAGATTTTGAAAATTAAATATTACTGAAGATTTCGACTTCCACTGT
TAAATGTACAAGATAC

Still further, a polycistron for expression of 5 hairpins (SEQ ID NO: 100) which has been next artificially constructed from a combination of artificial hairpin, natural miR4443 precursor, and portions of the miR 17-92 cluster. The silencing RNAs to be expressed in this example in order of hairpin (bold) from 5′ to 3′ are anti-PD1, anti-TIGIT, miR4443, anti-BTLA and anti LAG-3. Other combinations or combinatory libraries to be screened according to therapeutic target may be prepared by golden-gate or other cloning methods.

### Example 12

### Optimize Driver Gene-Editing Guides and Conditions for Gene-Replacement

The inventors first confirm the expression pattern of the Drivers and targets with qRT-PCR or NGS during in vivo exhaustion, which serve as a reference to quantify alterations after rewiring and also for discovering potential other candidate Drivers and Targets.

Due to complicated regulation of the Driver gene's expression, these patterns could change due to manipulations by the inventors. Thus, the transcript pattern is tested by qRT-PCR with each KO and amiRNA KI step added. Functional Driver-Target rewiring combinations are tested first in vitro for preserving acute cytotoxicity, then in rechallenge model systems for activity, differentiation/maturation dynamics, endurance, exhaustion and accompanying changes in gene expression and epigenetic alterations. The most promising combinations are tested on pre-clinical tumor models of varying levels of therapy resistance and compared against the performance of non-edited CAR T cells.

Transcriptome changes during in vivo exhaustion (or persistence) are then characterized.

Next, gRNA pairs for each Driver are designed and the KO efficiency is tested. PDCD1 (PD1) has already been verified as a Driver and is used for parallel progress. After KO has been optimized, a generic amiRNA expressing cassette is produced and inserted to test replacement (KI) efficiency. DNA is thus inserted, by directional NHEJ or HDR, either containing an artificial miRNA precursor or a polycistronic set of miRNA precursors (FIG. 5), relevant spacers and a terminator (BGH-T or SV40-T) in the 3′ of the insert to ensure knockout of the gene into which it is inserted and to overcome nonsense mediated decay (NMD). An additional terminator may be added in the reverse direction to prevent transcription of anti-sense RNA if the construct is inadvertently inserted in the reverse direction. The cassette is flanked on either side by 6-frame stop codons. A polycistron cassette scaffold of up to 5 miRNAs disclosed in Example 11, was designed to simultaneously KD several Target genes (or multiple locations within a single Target), to be transcribed under a common regulatory Driver element.

Optimizing Target KD amiRNAs

As not all miRNAs are expressed, processed or accumulated as desired, the proper processing and accumulation of these miRNAs are tested without the gene-editing step.

The candidate amiRNA precursors are first cloned into a strong promoter expression vector to test processing by Northern blot. Next, to test single knockdown of targeted genes in vitro, qRT-PCR assays are performed to test the change of target mRNA levels in activated vs exhausted T cells, in comparison to the necessary level of the amiRNA to achieve the desired KD. Level of the targeted protein may also be evaluated using FACs, ELISA or Western Blot. For each target, the best amiRNA are than selected. The amiRNA combinations (single or polycistrons) are cloned by Golden Gate, constructs for insertion via NHEJ and via HDR are built, and the delivery of these constructs is verified.

### Verifying Gene-Editing Safety

The presence of off-target edits of the selected Driver guide-RNAs in comparison to CRISPR-Cas9, is investigated by NGS.

Still further, Human PBL carrying 2^{nd} or 3^{rd} Generation CAR constructs are rewired using the systems of the present disclosure (the donors and the machines disclosed herein, also indicated herein as genome modifier protein/s). Rewired CAR T cells are compared to conventional ones in function and underlying genomic changes both in vitro and in vivo.

Target tumor cell lines of different tumor sources (e.g., breast, lung, gastric), that are known to express the target antigen of the CAR used (e.g., HER2, CD19), are used for repeated stimulation. Other tumor cell lines as well as similar tumor cell lines that do not express the specific antigen, are used as a control. The tumor cells used stably express at least one detectable moiety, for example, Luciferase and/or a Fluorescent Protein for use in small animal imaging models. Cells are tested for (i) phenotype markers using flow cytometry, (ii) IL-2 and IFN-γ release, by ELISA, (iii) acute killing, by Real time impedance analysis, (iv) immune synapse formation and stability, by Airy-scan microscopy, z. Movi cell avidity analyser, (v) migration, by penetration of tumor spheroids, (vi) long term survival and expansion, by repeated bleeding and organs yield, as well as secondary transplant to tumor bearing animals or tumor rechallenge. Modified CAR Ts with equal or better function than non-modified counterparts are progressed to further in vitro functions tests.

### In-Vitro Functional Analysis:

T-Cells are consistently counted to monitor expansion kinetics and monitored for surface expressed exhaustion markers (e.g., CD3, CD4, CD8, CCR7, CD45RA, CD39, PD1, LAG3, TIM3) by FACS and assigned exhaustion phenotype (i.e stimulated, expanding, reversibly- or terminally-exhausted) accordingly. Similarly, expression of rewired genes is tested by qRT-PCR before and after target engagement.

### Testing Time-Dependent In Vivo Functions and Underlying Genomic Changes

About 2×10⁶ CAR T or their rewired counterparts injected once i.v. are used to treat large (0.25 cm³) tumors in mice. Tumor growth is tracked in an IVIS spectrum CT using ffLuc stable expressing tumor cells. If tumors are eradicated, after two months tumor rechallenge is used to test presence of memory T cells. Samples for single cell transcriptome analysis are taken and processed. Animals are monitored for signs of systemic toxicity.

The following table 4 discloses all sequences that are disclosed in the present disclosure and included in the sequence listing.

TABLE 4
sequence listing

| SEQ ID | |
|---|---|
| NO: | SEQUENCE DESCRIPTION |
| 1 | Protein, HOMO SAPIENS IL7 |
| 2 | Protein, HOMO SAPIENS CCL21 |
| 3 | Protein, T2A peptide |
| 4 | Protein, P2A peptide |
| 5 | DNA, BGHT terminator |
| 6 | DNA, NHEJ Integration cassette |
| 7 | DNA, PD1 exon3 dCasFok guide 1 |
| 8 | DNA, PD1 exon3 dCasFok guide 2 |
| 9 | DNA, PD1 exon3 dCasFok guide 3 |
| 10 | DNA, PD1 exon3 dCasFok guide 4 |
| 11 | DNA, PD1 exon3 dCasFok guide 5 |
| 12 | DNA, PD1 exon3 dCasFok guide 6 |
| 13 | DNA, PD1 exon3 dCasFok guide 7 |
| 14 | DNA, PD1 exon3 dCasFok guide 8 |
| 15 | DNA, PD1 exon3 dCasFok guide 9 |
| 16 | DNA, PD1 exon3 Cas9 guide 1 |
| 17 | DNA, PD1 exon3 Cas9 guide 2 |
| 18 | DNA, PD1 exon3 Cas9 guide 3 |
| 19 | DNA, PD-1 exon 3 |
| 20 | Protein, PD-1 -1 reframe product |
| 21 | Protein, PD-1 -2 reframe product |
| 22 | DNA, 41BBtransmembrane-EGFP-T2A-EBFP-P2A-luciferase-BGHT PD1 |
| | integration cassette |
| 23 | DNA, 41BBtransmembrane-EGFP-T2A-IL7-P2A-luciferase-BGHT PD1 |
| | integration cassette |
| 24 | DNA, 41BB ICD-T2A-EBFP-P2A-luciferase-BGHT PD1 integration cassette |
| 25 | DNA, 41BBtransmembrane-EGFP-T2A-mCherry-P2A-luciferase-BGHT PD1 |
| | integration cassette |
| 26 | DNA, 41BBICD-T2A-CCL21-P2A-IL7-BGHT PD1 integration cassette |
| 27 | Protein, PD1Nterm-41BBICD-T2A-CCL21-P2A-IL7 |
| 28 | Protein, EGFP |
| 29 | Protein, EBFP |
| 30 | Protein, Luciferase |
| 31 | Protein, PD1Nterm-41BBtransmembrane-EGFP-T2A-EBFP-P2A-luciferase |
| 32 | Protein, >PD1Nterm-41BBtransmembrane-EGFP-T2A-IL7-P2A-luciferase |
| 33 | Protein, PD1Nterm-41BB ICD-T2A-EBFP-P2A-luciferase |
| 34 | Protein, mCherry |
| 35 | Protein, PD1Nterm-41BBtransmembrane-EGFP-T2A-mCherry-P2A- |
| | luciferase |
| 36 | DNA, PD1-Fc integration cassette |
| 37 | Protein, PD1-Fc-OX40 |
| 38 | DNA, NHEJ Integration cassette, GFP control |
| 39 | RNA, miR-4443 precursor: >hsa-mir-4443 MI0016786 |
| 40 | RNA, mature miR-4443 sequence: >hsa-miR-4443 MIMAT0018961 |
| 41 | RNA, Exon 2 gRNA Pair#6: LIF-ex2-6L |
| 42 | RNA, LIF-ex2-6R |
| 43 | RNA, Exon 2 gRNA Pair#5: LIF-ex2-5L |
| 44 | RNA, LIF-ex2-5R |
| 45 | DNA, HDR Donor Construct LIF exon 2 |
| 46 | DNA, The LIF Left homology arm exon 2 |
| 47 | DNA, First P2A |
| 48 | DNA, homo sapience, CCL21 |
| 49 | DNA, Second P2A |
| 50 | DNA, homo sapience, IL7 |
| 51 | DNA, LIF-Right homology arm exon 2 |
| 52 | RNA, Exon 3 gRNA Pair#5: LIF-ex3-5L |
| 53 | RNA, LIF-ex3-5R |
| 54 | RNA, Exon 3 gRNA Pair#1, LIF-ex3-1L |
| 55 | RNA, LIF-ex3-1R |
| 56 | DNA, HDR Donor Construct LIF exon 3 |
| 57 | DNA, LIF Left homology arm exon 3 |
| 58 | DNA, LIF Right homology arm exon 3 |
| 59 | DNA, Guide pairs for knocking out these genes in their coding sequence: |
| | LAG3ex2p1_L |
| 60 | DNA, LAG3ex2p1_R |
| 61 | DNA, HAVCR2ex2p1_L |
| 62 | DNA, HAVCR2ex2p1_R |
| 63 | DNA, Guide pairs for utilizing their promoters before the coding sequence in |
| | the 5′-UTR: LAG3-5UTR1_L |
| 64 | DNA, LAG3-5UTR1_R |
| 65 | DNA, HAVCR2-5UTR1_L |
| 66 | DNA, HAVCR2-5UTR1_R |
| 67 | DNA, amiR-PD1ex2 |
| 68 | DNA, Pre-amiR-PD1ex2 |
| 69 | RNA, amiR-PD1ex1 |
| 70 | RNA, Pri-amiR-PD1ex1 |
| 71 | RNA, amiR-TIGITex2 |
| 72 | RNA, Pre-amiR-TIGITex2 |
| 73 | RNA, amiR-BTLAex1 |
| 74 | RNA, Pri-amiR-BTLAex1 |
| 75 | RNA, amiR-CTLA4ex2 |
| 76 | RNA, Pri-amiRCTLA4ex2 |
| 77 | RNA, amiR-LAG3ex1 |
| 78 | RNA, Pre-amiR-LAG3ex1 |
| 79 | RNA, amiR-TIM3ex2 |
| 80 | RNA, Pri-amiR-TIM3ex2 |
| 81 | RNA, si-TGFBR duplex |
| 82 | RNA, miR-4443 |
| 83 | RNA, pre-miR4443 |
| 84 | RNA, si-IL6 duplex#1; si-IL6 duplex#2 #1 sense sequence of Homo sapiens IL- |
| | 6 siRNA |
| 85 | RNA, si-IL6 duplex#1; si-IL6 duplex#2 #2 sense sequence of Homo sapiens IL- |
| | 6 siRNA |
| 86 | RNA, si-CD38 |
| 87 | RNA, amiR-TCRa-const |
| 88 | RNA, pri-amiR-TCRa-const |
| 89 | RNA, amiR-B2Mex1 |
| 90 | RNA, pri-miR-B2Mex1 |
| 91 | RNA, KIR (killer cell Immunoglobulin-like receptor) |
| 92 | DNA, KIR (killer cell Immunoglobulin-like receptor) |
| 93 | DNA, SIGLEC9 (Sialic acid-binding immunoglobulin-type lectin 9, also |
| | designated as CD329) |
| 94 | DNA, SIGLEC9 (Sialic acid-binding immunoglobulin-type lectin 9, also |
| | designated as CD329) |
| 95 | DNA, CD3 Gamma |
| 96 | DNA, CD3 Gamma |
| 97 | DNA, CD3 Delta |
| 98 | DNA, CD3 Delta |
| 99 | DNA, scaffold |
| 100 | DNA, siPolycistron PD1/TIGIT/MIR4443/BTLA/LAG3 |
| 101 | DNA, SV40 terminator |
| 102 | Protein, Flavobacterium Okeanokoites, FokI |
| 103 | Protein, Consensus FokI mutant |
| 104 | PROTEIN, Enhanced FokI nuclease domain |
| 105 | DNA, Primer3718 |
| 106 | DNA, drop off probe 3732 |
| 107 | DNA, Primer 3733 |
| 108 | DNA, drop off probe 3736 |
| 109 | DNA, drop off probe 3737 |
| 110 | DNA, PD1 exon 3 reference probe 3762 |
| 111 | Protein, >dScCas9-FokI-ZFQ |
| 112 | Protein, >dScCas9-FokI-Lam |
| 113 | Protein, >dScCas9-FokI-Strep |
| 114 | Protein, >dScCas9-FokI-Vir |
| 115 | Protein, >dScCas9-FokI-BRC2A2 |
| 116 | Protein, >dScCas9-FokI-DSS1 |
| 117 | Protein, >dScCas9-FokI-BRCA2-Strep |
| 118 | Protein, >dScCas9-FokI-DSS1-Strep |
| 119 | Protein, >dScCas9-FokI-BRCA2-virD2 |
| 120 | Protein, >dScCas9-FokI-DSS1-virD2 |
| 121 | PROTEIN, >ancestral dCas9-BfiI |
| 122 | PROTEIN, >ancestral dCas9-MnlI |
| 123 | PROTEIN, >ancestral dCas9-MmeI |
| 124 | PROTEIN, >RAD54 N-terminal peptide |
| 125 | PROTEIN, >ancestral dCas9-FokI-RAD54ntd (ancestral dCas9-FokI fused to |
| | a RAD54 N-terminal domain peptide) |
| 126 | Protein, >dScCasFok-BRCA2, 3NLS variant, Construct 13656 |
| 127 | Protein, >dScCasFok-DSS1, 3NLS variant, Construct 13659 |
| 128 | Protein, >dScCasFok-ZFQ, 3NLS variant, Construct 13648 |
| 129 | Protein, >dScCasFok-Strep, 1NLS variant, Construct 13665 |
| 130 | PROTEIN, >dCasFok, 2NLS, N-terminal BRCA2 |
| 131 | PROTEIN, >dCasFok, 2NLS, N-terminal BRCA2, 6His |
| 132 | PROTEIN, >dCasFok, 2NLS, N-terminal Streptavidin |
| 133 | PROTEIN, >dCasFok, 2NLS, N-terminal Streptavidin, 6His |
| 134 | PROTEIN, >dCasFok, 2NLS, N-terminal Pot |
| 1345 | PROTEIN, >dCasFok, 2NLS, N-terminal Pot, 6His |
| 136 | PROTEIN, >dCasFok, INLS, N-terminal Streptavidin, C-terminal BRCA2 |
| 137 | PROTEIN, >dCasFok, INLS, N-terminal Pot, C-terminal BRCA2 |
| 138 | PROTEIN, >dCasFok, INLS, ancestral RuvC + REC1/2, N- and C-terminal |
| | BRCA2 |
| 139 | PROTEIN, >dCasFok, 2NLS, ancestral RuvC + REC1/2, N-terminal BRCA2 |
| 140 | PROTEIN, >dCasFok, 2NLS, ancestral RuvC + REC1/2, N-terminal BRCA2, |
| | 6His |
| 141 | PROTEIN, >ancestral dCas9-FokI-RAD52id (ancestral dCas9-FokI fused to a |
| | RAD52 peptide that interacts with RAD51) |
| 142 | PROTEIN, >DSS1 peptide(n-term), ancestral dCasFok |
| 143 | PROTEIN, >BRCA2 peptide 2(n-term), ancestral dCasFok |
| 144 | PROTEIN, >RAD52 peptide(n-term), ancestral dCasFok |
| 145 | PROTEIN, >Streptavidin(n-term), ancestral dCasFok |
| 146 | PROTEIN, >BRCA2 peptide (c-term), ancestral dCasFok |
| 147 | PROTEIN, >DSS1 peptide (c-term), ancestral dCasFok |
| 148 | PROTEIN, >BRCA2 peptide 2 (c-term), ancestral dCasFok |
| 149 | PROTEIN, >RAD54 peptide (c-term), ancestral dCasFok |
| 150 | PROTEIN, >RAD52 peptide (c-term), ancestral dCasFok |
| 151 | PROTEIN, >Mdm2 peptide (c-term), ancestral dCasFok |
| 152 | PROTEIN, >Streptavidin (c-term), ancestral dCasFok |
| 153 | PROTEIN, >BRCA2 peptide(n-term), ancestral dCasFok, his-tagged |
| 154 | PROTEIN, >DSS1 peptide(n-term), ancestral dCasFok, his-tagged |
| 155 | PROTEIN, >BRCA2 peptide 2(n-term), ancestral dCasFok, his-tagged |
| 156 | PROTEIN, >RAD54 peptide(n-term), ancestral dCasFok, his-tagged |
| 157 | PROTEIN, >RAD52 peptide(n-term), ancestral dCasFok, his-tagged |
| 158 | PROTEIN, >Streptavidin(n-term), ancestral dCasFok, his-tagged |
| 159 | PROTEIN, >BRCA2 peptide (n and c-term), ancestral dCasFok |
| 160 | PROTEIN, >BRCA2 peptide (n-term), DSS1 peptide (c-term), ancestral |
| | dCasFok |
| 161 | PROTEIN, >BRCA2 peptide (n-term), BRCA2 peptide 2 (c-term), ancestral |
| | dCasFok |
| 162 | PROTEIN, >BRCA2 peptide (n-term), RAD52 peptide (c-term), ancestral |
| | dCasFok |
| 163 | PROTEIN, >BRCA2 peptide (n-term), Mdm2 peptide (c-term), ancestral |
| | dCasFok |
| 164 | PROTEIN, >BRCA2 peptide (n-term), Streptavidin (c-term), ancestral |
| | dCasFok |
| 156 | PROTEIN, >DSS1 peptide(n-term), BRCA2 peptide (c-term), ancestral |
| | dCasFok |
| 166 | PROTEIN, >DSS1 peptide(n-term), BRCA2 peptide 2 (c-term), ancestral |
| | dCasFok |
| 167 | PROTEIN, >DSS1 peptide(n-term), RAD54 peptide (c-term), ancestral |
| | dCasFok |
| 168 | PROTEIN, >DSS1 peptide(n-term), Streptavidin (c-term), ancestral dCasFok |
| 169 | PROTEIN, >Streptavidin(n-term), BRCA2 peptide (c-term), ancestral |
| | dCasFok |
| 170 | PROTEIN, >Streptavidin(n-term), DSS1 peptide (c-term), ancestral dCasFok |
| 171 | PROTEIN, >Streptavidin(n-term), BRCA2 peptide 2 (c-term), ancestral |
| | dCasFok |
| 172 | PROTEIN, >Streptavidin(n-term), RAD52 peptide (c-term), ancestral dCasFok |
| 173 | PROTEIN, >ancestral dCasFok, his-tagged |
| 174 | PROTEIN, >SpCas9, SV40 and nucleoplasmin NLS |
| 175 | Protein, >monomeric streptavidin domain |
| 176 | DNA, Homo sapiens DNMT3A CDS: >NC_000002.12: c25342590-25227874 |
| | Homo sapiens chromosome 2, GRCh38 |
| 177 | PROTEIN, Homo sapiens, DNMT3A protein: >NP_001307821.1 DNA |
| | (cytosine-5)-methyltransferase 3A isoform c |
| 178 | DNA, PD1 exon1 sgRNA #3478 |
| 179 | DNA, PD1 exon1 sgRNA #3479 |
| 180 | DNA, PD1 exon1 sgRNA for Cas9 #3480 |
| 181 | DNA, Blunt (B) NHEJ donor |
| 182 | DNA, W HDR donor with SCNA binding sites |
| 183 | PDCD1 (PD1) DNA:, DNA, Homo sapiens >NC_000002.12: c241858894- |
| | 241849884 Homo sapiens chromosome 2, GRCh38.p14 Primary Assembly |
| 184 | DNA, 4004 RHA flanking Right (PD1rev)2 |
| 185 | DNA, 4008 LHA flanking eGFPrev 2 |
| 186 | DNA, Homo sapiens, CXCR6 cds: >AF007545.1 Homo sapiens SIV/HIV |
| | receptor Bonzo (Bonzo) mRNA, complete cds |
| 187 | PROTEIN, Homo sapiens, CXCR6 |
| 188 | DNA, Homo sapiens, TIM3 (HAVCR2) DNA: >NC_000005.10: |
| 189 | PROTEIN, Homo sapiens, TIM3 (HAVCR2) protein: >NP_116171.3 |
| | hepatitis A virus cellular receptor 2 precursor [Homo sapiens] |
| 190 | DNA, Homo sapiens, LAG3 CDS: >NC_000012.12: 6772520-6778455 Homo |
| | sapiens chromosome 12, GRCh38.p14 Primary Assembly |
| 191 | PROTEIN, Homo sapiens, LAG3 protein: >NP_002277.4 lymphocyte |
| | activation gene 3 protein precursor [Homo sapiens] |
| 192 | DNA, Homo sapiens, TRAF4 DNA: >NC_000017.11: 28744011-28750956 |
| | Homo sapiens chromosome 17, GRCh38.p14 Primary Assembly CDS: >NM_004295.4 |
| | Homo sapiens TNF receptor associated factor 4 (TRAF4), |
| | mRNA |
| 193 | PROTEIN, Homo sapiens, TRAF4 protein: >NP_004286.2 TNF receptor- |
| | associated factor 4 [Homo sapiens] |
| 194 | DNA, Homo sapiens, LIF DNA: >NC_000022.11: c30246759-30240453 |
| | Homo sapiens chromosome 22, GRCh38.p14 Primary Assembly |
| 195 | DNA, Homo sapiens, LIF cds: 267 nt |
| 196 | PROTEIN, Homo sapiens, LIF protein: >NP_001244064.1 leukemia |
| | inhibitory factor isoform 2 [Homo sapiens] |
| 197 | DNA, Homo sapiens, TIGIT CDS: >NC_000003.12: 114294028-114310288 |
| | Homo sapiens chromosome 3, GRCh38.p14 Primary Assembly |
| 198 | PROTEIN, Homo sapiens, TIGIT protein (canonical), >NP_776160.2 T-cell |
| | immunoreceptor with Ig and ITIM domains precursor [Homo sapiens] |
| 199 | DNA, Homo sapiens, PDCD1 (PD1) CDS: >NC_000002.12: c241858894- |
| | 241849884 Homo sapiens chromosome 2, GRCh38.p14 Primary Assembly |
| | PDCD1 cds 867 nt: |
| 200 | PROTEIN, Homo sapiens, PDCD1 protein, >NP_005009.2 programmed cell |
| | death protein 1 precursor [Homo sapiens] |
| 201 | RNA, sgRNA1 |
| 202 | RNA, sgRNA2 |

1-56. (canceled)

57. An immune trans-regulatory and/or modulatory system comprising:
(a) at least one promoter-less donor nucleic acid molecule comprising at least one replacement nucleic acid sequence, wherein when inserted/introduced into a target sequence within at least one target first immune-regulatory gene/s of interest, said replacement nucleic acid sequence replaces at least part of the endogenous coding region of said target first immune-regulatory gene/s, and/or is introduced into the endogenous non-coding region of said target first immune-regulatory gene, said replacement and/or introduction results in at least one of: (i) the production of at least one immune-modulatory product, controlled by at least one endogenous control element of said target immune-regulatory gene; and (ii) inhibition and/or reduction and/or modulation of the expression and/or activity and/or stability of said at least one target first immune-regulatory gene; and
(b) at least one target recognition element targeted at a target sequence within said at least one target first immune-regulatory gene, or any nucleic acid sequence encoding said target recognition element.

58. The system according to claim 57, wherein at least one of:
(i) the system further comprises (c), at least one nucleic acid guided genome modifier protein, chimeric protein, complex or conjugate, or at least one nucleic acid sequence encoding said guided genome modifier protein;
(ii) said insertion of said at least one replacement nucleic acid sequence into said target sequence is mediated by at least one of homology-directed repair (HDR) recombination and Non Homologous End Joining (NHEJ), wherein when said insertion of the at least one replacement nucleic acid sequence into said target sequence is mediated by HDR, the donor nucleic acid molecule is flanked by at least one homology arm.

59. The system according to claim 57, wherein said at least one replacement nucleic acid sequence encodes at least one of: at least one molecule participating in at least one immune-related signal transducing pathway, or any parts or fragments thereof, at least one inhibitory and/or modulatory non-coding nucleic acid molecule, at least one modulator of hematopoietic cell proliferation, recruitment and/or survival, and at least one detectable moiety.

60. The system according to claim 59, wherein said at least one inhibitory and/or modulatory non-coding nucleic acid molecule is a ribonucleic acid (RNA) molecule, said RNA molecule is at least one of a double-stranded RNA (dsRNA), an antisense RNA, a single-stranded RNA (ssRNA), and a Ribozyme, optionally, at least one of:
(a) wherein said at least one inhibitory and/or modulatory non-coding nucleic acid molecule is at least one of a microRNA (miRNA), MicroRNA-like RNAs (milRNA), artificial miRNAs (amiRNA), small interfering RNA (siRNA), and short hairpin RNA (shRNA); and
(b) wherein at least one of:
(i) said at least one inhibitory and/or modulatory nucleic acid molecule that acts as a T cell Exhaustion tolerating component, is specifically directed against at least one of: at least one inhibitory receptor, at least one phosphatase, at least one methylase, optionally said inhibitory receptor is at least one of Programmed Death-1 (PD-1, CD279), cytotoxtic T lymphocyte antigen-4 (CTLA-4, CD152), Lymphocyte Activating 3 (Lag-3), T-cell immunoglobulin and mucin domain-3 (TIM-3), Cluster of Differentiation 244 (CD244/2B4), Cluster of Differentiation 160 (CD160) and T cell immunoreceptor with Ig and ITIM domains (TIGIT);
(ii) said at least one inhibitory and/or modulatory nucleic acid molecule that acts as an allogeneic T cell safety component is specifically directed against at least one of p2 microglobulin (B2M), T-cell receptor alpha (TCRα) and/or beta (TCRβ);
(iii) said at least one inhibitory and/or modulatory nucleic acid molecule that acts as Fratricide reducing/preventing component is specifically directed against at least one of cluster of differentiation 38 (CD38), T-cell receptor alpha (TCRα) and/or beta (TCRβ);
(iv) said at least one inhibitory and/or modulatory nucleic acid molecule that acts as a CRS preventing component is specifically directed against Interleukin 6 (IL-6); and
(v) said at least one inhibitory and/or modulatory nucleic acid molecule that acts as an activating component for NF-κB signaling pathway, is specifically directed against tumor necrosis factor receptor (TNFR)-associated factor 4 (TRAF4), optionally, said inhibitory and/or modulatory nucleic acid molecule is miR-4443.

61. The system according to claim 59, wherein said at least one inhibitory and/or modulatory nucleic acid molecule is specific for at least one nucleic acid sequence encoding and/or controlling the expression of at least one second immune-regulatory gene, and wherein said inhibitory and/or modulatory nucleic acid molecule acts as at least one of: (i) an allogeneic T cell safety component; (ii) Fratricide reducing/preventing component; (iii) cytokine release syndrome (CRS) preventing component; (iv) activating component for nuclear factor kappa-light-chain-enhancer of activated B cells (NF-κB) signaling pathway; and (v) T cell Exhaustion tolerating component.

62. The system according to claim 57, wherein said at least one target first immune-regulatory gene is one of: at least one gene encoding at least one immune-checkpoint protein, at least one gene encoding an immune-modulator of hematopoietic cell proliferation, recruitment and/or survival, at least one gene encoding at least one signal transduction molecule, at least one gene encoding at least one immuno-modulatory receptor, at least one hypoxia inducible gene, optionally, at least one of:
(i) wherein said at least one target first immune-regulatory gene is at least one gene encoding an immune-modulator of hematopoietic cell proliferation, recruitment and/or survival, optionally said target gene is Leukemia inhibitory factor (LIF-1); and
(ii) wherein said target sequence within said at least one target first immune-regulatory gene is a target sequence within at least one of exon 2 and exon 3 of said LIF-1 gene, and wherein said insertion is mediated by HDR or NHEJ.

63. The system according to claim 57, wherein said at least one target first immune-regulatory gene is at least one gene encoding at least one immune-checkpoint protein, optionally said immune-checkpoint protein is at least one of Programmed cell death protein 1 (PDCD1/PD1); T Cell Immunoreceptor With Ig And ITIM Domains (TIGIT); B And T Lymphocyte Associated (BTLA); Cytotoxic T-Lymphocyte-Associated protein 4 (CD152/CTLA4); Lymphocyte activation gene 3 (LAG-3) (CD223); and T cell immunoglobulin and mucin domain 3 (TIM-3)(HAVCR2).

64. The system according to claim 63, wherein said at least one target first immune-regulatory gene of interest is Programmed cell death protein 1 (PDCD1/PD-1), wherein said target sequence within said target immune-regulatory gene is a target sequence within at least one of: (i) exon 3 of said PD-1 gene; and (ii) exon 1 of said PD-1 gene, and wherein said insertion is mediated by HDR or NHEJ, optionally, at least one of:
(a) wherein said target sequence within said at least one target first immune-regulatory gene is within exon 3, wherein said insertion is mediated by NHEJ and wherein said insertion results in production of a soluble PD-1 truncated product;
(b) wherein said target sequence within said at least one target first immune-regulatory gene is within exon 3, wherein said insertion is mediated by NHEJ, and wherein said insertion results in knockout of the endogenous PD-1 gene; and
(c) wherein said target sequence within said at least one target first immune-regulatory gene is within exon 1, wherein said insertion is mediated by HDR, and wherein said insertion results in at least one of: (i) the production of at least one immune-modulatory product, controlled by at least one endogenous control element of said PD-1 gene; and (ii) inhibition and/or reduction and/or modulation of the expression and/or activity and/or stability of said PD-1 gene.

65. The system according to claim 64, wherein said target sequence within said at least one target first immune-regulatory gene is within exon 3, wherein said insertion is mediated by HDR, and wherein said replacement sequence comprises an in-frame nucleic acid sequence encoding a hematopoietic cell signaling domain, and wherein said insertion results in formation of at least one soluble or non-soluble PD-1 chimeric protein, optionally, at least one of:
(a) wherein said hematopoietic cell signaling domain encoded by said replacement sequence comprises at least one transmembrane and/or intracellular domain of at least one co-stimulatory receptor, and wherein said insertion results in the formation of at least one membrane-anchored chimera that transduces stimulatory signals, optionally, said co-stimulatory receptor is at least one of 4-1BB (CD137; TNFRS9), Cluster of Differentiation 28 (CD28) and Tumor necrosis factor receptor superfamily, member 4 (TNFRSF4, OX40); and
(b) wherein said hematopoietic cell signaling domain encoded by said replacement sequence is at least one immunoglobulin Fragment crystallizable region (Fc region), and wherein said insertion results in the formation of a soluble chimera that binds PD-1 ligand, blocks inhibitory signals and/or activates immune cells.

66. The system according to claim 59, wherein said replacement nucleic acid sequence further comprises at least one nucleic acid sequence encoding at least one modulator of hematopoietic cell proliferation, recruitment and/or survival, optionally, said modulator comprises at least one of Interleukin 1 (IL-1), Interleukin 7 (IL-7), Interleukin 15 (IL-15), Chemokine ligand 19 (CCL19) and Chemokine ligand 21 (CCL21).

67. The system according to claim 57, wherein said at least one target recognition element is at least one of a single strand ribonucleic acid (RNA) molecule, a double strand RNA molecule, a single-strand DNA molecule (ssDNA), a double strand DNA (dsDNA), a modified deoxy ribonucleotide (DNA) molecule, a modified RNA molecule, a locked-nucleic acid molecule (LNA), a peptide-nucleic acid molecule (PNA) and any hybrids or combinations thereof.

68. The system according to claim 58, wherein, at least one of:
(a) said at least one nucleic acid guided genome modifier protein, chimeric protein, complex or conjugate, comprises at least one nucleic acid modifier component and at least one component capable of binding said at least one target recognition element, optionally, at least one of:
(i) wherein said at least one nucleic acid modifier component is a protein-based modifier, a nucleic acid-based modifier or any combinations thereof, and wherein said protein-based modifier is at least one of a nuclease, a methyltransferase, a methylated DNA binding factor, a transcription factor, a transcription repressor, a chromatin remodeling factor, a polymerase, a demethylase, an acetylase, a deacetylase, a kinase, a phosphatase, an integrase, a recombinase, a ligase, a topoisomerase, a girase, a helicase, and any combinations thereof;
(ii) wherein said nucleic acid modifier component is at least one nuclease, optionally, said nuclease is a Type IIS restriction endonuclease or any fragment, variant, mutant, fusion protein or conjugate thereof, and
(iii) wherein said Type IIS restriction endonuclease is FokI or any fragment, variant, mutant, fusion protein or conjugate thereof; and
(b) wherein at least one of said at least one nucleic acid modifier component and at least one component capable of binding said at least one target recognition element of said at least one nucleic acid guided genome modifier protein, chimeric protein, complex or conjugate, comprises at least one clustered regularly interspaced short palindromic repeats (CRISPR)-Cas protein, cas protein derived domain and/or any variant and mutant thereof.

69. At least one cell comprising and/or modified by at least one immune trans-regulatory/modulatory system, or a population of cells comprising said at least one cell, said system is as defined by claim 57, comprising:
(a) at least one promoter-less donor nucleic acid molecule comprising at least one replacement nucleic acid sequence, wherein when inserted and/or introduced into a target sequence within at least one target first immune-regulatory gene of interest in said cell, said replacement nucleic acid sequence replaces at least part of the endogenous coding region of said target immune-regulatory gene, and/or is introduced into the endogenous non-coding region of said target first immune-regulatory gene, said replacement and/or introduction results in at least one of: (i) the production of at least one immune-modulatory product, controlled by at least one endogenous control element of said target immune-regulatory gene; and (ii) inhibition and/or reduction and/or modulation of the expression and/or activity and/or stability of said target immune-regulatory gene; and
(b) at least one target recognition element targeted at a target sequence within said at least one target first immune-regulatory gene, or any nucleic acid sequence encoding said target recognition element.

70. The cell according to claim 69, wherein at least one of:
(i) said system further comprising (c), at least one nucleic acid guided genome modifier protein, chimeric protein, complex or conjugate, or at least one nucleic acid sequence encoding said guided genome modifier protein;
(ii) said cell is at least one hematopoietic cell and/or at least one stem cell;
(iii) said hematopoietic cell is at least one lymphocyte, said lymphocyte is at least one genetically modified or unmodified cell of the T-cell-lineage;
(iv) wherein said T cell is at least one of regulatory T cell (Tregs), tumor-infiltrating lymphocyte (TIL) cell, cytotoxic T lymphocyte (CTL), and Natural Killer (NK) cells;
(v) wherein said genetically modified cells express at least one Chimeric Antigen Receptors (CAR) molecule; and
(vi) wherein said cell is of an autologous or an allogeneic source; optionally, said cell is of a subject suffering from at least one of an immune-related disorder or condition, and a proliferative disorder

71. A composition comprising at least one of:
(I) an immune trans-regulatory and/or modulatory system, as defined by claim 57, the system comprising:
(a) at least one promoter-less donor nucleic acid molecule comprising at least one replacement nucleic acid sequence, wherein when inserted and/or introduced into a target sequence within at least one target first immune-regulatory gene of interest, said replacement nucleic acid sequence replaces at least part of the endogenous coding region of said target first immune-regulatory gene, and/or is introduced into the endogenous non-coding region of said target first immune-regulatory gene, said replacement and/or introduction results in at least one of: (i) the production of at least one immune-modulatory product, controlled by at least one endogenous control element of said target immune-regulatory gene; and (ii) inhibition and/or reduction and/or modulation of the expression and/or activity and/or stability of said target first immune-regulatory gene;
(b) at least one target recognition element targeted at a target sequence within said target first immune-regulatory gene, or any nucleic acid sequence encoding said target recognition element; and optionally,
(c) at least one nucleic acid guided genome modifier protein, chimeric protein, complex or conjugate, or at least one nucleic acid sequence encoding said guided genome modifier protein;
(II) at least one nucleic acid cassette or any vector or vehicle comprising at least one of: (i) the nucleic acid sequence of (a); (i) the nucleic acid sequence of (b); (iii) the nucleic acid sequence of (c); and (iv) the nucleic acid sequence of (a) and at least one of (b) and (c); and
(III) at least one cell comprising and/or modified by at least one of: the nucleic acid the at least one system of (I), or the at least one nucleic acid cassette or any vector or vehicle of (II); or any matrix, nano- or micro-particle comprising at least one of (I) and (II), and/or a population of cells comprising at least one of said cell;
said composition optionally further comprises at least one of pharmaceutically acceptable carrier/s, diluent/s, excipient/s and additive/s.

72. A method of modulating at least one target cell, said method comprises the steps of contacting said cell with at least one of:
(I) an immune trans-regulatory and/or modulatory system comprising:
(a) at least one promoter-less donor nucleic acid molecule comprising at least one replacement nucleic acid sequence, wherein when inserted and/or introduced into a target sequence within at least one target first immune-regulatory gene of interest, said replacement nucleic acid sequence replaces at least part of the endogenous coding region of said target first immune-regulatory gene, and/or is introduced into the endogenous non-coding region of said target first immune-regulatory gene, said replacement and/or introduction results in at least one of: (i) the production of at least one immune-modulatory product, controlled by at least one endogenous control element of said target immune-regulatory gene; and (ii) inhibition and/or reduction and/or modulation of the expression and/or activity and/or stability of said target immune-regulatory gene;
(b) at least one target recognition element targeted at a target sequence within said target first immune-regulatory gene, or any nucleic acid sequence encoding said target recognition element; and optionally,
(c) at least one nucleic acid guided genome modifier protein, chimeric protein, complex or conjugate, or at least one nucleic acid sequence encoding said guided genome modifier protein;
(II) at least one nucleic acid cassette or any vector or vehicle comprising at least one of: (i) the nucleic acid sequence of (a); (i) the nucleic acid sequence of (b); (iii) the nucleic acid sequence of (c); and (iv) the nucleic acid sequence of (a) and at least one of (b) and (c);
(III) at least one genetically modified cell comprising and/or modified by at least one of: the nucleic acid the at least one system of (I), or the at least one nucleic acid cassette or any vector or vehicle of (II); or any matrix, nano- or micro-particle comprising at least one of (I) and (II), or a population of cells comprising said at least one cell; and
(IV) at least one composition comprising at least one of (I), (II), (III) or any combinations thereof.

73. The method according to claim 72, wherein at least one of:
(a) said target cell and/or said genetically modified cell is at least one hematopoietic cell and/or at least one stem cell;
(b) said hematopoietic cell is at least one lymphocyte, said lymphocyte is at least one genetically modified or unmodified cell of the T-cell-lineage;
(d) said T cell is at least one of Tregs, TIL cell, CTL, and NK cells; and
(e) said genetically modified cells express at least one chimeric antigen receptor (CAR) molecule.

74. The method according to claim 72, wherein said target cell is of a subject suffering from at least one immune-related disorder and/or at least one proliferative disorder, and wherein said contacting step is performed by administering to said subject an effective amount of at least one of said system of (I), at least one nucleic acid cassette or any vector or vehicle of (II), at least one genetically modified cell of (III), at least one composition of (IV), or any combinations thereof, optionally, at least one of:
(i) wherein said proliferative disorder is at least one neoplastic disorder, optionally, cancer, and wherein said immune-related disorder is at least one of an infectious disease, a graft versus host disease, an inflammatory disorder, an immune-cell mediated disorder and an autoimmune disorder; and
(ii) wherein said target cell is an immune cell or non-immune cell, and wherein said cell resides within at least one tissue of said subject affected by said immune-related disorder.

75. A method of curing or treating, preventing, inhibiting, reducing, eliminating, protecting or delaying the onset of a pathologic disorder in a mammalian subject, said method comprising the step of administering to said subject an effective amount of at least one of:
(I) an immune trans-regulatory and/or modulatory system comprising:
(a) at least one promoter-less donor nucleic acid molecule comprising at least one replacement nucleic acid sequence, wherein when inserted and/or introduced into a target sequence within at least one target first immune-regulatory gene of interest, said replacement nucleic acid sequence replaces at least part of the endogenous coding region of said target first immune-regulatory gene, and/or is introduced into the endogenous non-coding region of said target first immune-regulatory gene, said replacement and/or introduction results in at least one of: (i) the production of at least one immune-modulatory product, controlled by at least one endogenous control element of said target immune-regulatory gene; and (ii) inhibition and/or reduction and/or modulation of the expression and/or activity and/or stability of said target first immune-regulatory gene;
(b) at least one target recognition element targeted at a target sequence within said target first immune-regulatory gene, or any nucleic acid sequence encoding said target recognition element; and optionally,
(c) at least one nucleic acid guided genome modifier protein, chimeric protein, complex or conjugate, or at least one nucleic acid sequence encoding said guided genome modifier protein;
(II) at least one nucleic acid cassette or any vector or vehicle comprising at least one of: (i) the nucleic acid sequence of (a); (i) the nucleic acid sequence of (b); (iii) the nucleic acid sequence of (c); and (iv) the nucleic acid sequence of (a) and at least one of (b) and (c);
(III) at least one cell comprising and/or modified by at least one of: the nucleic acid the at least one system of (I), or the at least one nucleic acid cassette or any vector or vehicle of (II); or any matrix, nano- or micro-particle comprising at least one of (I) and (II), or a population of cells comprising said at least one cell; and
(IV) at least one composition comprising at least one of (I), (II), (III) or any combinations thereof.

76. The method according to claim 75, wherein at least one of:
(a) said immune trans-regulatory and/or modulatory system comprises:
(i) at least one promoter-less donor nucleic acid molecule comprising at least one replacement nucleic acid sequence, wherein when inserted/introduced into a target sequence within at least one target first immune-regulatory gene/s of interest, said replacement nucleic acid sequence replaces at least part of the endogenous coding region of said target first immune-regulatory gene/s, and/or is introduced into the endogenous non-coding region of said target first immune-regulatory gene, said replacement and/or introduction results in at least one of: (i) the production of at least one immune-modulatory product, controlled by at least one endogenous control element of said target immune-regulatory gene; and (ii) inhibition and/or reduction and/or modulation of the expression and/or activity and/or stability of said at least one target first immune-regulatory gene; and
(ii) at least one target recognition element targeted at a target sequence within said at least one target first immune-regulatory gene, or any nucleic acid sequence encoding said target recognition element;
(b) said pathologic disorder is at least one immune-related disorder and/or at least one proliferative disorder, wherein said proliferative disorder is at least one neoplastic disorder, specifically, cancer, and wherein said immune-related disorder is at least one of an infectious disease, a graft versus host disease, an inflammatory disorder, an immune-cell mediated disorder and an autoimmune disorder; and
(c) said cell is at least one hematopoietic cell and/or at least one stem cell; optionally, at least one of:
(i) wherein said hematopoietic cell is at least one lymphocyte, said lymphocyte is at least one genetically modified or unmodified cell of the T-cell-lineage;
(ii) wherein said T cell is at least one of Tregs, TIL cell, CTL, and NK cells;
(iii) wherein said method comprises the step of administering to said subject a therapeutically effective amount of at least one genetically modified cell comprising and/or modified by said at least one immune trans-regulatory/modulatory system;
(iv) wherein said at least one cell is of an autologous or allogeneic source; and
(v) wherein said method comprises the step of administering to said subject a therapeutically effective amount of said immune trans-regulatory/modulatory system, or any vehicle or vector comprising said system or any composition thereof.


SEQUENCE LISTING (202 sequences)

SEQ ID NO: 1 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 134 aa, Homo sapiens
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 3 - protein, 21 aa, synthetic construct
GSGEGRGSLLTCGDVEENPGP

SEQ ID NO: 4 - protein, 22 aa, synthetic construct
GSGATNFSLLKQAGDVEENPGP

SEQ ID NO: 5 - DNA, 226 bp, synthetic construct
  source 1..226: mol_type = other DNA
gtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggc

SEQ ID NO: 6 - DNA, 2027 bp, synthetic construct
  source 1..2027: mol_type = other DNA
ctaactaattagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtgatcatctcattctttctggccctgacctctacagccctgctgtttctgctgttcttcctgacactgcggttcagcgtggtcaagcggggcagaaagaagctgctgtacatcttcaagcagcccttcatgcggcccgtgcagaccacacaagaggaagatggctgctcctgcagattccccgaggaagaagaaggcggctgcgaactcggatctggcgaaggcagaggatccctgctgacatgtggcgacgtggaagagaaccccggacctatggctcagtctctggcactgtctctgctgatcctggtgctggccttcggcatccctagaacacagggatctgatggcggagcccaggactgctgtctgaagtacagccagagaaagatccccgccaaggtcgtgcggagctacagaaagcaagagcctagcctgggctgctctatccctgccatcctgtttctcccacggaagagatctcaggccgagctgtgcgccgatcctaaagaactgtgggtgcagcagctgatgcagcacctggacaagaccccatctcctcagaagcctgctcagggctgcagaaaggatagaggcgccagcaagaccggcaagaaaggcaagggatccaagggctgcaagcggaccgaaagaagccagacacctaaaggccctggcagcggctaagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcctaactaattag

SEQ ID NO: 7 - DNA, 695 bp, synthetic construct
  source 1..695: mol_type = other DNA
acgcgtaaaaaaaaagcaccgactcggtgccactttttcaagttgataacggactagccttattttaacttgctatttctagctctaaaacccaccagggtttggaactggggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaggggctggggtgggctgtgttttagagctagaaatagcaagttaaaataaggctagtccgttatcaacttgaaaaagtggcaccgagtcggtgctttttttacgcgt

SEQ ID NO: 8 - DNA, 695 bp, synthetic construct
  source 1..695: mol_type = other DNA
acgcgtaaaaaaaaagcaccgactcggtgccactttttcaagttgataacggactagccttattttaacttgctatttctagctctaaaaccagcccaccccagcccctcaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagttccaaaccctggtggtgttttagagctagaaatagcaagttaaaataaggctagtccgttatcaacttgaaaaagtggcaccgagtcggtgctttttttacgcgt

SEQ ID NO: 9 - DNA, 695 bp, synthetic construct
  source 1..695: mol_type = other DNA
acgcgtaaaaaaaaagcaccgactcggtgccactttttcaagttgataacggactagccttattttaacttgctatttctagctctaaaacgacaccaaccaccagggtttggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcctgggtgaggggctgggggttttagagctagaaatagcaagttaaaataaggctagtccgttatcaacttgaaaaagtggcaccgagtcggtgctttttttacgcgt

SEQ ID NO: 10 - DNA, 695 bp, synthetic construct
  source 1..695: mol_type = other DNA
acgcgtaaaaaaaaagcaccgactcggtgccactttttcaagttgataacggactagccttattttaacttgctatttctagctctaaaaccgacaccaaccaccagggttggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggcctgggtgaggggctggggttttagagctagaaatagcaagttaaaataaggctagtccgttatcaacttgaaaaagtggcaccgagtcggtgctttttttacgcgt

SEQ ID NO: 11 - DNA, 695 bp, synthetic construct
  source 1..695: mol_type = other DNA
acgcgtaaaaaaaaagcaccgactcggtgccactttttcaagttgataacggactagccttattttaacttgctatttctagctctaaaacccacgacaccaaccaccaggggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctggcctgggtgaggggctgttttagagctagaaatagcaagttaaaataaggctagtccgttatcaacttgaaaaagtggcaccgagtcggtgctttttttacgcgt

SEQ ID NO: 12 - DNA, 695 bp, synthetic construct
  source 1..695: mol_type = other DNA
acgcgtaaaaaaaaagcaccgactcggtgccactttttcaagttgataacggactagccttattttaacttgctatttctagctctaaaaccccacgacaccaaccaccagggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggctggcctgggtgaggggcgttttagagctagaaatagcaagttaaaataaggctagtccgttatcaacttgaaaaagtggcaccgagtcggtgctttttttacgcgt

SEQ ID NO: 13 - DNA, 695 bp, synthetic construct
  source 1..695: mol_type = other DNA
acgcgtaaaaaaaaagcaccgactcggtgccactttttcaagttgataacggactagccttattttaacttgctatttctagctctaaaacgccgcccacgacaccaaccaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggccggctggcctgggtgaggttttagagctagaaatagcaagttaaaataaggctagtccgttatcaacttgaaaaagtggcaccgagtcggtgctttttttacgcgt

SEQ ID NO: 14 - DNA, 695 bp, synthetic construct
  source 1..695: mol_type = other DNA
acgcgtaaaaaaaaagcaccgactcggtgccactttttcaagttgataacggactagccttattttaacttgctatttctagctctaaaaccaggccgcccacgacaccaaggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccactggccggctggcctgggtgttttagagctagaaatagcaagttaaaataaggctagtccgttatcaacttgaaaaagtggcaccgagtcggtgctttttttacgcgt

SEQ ID NO: 15 - DNA, 695 bp, synthetic construct
  source 1..695: mol_type = other DNA
acgcgtaaaaaaaaagcaccgactcggtgccactttttcaagttgataacggactagccttattttaacttgctatttctagctctaaaacccaggccagccggccagttcggtgtttcgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcaaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtgtcgtgggcggcctgctgttttagagctagaaatagcaagttaaaataaggctagtccgttatcaacttgaaaaagtggcaccgagtcggtgctttttttacgcgt

SEQ ID NO: 16 - DNA, 389 bp, synthetic construct
  source 1..389: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggccagttccaaaccctgggttttagagctagaaatagcaagttaaaataaggctagtccgttatcaacttgaaaaagtggcaccgagtcggtgctttttttacgcgtacttgcggccgcatattcctaggataccggt

SEQ ID NO: 17 - DNA, 389 bp, synthetic construct
  source 1..389: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggacaccaaccaccagggttgttttagagctagaaatagcaagttaaaataaggctagtccgttatcaacttgaaaaagtggcaccgagtcggtgctttttttacgcgtacttgcggccgcatattcctaggataccggt

SEQ ID NO: 18 - DNA, 389 bp, synthetic construct
  source 1..389: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcccacgacaccaaccaccagttttagagctagaaatagcaagttaaaataaggctagtccgttatcaacttgaaaaagtggcaccgagtcggtgctttttttacgcgtacttgcggccgcatattcctaggataccggt

SEQ ID NO: 19 - DNA, 156 bp, synthetic construct
  source 1..156: mol_type = other DNA
agagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgag

SEQ ID NO: 20 - protein, 183 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQSKPWWLVSWAACWAAWCC

SEQ ID NO: 21 - protein, 242 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQPNPGGWCRGRPAGQPGAASLGPGRHLLPGRTRDNRSQAHRPAPEGGPLSRACVLCGLWGAGFPVAREDPGAPRALCP

SEQ ID NO: 22 - DNA, 4547 bp, synthetic construct
  source 1..4547: mol_type = other DNA
ctaactaattaggccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttgtgcccttccagagagaagggcagaagtgccaacagcacacccctcacccagcccaaggcccgccggtcaattccagaccctggtcatcatctcattctttctggccctgacctctacagccctgctgtttctgctgttcttcctgacactgcggttcagcgttgtcggcggctctggcatggtttccaagggcgaagaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgcttcagcagataccccgaccacatgaagcagcacgatttcttcaagagcgccatgcctgagggctacgtgcaagagcggaccatcttcttcaaggacgacggcaactacaagaccagagccgaagtgaagttcgagggcgacaccctggtcaaccggatcgagctgaagggcatcgacttcaaagaggatggcaacatcctgggccacaaacttgagtacaactacaacagccacaacgtgtacatcatggccgacaagcagaaaaacggcatcaaagtgaacttcaagatccggcacaacatcgaggacggctctgtgcagctggccgatcactaccagcagaacacacccatcggagatggccctgtgctgctgcccgataaccactacctgagcacacagagcgccctgagcaaggaccccaacgagaagagggatcacatggtgctgctggaatttgtgaccgccgctggcatcaccctcggcatggatgaactgtacaaaggctctggcgaaggcaggggcagccttctgacatgcggagatgtggaagagaaccccggacctatggtgtccaaaggcgaggaactctttacaggggtcgtgcctatcctggtcgagctggatggcgacgtgaacggacataagttctccgtcagcggagaaggcgagggcgacgctacctatggcaagctgacactcaaattcatctgtacgacagggaaactgcccgttccatggccaacactcgtgacaaccctgactcatggggtgcagtgtttctctcgctaccctgatcacatgaagcaacatgacttttttaagtccgctatgcccgagggatatgtccaagagagaacgattttctttaaggatgatgggaattataagacacgggccgaagtcaaatttgaaggcgataccctcgtgaatcgcatcgaactgaaagggattgatttcaaagaggacgggaatattctcggacacaaattggagtataacttcaactcccacaatgtctatattatggctgataagcaaaagaatgggatcaaagtcaatttcaaaatcagacacaatatcgaagatggcagcgtccagctggctgaccattatcagcaaaacacccctatcggcgacggacccgttctgctgcctgacaatcactatctgtctacccagtccgctctgtctaaggaccctaatgagaaacgtgaccacatggtcttgcttgagttcgtgacagccgccggaattacactcggaatggacgagctttacaaaggttccggcgccaccaacttcagcctgctgaaacaggctggcgacgtcgaagagaatcctggtccaatggaagatgccaagaacatcaagaagggccctgctccattctaccctctggaagatggaacagccggcgagcagctgcataaggccatgaagagatacgccctggtgcctggcacaatcgcctttacagacgcccacatcgaagtgaacatcacctacgccgagtacttcgagatgagcgtgcggctggctgaagctatgaagcgctacggcctgaacaccaaccaccggatcgttgtgtgcagcgagaacagcctgcagttcttcatgcctgtgctgggcgccctgttcatcggagttgctgtggcccctgccaacgacatctacaacgagcgcgagctgctgaacagcatgaacatctcccagcctaccgtggtgttcgtgtccaagaagggactgcagaaaatcctgaacgtgcagaagaagctgccgatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgtccgtggtgccttttcaccacggcttcggcatgttcacaaccctgggctacctgatctgcggcttcagagtggtgctgatgtaccgcttcgaagaggaactgttcctgcggagcctgcaggattacaagatccagtctgccctgctggtgcctactctgttcagcttctttgccaagagcaccctgatcgataagtacgacctgagcaacctgcacgagatcgcctctggcggagcccctctgagcaaagaagtgggagaagccgtcgccaagcggttccatctgcctggcatcagacaaggctatggcctgaccgagacaacctccgccatcctgattacacccgaaggcgacgataagcctggcgccgttggaaaggtggtgccatttttcgaggccaaggtggtggacctggataccggcaaaacactgggcgttaaccagaggggcgagctgtgtgttagaggccctatgatcatgtccggctacgtgaacaaccccgaggccacaaatgccctgattgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgacgcaggcgttgcaggactgcccgatgatgatgctggcgaactgccagctgctgtggtcgtgctggaacacggcaagacaatgaccgagaaagaaattgtggactacgtggccagccaagtgaccaccgccaagaaactgagaggcggagtggtgtttgtggacgaggtgccaaaaggcctgacaggcaagctggatgcccggaagatcagagagattctgatcaaggccaagaaaggcggcaagagcaagctgtgagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgttggtgtcgtgggcggcctgctgggcagcctggtgctgctagtctgggtcctggccgtcatctgctcccgggccgcacgaggtaacgtcatcccagcccctcggcctgccctgccctaaccctgctggcggccctcactcccgcctccccttcctccacccttccctcaccccaccccacctccccccatctccccgccaggctaagtccctgatgaaggcccctggactaagaccccccacctaggagcacggctcagggtcggcctggtgaccccaagtgtgtttctctgcagggacaataggagccaggcgcaccggccagcccctggtgagtctcactcttttcctgcatgatccactgtgccttccttcctgggtgggcagaggtggaaggacaggctgggaccacacggcctgcaggactcacattctattatagccaggaccccacctccccagcccccaggcagcaacctcaatccctaaagccatgatct

SEQ ID NO: 23 - DNA, 4088 bp, synthetic construct
  source 1..4088: mol_type = other DNA
ctaactaattaggccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttgtgcccttccagagagaagggcagaagtgccaacagcacacccctcacccagcccaaggcccgccggtcaattccagaccctggtcatcatctcattctttctggccctgacctctacagccctgctgtttctgctgttcttcctgacactgcggttcagcgttgtcggcggctctggcatggtttccaagggcgaagaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgcttcagcagataccccgaccacatgaagcagcacgatttcttcaagagcgccatgcctgagggctacgtgcaagagcggaccatcttcttcaaggacgacggcaactacaagaccagagccgaagtgaagttcgagggcgacaccctggtcaaccggatcgagctgaagggcatcgacttcaaagaggatggcaacatcctgggccacaagcttgagtacaactacaacagccacaacgtgtacatcatggccgacaagcagaaaaacggcatcaaagtgaacttcaagatccggcacaacatcgaggacggctctgtgcagctggccgatcactaccagcagaacacacccatcggagatggccctgtgctgctgcccgataaccactacctgagcacacagagcgccctgagcaaggaccccaacgagaagagggatcacatggtgctgctggaatttgtgaccgccgctggcatcaccctcggcatggatgaactgtacaaaggctctggcgaaggcaggggcagccttctgacatgcggagatgtggaagagaaccccggacctatgacaatcctgggaaccacctttggcatggtgttcagcctgctgcaggtcgtgtctggcgagtctggctatgcccagaacggcgatctggaagatgccgagctggacgactacagcttcagctgctacagccagctggaagtgaacggcagccagcactctctgacctgcgcctttgaagatcccgacgtgaacatcaccaaccttgagttcgagatctgtggcgccctggtggaagtcaagtgcctgaatttccggaagctgcaagagatctactttatcgagacaaagaagttcctgctgatcggcaagagcaacatctgcgtgaaagtgggcgagaagtccctgacctgcaagaagatcgacctgaccaccatcgtgaagcccgaggctcctttcgatctgagcgtggtgtatagagagggcgccaacgacttcgtggtcaccttcaacaccagccacctccaaaagaaatacgtgaaggtgctgatgcacgacgtggcctaccggcaagagaaggacgagaacaagtggacccacgtgaacctgtccagcaccaaactgactctgctgcagagaaagctgcagcctgccgctatgtacgagatcaaagtgcggagcatccctgaccactactttaaaggcttttggagcgagtggtccccgagctactacttcagaacccctgagatcaacaacagcagcggcgagatggaccccatcctgctgacaatcagcatcctgagctttttcagcgtggccctgctggtcatcctggcctgtgtgctgtggaagaagcggatcaagcccatcgtgtggcctagcctgcctgaccacaagaaaaccctggaacacctgtgcaagaagccccggaagaacctgaacgtgtccttcaatcccgagagcttcctggactgccagatccacagagtggacgacatccaggccagggacgaagtggaaggctttctgcaggacacattccctcagcagctcgaagagagcgagaagcagagacttggaggcgacgtgcagagccctaattgcccttctgaggacgtggtcatcaccccagagagcttcggcagagatagcagcctgacatgtctggccggcaatgtgtccgcctgtgatgcccctatcctgagcagcagcagaagcctggattgcagagagagcggcaagaacggccctcacgtgtaccaggatctgctcctgtctctggggaccaccaatagcacactgcctccaccttttagcctgcaaagcggcatcctgactctgaaccctgtggctcagggacagccaatcctgacaagcctgggcagcaatcaagaagaggcctacgtcaccatgagcagcttctaccagaatcaaggcagcggcgccaccaacttcagtctgctgaaacaggctggcgacgtggaagaaaatccaggaccaatgttccacgtgtccttccggtacatcttcggcctgcctccactgatcctggtgctgctgcctgtggccagcagcgactgtgatatcgagggcaaagacggcaagcagtacgagagcgtgctgatggtgtccatcgaccagctgctggacagcatgaaggaaatcggcagcaactgcctgaacaacgagttcaacttcttcaagcggcacatctgcgacgccaacaaagaaggcatgttcctgttcagagccgccagaaagctgcggcagttcctgaagatgaacagcaccggcgacttcgacctgcatctgctgaaagtgtctgagggcaccaccatcctgctgaattgcaccggccaagtgaagggcagaaagcctgctgctctgggagaagcccagcctaccaagagcctggaagagaacaagtccctgaaagagcagaagaagctgaacgacctctgcttcctgaagcggctgctgcaagagatcaagacctgctggaacaagatcctgatgggcaccaaagagcactgagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgttggtgtcgtgggcggcctgctgggcagcctggtgctgctagtctgggtcctggccgtcatctgctcccgggccgcacgaggtaacgtcatcccagcccctcggcctgccctgccctaaccctgctggcggccctcactcccgcctccccttcctccacccttccctcaccccaccccacctccccccatctccccgccaggctaagtccctgatgaaggcccctggactaagaccccccacctaggagcacggctcagggtcggcctggtgaccccaagtgtgtttctctgcagggacaataggagccaggcgcaccggccagcccctggtgagtctcactcttttcctgcatgatccactgtgccttccttcctgggtgggcagaggtggaaggacaggctgggaccacacggcctgcaggactcacattctattatagccaggaccccacctccccagcccccaggcagcaacctcaatccctaaagccatgatct

SEQ ID NO: 24 - DNA, 3944 bp, synthetic construct
  source 1..3944: mol_type = other DNA
ctaactaattaggccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttgtgcccttccagagagaagggcagaagtgccaacagcacacccctcacccagcccaaggcccgccggtcaattccagaccctggtcatcatctcattctttctggccctgacctctacagccctgctgtttctgctgttcttcctgacactgcggttcagcgtggtcaagcggggcagaaagaagctgctgtacatcttcaagcagcccttcatgcggcccgtgcagaccacacaagaggaagatggctgctcctgcagattccccgaggaagaagaaggcggctgcgaactcggatctggcgaaggcagaggatctctgctgacatgcggcgacgtggaagagaaccctggacctatggtgtccaagggcgaagaactgttcaccggcgtggtgcccattctggtggaactggacggggatgtgaacggccacaagtttagcgttagcggagaaggcgaaggcgacgccacatacggaaagctgaccctgaagttcatctgcaccaccggcaagctgcctgtgccttggcctacactggtcaccacactgacacacggcgtgcagtgcttcagcagataccccgaccacatgaagcagcacgatttcttcaagagcgccatgcctgagggctacgtgcaagagcggaccatcttcttcaaggacgacggcaactacaagaccagagccgaagtgaagttcgagggcgacaccctggtcaaccggatcgagctgaagggcatcgacttcaaagaggatggcaacatcctgggccacaaacttgagtacaacttcaactcccacaacgtgtacatcatggccgacaagcagaaaaacggcatcaaagtgaacttcaagatccggcacaacatcgaggacggctctgtgcagctggccgatcactaccagcagaacacacccatcggagatggccctgtgctgctgcccgataaccactacctgagcacacagagcgccctgagcaaggaccccaacgagaagagggatcacatggtgctgctggaatttgtgaccgccgctggcatcaccctcggcatggatgaactgtacaaaggcagcggcgccaccaacttcagcctgctgaaacaggctggcgacgtcgaagaaaatccaggtccaatggaagatgccaagaacatcaagaagggccctgctccattctaccctctggaagatggaacagccggcgagcagctgcataaggccatgaagagatacgccctggtgcctggcacaatcgccttcacagatgcccacatcgaagtgaacatcacctacgccgagtacttcgagatgagcgtgcggctggctgaagctatgaagcgctacggcctgaacaccaaccaccggatcgttgtgtgcagcgagaacagcctgcagttcttcatgcctgtgctgggcgccctgttcatcggagttgctgtggcccctgccaacgacatctacaacgagcgcgagctgctgaacagcatgaacatctcccagcctaccgtggtgttcgtgtccaagaagggactgcagaaaatcctgaacgtccagaagaaactgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgtccgtggtgccttttcaccacggcttcggcatgttcacaaccctgggctacctgatctgcggctttagagtggtgctgatgtaccgcttcgaagaggaactgttcctgcggagcctgcaggattacaagatccagtctgccctgctggtgcctactctgttcagcttcttcgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaacctccgccatcctgattacacctgagggcgacgataagcctggcgccgttggaaaggtggtgccatttttcgaggccaaggtggtggatctggataccggcaagacactgggcgttaaccagaggggcgagctgtgtgttagaggccctatgatcatgtccggctacgtgaacaaccccgaggccacaaatgccctgattgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtcctggaacacggaaagaccatgaccgagaaagaaattgtggactacgtggccagccaagtgaccaccgccaagaaactgagaggcggagtggtgtttgtggacgaggtgccaaaaggcctgacaggcaagctggatgcccggaagatccgcgagattctgatcaaggccaagaaaggcggcaagagcaagctgtgagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgttggtgtcgtgggcggcctgctgggcagcctggtgctgctagtctgggtcctggccgtcatctgctcccgggccgcacgaggtaacgtcatcccagcccctcggcctgccctgccctaaccctgctggcggccctcactcccgcctccccttcctccacccttccctcaccccaccccacctccccccatctccccgccaggctaagtccctgatgaaggcccctggactaagaccccccacctaggagcacggctcagggtcggcctggtgaccccaagtgtgtttctctgcagggacaataggagccaggcgcaccggccagcccctggtgagtctcactcttttcctgcatgatccactgtgccttccttcctgggtgggcagaggtggaaggacaggctgggaccacacggcctgcaggactcacattctattatagccaggaccccacctccccagcccccaggcagcaacctcaatccctaaagccatgatct

SEQ ID NO: 25 - DNA, 4538 bp, synthetic construct
  source 1..4538: mol_type = other DNA
ctaactaattaggccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttgtgcccttccagagagaagggcagaagtgccaacagcacacccctcacccagcccaaggcccgccggtcaattccagaccctggtcatcatctcattctttctggccctgacctctacagccctgctgtttctgctgttcttcctgacactgcggttcagcgttgtcggcggctctggcatggtttccaagggcgaagaactgttcaccggcgtggtgcccattctggtggaactggacggggatgtgaacggccacaagtttagcgttagcggcgaaggcgaaggggatgccacatacggaaagctgaccctgaagttcatctgcaccaccggcaagctgcctgtgccttggcctacactggtcaccacactgacatacggcgtgcagtgcttcagcagataccccgaccacatgaagcagcacgatttcttcaagagcgccatgcctgagggctacgtgcaagagcggaccatcttcttcaaggacgacggcaactacaagaccagagccgaagtgaagttcgagggcgacaccctggtcaaccggatcgagctgaagggcatcgacttcaaagaggatggcaacatcctgggccacaaacttgagtacaactacaacagccacaacgtgtacatcatggccgacaagcagaaaaacggcatcaaagtgaacttcaagatccggcacaacatcgaggacggctctgtgcagctggccgatcactaccagcagaacacacccatcggagatggccctgtgctgctgcccgataaccactacctgagcacacagagcgccctgagcaaggaccccaacgagaagagggatcacatggtgctgctggaatttgtgaccgccgctggcatcaccctcggcatggatgaactgtacaaaggctctggcgaaggcaggggcagccttctgacatgcggagatgtggaagagaaccccggacctatggtgtctaaaggcgaagaggacaacatggccatcatcaaagagttcatgcggttcaaggtgcacatggaaggctccgtgaatggccacgagttcgagattgaaggcgaaggcgagggcagaccttacgagggaacacagaccgccaagctgaaagtgaccaaaggcggccctctgccatttgcctgggacattctgagccctcagtttatgtacggcagcaaggcctacgtgaagcaccccgccgatattcccgactacctgaagctgagcttccccgagggcttcaagtgggagagagtgatgaactttgaggacggcggcgtggtcaccgtgacacaagatagttctctgcaggacggcgagttcatctacaaagtgaagctgcggggcaccaactttccctctgacggccctgtgatgcagaaaaagaccatgggctgggaagccagcagcgagagaatgtaccctgaagatggcgccctgaagggcgagatcaagcagcggctgaaactgaaggatggcggccactacgacgccgaagtcaagaccacctacaaggccaagaaacccgtgcagctgcctggcgcctacaatgtgaacatcaagctggacatcaccagccacaatgaggactacaccatcgtggaacagtacgagagagccgaggggagacactctaccggcggaatggacgagctttacaaaggcagcggcgccaccaacttcagcctgctgaaacaggctggcgacgtggaagaaaatcctggaccaatggaagatgccaagaatatcaagaagggccctgctccattctaccctctggaagatggaacagccggcgagcagctgcataaggccatgaagagatacgccctggtgcctggcacaatcgccttcacagatgcccacatcgaagtgaatatcacctacgccgagtacttcgagatgagcgtgcggctggctgaagctatgaagcgctacggcctgaacaccaaccaccggatcgttgtgtgcagcgagaacagcctgcagttcttcatgcctgtgctgggcgccctgttcatcggagttgctgtggcccctgccaacgacatctacaacgagcgcgagctgctgaacagcatgaatatctcccagcctaccgtggtgttcgtgtccaagaagggactgcagaagatcctgaacgtccagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggcagcaccggactgcctaaaggtgttgccctgcctcacagaactgcctgcgtgcggtttagccacgccagagatcccatcttcggcaaccagatcatccccgacacagccatcctgtccgtggtgccttttcaccacggcttcggcatgttcacaaccctgggctacctgatctgcggcttcagagtggtgctgatgtaccgcttcgaagaggaactgttcctgcggagcctgcaggattacaagatccagtctgccctgctggtgcctactctgttcagcttctttgccaagagcaccctgatcgataagtacgacctgagcaacctgcacgagatcgcctctggcggagcccctctgtctaaagaagtgggcgaagccgtcgccaagcggtttcatctgcctggcatcagacaaggctatggcctgaccgagacaaccagcgccattctgattacccctgagggcgacgacaaacctggcgctgtgggaaaagtggtgccatttttcgaggccaaggtggtggacctggataccggcaaaacactgggcgttaaccagaggggcgagctgtgtgttagaggccctatgatcatgtccggctacgtgaacaaccctgaggccaccaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaacttcctgctgccgtggtcgtgctggaacacggcaagaccatgaccgagaaagaaattgtggactacgtggccagccaagtgaccaccgccaaaaagctgagaggcggagtggtgtttgtggacgaggtgccaaaaggcctgacaggcaagctggatgcccggaagatccgcgagatcctgatcaaagccaagaaaggcggcaagagcaagctgtgagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgttggtgtcgtgggcggcctgctgggcagcctggtgctgctagtctgggtcctggccgtcatctgctcccgggccgcacgaggtaacgtcatcccagcccctcggcctgccctgccctaaccctgctggcggccctcactcccgcctccccttcctccacccttccctcaccccaccccacctccccccatctccccgccaggctaagtccctgatgaaggcccctggactaagaccccccacctaggagcacggctcagggtcggcctggtgaccccaagtgtgtttctctgcagggacaataggagccaggcgcaccggccagcccctggtgagtctcactcttttcctgcatgatccactgtgccttccttcctgggtgggcagaggtggaaggacaggctgggaccacacggcctgcaggactcacattctattatagccaggaccccacctccccagcccccaggcagcaacctcaatccctaaagccatgatct

SEQ ID NO: 26 - DNA, 2510 bp, synthetic construct
  source 1..2510: mol_type = other DNA
ctaactaattaggccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttgtgcccttccagagagaagggcagaagtgccaacagcacacccctcacccagcccaaggcccgccggtcaattccagaccctggtcatcatctcattctttctggccctgacctctacagccctgctgtttctgctgttcttcctgacactgcggttcagcgtggtcaagcggggcagaaagaagctgctgtacatcttcaagcagcccttcatgcggcccgtgcagaccacacaagaggaagatggctgctcctgcagattccccgaggaagaagaaggcggctgcgaactcggatctggcgaaggcagaggatccctgctgacatgtggcgacgtggaagagaaccccggacctatggctcagtctctggcactgtctctgctgatcctggtgctggccttcggcatccctagaacacagggatctgatggcggagcccaggactgctgtctgaagtacagccagagaaagatccccgccaaggtcgtgcggagctacagaaagcaagagcctagcctgggctgctctatccctgccatcctgtttctcccacggaagagatctcaggccgagctgtgcgccgatcctaaagaactgtgggtgcagcagctgatgcagcacctggacaagaccccatctcctcagaagcctgctcagggctgcagaaaggatagaggcgccagcaagaccggcaagaaaggcaagggatccaagggctgcaagcggaccgaaagaagccagacacctaaaggccctggcagcggcgccacaaatttcagcctgctgaaacaggccggcgacgtcgaagaaaatcctgggcctatgttccacgtgtccttccggtacatcttcggcctgcctccactgatcctggtgctgctgcctgtggccagcagcgactgtgatatcgagggcaaagacggcaagcagtacgagagcgtgctgatggtgtccatcgaccagctgctggacagcatgaaggaaatcggcagcaactgcctgaacaacgagttcaacttcttcaagcggcacatctgcgacgccaacaaagaaggcatgttcctgttcagagccgccagaaagctgcggcagttcctgaagatgaacagcaccggcgacttcgacctgcatctgctgaaagtgtctgagggcaccaccatcctgctgaattgcaccggccaagtgaagggcagaaagcctgctgctctgggagaagcccagcctaccaagagcctggaagagaacaagtccctgaaagagcagaagaagctgaacgacctctgcttcctgaagcggctgctgcaagagatcaagacctgctggaacaagatcctgatgggcaccaaagagcactgagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgttggtgtcgtgggcggcctgctgggcagcctggtgctgctagtctgggtcctggccgtcatctgctcccgggccgcacgaggtaacgtcatcccagcccctcggcctgccctgccctaaccctgctggcggccctcactcccgcctccccttcctccacccttccctcaccccaccccacctccccccatctccccgccaggctaagtccctgatgaaggcccctggactaagaccccccacctaggagcacggctcagggtcggcctggtgaccccaagtgtgtttctctgcagggacaataggagccaggcgcaccggccagcccctggtgagtctcactcttttcctgcatgatccactgtgccttccttcctgggtgggcagaggtggaaggacaggctgggaccacacggcctgcaggactcacattctattatagccaggaccccacctccccagcccccaggcagcaacctcaatccctaaagccatgatct

SEQ ID NO: 27 - protein, 593 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVIISFFLALTSTALLFLLFFLTLRFSVVKRGRKKLLYIFKQPFMRPVQTTQEEDGCSCRFPEEEEGGCELGSGEGRGSLLTCGDVEENPGPMAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGPGSGATNFSLLKQAGDVEENPGPMFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 28 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 29 - protein, 239 aa, synthetic construct
MVSKGEELFTGVVPILVELDGDVNGHKFSVSGEGEGDATYGKLTLKFICTTGKLPVPWPTLVTTLTHGVQCFSRYPDHMKQHDFFKSAMPEGYVQERTIFFKDDGNYKTRAEVKFEGDTLVNRIELKGIDFKEDGNILGHKLEYNFNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 30 - protein, 550 aa, synthetic construct
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 31 - protein, 1272 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVIISFFLALTSTALLFLLFFLTLRFSVVGGSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGEGRGSLLTCGDVEENPGPMVSKGEELFTGVVPILVELDGDVNGHKFSVSGEGEGDATYGKLTLKFICTTGKLPVPWPTLVTTLTHGVQCFSRYPDHMKQHDFFKSAMPEGYVQERTIFFKDDGNYKTRAEVKFEGDTLVNRIELKGIDFKEDGNILGHKLEYNFNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGATNFSLLKQAGDVEENPGPMEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 32 - protein, 1210 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVIISFFLALTSTALLFLLFFLTLRFSVVGGSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGEGRGSLLTCGDVEENPGPMFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHGSGATNFSLLKQAGDVEENPGPMEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 33 - protein, 1071 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVIISFFLALTSTALLFLLFFLTLRFSVVKRGRKKLLYIFKQPFMRPVQTTQEEDGCSCRFPEEEEGGCELGSGEGRGSLLTCGDVEENPGPMVSKGEELFTGVVPILVELDGDVNGHKFSVSGEGEGDATYGKLTLKFICTTGKLPVPWPTLVTTLTHGVQCFSRYPDHMKQHDFFKSAMPEGYVQERTIFFKDDGNYKTRAEVKFEGDTLVNRIELKGIDFKEDGNILGHKLEYNFNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGATNFSLLKQAGDVEENPGPMEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 34 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 35 - protein, 1269 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVIISFFLALTSTALLFLLFFLTLRFSVVGGSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKGSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGSGATNFSLLKQAGDVEENPGPMEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 36 - DNA, 2510 bp, synthetic construct; identical to SEQ ID NO: 26
  source 1..2510: mol_type = other DNA

SEQ ID NO: 37 - protein, 588 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQGGSGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 38 - DNA, 2141 bp, synthetic construct
  source 1..2141: mol_type = other DNA
ctaactaattagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtgatcatctcattctttctggccctgacctctacagccctgctgtttctgctgttcttcctgacactgcggttcagcgtggtcgtgtccaaaggcgaggaactttttaccggcgtggtgcccatcctggtggaactggatggggatgtgaacggccacaagttcagcgtcagcggagaaggcgaaggcgacgccacatacggaaagctgaccctgaagttcatctgcaccaccggcaagctgcctgtgccttggcctacactggtcaccacactgacatacggcgtgcagtgcttcagcagataccccgaccacatgaagcagcacgatttcttcaagagcgccatgcctgagggctacgtgcaagagagaaccatcttcttcaaggacgacggcaactacaagaccagagccgaagtgaagttcgagggcgacaccctggtcaacagaatcgagctgaagggcatcgacttcaaagaggatggcaacatcctgggccacaaacttgagtacaactacaacagccacaacgtgtacatcatggccgacaagcagaaaaacggcatcaaagtgaacttcaagatccggcacaacatcgaggacggctctgtgcagctggccgatcactaccagcagaacacacctatcggcgacggacctgtgctgctgcctgataaccactacctgagcacacagagcgccctgagcaaggaccctaacgagaagagggaccacatggtgctgctggaatttgtgaccgccgctggcatcacactcggcatggatgagctgtacaagtaagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcctaactaattag

SEQ ID NO: 39 - RNA, 53 nt, synthetic construct
  source 1..53: mol_type = other RNA
ggtgggggttggaggcgtgggttttagaacctatccctttctagccctgagca

SEQ ID NO: 40 - RNA, 17 nt, synthetic construct
  source 1..17: mol_type = other RNA
ttggaggcgtgggtttt

SEQ ID NO: 41 - RNA, 20 nt, synthetic construct
  source 1..20: mol_type = other RNA
atctggttcatgaggttgtt

SEQ ID NO: 42 - RNA, 20 nt, synthetic construct
  source 1..20: mol_type = other RNA
acagctcaatggcagtgcca

SEQ ID NO: 43 - RNA, 20 nt, synthetic construct
  source 1..20: mol_type = other RNA
tcatgaggttgttgtgacat

SEQ ID NO: 44 - RNA, 20 nt, synthetic construct
  source 1..20: mol_type = other RNA
actggcacagctcaatggca

SEQ ID NO: 45 - DNA, 2282 bp, synthetic construct
  source 1..2282: mol_type = other DNA
ctgggccaccacgggaggtgggaagagaggggtctcctttctcctggtgcctgctgggcctggggcctggtgccttccagccagagggccagggggccttaggacctttgccttcctgagggaaagggtgggatggctgggagtctctcctggaccctgcaccctttgggtggaaatggcttgtgtcttgccctcatctttacgtcatcccagaagagagcagggagaactaaggtagagaaagggagacagagaaacacacgcagtgacagagtgaagactaggccccaggaagacagctgcaggtggtgagggggaaccaggagtcctctcccatgcggccattgttggacccccatccggtgtgccatgaccccaggccaccctttcctgcctttctactcatggcttcttcctgactgtccccaggagttgtgcccctgctgttggttctgcactggaaacatggggcggggagccccctccccatcacccctgtcgaaggcagaggatccctgctgacatgtggcgacgtggaagagaaccccggacctatggctcagtctctggcactgtctctgctgatcctggtgctggccttcggcatccctagaacacagggatctgatggcggagcccaggactgctgtctgaagtacagccagagaaagatccccgccaaggtcgtgcggagctacagaaagcaagagcctagcctgggctgctctatccctgccatcctgtttctcccacggaagagatctcaggccgagctgtgcgccgatcctaaagaactgtgggtgcagcagctgatgcagcacctggacaagaccccatctcctcagaagcctgctcagggctgcagaaaggatagaggcgccagcaagaccggcaagaaaggcaagggatccaagggctgcaagcggaccgaaagaagccagacacctaaaggccctggcagcggcgccacaaatttcagcctgctgaaacaggccggcgacgtcgaagaaaatcctgggcctatgttccacgtgtccttccggtacatcttcggcctgcctccactgatcctggtgctgctgcctgtggccagcagcgactgtgatatcgagggcaaagacggcaagcagtacgagagcgtgctgatggtgtccatcgaccagctgctggacagcatgaaggaaatcggcagcaactgcctgaacaacgagttcaacttcttcaagcggcacatctgcgacgccaacaaagaaggcatgttcctgttcagagccgccagaaagctgcggcagttcctgaagatgaacagcaccggcgacttcgacctgcatctgctgaaagtgtctgagggcaccaccatcctgctgaattgcaccggccaagtgaagggcagaaagcctgctgctctgggagaagcccagcctaccaagagcctggaagagaacaagtccctgaaagagcagaagaagctgaacgacctctgcttcctgaagcggctgctgcaagagatcaagacctgctggaacaagatcctgatgggcaccaaagagcactgagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcctctttattctctatgtaagttacccctgggatactgacaggagatggcagggagggggcttgtaaatatcattaggggctgtcctgatctgggttgaggggaccttttggggctggaaggagagaatggggagagggcttgattaaaccacccccagactcccgccacttcctgcccaagcttccccagggaagcttccccagggtgcccagttagcaaggggagaactgagtgcaaaggtggggacctggcacttcttatcttgtgattgtcctgctgcagggagcgagggatggaggggaaatgggcgtgaggcaccagggagatgcggttgagaggcagtgggctgtgggtgctgggcatggaggggcgtcccggaacattgtgagtgcagggatggaagtacttgtgtgtggtgccccagctagggctagacaccgagttttcccttctgtccccttagggtggtgatgatgatgatgatgataatgatgactgc

SEQ ID NO: 46 - DNA, 500 bp, synthetic construct
  source 1..500: mol_type = other DNA
ctgggccaccacgggaggtgggaagagaggggtctcctttctcctggtgcctgctgggcctggggcctggtgccttccagccagagggccagggggccttaggacctttgccttcctgagggaaagggtgggatggctgggagtctctcctggaccctgcaccctttgggtggaaatggcttgtgtcttgccctcatctttacgtcatcccagaagagagcagggagaactaaggtagagaaagggagacagagaaacacacgcagtgacagagtgaagactaggccccaggaagacagctgcaggtggtgagggggaaccaggagtcctctcccatgcggccattgttggacccccatccggtgtgccatgaccccaggccaccctttcctgcctttctactcatggcttcttcctgactgtccccaggagttgtgcccctgctgttggttctgcactggaaacatggggcggggagccccctccccatcacccctgtc

SEQ ID NO: 47 - DNA, 54 bp, synthetic construct
  source 1..54: mol_type = other DNA
gaaggcagaggatccctgctgacatgtggcgacgtggaagagaaccccggacct

SEQ ID NO: 48 - DNA, 411 bp, Homo sapiens
  source 1..411: mol_type = other DNA
atggctcagtctctggcactgtctctgctgatcctggtgctggccttcggcatccctagaacacagggatctgatggcggagcccaggactgctgtctgaagtacagccagagaaagatccccgccaaggtcgtgcggagctacagaaagcaagagcctagcctgggctgctctatccctgccatcctgtttctcccacggaagagatctcaggccgagctgtgcgccgatcctaaagaactgtgggtgcagcagctgatgcagcacctggacaagaccccatctcctcagaagcctgctcagggctgcagaaaggatagaggcgccagcaagaccggcaagaaaggcaagggatccaagggctgcaagcggaccgaaagaagccagacacctaaaggccctggcagcggc

SEQ ID NO: 49 - DNA, 57 bp, synthetic construct
  source 1..57: mol_type = other DNA
gccacaaatttcagcctgctgaaacaggccggcgacgtcgaagaaaatcctgggcct

SEQ ID NO: 50 - DNA, 534 bp, Homo sapiens
  source 1..534: mol_type = other DNA
atgttccacgtgtccttccggtacatcttcggcctgcctccactgatcctggtgctgctgcctgtggccagcagcgactgtgatatcgagggcaaagacggcaagcagtacgagagcgtgctgatggtgtccatcgaccagctgctggacagcatgaaggaaatcggcagcaactgcctgaacaacgagttcaacttcttcaagcggcacatctgcgacgccaacaaagaaggcatgttcctgttcagagccgccagaaagctgcggcagttcctgaagatgaacagcaccggcgacttcgacctgcatctgctgaaagtgtctgagggcaccaccatcctgctgaattgcaccggccaagtgaagggcagaaagcctgctgctctgggagaagcccagcctaccaagagcctggaagagaacaagtccctgaaagagcagaagaagctgaacgacctctgcttcctgaagcggctgctgcaagagatcaagacctgctggaacaagatcctgatgggcaccaaagagcactga

SEQ ID NO: 51 - DNA, 500 bp, synthetic construct
  source 1..500: mol_type = other DNA
ctctttattctctatgtaagttacccctgggatactgacaggagatggcagggagggggcttgtaaatatcattaggggctgtcctgatctgggttgaggggaccttttggggctggaaggagagaatggggagagggcttgattaaaccacccccagactcccgccacttcctgcccaagcttccccagggaagcttccccagggtgcccagttagcaaggggagaactgagtgcaaaggtggggacctggcacttcttatcttgtgattgtcctgctgcagggagcgagggatggaggggaaatgggcgtgaggcaccagggagatgcggttgagaggcagtgggctgtgggtgctgggcatggaggggcgtcccggaacattgtgagtgcagggatggaagtacttgtgtgtggtgccccagctagggctagacaccgagttttcccttctgtccccttagggtggtgatgatgatgatgatgataatgatgactgc

SEQ ID NO: 52 - RNA, 20 nt, synthetic construct
  source 1..20: mol_type = other RNA
gtggactggccaggcaccct

SEQ ID NO: 53 - RNA, 20 nt, synthetic construct
  source 1..20: mol_type = other RNA
ctgtgatgaagctgagcaga

SEQ ID NO: 54 - RNA, 20 nt, synthetic construct
  source 1..20: mol_type = other RNA
accgagggatctcaggagtt

SEQ ID NO: 55 - RNA, 20 nt, synthetic construct
  source 1..20: mol_type = other RNA
agacctcctgctagaaggcc

SEQ ID NO: 56 - DNA, 2281 bp, synthetic construct
  source 1..2281: mol_type = other DNA
gggaaacagacgcaagagcttgcccaaagggttggcggcagggctgggacactgacccctgactcccacgtcacctcccttctgcccctcagtacacagcccagggggagccgttccccaacaacctggacaagctatgtggccccaacgtgacggacttcccgcccttccacgccaacggcacggagaaggccaagctggtggagctgtaccgcatagtcgtgtaccttggcacctccctgggcaacatcacccgggaccagaagatcctcaaccccagtgccctcagcctccacagcaagctcaacgccaccgccgacatcctgcgaggcctccttagcaacgtgctgtgccgcctgtgcagcaagtaccacgtgggccatgtggacgtgacctacggccctgacacctcgggtaaggatgtcttccagaagaagaagctgggctgtcaactcctggggaagtataagcagatcatcgccgtgttggcccaggccttgaaggcagaggatccctgctgacatgtggcgacgtggaagagaaccccggacctatggctcagtctctggcactgtctctgctgatcctggtgctggccttcggcatccctagaacacagggatctgatggcggagcccaggactgctgtctgaagtacagccagagaaagatccccgccaaggtcgtgcggagctacagaaagcaagagcctagcctgggctgctctatccctgccatcctgtttctcccacggaagagatctcaggccgagctgtgcgccgatcctaaagaactgtgggtgcagcagctgatgcagcacctggacaagaccccatctcctcagaagcctgctcagggctgcagaaaggatagaggcgccagcaagaccggcaagaaaggcaagggatccaagggctgcaagcggaccgaaagaagccagacacctaaaggccctggcagcggcgccacaaatttcagcctgctgaaacaggccggcgacgtcgaagaaaatcctgggcctatgttccacgtgtccttccggtacatcttcggcctgcctccactgatcctggtgctgctgcctgtggccagcagcgactgtgatatcgagggcaaagacggcaagcagtacgagagcgtgctgatggtgtccatcgaccagctgctggacagcatgaaggaaatcggcagcaactgcctgaacaacgagttcaacttcttcaagcggcacatctgcgacgccaacaaagaaggcatgttcctgttcagagccgccagaaagctgcggcagttcctgaagatgaacagcaccggcgacttcgacctgcatctgctgaaagtgtctgagggcaccaccatcctgctgaattgcaccggccaagtgaagggcagaaagcctgctgctctgggagaagcccagcctaccaagagcctggaagagaacaagtccctgaaagagcagaagaagctgaacgacctctgcttcctgaagcggctgctgcaagagatcaagacctgctggaacaagatcctgatgggcaccaaagagcactgagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcgagcagagtggaaacttccatagggagggagctagaagaaggtgccccttcctctgggagattgtggactggggagcgtgggctggacttctgcctctacttgtccctttggccccttgctcactttgtgcagtgaacaaactacacaagtcatctacaagagccctgaccacagggtgagacagcagggcccaggggagtggaccagcccccagcaaattatcaccatctgtgcctttgctgccccttaggttgggacttaggtgggccagaggggctaggatcccaaaggactccttgtcccctagaagtttgatgagtggaagatagagaggggcctctgggatggaaggctgtcttcttttgaggatgatcagagaacttgggcataggaacaatctggcagaagtttccagaaggaggtcacttggcattcaggctcttggggaggcagagaagccaccttcaggcctgggaaggaagacactgggaggaggaga

SEQ ID NO: 57 - DNA, 500 bp, synthetic construct
  source 1..500: mol_type = other DNA
gggaaacagacgcaagagcttgcccaaagggttggcggcagggctgggacactgacccctgactcccacgtcacctcccttctgcccctcagtacacagcccagggggagccgttccccaacaacctggacaagctatgtggccccaacgtgacggacttcccgcccttccacgccaacggcacggagaaggccaagctggtggagctgtaccgcatagtcgtgtaccttggcacctccctgggcaacatcacccgggaccagaagatcctcaaccccagtgccctcagcctccacagcaagctcaacgccaccgccgacatcctgcgaggcctccttagcaacgtgctgtgccgcctgtgcagcaagtaccacgtgggccatgtggacgtgacctacggccctgacacctcgggtaaggatgtcttccagaagaagaagctgggctgtcaactcctggggaagtataagcagatcatcgccgtgttggcccaggccttc

SEQ ID NO: 58 - DNA, 500 bp, synthetic construct
  source 1..500: mol_type = other DNA
gagcagagtggaaacttccatagggagggagctagaagaaggtgccccttcctctgggagattgtggactggggagcgtgggctggacttctgcctctacttgtccctttggccccttgctcactttgtgcagtgaacaaactacacaagtcatctacaagagccctgaccacagggtgagacagcagggcccaggggagtggaccagcccccagcaaattatcaccatctgtgcctttgctgccccttaggttgggacttaggtgggccagaggggctaggatcccaaaggactccttgtcccctagaagtttgatgagtggaagatagagaggggcctctgggatggaaggctgtcttcttttgaggatgatcagagaacttgggcataggaacaatctggcagaagtttccagaaggaggtcacttggcattcaggctcttggggaggcagagaagccaccttcaggcctgggaaggaagacactgggaggaggaga

SEQ ID NO: 59 - DNA, 20 bp, synthetic construct
  source 1..20: mol_type = other DNA
gcagaaggctgagatcctgg

SEQ ID NO: 60 - DNA, 20 bp, synthetic construct
  source 1..20: mol_type = other DNA
cttggcagcatcagccagac

SEQ ID NO: 61 - DNA, 20 bp, synthetic construct
  source 1..20: mol_type = other DNA
ggtgtagaagcagggcagat

SEQ ID NO: 62 - DNA, 20 bp, synthetic construct
  source 1..20: mol_type = other DNA
aacctcgtgcccgtctgctg

SEQ ID NO: 63 - DNA, 20 bp, synthetic construct
  source 1..20: mol_type = other DNA
atgccgttctgctggtctct

SEQ ID NO: 64 - DNA, 20 bp, synthetic construct
  source 1..20: mol_type = other DNA
cactttgctctgtctgctct

SEQ ID NO: 65 - DNA, 20 bp, synthetic construct
  source 1..20: mol_type = other DNA
aagtcagaggacacctctgt

SEQ ID NO: 66 - DNA, 20 bp, synthetic construct
  source 1..20: mol_type = other DNA
catgttttcacatcttccct

SEQ ID NO: 67 - RNA, 23 nt, synthetic construct
  source 1..23: mol_type = other RNA
aagctgcaggtgaaggtggcgtt

SEQ ID NO: 68 - RNA, 71 nt, synthetic construct
  source 1..71: mol_type = other RNA
gtagcacaagctgcaggtgaaggtggcgtttgtttagttataacccaccttcacctgcagcaatgtactgc

SEQ ID NO: 69 - RNA, 19 nt, synthetic construct
  source 1..19: mol_type = other RNA
ttctaagaaccatcctggc

SEQ ID NO: 70 - RNA, 136 nt, synthetic construct
  source 1..136: mol_type = other RNA
ctcgagaagaaggtatattgctgttgacagtgagcgacgccaggatggttcttagaatagtgaagcttcagatgtattctaagaaccatcctggcgctgcctactgcctcggacttcaaggggtcagtcagaattc

SEQ ID NO: 71 - RNA, 22 nt, synthetic construct
  source 1..22: mol_type = other RNA
tttctattgtgcctgtcatcat

SEQ ID NO: 72 - RNA, 72 nt, synthetic construct
  source 1..72: mol_type = other RNA
tgttctttctattgtgcctgtcatcatgtgaagtagattagcatcatgatgtcagggtcaatagaatggcat

SEQ ID NO: 73 - RNA, 18 nt, synthetic construct
  source 1..18: mol_type = other RNA
tatataaagctgtacatc

SEQ ID NO: 74 - RNA, 136 nt, synthetic construct
  source 1..136: mol_type = other RNA
ctcgagaagaaggtatattgctgttgacagtgagcgacggatgtacagctttatatatagtgaagcttcagatgtatatataaagctgtacatccgctgcctactgcctcggacttcaaggggtcagtcagaattc

SEQ ID NO: 75 - RNA, 19 nt, synthetic construct
  source 1..19: mol_type = other RNA
tatagtgaggttcacttgc

SEQ ID NO: 76 - RNA, 136 nt, synthetic construct
  source 1..136: mol_type = other RNA
ctcgagaagaaggtatattgctgttgacagtgagcgacgcaagtgaacctcactatatagtgaagcttcagatgtatatagtgaggttcacttgcgctgcctactgcctcggacttcaaggggtcagtcagaattc

SEQ ID NO: 77 - RNA, 21 nt, synthetic construct
  source 1..21: mol_type = other RNA
ttatacatgatggagacgttg

SEQ ID NO: 78 - RNA, 76 nt, synthetic construct
  source 1..76: mol_type = other RNA
gcagtcctctgcaacgtctccacatgtataacgaagaatgtagtttatacatgatggagacgttgtggtggcctgc

SEQ ID NO: 79 - RNA, 19 nt, synthetic construct
  source 1..19: mol_type = other RNA
taataattcacatcccttc

SEQ ID NO: 80 - RNA, 136 nt, synthetic construct
  source 1..136: mol_type = other RNA
ctcgagaagaaggtatattgctgttgacagtgagcgacgaagggatgtgaattattatagtgaagcttcagatgtataataattcacatcccttcgctgcctactgcctcggacttcaaggggtcagtcagaattc

SEQ ID NO: 81 - RNA, 19 nt, synthetic construct
  source 1..19: mol_type = other RNA
ccatcgagtgccaaatgaa

SEQ ID NO: 82 - RNA, 17 nt, synthetic construct
  source 1..17: mol_type = other RNA
ttggaggcgtgggtttt

SEQ ID NO: 83 - RNA, 53 nt, synthetic construct; identical to SEQ ID NO: 39
  source 1..53: mol_type = other RNA

SEQ ID NO: 84 - RNA, 19 nt, synthetic construct
  source 1..19: mol_type = other RNA
agacatgtaacaagagtaa

SEQ ID NO: 85 - RNA, 19 nt, synthetic construct
  source 1..19: mol_type = other RNA
ggagacttgcctggtgaaa

SEQ ID NO: 86 - RNA, 25 nt, synthetic construct
  source 1..25: mol_type = other RNA
tttggcagtctacatgtctcatctc

SEQ ID NO: 87 - RNA, 19 nt, synthetic construct
  source 1..19: mol_type = other RNA
tatatacacatcagaatcc

SEQ ID NO: 88 - RNA, 138 nt, synthetic construct
  source 1..138: mol_type = other RNA
ctcgagaagaaggtatattgctgttgacagtgagcgacggattctgatgtgtatatataagtgaagcttcagatgttatatatacacatcagaatccgctgcctactgcctcggacttcaaggggtcagtcagaattc

SEQ ID NO: 89 - RNA, 19 nt, synthetic construct
  source 1..19: mol_type = other RNA
tagaaagagagagtagcgc

SEQ ID NO: 90 - RNA, 135 nt, synthetic construct
  source 1..135: mol_type = other RNA
tcgagaagaaggtatattgctgttgacagtgagcgacgcgctactctctctttctatagtgaagcttcagatgtatagaaagagagagtagcgcgctgcctactgcctcggacttcaaggggtcagtcagaattc

SEQ ID NO: 91 - RNA, 19 nt, synthetic construct
  source 1..19: mol_type = other RNA
tatatttgcaggagatgcc

SEQ ID NO: 92 - DNA, 136 bp, synthetic construct
  source 1..136: mol_type = other DNA
ctcgagaagaaggtatattgctgttgacagtgagcgacggcatctcctgcaaatatatagtgaagcttcagatgtatatatttgcaggagatgccgctgcctactgcctcggacttcaaggggtcagtcagaattc

SEQ ID NO: 93 - DNA, 19 bp, synthetic construct
  source 1..19: mol_type = other DNA
tttatacttcctttctccc

SEQ ID NO: 94 - DNA, 68 bp, synthetic construct
  source 1..68: mol_type = other DNA
agatctgggagaaaggaagtataaatacctgacccatatttatacttcctttctccctttttggtacc

SEQ ID NO: 95 - DNA, 21 bp, synthetic construct
  source 1..21: mol_type = other DNA
ttatcttcagttaggaagccg

SEQ ID NO: 96 - DNA, 67 bp, synthetic construct
  source 1..67: mol_type = other DNA
gtagcacttatcttcagttaggaagccgtgtttagttatcggttcctaactgaagatttagtactgc

SEQ ID NO: 97 - RNA, 21 nt, synthetic construct
  source 1..21: mol_type = other RNA
attcacaaacactctgtcctc

SEQ ID NO: 98 - DNA, 67 bp, synthetic construct
  source 1..67: mol_type = other DNA
gtagcacattcacaaacactctgtcctctgtttagttatgagacagagtgtttgtgatttgtactgc

SEQ ID NO: 99 - DNA, 847 bp, synthetic construct
  source 1..847: mol_type = other DNA
ctgtaaagaattcttaaggcataaatacgtgtctaaatggacctcatatctttgagataattaaactaattttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgttgatatgtgcatctcattatggtgacagctgcctcgggaagccaagttgggctttaaagtgcagggcctgctgatgttgagtgctttttgttctgaagtagattagcatctcataagaagttatgtattcatccaataattcaagccaagcaagtatataggtgttttaatagtttttgtttgcagtcctctgttagaagaatgtagttggtggcctgctatttccttcaaatgaatgatttttactaattttgtgtacttttattgtgtcgatgtagaatctgcctggtctatctgatgtgacagcttctgtagcactgtttagttatcttactgctagctgtagaactccagcttcggcctgtcgcccaatcaaactgtcctgttactgaacactgttctatggtttgtgtgatattctgcctgtggtagtgaaaagtctgtagaaaagtaagggaaactcaaacccctttctacacaggttgggatcggttgcaatgctgtgtttctgtatggtattgcacttgtcccggcctgttgagtttggtggggattgtgaccagaagattttgaaaattaaatattactgaagatttcgacttccactgttaaatgtacaagatac

SEQ ID NO: 100 - DNA, 1169 bp, synthetic construct
  source 1..1169: mol_type = other DNA
ctgtaaagaattcttaaggcataaatacgtgtctaaatggacctcatatctttgagataattaaactaattttttcttccccattagggattatgctgaatttgtatggtttatagttgttagagtttgaggtgttaattctaattatctatttcaaatttagcaggaaaaaagagaacatcaccttgtaaaactgaagattgtgaccagtcagaataatgttagcacaagctgcaggtgaaggtggcgtttgtttagttataacccaccttcacctgcagcaatgtactgccattatggtgacagctgcctcgggaagccaagttgggctttaaagtgcagggcctgctgatgttgagtgctttttgttctgttctttctattgtgcctgtcatcatgtgaagtagattagcatcatgatgtcagggtcaatagaatggcatcataagaagttatgtattcatccaataattcaagccaagcaagtatataggtgttttaatagtttttgtttgcagtcctctgttggtgggggttggaggcgtgggttttagaacctatccctttctagccctgagcatggtggcctgctatttccttcaaatgaatgatttttactaattttgtgtacttttattgtgtcgatgtagaatctgcctggtctatctgatgtgacagcttctgtagcacaagaaggtatattgctgttgacagtgagcgacggatgtacagctttatatatagtgaagcttcagatgtatatataaagctgtacatccgctgcctactgcctcggacttcaaggggtcagtcatactgctagctgtagaactccagcttcggcctgtcgcccaatcaaactgtcctgttactgaacactgttctatggttgcagtcctctgcaacgtctccacatgtataacgaagaatgtagtttatacatgatggagacgttgtggtggcctgcctgtggtagtgaaaagtctgtagaaaagtaagggaaactcaaacccctttctacacaggttgggatcggttgcaatgctgtgtttctgtatggtattgcacttgtcccggcctgttgagtttggtggggattgtgaccagaagattttgaaaattaaatattactgaagatttcgacttccactgttaaatgtacaagatac

SEQ ID NO: 101 - DNA, 122 bp, synthetic construct
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 102 - protein, 194 aa, Flavobacterium Okeanokoites
VKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 103 - protein, 194 aa, synthetic construct
VKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINF

SEQ ID NO: 104 - protein, 194 aa, synthetic construct
VKSELEEKKSELRHKLKYVPHEYIELIEIARNPTQDRILEMKVMEFFMKVYGYRGEHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINF

SEQ ID NO: 105 - DNA, 19 bp, synthetic construct
  source 1..19: mol_type = other DNA
tgggatgacgttacctcgt

SEQ ID NO: 106 - DNA, 18 bp, synthetic construct
  source 1..18: mol_type = other DNA
  misc_feature 1: note = HEX modification at 5'
  misc_feature 18: note = Iowa Black Quencher in the 3' end
  misc_feature 8: note = locked nucleic acid (LNA) nucleotide
  misc_feature 9: note = locked nucleic acid (LNA) nucleotide
ccagttccaaaccctggt

SEQ ID NO: 107 - DNA, 19 bp, synthetic construct
  source 1..19: mol_type = other DNA
cctctagctctggaagcac

SEQ ID NO: 108 - DNA, 18 bp, synthetic construct
  source 1..18: mol_type = other DNA
  misc_feature 1: note = HEX modification in the 5' end
  misc_feature 18: note = Iowa Black Quencher in the 3' end
  misc_feature 8: note = locked nucleic acid (LNA) nucleotide
  misc_feature 9: note = locked nucleic acid (LNA) nucleotide
agccggccagttccaaac

SEQ ID NO: 109 - DNA, 16 bp, synthetic construct
  source 1..16: mol_type = other DNA
  misc_feature 1: note = HEX modification in the 5' end
  misc_feature 16: note = Iowa Black Quencher in the 3' end
  misc_feature 8: note = locked nucleic acid (LNA) nucleotide
  misc_feature 9: note = locked nucleic acid (LNA) nucleotide
tcacccaggccagccg

SEQ ID NO: 110 - DNA, 18 bp, synthetic construct
  source 1..18: mol_type = other DNA
  misc_feature 1: note = FAM modification in the 5' end
  misc_feature 18: note = Iowa Black Quencher in the 3' end
  misc_feature 9..10: note = Internal ZEN modification between nucleotides
cctgacctttgtgccctt

SEQ ID NO: 111 - protein, 1719 aa, synthetic construct
MAVTPKKKRKVGGSGSEKLRNGSGDPGKKKQHACPECGKSFSQSSNLQKHQRTHTGEKPYKCPECGKSFSQSSNLQKHQRTHTGEKPYKCPECGKSFSRSDHLQRHQRTHQNKKSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGA

SEQ ID NO: 112 - protein, 1730 aa, synthetic construct
MAVTAPKKKRKVGGSGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGSETPGTSESATPESTKKKPLTQEQLEDARRLKAIYEKKKNELGLSQESVADKMGMGQSGVGALFNGINALNAYNAALLAKILKVSVEEFSPSIAREIYEMYEAVSKRPAATKKAGQAKKKKGA

SEQ ID NO: 113 - protein, 1735 aa, synthetic construct
MAVTPKKKRKVGGSGSAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGA

SEQ ID NO: 114 - protein, 900 aa, synthetic construct
MAATPKKKRKVGGSGSPDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQPWSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKI

SEQ ID NO: 115 - protein, 1647 aa, synthetic construct
MAVTPKKKRKVPTLLGFHTASGKKVKIAKESLDKVKNLFDEKEQVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDKRPAATKKAGQAKKKKGA

SEQ ID NO: 116 - protein, 1683 aa, synthetic construct
MAVTPKKKRKVSEKKQPVDLGLLEEDDEFEEFPAEDWAGLDEDEDAHVWEDNWDDDNVEDDFSNQLRAELEKHGYKMETSVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDKRPAATKKAGQAKKKKGA

SEQ ID NO: 117 - protein, 1761 aa, synthetic construct
MAVTPKKKRKVPTLLGFHTASGKKVKIAKESLDKVKNLFDEKEQVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKKRPAATKKAGQAKKKKGA

SEQ ID NO: 118 - protein, 1810 aa, synthetic construct
MAVTPKKKRKVSEKKQPVDLGLLEEDDEFEEFPAEDWAGLDEDEDAHVWEDNWDDDNVEDDFSNQLRAELEKHGYKMETSVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGSETPGTSESATPEAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKKRPAATKKAGQAKKKK

SEQ ID NO: 119 - protein, 1853 aa, synthetic construct
MAVTPKKKRKVPTLLGFHTASGKKVKIAKESLDKVKNLFDEKEQVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDPDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQPWKRPAATKKAGQAKKKKGA

SEQ ID NO: 120 - protein, 1902 aa, synthetic construct
MAVTPKKKRKVSEKKQPVDLGLLEEDDEFEEFPAEDWAGLDEDEDAHVWEDNWDDDNVEDDFSNQLRAELEKHGYKMETSVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGSETPGTSESATPEPDRAQVIIRIVPGGGTKTLQQIINQLEYLSRKGKLELQRSARHLDIPVPPDQIRELAQSWVTEAGIYDESQSDDDRQQDLTTHIIVSFPAGTDQTAAYEASREWAAEMFGSGYGGGRYNYLTAYHVDRDHPHLHVVVNRRELLGHGWLKISRRHPQLNYDGLRKKMAEISLRHGIVLDATSRAERGIAERPITYAEHRRLERMQPWKRPAATKKAGQAKKKK

SEQ ID NO: 121 - protein, 1611 aa, synthetic construct
MAVTPKKKRKNFFSLHPNVYATGRPKGLIGMLENVWVSNHTPGEGTLYLISGFSNYNGGVRFYETFTEHINQGGRVIAILGGSTSQRLSSRQVVEELLNRGVEVHIINRKRILHAKLYGTSNNLGESLVVSSGNFTGPGMSQNIEASLLLDNNTTQSMGFSWNDMISEMLNQNWHIHNMTNATDASPGWNLLSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 122 - protein, 1550 aa, synthetic construct
MAVTPKKKRKKTTRNQGIVQYVRAYFPDECACCKNQYNIKDRSFTYRHSDRYYLEVHHVISFASDRTLDQIDNLVKVCPTCHRALSKNRADEQYQKELISEILINAPKAKEFCLNFTDENDCIQFIYDRLRSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 123 - protein, 1581 aa, synthetic construct
MAVTPKKKRKALSWNEIRRKAIEFSKRWEDASDENSQAKPFLIDFFEVFGITNKRVATFEHAVKKFAKAHKEQSRGFVDLFWPGILLIEMKSRGKDLDKAYDQALDYFSGIAERDLPRYVLVCDFQRFRLTDLITKESVEFLLKDLYQNVRSFGFIAGYQTQSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 124 - protein, 141 aa, synthetic construct
RRSLAPSQLAKRKPEGRSCDDEDWQPGLVTPRKRKSSSETQIQECFLSPFRKPLSQLTNQPPCLDSSQHEAFIRSILSKPFKVPIPNYQGPLGSRALGLKRAGVRRALHDPLEKDALVLYEPPPLSAHDQLKLDKEKLPVH

SEQ ID NO: 125 - protein, 1763 aa, synthetic construct
MAVTPKKKRKVRRSLAPSQLAKRKPEGRSCDDEDWQPGLVTPRKRKSSSETQIQECFLSPFRKPLSQLTNQPPCLDSSQHEAFIRSILSKPFKVPIPNYQGPLGSRALGLKRAGVRRALHDPLEKDALVLYEPPPLSAHDQLKLDKEKLPVHGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 126 - protein, 1663 aa, synthetic construct
MAVTAPKKKRKVGGSGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDPTLLGFHTASGKKVKIAKESLDKVKNLFDEKEQKRPAATKKAGQAKKKKPKKKRKVGA

SEQ ID NO: 127 - protein, 1681 aa, synthetic construct
MAVTPKKKRKVSEKKQPVDLGLLEEDDEFEEFPAEDWAGLDEDEDAHVWEDNWDDDNVEDDFSNQLRAELEKHGYKMETSVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDKRPAATKKAGQAKKKK

SEQ ID NO: 128 - protein, 1717 aa, synthetic construct
MAVTPKKKRKVGGSGSEKLRNGSGDPGKKKQHACPECGKSFSQSSNLQKHQRTHTGEKPYKCPECGKSFSQSSNLQKHQRTHTGEKPYKCPECGKSFSRSDHLQRHQRTHQNKKSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGD

SEQ ID NO: 129 - protein, 1733 aa, synthetic construct
MAVTPKKKRKVGGSGSAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGD

SEQ ID NO: 130 - protein, 1651 aa, synthetic construct
MAVTPKKKRKVPTLLGFHTASGKKVKIAKESLDKVKNLFDEKEQVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 131 - protein, 1657 aa, synthetic construct
MAVTPKKKRKVPTLLGFHTASGKKVKIAKESLDKVKNLFDEKEQVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 132 - protein, 1755 aa, synthetic construct
MAVTPKKKRKVGGSGSAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 133 - protein, 1761 aa, synthetic construct
MAVTPKKKRKVGGSGSAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 134 - protein, 1783 aa, synthetic construct
MAVTPKKKRKVGGSGSSDSFSLLSQITPHQRCSFYAQVIKTWYSDKNFTLYVTDYTENELFFPMSPYTSSSRWRGPFGRFSIRCILWDEHDFYCRNYIKEGDYVVMKNVRTKIDHLGYLECILHGDSAKRYNMSIEKVDSEEPELNEIKSRKRLYVQNSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 135 - protein, 1789 aa, synthetic construct
MAVTPKKKRKVGGSGSSDSFSLLSQITPHQRCSFYAQVIKTWYSDKNFTLYVTDYTENELFFPMSPYTSSSRWRGPFGRFSIRCILWDEHDFYCRNYIKEGDYVVMKNVRTKIDHLGYLECILHGDSAKRYNMSIEKVDSEEPELNEIKSRKRLYVQNSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 136 - protein, 1766 aa, synthetic construct
MAVTPKKKRKVGGSGSAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDPTLLGFHTASGKKVKIAKESLDKVKNLFDEKEQ

SEQ ID NO: 137 - protein, 1794 aa, synthetic construct
MAVTPKKKRKVGGSGSSDSFSLLSQITPHQRCSFYAQVIKTWYSDKNFTLYVTDYTENELFFPMSPYTSSSRWRGPFGRFSIRCILWDEHDFYCRNYIKEGDYVVMKNVRTKIDHLGYLECILHGDSAKRYNMSIEKVDSEEPELNEIKSRKRLYVQNSGSETPGTSESATPEQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPESGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQFPEKYAEIFKDDTKNGYAGYVGIGIKHRKRTTKLATQEEFYKFIKPILEKMDGAEELLAKLNRDDLLRKQRTFDNGSIPHQIHLKELHAILRRQEEFYPFLKENREKIEKILTFRIPYYVGPLARGNSRFAWLTRKSEEAITPWNFEEVVDKGASAQSFIERMTNFDEQLPNKKVLPKHSLLYEYFTVYNELTKVKYVTERMRKPEFLSGEQKKAIVDLLFKTNRKVTVKQLKEDYFKKIECFDSVEIIGVEDRFNASLGTYHDLLKIIKDKDFLDNEENEDILEDIVLTLTLFEDREMIEERLKTYAHLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDPTLLGFHTASGKKVKIAKESLDKVKNLFDEKEQ

SEQ ID NO: 138 - protein, 1662 aa, synthetic construct
MAVTPKKKRKVPTLLGFHTASGKKVKIAKESLDKVKNLFDEKEQVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDPTLLGFHTASGKKVKIAKESLDKVKNLFDEKEQ

SEQ ID NO: 139 - protein, 1651 aa, synthetic construct
MAVTPKKKRKVPTLLGFHTASGKKVKIAKESLDKVKNLFDEKEQVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 140 - protein, 1657 aa, synthetic construct
MAVTPKKKRKVPTLLGFHTASGKKVKIAKESLDKVKNLFDEKEQVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFSGSETPGTSESATPEGTEKKYSIGLAIGTNSVGWAVITDDYKVPSKKFKVLGNTNRKSIKKNLMGALLFDSGETAEATRLKRTARRRYTRRKNRIRYLQEIFANEMAKLDDSFFQRLEESFLVEEDKKNERHPIFGNLADEVAYHRNYPTIYHLRKKLADSPEKADLRLIYLALAHIIKFRGHFLIEGKLNAENSDVAKLFYQLIQTYNQLFEESPLDEIEVDAKGILSARLSKSKRLEKLIAVFPNEKKNGLFGNIIALALGLTPNFKSNFDLTEDAKLQLSKDTYDDDLDELLGQIGDQYADLFSAAKNLSDAILLSDILRSNSEVTKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 141 - protein, 1662 aa, synthetic construct
MAVTPKKKRKVPPAPPVTHSTPVTVSEPLLEKDFLAGVTQELIKTLEDNSEGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 142 - protein, 1655 aa, synthetic construct
MAVTPKKKRKVPTLLGFHTASGKKVKIAKESLDKVKNLFDEKEQGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 143 - protein, 1647 aa, synthetic construct
MAVTPKKKRKVFHTASGKKVKIAKESLDKRKNLFDEGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 144 - protein, 1662 aa, synthetic construct; identical to SEQ ID NO: 141

SEQ ID NO: 145 - protein, 1736 aa, synthetic construct
MAVTPKKKRKVAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKV

SEQ ID NO: 146 - protein, 1662 aa, synthetic construct
MAVTPKKKRKVGGSGSQLVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGPTLLGFHTASGKKVKIAKESLDKVKNLFDEKEQSGGSKRTADGSEFESPKKKRKV

SEQ ID NO: 147 - protein, 1698 aa, synthetic construct
MAVTPKKKRKVGGSGSQLVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGSEKKQPVDLGLLEEDDEFEEFPAEDWAGLDEDEDAHVWEDNWDDDNVEDDFSNQLRAELEKHGYKMETSSGGSKRTADGSEFESPKKKRKV

SEQ ID NO: 148 - protein, 1654 aa, synthetic construct
MAVTPKKKRKVGGSGSQLVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGFHTASGKKVKIAKESLDKRKNLFDESGGSKRTADGSEFESPKKKRKV

SEQ ID NO: 149 - protein, 1770 aa, synthetic construct
MAVTPKKKRKVGGSGSQLVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGRRSLAPSQLAKRKPEGRSCDDEDWQPGLVTPRKRKSSSETQIQECFLSPFRKPLSQLTNQPPCLDSSQHEAFIRSILSKPFKVPIPNYQGPLGSRALGLKRAGVRRALHDPLEKDALVLYEPPPLSAHDQLKLDKEKLPVHSGGSKRTADGSEFESPKKKRKV

SEQ ID NO: 150 - protein, 1669 aa, synthetic construct
MAVTPKKKRKVGGSGSQLVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGPPAPPVTHSTPVTVSEPLLEKDFLAGVTQELIKTLEDNSESGGSKRTADGSEFESPKKKRKV

SEQ ID NO: 151 - protein, 1759 aa, synthetic construct
MAVTPKKKRKVGGSGSQLVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVSGGSKRTADGSEFESPKKKRKV

SEQ ID NO: 152 - protein, 1743 aa, synthetic construct
MAVTPKKKRKVGGSGSQLVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKSGGSKRTADGSEFESPKKKRKV

SEQ ID NO: 153 - protein, 1661 aa, synthetic construct
MAVTPKKKRKVPTLLGFHTASGKKVKIAKESLDKVKNLFDEKEQGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 154 - protein, 1697 aa, synthetic construct
MAVTPKKKRKVSEKKQPVDLGLLEEDDEFEEFPAEDWAGLDEDEDAHVWEDNWDDDNVEDDFSNQLRAELEKHGYKMETSGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 155 - protein, 1653 aa, synthetic construct
MAVTPKKKRKVFHTASGKKVKIAKESLDKRKNLFDEGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 156 - protein, 1769 aa, synthetic construct
MAVTPKKKRKVRRSLAPSQLAKRKPEGRSCDDEDWQPGLVTPRKRKSSSETQIQECFLSPFRKPLSQLTNQPPCLDSSQHEAFIRSILSKPFKVPIPNYQGPLGSRALGLKRAGVRRALHDPLEKDALVLYEPPPLSAHDQLKLDKEKLPVHGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 157 - protein, 1668 aa, synthetic construct
MAVTPKKKRKVPPAPPVTHSTPVTVSEPLLEKDFLAGVTQELIKTLEDNSEGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 158 - protein, 1742 aa, synthetic construct
MAVTPKKKRKVAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 159 - protein, 1692 aa, synthetic construct
MAVTPKKKRKVPTLLGFHTASGKKVKIAKESLDKVKNLFDEKEQGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGPTLLGFHTASGKKVKIAKESLDKVKNLFDEKEQSGGSKRTADGSEFESPKKKRKV

SEQ ID NO: 160 - protein, 1728 aa, synthetic construct
MAVTPKKKRKVPTLLGFHTASGKKVKIAKESLDKVKNLFDEKEQGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGSEKKQPVDLGLLEEDDEFEEFPAEDWAGLDEDEDAHVWEDNWDDDNVEDDFSNQLRAELEKHGYKMETSSGGSKRTADGSEFESPKKKRKV

SEQ ID NO: 161 - protein, 1684 aa, synthetic construct
MAVTPKKKRKVPTLLGFHTASGKKVKIAKESLDKVKNLFDEKEQGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGFHTASGKKVKIAKESLDKRKNLFDESGGSKRTADGSEFESPKKKRKV

SEQ ID NO: 162 - protein, 1699 aa, synthetic construct
MAVTPKKKRKVPTLLGFHTASGKKVKIAKESLDKVKNLFDEKEQGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGPPAPPVTHSTPVTVSEPLLEKDFLAGVTQELIKTLEDNSESGGSKRTADGSEFESPKKKRKV

SEQ ID NO: 163 - protein, 1789 aa, synthetic construct
MAVTPKKKRKVPTLLGFHTASGKKVKIAKESLDKVKNLFDEKEQGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVSGGSKRTADGSEFESPKKKRKV

SEQ ID NO: 164 - protein, 1773 aa, synthetic construct
MAVTPKKKRKVPTLLGFHTASGKKVKIAKESLDKVKNLFDEKEQGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKSGGSKRTADGSEFESPKKKRKV

SEQ ID NO: 165 - protein, 1728 aa, synthetic construct
MAVTPKKKRKVSEKKQPVDLGLLEEDDEFEEFPAEDWAGLDEDEDAHVWEDNWDDDNVEDDFSNQLRAELEKHGYKMETSGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGPTLLGFHTASGKKVKIAKESLDKVKNLFDEKEQSGGSKRTADGSEFESPKKKRKV

SEQ ID NO: 166 - protein, 1720 aa, synthetic construct
MAVTPKKKRKVSEKKQPVDLGLLEEDDEFEEFPAEDWAGLDEDEDAHVWEDNWDDDNVEDDFSNQLRAELEKHGYKMETSGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGFHTASGKKVKIAKESLDKRKNLFDESGGSKRTADGSEFESPKKKRKV

SEQ ID NO: 167 - protein, 1836 aa, synthetic construct
MAVTPKKKRKVSEKKQPVDLGLLEEDDEFEEFPAEDWAGLDEDEDAHVWEDNWDDDNVEDDFSNQLRAELEKHGYKMETSGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGRRSLAPSQLAKRKPEGRSCDDEDWQPGLVTPRKRKSSSETQIQECFLSPFRKPLSQLTNQPPCLDSSQHEAFIRSILSKPFKVPIPNYQGPLGSRALGLKRAGVRRALHDPLEKDALVLYEPPPLSAHDQLKLDKEKLPVHSGGSKRTADGSEFESPKKKRKV

SEQ ID NO: 168 - protein, 1809 aa, synthetic construct
MAVTPKKKRKVSEKKQPVDLGLLEEDDEFEEFPAEDWAGLDEDEDAHVWEDNWDDDNVEDDFSNQLRAELEKHGYKMETSGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKSGGSKRTADGSEFESPKKKRKV

SEQ ID NO: 169 - protein, 1773 aa, synthetic construct
MAVTPKKKRKVAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGPTLLGFHTASGKKVKIAKESLDKVKNLFDEKEQSGGSKRTADGSEFESPKKKRKV

SEQ ID NO: 170 - protein, 1809 aa, synthetic construct
MAVTPKKKRKVAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGSEKKQPVDLGLLEEDDEFEEFPAEDWAGLDEDEDAHVWEDNWDDDNVEDDFSNQLRAELEKHGYKMETSSGGSKRTADGSEFESPKKKRKV

SEQ ID NO: 171 - protein, 1765 aa, synthetic construct
MAVTPKKKRKVAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGFHTASGKKVKIAKESLDKRKNLFDESGGSKRTADGSEFESPKKKRKV

SEQ ID NO: 172 - protein, 1780 aa, synthetic construct
MAVTPKKKRKVAEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVKGGSGVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDGGSGPPAPPVTHSTPVTVSEPLLEKDFLAGVTQELIKTLEDNSESGGSKRTADGSEFESPKKKRKV

SEQ ID NO: 173 - protein, 1631 aa, synthetic construct
MAVTPKKKRKVGGSGSQLVKSELEEKKSELRHKLKYLPHEYLELIDIARDPTQNRIFEMKTMELFMKVYGFRGEHLGGSRKPDGAIYTVGLPIDYGVIVDTKAYSGGYNLPIGQADEMQRYIEENQTRNKHINPNEWWKVFPSSITEFKFLFVSGHFKGNFKAQLTRLNHITNCNGAALNVEELLIGAEMIKAGTLTLEEVRRKFNNGEINFSGSETPGTSESATPESMDKKYSIGLAIGTNSVGWAVITDDYKVPSKKMKVLGNTDRQSIKKNLIGALLFDSGETAEATRLKRTARRRYTRRKNRICYLQEIFANEMAKLDDSFFHRLEESFLVEEDKKHERHPIFGNLVDEVAYHENYPTIYHLRKKLADSTEKADLRLIYLALAHMIKFRGHFLIEGDLNAENSDVDKLFIQLVQTYNQLFEENPINESGVDAKAILSARLSKSRRLENLIAQFPGEKKNGLFGNIIALALGLTPNFKSNFDLTEDAKLQLSKDTYDDDLDELLGQIGDQYADLFLAAKNLSDAILLSDILRVNSEITKAPLSASMVKRYDEHHQDLALLKTLVRQQLPEKYKEIFSDQSKNGYAGYIDIGIKHRKRTTKRTSQEEFYKFIKPILEKMDGAEELLAKLNREDLLRKQRTFDNGSIPHQIHLGELHAILRRQEEFYPFLKDNREKIEKILTFRIPYYVGPLARGNSRFAWMTRKSDEAITPWNFEEVVDKGASAQSFIERMTNFDEYLPNKKVLPKHSLLYEYFTVYNELTKVKYVTEGMRKPEFLSGEQKEAIVDLLFKTNRKVTVKQLKEDYFKKIECFDSVEIIGVEDRFNASLGTYHDLLKIIKDKDFLDNEENEDILEDIVLTLTLFEDREMIEERLKKYANLFDDKVMKQLKRRHYTGWGRLSRKMINGIRDKQSGKTILDFLKSDGFSNRNFMQLIHDDSLTFKEEIEKAQVSGQGDSLHEQIADLAGSPAIKKGILQTVKIVDELVKVMGHKPENIVIEMARENQTTTKGLQQSRERKKRIEEGIKELESQILKENPVENTQLQNEKLYLYYLQNGRDMYVDQELDINRLSDYDVDAIVPQSFIKDDSIDNKVLTRSVENRGKSDNVPSEEVVKKMKNYWRQLLNAKLITQRKFDNLTKAERGGLSEADKAGFIKRQLVETRQITKHVARILDSRMNTKRDKNDKPIREVKVITLKSKLVSDFRKDFQLYKVRDINNYHHAHDAYLNAVVGTALIKKYPKLESEFVYGDYKVYDVRKMIAKSEQEIGKATAKRFFYSNIMNFFKTEVKLANGEIRKRPLIETNGETGEVVWNKEKDFATVRKVLAMPQVNIVKKTEVQTGGFSKESILSKRESAKLIPRKKGWDTRKYGGFGSPTVAYSILVVAKVEKGKAKKLKSVKVLVGITIMEKGSYEKDPIGFLEAKGYKDIKKELIFKLPKYSLFELENGRRRMLASATELQKANELVLPQHLVRLLYYTQNISATTGSNNLGYIEQHREEFKEIFEKIIDFSEKYILKNKVNSNLKSSFDEQFAVSDSILLSNSFVSLLKYTSFGASGGFTFLDLDVKQGRLRYQTVTEVLDATLIYQSITGLYETRTDLSQLGGDSGGSKRTADGSEFESPKKKRKVHHHHHH

SEQ ID NO: 174 - protein, 1368 aa, synthetic construct [sequence omitted]

SEQ ID NO: 175 - protein, 114 aa, synthetic construct
AEAGITGTWYNQSGSTFTVTAGADGNLTGQYENRAQGTGCQNSPYTLTGRYNGTKLEWRVEWNNSTENCHSRTEWRGQYQGGAEARINTQWNLTYEGGSGPATEQGQDTFTKVK

SEQ ID NO: 176 - DNA, 501 bp, Homo sapiens
  source 1..501: mol_type = genomic DNA
  CDS 1..501: protein_id = 177; translation = MPAMPSSGPGDTSSSAAEREEDRKDGEEQEEPRGKEERQEPSTT ARKVGRPGRKRKHPPVESGDTPKDPAVISKSPSMAQDSGASELLPNGDLEKRSEPQPE EGSPAGGQKGGAPAEGEGAAETLPEASRAVENGCCTPKEGRGAPAEAGESSAPGAASS GPTSIP
atgcccgccatgccctccagcggccccggggacaccagcagctctgctgcggagcgggaggaggaccgaaaggacggagaggagcaggaggagccgcgtggcaaggaggagcgccaagagcccagcaccacggcacggaaggtggggcggcctgggaggaagcgcaagcaccccccggtggaaagcggtgacacgccaaaggaccctgcggtgatctccaagtccccatccatggcccaggactcaggcgcctcagagctattacccaatggggacttggagaagcggagtgagccccagccagaggaggggagccctgctggggggcagaagggcggggccccagcagagggagagggtgcagctgagaccctgcctgaagcctcaagagcagtggaaaatggctgctgcacccccaaggagggccgaggagcccctgcagaagcgggtgagtcctcagcaccaggggcagcctcttctgggcccaccagcataccctga

SEQ ID NO: 177 - protein, 166 aa, Homo sapiens
MPAMPSSGPGDTSSSAAEREEDRKDGEEQEEPRGKEERQEPSTTARKVGRPGRKRKHPPVESGDTPKDPAVISKSPSMAQDSGASELLPNGDLEKRSEPQPEEGSPAGGQKGGAPAEGEGAAETLPEASRAVENGCCTPKEGRGAPAEAGESSAPGAASSGPTSIP

SEQ ID NO: 178 - RNA, 20 nt, synthetic construct
  source 1..20: mol_type = other RNA
gtctgggcggtgctacaact

SEQ ID NO: 179 - RNA, 20 nt, synthetic construct
  source 1..20: mol_type = other RNA
ctgtgggatctgcatgcctg

SEQ ID NO: 180 - RNA, 20 nt, synthetic construct
  source 1..20: mol_type = other RNA
ggcgccctggccagtcgtct

SEQ ID NO: 181 - DNA, 1053 bp, synthetic construct
  source 1..1053: mol_type = other DNA
aaaggtctcactgggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttactggtgagaccttt

SEQ ID NO: 182 - DNA, 1905 bp, synthetic construct
  source 1..1905: mol_type = other DNA
gcttaactatgcggcatcagacacccggcagcctcaggaggggcagctcgggcgggatatggaaagaggccacagcagtgagcagagacacagaggaggaaggggccctgagctggggagacccccacggggtagggcgtgggggccacgggcccacctcctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatgcagatcccacaggcgcccactagtgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttacagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacatggtcatagctgtttcctg

SEQ ID NO: 183 - DNA, 9011 bp, Homo sapiens
  source 1..9011: mol_type = genomic DNA
gctcacctccgcctgagcagtggagaaggcggcactctggtggggctgctccaggcatgcagatcccacaggcgccctggccagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcaggctggccagaaccccaaaccttggccgtggggagtctgcgtggcggctctgccttgcccaggcatccttggtcctcactcgagttttcctaaggatgggatgagccccatgtgggactaaccttggctttacgacgtcaaagtttagatgagctggtgatatttttctcattatatccaaagtgtacctgttcgagtgaggacagttcttctgtctccaggatccctcctgggtggggattgtgcccgcctgggtctctgcccagattccagggctctccccgagccctgttcagaccatccgtgggggaggccttggcctcactctcccggatcgaggagagagggagcctcttcctgggctgcccgtgaccctgggccctctgtgtacactgtgaccacagcccgctcctggaccctctgtgcccggctggccctctgtgcccagccagcctgcacctggggatgccaaggcctggggagggtggtttcacccaggccaagcctaagacagtccctctgggccctgctgggtaccggggtgtgacaccactgggaggacaagatgaggggcacccctggggccgccctgacaccccctcgaggctcctgccccgggggtcctggtgccccttcactgtggcaggcgactgggggttccccacctcggcccctctcccggggcctgctccccggcacctgaggcagcatccttgtcagggccgtgccttcctgcctcagcgccacctcttaaggttggcccgtgggtcactcaggactcagaactggagattctgggcaaaaggcaaagagcaaagggccaaaaggcatcccagggagacgactgcgggggaaccagagggcagaggggcgctcgtcacaggggagggggagctgagcgaggcaggaggggagccgagcctctccccccgtgtcccggctcttcaggcacgccctcgggacgccaccctccccgacccaggcgggaaagataagagcaaggtgtccgcagcctgacactcgtgcctcaggtgcccgcgcttgtgccggacaagactctcacaggtggcatgcctcggtttccccactggtaacagcacagggcactcagcaaggcgcagtgggcatgactggggtcctgtgggtcctgacccagatgtggccaccccggccgcagtggtcttcattccaggatgcctcttttccctcctgatctattcactgcgttcgccattcggtcattcccggggccaccactcccacctcaggtgtgtgcttcccttgtgttttatgagatatccccaacccggctgcttattggccccgtccgagggcaggagcataaataagagcctctgctttggcgtgggaccactgtgagctccagtcagcgctgccactgctgagctctgggccttcgacaggacttggccccttactgacttctccgtgtgctttgggtcatgggtgaggacgcctcctggcaaggctgcgtcctgaggattaaatcgggtcatctgtgaaaactacccagcccagcccctgacacttttttgtttgtttcttttagtgacagggtcttgctctgtcacccaggctggagtgcagtggtgtgatctcggctcactcgacctccggggctcaagcaattctcccacctctgcctccagagtagctgggactataggcacgtgccaccctgccaggctaatttcttccattttttttagagacagggtctcgctatgttccccaggctgggctcaaatggtcctcccacctcagcctccccaagtactgggattacaggcataagccactgcatctggcctccatgacacatatttttaaagtctgatttttaaagtcaaacttttgaagtcagattttaaacggactattttgaaaaatatacaaaaacgtttaaaaacaatgaatatccctcacctagaatcaataactaagaatattgacacatttgctttggggactgggcggctggagctgccatgacaaagctccgccgaccgagtggcttttaaacagagcctgccctctcgccgactgagggctggacgtgcaggatggagctccgcagggtcggctcccctgtgctctgaggggctctgctcagcctctcccggctgtggcttaaaaacagagcctgtcctcccgccgtggggggctggacatgcaggaccgaggggccacagggtcggctccctgtgctccgagagggctctgctcagcttctcctggctggggggttttgtggccaccctctgtgttcctgggttcagaagcatcccccaggctctgccttcatctctgcacgggtgactctgtacaggaagccaggcctgctggtcaatggccacccagccctgtgccctcatcttacctagtcccagctgccgtcaccctattcctaataaggccgccttctgaggtcatggggttaggacttccacataggaatctgtggggacacggttcggcccacagcccttcccacctccacacacacacacgactgtgaggagttggaagacctcactcctcacccctgccaggtcctctagggacaagctcgctgtcctcatcccagcacagcccgtgggacggtttccttgtccctaatgggaccacggtcagagatgccgggtctggtctgggccagcaggttcctccgcccggggcaggcagccttcttctgtgcgcttctggaaagcaatgtcctgtaatgcggtctctctgcgggagcacccccaccgccacctcacaggcctgttccacagccccgggatgggctctgtctccctcctgaccctgcatagggcacagccctctctcatcaacccacgatcctacgtggatccgagagggagcacctggggaaacaatggaatcccatagaaacaccccaaatctaacttgatccaggaccagccagtggtcacttctgaatattcaccttcctagtagacactaccagccaagggaggccaggaagccttcctggaggaggtggcctgaggactggggtgaggcaggccctgcgtgggggtcgccacccagcacccccacactgggtgggagccagtctctgagactggctgggggaggtgggagagggggctgcttgaactgcagacaccgaggtctagcccccaccccacccagccagttggtggaggcaggggaggccgaggggcccagctggacctgctccccggggtggattccaaaataggggggttggggggggcggaacaggagcccagggtcctggcttgaggcccagtggctgagggctggtgcaagccagacaggaaaagggttgagcctgtcagcgccagcacagatcaagtcaggagcaggtccctccaccaatgtgtgcaaataaatagcagctaagtttccagttacaagaacaatgcacagatggtcccagggacattgcggtgtggacacacagcggccattgtcctgtcgccagcacctcgccctacagctggggggtcccttagcacttcctagccatgcagggtccctgctcacagtacccgtgatgacttctgttcctcacctgcctgtctgtcccgacagctgcatggcagccctggcctgggagatggagaccccgaggggctgcctgcggtggtggggcccctgggtccccactgcattcccagaaacccagagggcagggcatttcccctgctctgtgccgagtccacccagccccagcctaggcccagtaagggctgcagcccaccctgtcccaggctgcctcccaggagccctcttggccctgatgccagaagcccatcttcctccattcaggcaggtctctgagtgccctggcctggctgcctgctggccctgagagtcacactaccccacagccctccttggtcaaaatccactctggagtggctggaagattccccgggcccacgccgcacacgcctatgcagggagcttcccctggccggccggcagacaagggcggtctcagagagggggctcacctcagcagccccttgtgtagctggccctcgcccctgccacctctgggaacaccaccaggaagctgggggacaggcacgcaggtgaaggaggcgagcgcttgtcagccgggaggccatgggcacagagggaacagggacaccctgggtggcctcaaggtcacttcaaacccctcactcgtcccctgggagggtgcccagtgaggttggcactaggagttggtcctggtcacatgacagacccacccacctctggtgtccagccagcacgccgtgggccagcctggctgcagggacacgagggcagcagccccctcctcctctgagctggttgctccttgagtcatcaccaccgcctgccacggaggccgcctgtcccaggaagcagagggaccgcagctgtggcaaccagggcctggtctctgtgtcacctcgctggggggccgtgcccaggcctgagacggaactgagtgacagtgcactgggtctgacagtgtggggctggcgccatgtttggggaaccctgtggcatgggacctgtgggtgagccgggaaaatcaccccgttgcatggcatctcgggcctggatcttaagcgcctgtgttggtgcctccgcctggcggaagagccgcgacccccacgttgccatgcgggtatcccaagccctgaccctggcaggcatatgtttcaggaggtccttgtcttgggagcccagggtcgggggccccgtgtctgtccacatccgagtcaatggcccatctcgtctctgaagcatctttgctgtgagctctagtccccactgtcttgctggaaaatgtggaggccccactgcccactgcccagggcagcaatgcccataccacgtggtcccagctccgagcttgtcctgaaaagggggcaaagactggaccctgagcctgccaaggggccacactcctcccagggctggggtctccatgggcagccccccacccacccagaccagttacactcccctgtgccagagcagtgcagacaggaccaggccaggatgcccaagggtcaggggctggggatgggtagcccccaaacagccctttctgggggaactggcctcaacggggaagggggtgaaggctcttagtaggaaatcagggagacccaagtcagagccaggtgctgtgcagaagctgcagcctcacgtagaaggaagaggctctgcagtggaggccagtgcccatccccgggtggcagaggccccagcagagacttctcaatgacattccagctggggtggcccttccagagcccttgctgcccgagggatgtgagcaggtggccggggaggctttgtggggccacccagccccttcctcacctctctccatctctcagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacaggtgcggcctcggaggccccggggcaggggtgagctgagccggtcctggggtgggtgtcccctcctgcacaggatcaggagctccagggtcgtagggcagggaccccccagctccagtccagggctctgtcctgcacctggggaatggtgaccggcatctctgtcctctagctctggaagcaccccagcccctctagtctgccctcacccctgaccctgaccctccaccctgaccccgtcctaacccctgacctttgtgcccttccagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgaggtaacgtcatcccagcccctcggcctgccctgccctaaccctgctggcggccctcactcccgcctccccttcctccacccttccctcaccccaccccacctccccccatctccccgccaggctaagtccctgatgaaggcccctggactaagaccccccacctaggagcacggctcagggtcggcctggtgaccccaagtgtgtttctctgcagggacaataggagccaggcgcaccggccagcccctggtgagtctcactcttttcctgcatgatccactgtgccttccttcctgggtgggcagaggtggaaggacaggctgggaccacacggcctgcaggactcacattctattatagccaggaccccacctccccagcccccaggcagcaacctcaatccctaaagccatgatctggggccccagcccacctgcggtctccgggggtgcccggcccatgtgtgtgcctgcctgcggtctccaggggtgcctggcccacgcgtgtgcccgcctgcggtctctgggggtgcccggcccacatatgtgcctgcctgcggtctccaggtgtgcccggcccatgcgtgtgcccacctgcgagggcgtggggtgggcttggtcatttcttatcttacattggagacaggagagcttgaaaagtcacattttggaatcctaaatctgcaagaatgccagggacatttcagagggggacattgagccagagaggaggggtggtgtccccagatcacacagagggcagtggtgggacagctcagggtaagcagctcatagtggggggcccaggttcggtgccggtactgcagccaggctgtggagccgcgggcctccttcctgcggtgggccgtggggctgactccctctccctttctcctcaaagaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaa

SEQ ID NO: 184 - DNA, 20 bp, synthetic construct
  source 1..20: mol_type = other DNA
agcctgacccgtcattctac

SEQ ID NO: 185 - DNA, 20 bp, synthetic construct
  source 1..20: mol_type = other DNA
gctgaacttgtggccgttta

SEQ ID NO: 186 - DNA, 1029 bp, Homo sapiens
  source 1..1029: mol_type = other DNA
  CDS 1..1029: protein_id = 187; translation = MAEHDYHEDYGFSSFNDSSQEEHQDFLQFSKVFLPCMYLVVFVC GLVGNSLVLVISIFYHKLQSLTDVFLVNLPLADLVFVCTLPFWAYAGIHEWVFGQVMC KSLLGIYTINFYTSMLILTCITVDRFIVVVKATKAYNQQAKRMTWGKVTSLLIWVISL LVSLPQIIYGNVFNLDKLICGYHDEAISTVVLATQMTLGFFLPLLTMIVCYSVIIKTL LHAGGFQKHRSLKIIFLVMAVFLLTQMPFNLMKFIRSTHWEYYAMTSFHYTIMVTEAI AYLRACLNPVLYAFVSLKFRKNFWKLVKDIGCLPYLGVSHQWKSSEDNSKTFSASHNV EATSMFQL
atggcagagcatgattaccatgaagactatgggttcagcagtttcaatgacagcagccaggaggagcatcaagacttcctgcagttcagcaaggtctttctgccctgcatgtacctggtggtgtttgtctgtggtctggtggggaactctctggtgctggtcatatccatcttctaccataagttgcagagcctgacggatgtgttcctggtgaacctacccctggctgacctggtgtttgtctgcactctgcccttctgggcctatgcaggcatccatgaatgggtgtttggccaggtcatgtgcaagagcctactgggcatctacactattaacttctacacgtccatgctcatcctcacctgcatcactgtggatcgtttcattgtagtggttaaggccaccaaggcctacaaccagcaagccaagaggatgacctggggcaaggtcaccagcttgctcatctgggtgatatccctgctggtttccttgccccaaattatctatggcaatgtctttaatctcgacaagctcatatgtggttaccatgacgaggcaatttccactgtggttcttgccacccagatgacactggggttcttcttgccactgctcaccatgattgtctgctattcagtcataatcaaaacactgcttcatgctggaggcttccagaagcacagatctctaaagatcatcttcctggtgatggctgtgttcctgctgacccagatgcccttcaacctcatgaagttcatccgcagcacacactgggaatactatgccatgaccagctttcactacaccatcatggtgacagaggccatcgcatacctgagggcctgccttaaccctgtgctctatgcctttgtcagcctgaagtttcgaaagaacttctggaaacttgtgaaggacattggttgcctcccttaccttggggtctcacatcaatggaaatcttctgaggacaattccaagactttttctgcctcccacaatgtggaggccaccagcatgttccagttatag

SEQ ID NO: 187 - protein, 342 aa, Homo sapiens
MAEHDYHEDYGFSSFNDSSQEEHQDFLQFSKVFLPCMYLVVFVCGLVGNSLVLVISIFYHKLQSLTDVFLVNLPLADLVFVCTLPFWAYAGIHEWVFGQVMCKSLLGIYTINFYTSMLILTCITVDRFIVVVKATKAYNQQAKRMTWGKVTSLLIWVISLLVSLPQIIYGNVFNLDKLICGYHDEAISTVVLATQMTLGFFLPLLTMIVCYSVIIKTLLHAGGFQKHRSLKIIFLVMAVFLLTQMPFNLMKFIRSTHWEYYAMTSFHYTIMVTEAIAYLRACLNPVLYAFVSLKFRKNFWKLVKDIGCLPYLGVSHQWKSSEDNSKTFSASHNVEATSMFQL

SEQ ID NO: 188 - DNA, 906 bp, Homo sapiens
  source 1..906: mol_type = other DNA
  CDS 1..906: protein_id = 189; translation = MFSHLPFDCVLLLLLLLLTRSSEVEYRAEVGQNAYLPCFYTPAA PGNLVPVCWGKGACPVFECGNVVLRTDERDVNYWTSRYWLNGDFRKGDVSLTIENVTL ADSGIYCCRIQIPGIMNDEKFNLKLVIKPAKVTPAPTRQRDFTAAFPRMLTTRGHGPA ETQTLGSLPDINLTQISTLANELRDSRLANDLRDSGATIRIGIYIGAGICAGLALALI FGALIFKWYSHSKEKIQNLSLISLANLPPSGLANAVAEGIRSEENIYTIEENVYEVEE PNEYYCYVSSRQQPSQPLGCRFAMP
atgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactcggcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcatctacatcggagcagggatctgtgctgggctggctctggctcttatcttcggcgctttaattttcaaatggtattctcatagcaaagagaagatacagaatttaagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgccatag

SEQ ID NO: 189 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 190 - DNA, 1578 bp, Homo sapiens
  source 1..1578: mol_type = other DNA
  CDS 1..1578: protein_id = 191; translation = MWEAQFLGLLFLQPLWVAPVKPLQPGAEVPVVWAQEGAPAQLPC SPTIPLQDLSLLRRAGVTWQHQPDSGPPAAAPGHPLAPGPHPAAPSSWGPRPRRYTVL SVGPGGLRSGRLPLQPRVQLDERGRQRGDFSLWLRPARRADAGEYRAAVHLRDRALSC RLRLRLGQASMTASPPGSLRASDWVILNCSFSRPDRPASVHWFRNRGQGRVPVRESPH HHLAESFLFLPQVSPMDSGPWGCILTYRDGFNVSIMYNLTVLGLEPPTPLTVYAGAGS RVGLPCRLPAGVGTRSFLTAKWTPPGGGPDLLVTGDNGDFTLRLEDVSQAQAGTYTCH IHLQEQQLNATVTLAIITVTPKSFGSPGSLGKLLCEVTPVSGQERFVWSSLDTPSQRS FSGPWLEAQEAQLLSQPWQCQLYQGERLLGAAVYFTELSSPGAQRSGRAPGALPAGHL LLFLILGVLSLLLLVTGAFGFHLWRRQWRPRRFSALEQGIHPPQAQSKIEELEQEPEP EPEPEPEPEPEPEPEQL
atgtgggaggctcagttcctgggcttgctgtttctgcagccgctttgggtggctccagtgaagcctctccagccaggggctgaggtcccggtggtgtgggcccaggagggggctcctgcccagctcccctgcagccccacaatccccctccaggatctcagccttctgcgaagagcaggggtcacttggcagcatcagccagacagtggcccgcccgctgccgcccccggccatcccctggcccccggccctcacccggcggcgccctcctcctgggggcccaggccccgccgctacacggtgctgagcgtgggtcccggaggcctgcgcagcgggaggctgcccctgcagccccgcgtccagctggatgagcgcggccggcagcgcggggacttctcgctatggctgcgcccagcccggcgcgcggacgccggcgagtaccgcgccgcggtgcacctcagggaccgcgccctctcctgccgcctccgtctgcgcctgggccaggcctcgatgactgccagccccccaggatctctcagagcctccgactgggtcattttgaactgctccttcagccgccctgaccgcccagcctctgtgcattggttccggaaccggggccagggccgagtccctgtccgggagtccccccatcaccacttagcggaaagcttcctcttcctgccccaagtcagccccatggactctgggccctggggctgcatcctcacctacagagatggcttcaacgtctccatcatgtataacctcactgttctgggtctggagcccccaactcccttgacagtgtacgctggagcaggttccagggtggggctgccctgccgcctgcctgctggtgtggggacccggtctttcctcactgccaagtggactcctcctgggggaggccctgacctcctggtgactggagacaatggcgactttacccttcgactagaggatgtgagccaggcccaggctgggacctacacctgccatatccatctgcaggaacagcagctcaatgccactgtcacattggcaatcatcacagtgactcccaaatcctttgggtcacctggatccctggggaagctgctttgtgaggtgactccagtatctggacaagaacgctttgtgtggagctctctggacaccccatcccagaggagtttctcaggaccttggctggaggcacaggaggcccagctcctttcccagccttggcaatgccagctgtaccagggggagaggcttcttggagcagcagtgtacttcacagagctgtctagcccaggtgcccaacgctctgggagagccccaggtgccctcccagcaggccacctcctgctgtttctcatccttggtgtcctttctctgctccttttggtgactggagcctttggctttcacctttggagaagacagtggcgaccaagacgattttctgccttagagcaagggattcaccctccgcaggctcagagcaagatagaggagctggagcaagaaccggagccggagccggagccggaaccggagcccgagcccgagcccgagccggagcagctctga

SEQ ID NO: 191 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 192 - RNA, 2894 nt, Homo sapiens
  source 1..2894: mol_type = mRNA
agtcggcgccgcccggagccgggagcgccgctccagcgaggcgcgggctgtggggccgccgcgtgcctggccccgctcgcccgtgccggccgctcgcccgccatgcctggcttcgactacaagttcctggagaagcccaagcgacggctgctgtgcccactgtgcgggaagcccatgcgcgagcctgtgcaggtttccacctgcggccaccgtttctgcgatacctgcctgcaggagttcctcagtgaaggagtcttcaagtgccctgaggaccagcttcctctggactatgccaagatctacccagacccggagctggaagtacaagtattgggcctgcctatccgctgcatccacagtgaggagggctgccgctggagtgggccactacgtcatctacagggccacctgaatacctgcagcttcaatgtcattccctgccctaatcgctgccccatgaagctgagccgccgtgatctacctgcacacttgcagcatgactgccccaagcggcgcctcaagtgcgagttttgtggctgtgacttcagtggggaggcctatgagagccatgagggtatgtgcccccaggagagtgtctactgtgagaataagtgtggtgcccgcatgatgcggcggctgctggcccagcatgccacctctgagtgccccaagcgcactcagccctgcacctactgcactaaggagttcgtctttgacaccatccagagccaccagtaccagtgcccaaggctgcctgttgcctgccccaaccaatgtggtgtgggcactgtggctcgggaggacctgccaggccatctgaaggacagctgtaacaccgccctggtgctctgcccattcaaagactccggctgcaagcacaggtgccctaagctggcaatggcacggcatgtggaggagagtgtgaagccacatctggccatgatgtgtgccctggtgagccggcaacggcaggagctgcaggagcttcggcgagagctggaggagctatcagtgggcagtgatggcgtgctcatctggaagattggcagctatggacggcggctacaggaggccaaggccaagcccaaccttgagtgcttcagcccagccttctacacacataagtatggttacaagctgcaggtgtctgcattcctcaatggcaatggcagtggtgagggcacacacctctcactgtacattcgtgtgctgcctggtgcctttgacaatctccttgagtggccctttgcccgccgtgtcaccttctccctgctggatcagagcgaccctgggctggctaaaccacagcacgtcactgagaccttccaccccgacccaaactggaagaatttccagaagccaggcacgtggcggggctccctggatgagagttctctgggctttggttatcccaagttcatctcccaccaggacattcgaaagcgaaactatgtgcgggatgatgcagtcttcatccgtgctgctgttgaactgccccggaagatcctcagctgagtgcaggtggggttcgaggggaaaggacgatggggcatgacctcagtcaggcactggctgaacttggagagggggccggacccccgtcagctgcttctgctgcctaggttctgttaccccatcctccctcccccagccaccaccctcaggtgcctccaattggtgcttcagccctggcccctgtggggaacaggtcttggggtcatgaagggctggaaacaagtgaccccagggcctgtctcccttcttgggtagggcagacatgccttggtgccggtcacactctacacggactgaggtgcctgctcaggtgctatgtcccaagagccataagggggtgggaattggggagggagaaagggtagttcaaagagtctgtcttgagatctgattttttccccctttacctagctgtgccccctctggttatttatttccttagtgccaggagggcacagcaggggagccctgatttttaataaatccggaattgtatttattaatttgcttccagcctgacttacctgggttggttaggtccctgggaggctcaaccaaactgaaggcaaagaaaggaccagtcagagaagggccgctgcctgggtctggccccaggatccagcttacctgctggctcgccctctgatggacgccgggaaaactgcatcgggctttgtgtggaagacggtccctgccactgccctctgccgatgaaatgcgggaagtgtatggcctagatgtttcataaggctggagtccctggtcagccccaccagattagtgcttctaccctaggcagggctttcttggtctaatggtaataagcaccgacactgctaagcactttacgtgcattattatttcattgaaaagaactgtcaagtgaaatactttttagcacagtaactggctttgtgggctctagagaagagttaatgaggcagtgcatgttcttggcccagagtaagtgcttagtgaatgctttctaactccgaaccccagccacatccagggactgggtgttgagcaaaaggggccttcaagatgttcaaggcacttggattttctcctgtctctcatcggcttttcttaacgggcctcagtgggtgcatgtgattatccacgtttcacctatgaaacatgaacagaggagactgacttatcagtgattcttccgcgggttcggacagggcctcgattctgttttaaactccagtagtccctagaaattgtagctccctctagttgtggcaataggtgtgggtccttgtgcttgcttttggcaagtttctgagctacacagggcctccattaccgtcactggtgaaatgcggctcacctcccagatttgttgtaaagattaaatgaactggtcagca

SEQ ID NO: 193 - protein, 470 aa, Homo sapiens
MPGFDYKFLEKPKRRLLCPLCGKPMREPVQVSTCGHRFCDTCLQEFLSEGVFKCPEDQLPLDYAKIYPDPELEVQVLGLPIRCIHSEEGCRWSGPLRHLQGHLNTCSFNVIPCPNRCPMKLSRRDLPAHLQHDCPKRRLKCEFCGCDFSGEAYESHEGMCPQESVYCENKCGARMMRRLLAQHATSECPKRTQPCTYCTKEFVFDTIQSHQYQCPRLPVACPNQCGVGTVAREDLPGHLKDSCNTALVLCPFKDSGCKHRCPKLAMARHVEESVKPHLAMMCALVSRQRQELQELRRELEELSVGSDGVLIWKIGSYGRRLQEAKAKPNLECFSPAFYTHKYGYKLQVSAFLNGNGSGEGTHLSLYIRVLPGAFDNLLEWPFARRVTFSLLDQSDPGLAKPQHVTETFHPDPNWKNFQKPGTWRGSLDESSLGFGYPKFISHQDIRKRNYVRDDAVFIRAAVELPRKILS

SEQ ID NO: 194 - DNA, 6307 bp, Homo sapiens
  source 1..6307: mol_type = genomic DNA
atgaacctctgaaaactgccggcatctgaggtttcctccaaggccctctgaagtgcagcccataatgaaggtcttggcggcaggtaaatacacccgccccgcgccggcttcgcgtccccgctgcggggcgcggcggcaacttggggcgcttggcagcgcgagccggacgcccacccgccgcagacacacgaacacttggggcgcccgcgcagccaccggaggcgctgggtggcggcccggagcgagcgcgggcacatggtcccaagcaccgcacgccccgcggcaggttggcggcgagggagggggcgccgagccttcctcctcctcctcttcttccccctctccttctcctcctcctcctcctcgctccggacactcgctggctgctcttccctctgcgctttctcccagattcactctccctctctctttttcttttctttctttctttccgctttctcctttccaaccgcggtcccgggctgctcccagggaggggcgcgggcggcgagcagcttgcaaactccggcctgggacgggagcaggtgccgcctccatctgctggtgtctggaagcgtgtggtctgcgctaggtgagatataggggtgtggcccccctcccgcagccaccccggggcctcagcactgccttgggatgctgggacgaacaggggacccaggagaagtgaacttgaggaggcccctgtccccgctgttccagcatgggggatccgggggaggtctccagcctcactcgcccgctgaccccggcccccaccatcttcaggtgcccttctgcagagccctggaaaagcgtctaagggggcctggggggagtcgggagaacaggcccccctgggggaggggcaaaccaggacatgtcgggacagctcccagctctcctgtcacctttcactttccttcctccccgcccacccgactgcctatgaccttttgccttttctctctcctggtgcaccatttcctctccctccctgagccggtgtgtgtgtgttaggagggaggggaacccctgggacaagggacagcggatgagtcgggagaaggcatggagtcaggggctgtccggagctgggggaacagagagttttgaatgatgatttggggatggagagtggggacagccaggcagaaatggggtgagcttgagtgagataggggacactagggaaggaagagatagaggatgatgggggcgggggcagaattgggggcaattggcccagggagccagaatcaagtggcaggtttgggagggagatgagggtcaaccgaggactcctccccactcccccatgccccgtggcccccatgggtgcctggcttcggagatttgggctgcaatgggccagtgagtgggaagcgctgctgaggaacctgggccaccacgggaggtgggaagagaggggtctcctttctcctggtgcctgctgggcctggggcctggtgccttccagccagagggccagggggccttaggacctttgccttcctgagggaaagggtgggatggctgggagtctctcctggaccctgcaccctttgggtggaaatggcttgtgtcttgccctcatctttacgtcatcccagaagagagcagggagaactaaggtagagaaagggagacagagaaacacacgcagtgacagagtgaagactaggccccaggaagacagctgcaggtggtgagggggaaccaggagtcctctcccatgcggccattgttggacccccatccggtgtgccatgaccccaggccaccctttcctgcctttctactcatggcttcttcctgactgtccccaggagttgtgcccctgctgttggttctgcactggaaacatggggcggggagccccctccccatcacccctgtcaacgccacctgtgccatacgccacccatgtcacaacaacctcatgaaccagatcaggagccaactggcacagctcaatggcagtgccaatgccctctttattctctatgtaagttacccctgggatactgacaggagatggcagggagggggcttgtaaatatcattaggggctgtcctgatctgggttgaggggaccttttggggctggaaggagagaatggggagagggcttgattaaaccacccccagactcccgccacttcctgcccaagcttccccagggaagcttccccagggtgcccagttagcaaggggagaactgagtgcaaaggtggggacctggcacttcttatcttgtgattgtcctgctgcagggagcgagggatggaggggaaatgggcgtgaggcaccagggagatgcggttgagaggcagtgggctgtgggtgctgggcatggaggggcgtcccggaacattgtgagtgcagggatggaagtacttgtgtgtggtgccccagctagggctagacaccgagttttcccttctgtccccttagggtggtgatgatgatgatgatgataatgatgactgcgtgcatggctcagtctttgatctttagcaagggcactcacattacaattagttttggctctcatgacaattccagatgcttacagggcaaggagttgggtcctcatgcgctagatggggaaacagacgcaagagcttgcccaaagggttggcggcagggctgggacactgacccctgactcccacgtcacctcccttctgcccctcagtacacagcccagggggagccgttccccaacaacctggacaagctatgtggccccaacgtgacggacttcccgcccttccacgccaacggcacggagaaggccaagctggtggagctgtaccgcatagtcgtgtaccttggcacctccctgggcaacatcacccgggaccagaagatcctcaaccccagtgccctcagcctccacagcaagctcaacgccaccgccgacatcctgcgaggcctccttagcaacgtgctgtgccgcctgtgcagcaagtaccacgtgggccatgtggacgtgacctacggccctgacacctcgggtaaggatgtcttccagaagaagaagctgggctgtcaactcctggggaagtataagcagatcatcgccgtgttggcccaggccttctagcaggaggtcttgaagtgtgctgtgaaccgagggatctcaggagttgggtccagatgtgggggcctgtccaagggtggctggggcccagggcatcgctaaacccaaatgggggctgctggcagaccccgagggtgcctggccagtccactccactctgggctgggctgtgatgaagctgagcagagtggaaacttccatagggagggagctagaagaaggtgccccttcctctgggagattgtggactggggagcgtgggctggacttctgcctctacttgtccctttggccccttgctcactttgtgcagtgaacaaactacacaagtcatctacaagagccctgaccacagggtgagacagcagggcccaggggagtggaccagcccccagcaaattatcaccatctgtgcctttgctgccccttaggttgggacttaggtgggccagaggggctaggatcccaaaggactccttgtcccctagaagtttgatgagtggaagatagagaggggcctctgggatggaaggctgtcttcttttgaggatgatcagagaacttgggcataggaacaatctggcagaagtttccagaaggaggtcacttggcattcaggctcttggggaggcagagaagccaccttcaggcctgggaaggaagacactgggaggaggagaggcctggaaagctttggtaggttcttcgttctcttccccgtgatcttccctgcagcctgggatggccagggtctgatggctggacctgcagcaggggtttgtggaggtgggtagggcaggggcaggttgctaagtcaggtgcagaggttctgagggacccaggctcttcctctgggtaaaggtctgtaagaaggggctggggtagctcagagtagcagctcacatctgaggccctgggaggccttgtgaggtcacacagaggtacttgagggggactggaggccgtctctggtccccagggcaagggaacagcagaacttagggtcagggtctcagggaaccctgagctccaagcgtgctgtgcgtctgacctggcatgatttctatttattatgatatcctatttatattaacttattggtgctttcagtggccaagttaattcccctttccctggtccctactcaacaaaatatgatgatggctcccgacacaagcgccagggccagggcttagcagggcctggtctggaagtcgacaatgttacaagtggaataagccttacgggtgaagctcagagaagggtcggatctgagagaatggggaggcctgagtgggagtggggggccttgctccacccccccccatcccctactgtgacttgctttagggtgtcagggtccaggctgcaggggctgggccaatttgtggagaggccgggtgcctttctgtcttgattccagggggctggttcacactgttcttgggcgccccagcattgtgttgtgaggcgcactgttcctggcagatattgtgccccctggagcagtgggcaagacagtccttgtggcccaccctgtccttgtttctgtgtccccatgctgcctctgaaatagcgccctggaacaaccctgcccctgcacccagcatgctccgacacagcagggaagctcctcctgtggcccggacacccatagacggtgcggggggcctggctgggccagaccccaggaaggtggggtagactggggggatcagctgcccattgctcccaagaggaggagagggaggctgcagatgcctgggactcagaccaggaagctgtgggccctcctgctccacccccatcccactcccacccatgtctgggctcccaggcagggaacccgatctcttcctttgtgctggggccaggcgagtggagaaacgccctccagtctgagagcaggggagggaaggaggcagcagagttggggcagctgctcagagcagtgttctggcttcttctcaaaccctgagcgggctgccggcctccaagttcctccgacaagatgatggtactaattatggtacttttcactcactttgcacctttccctgtcgctctctaagcactttacctggatggcgcgtgggcagtgtgcaggcaggtcctgaggcctggggttggggtggagggtgcggcccggagttgtccatctgtccatcccaacagcaagacgaggatgtggctgttgagatgtgggccacactcacccttgtccaggatgcagggactgccttctccttcctgcttcatccggcttagcttggggctggctgcattcccccaggatgggcttcgagaaagacaaacttgtctggaaaccagagttgctgattccacccggggggcccggctgactcgcccatcacctcatctccctgtggacttgggagctctgtgccaggcccaccttgcggccctggctctgagtcgctctcccacccagcctggacttggccccatgggacccatcctcagtgctccctccagatcccgtccggcagcttggcgtccaccctgcacagcatcactgaatcacagagcctttgcgtgaaacagctctgccaggccgggagctgggtttctcttccctttttatctgctggtgtggaccacacctgggcctggccggaggaagagagagtttaccaagagagatgtctccgggcccttatttattatttaaacatttttttaaaaagcactgctagtttacttgtctctcctccccatcgtccccatcgtcctccttgtccctgacttggggcacttccaccctgacccagccagtccagctctgccttgccggctctccagagtagacatagtgtgtggggttggagctctggcacccggggaggtagcatttccctgcagatggtacagatgttcctgccttagagtcatctctagttccccacctcaatcccggcatccagccttcagtcccgcccacgtgctagctccgtgggcccaccgtgcggccttagaggtttccctccttcctttccactgaaaagcacatggccttgggtgacaaattcctctttgatgaatgtaccctgtggggatgtttcatactgacagattatttttatttattcaatgtcatatttaaaatatttattttttataccaaatgaatacttttttttttaagaaaaaaaagagaaatgaataaagaatctactcttg

SEQ ID NO: 195 - DNA, 267 bp, Homo sapiens
  source 1..267: mol_type = other DNA
  CDS 1..267: protein_id = 196; translation = MKVLAAVHSPGGAVPQQPGQAMWPQRDGLPALPRQRHGEGQAGG AVPHSRVPWHLPGQHHPGPEDPQPQCPQPPQQAQRHRRHPARPP
atgaaggtcttggcggcagtacacagcccagggggagccgttccccaacaacctggacaagctatgtggccccaacgtgacggacttcccgcccttccacgccaacggcacggagaaggccaagctggtggagctgtaccgcatagtcgtgtaccttggcacctccctgggcaacatcacccgggaccagaagatcctcaaccccagtgccctcagcctccacagcaagctcaacgccaccgccgacatcctgcgaggcctccttag

SEQ ID NO: 196 - protein, 88 aa, Homo sapiens
MKVLAAVHSPGGAVPQQPGQAMWPQRDGLPALPRQRHGEGQAGGAVPHSRVPWHLPGQHHPGPEDPQPQCPQPPQQAQRHRRHPARPP

SEQ ID NO: 197 - DNA, 735 bp, Homo sapiens
  source 1..735: mol_type = other DNA
  CDS 1..735: protein_id = 198; translation = MRWCLLLIWAQGLRQAPLASGMMTGTIETTGNISAEKGGSIILQ CHLSSTTAQVTQVNWEQQDQLLAICNADLGWHISPSFKDRVAPGPGLGLTLQSLTVND TGEYFCIYHTYPDGTYTGRIFLEVLESSVAEHGARFQIPLLGAMAATLVVICTAVIVV VALTRKKKALRIHSVEGDLRRKSAGQEEWSPSAPSPPGSCVQAEAAPAGLCGEQRGED CAELHDYFNVLSYRSLGNCSFFTETG
atgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggttag

SEQ ID NO: 198 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 199 - DNA, 867 bp, Homo sapiens
  source 1..867: mol_type = other DNA
  CDS 1..867: protein_id = 200; translation = MQIPQAPWPVVWAVLQLGWRPGWFLDSPDRPWNPPTFSPALLVV TEGDNATFTCSFSNTSESFVLNWYRMSPSNQTDKLAAFPEDRSQPGQDCRFRVTQLPN GRDFHMSVVRARRNDSGTYLCGAISLAPKAQIKESLRAELRVTERRAEVPTAHPSPSP RPAGQFQTLVVGVVGGLLGSLVLLVWVLAVICSRAARGTIGARRTGQPLKEDPSAVPV FSVDYGELDFQWREKTPEPPVPCVPEQTEYATIVFPSGMGTSSPARRGSADGPRSAQP LRPEDGHCSWPL
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctga

SEQ ID NO: 200 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 201 - RNA, 20 nt, synthetic construct
  source 1..20: mol_type = other RNA
cctgctgctcccagttgacc

SEQ ID NO: 202 - RNA, 20 nt, synthetic construct
  source 1..20: mol_type = other RNA
tttgtaatgctgacttgggg